(19) United States
(12) Patent Application Publication
(10) Pub. No.: US 2007/0111231 A1
(43) Pub. Date: May 17, 2007

(54) Detection of mutations in a gene associated with resistance to viral infection, OAS2 and OAS3

(75) Inventors: Charles L. Magness, Seattle, WA (US); Shawn P. Iadonato, Seattle, WA (US); Christina A. Scherer, Seattle, WA (US); Phillip C. Fellin, Seattle, WA (US); Kathryn V. Steiger, Bellevue, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Illumigen Biosciences, Inc., Seattle, WA (US)

(21) Appl. No.: 11/509,335

(22) Filed: Aug. 23, 2006

Related U.S. Application Data
(60) Provisional application No. 60/710,704, filed on Aug. 23, 2005.

(51) Int. Cl. C12Q 1/68

(52) U.S. Cl. 435/6

(57) ABSTRACT

Compositions and methods are provided for detecting a mutation in a human oligoadenylate synthetase gene, particularly OAS2 or OAS3, wherein the mutation confers resistance to flavivirus infection, including infection by hepatitis C virus, and the mutation relates to other disease states including prostate cancer and diabetes, and uses of the encoded proteins and antibodies thereto.

### TECHNICAL FIELD

The present invention relates to a method for detecting a mutation in a human oligoadenylate synthetase gene, wherein a mutation confers resistance to flavivirus infection, including infection by hepatitis C virus, and a mutation relates to other disease states including prostate cancer and diabetes, and uses of the encoded proteins and antibodies thereto.

### BACKGROUND OF THE INVENTION

A number of diseases have been identified to date in which natural resistance to infection exists in the human population. Alter and Moyer, J. Acquir. Immune Defic. Syndr. Hum Retrovirol. 18 Suppl. 1:S6-10 (1998) report hepatitis C viral infection (HCV) rates as high as 90% in high-risk groups such as injecting drug users. However, the mechanism by which the remaining 10% are apparently resistant to infection has not been identified in the literature. Proteins that play a role in HCV infection include the 2-prime, 5-prime oligoadenylate synthetases. OASs are interferon-induced proteins characterized by their capacity to catalyze the synthesis of 2-prime,5-prime oligomers of adenosine (2-5As). Hovanessian et al., EMBO 6: 1273-1280 (1987) found that interferon-treated human cells contain several OASs corresponding to proteins of 40 (OAS1), 46 (OAS1), 69, and 100 kD. Marie et al., Biochem. Biophys. Res. Commun. 160:580-587 (1989) generated highly specific polyclonal antibodies against p69, the 69-kD OAS. By screening an interferon-treated human cell expression library with the anti-p69 antibodies, Marie and Hovanessian, J. Biol. Chem. 267: 9933-9939 (1992) isolated a partial OAS2 cDNA. They screened additional libraries with the partial cDNA and recovered cDNAs encoding two OAS2 isoforms. The smaller isoform is encoded by two mRNAs that differ in the length of the 3-prime untranslated region.

Northern blot analysis revealed that OAS2 is expressed as four interferon-induced mRNAs in human cells. The predicted OAS2 proteins have a common 683-amino acid sequence and different 3-prime termini. According to SDS-PAGE of in vitro transcription/translation products, two isoforms have molecular masses of 69 and 71 kD. Both isoforms exhibited OAS activity in vitro. Sequence analysis indicated that OAS2 contains two OAS1-homologous domains separated by a proline-rich putative linker region. The N- and C-terminal domains are 41% and 53% identical to OAS1, respectively.

By fluorescence in situ hybridization and by inclusion within mapped clones, Hovanian et al., Genomics 52: 267-277 (1998) determined that the OAS1, OAS2, and OAS3 genes are clustered with a 130-kb region on 12q24.2. 2-5As bind to and activate RNase L, which degrades viral and cellular RNAs, leading to inhibition of cellular protein synthesis and impairment of viral replication.

A fourth human OAS gene, referred to as OASL, differs from OAS1, OAS2 and OAS3 in that OASL lacks enzyme activity. The OASL gene encodes a two-domain protein composed of an OAS unit fused to a 164 amino acid C-terminal domain that is homologous to a tandem repeat of ubiquitin. (Eskildsen et al., Nuc. Acids Res. 31:3166-3173, 2003; Kakuta et al., J. Interferon & Cytokine Res. 22:981-993, 2002.)

Because of their role in inhibiting viral replication and viral infection, there is a need in the art for methods and compositions that suppress viral replication related to OAS2 or OAS3 activity, including a profound need for inhibitor-based therapies that suppress HCV replication.

### BRIEF SUMMARY OF THE INVENTION

The present invention relates to detecting hepatitis C resistance-related mutations which are characterized as mutations in oligoadenylate synthetase 2 or oligoadenylate synthetase 3 gene.

In one embodiment, a human genetic screening method is contemplated. The method comprises assaying a nucleic acid sample isolated from a human for the presence of an oligoadenylate synthetase 2 or oligoadenylate synthetase 3 gene mutation at nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 with reference to Genbank Sequence Accession No. NT_{—}009775.15. Consecutive bases 3,940,021- 3,981,000 of NT_{—}009775.15 are shown as FIG. 1 and correspond to OAS3. Consecutive bases 3,985,021- 4,020,000 of NT_{—}009775.15 are shown in FIG. 2 and correspond to OAS2.

In a preferred embodiment, the method comprises treating, under amplification conditions, a sample of genomic DNA from a human with a polymerase chain reaction (PCR) primer pair for amplifying a region of human genomic DNA containing nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15. The PCR treatment produces an amplification product containing the region, which is then assayed for the presence of a mutation.

In a further embodiment, the invention provides a protein encoded by a gene having at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411,or 4018625of NT_{—}009775.15,and use of the protein to prepare a diagnostic for resistance to viral infection, preferably flaviviral infection, most preferably hepatitis C infection. In specific embodiments, the diagnostic is an antibody.

In a still further embodiment, the invention provides a therapeutic compound for preventing or inhibiting infection by a virus, preferably a flavivirus, most preferably hepatitis C virus, wherein the therapeutic compound is a protein encoded by an OAS2 or OAS3 gene having at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15. In other embodiments the therapeutic compound is a polynucleotide, such as DNA or RNA, encoding the protein.

In a still further embodiment, the invention provides a therapeutic compound for preventing or inhibiting infection by a virus, preferably a flavivirus, most preferably a hepatitis C virus, wherein the therapeutic compound is a protein of the sequence: SEQUENCE:6, SEQUENCE:7, SEQUENCE:8, SEQUENCE:9, SEQUENCE: 10, SEQUENCE: 11, SEQUENCE: 12, SEQUENCE: 14, SEQUENCE: 15, and/or SEQUENCE:227.

In a still further embodiment, the invention provides a therapeutic compound for preventing or inhibiting infection by a virus, preferably a flavivirus, most preferably hepatitis C virus, wherein the therapeutic compound mimics the beneficial effects of at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15. The therapeutic compound can be a small molecule, protein, peptide, DNA or RNA molecule, or antibody.

In a still further embodiment, the invention provides a therapeutic compound for preventing or treating cancer, preferably prostate cancer, wherein the therapeutic compound is a protein encoded by an OAS gene having at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 401841 1, or 4018625 of NT_{—}009775.15. In other embodiments the therapeutic compound is a polynucleotide, such as DNA or RNA, encoding the protein.

In a still further embodiment, the invention provides a therapeutic compound for preventing or treating cancer, preferably prostate cancer, wherein the therapeutic compound is a protein of the sequence: SEQUENCE:6, SEQUENCE:7, SEQUENCE:8, SEQUENCE:9, SEQUENCE: 10, SEQUENCE: 11, SEQUENCE:12, SEQUENCE:14, SEQUENCE:15, and/or SEQUENCE:227.

In a still further embodiment, the invention provides a therapeutic compound for preventing or treating cancer, preferably prostate cancer, wherein the therapeutic compound mimics the beneficial effects of at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15. The therapeutic compound can be a small molecule, protein, peptide, DNA or RNA molecule, or antibody.

In further embodiments, the therapeutic compound is capable of inhibiting the activity of an OAS2 or OAS3 protein or at least one sub-region or sub-function of said entire protein, and such compounds are represented by antisense molecules, ribozymes, and RNAi molecules capable of specifically binding to the respective OAS2 or OAS3 polynucleotides, and by antibodies and fragments thereof capable of specifically binding to the respective OAS2 or OAS3 proteins and polypeptides.

The present invention provides, in another embodiment, inhibitors of OAS2 or OAS3. Inventive inhibitors include, but are not limited to, antisense molecules, ribozymes, RNAi, antibodies or antibody fragments, proteins or polypeptides as well as small molecules. Exemplary antisense molecules comprise at least 10, 15 or 20 consecutive nucleotides of, or that hybridize under stringent conditions to the polynucleotide of SEQUENCE: 1 (for OAS3) or SEQUENCE:2 (for OAS2). More preferred are antisense molecules that comprise at least 25 consecutive nucleotides of, or that hybridize under stringent conditions to the sequence of SEQUENCE: 1 or SEQUENCE:2.

In a still further embodiment, inhibitors of OAS proteins are envisioned that specifically bind to the region of any one of the proteins SEQUENCE:6-12 or SEQUENCE: 14-15 that are not conserved with any other forms of the same protein. Inventive inhibitors include but are not limited to antibodies, antibody fragments, small molecules, proteins, or polypeptides.

In a still further embodiment, inhibitors of OAS proteins are envisioned that are comprised of antisense or RNAi molecules that specifically bind or hybridize to the polynucleotide encoding the non-conserved regions of SEQUENCE:6-12 or SEQUENCE: 14-15.

In further embodiments, compositions are provided that comprise one or more OAS protein (either OAS2 or OAS3) inhibitors in a pharmaceutically acceptable carrier.

Additional embodiments provide methods of decreasing OAS2 or OAS3 gene expression or biological activity.

Additional embodiments provide for methods of specifically increasing or decreasing the expression of certain forms of the OAS2 or OAS3 genes having at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15.

The invention provides an antisense oligonucleotide comprising at least one modified internucleoside linkage.

The invention further provides an antisense oligonucleotide having a phosphorothioate linkage.

The invention still further provides an antisense oligonucleotide comprising at least one modified sugar moiety.

The invention also provides an antisense oligonucleotide comprising at least one modified sugar moiety which is a 2′-O-methyl sugar moiety.

The invention further provides an antisense oligonucleotide comprising at least one modified nucleobase.

The invention still further provides an antisense oligonucleotide having a modified nucleobase wherein the modified nucleobase is 5-methylcytosine.

The invention also provides an antisense compound wherein the antisense compound is a chimeric oligonucleotide.

The invention provides a method of inhibiting the expression of human OAS2 or OAS3 in human cells or tissues comprising contacting the cells or tissues in vivo with an antisense compound or a ribozyme of 8 to 35 nucleotides in length targeted to a nucleic acid molecule encoding the respective human OAS so that expression of the target OAS is inhibited.

The invention further provides a method of decreasing or increasing expression of specific forms of OAS2 or OAS3 in vivo, such forms being defined by having at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15, using antisense or RNAi compounds or ribozymes.

The invention further provides a method of modulating growth of cancer cells comprising contacting the cancer cells in vivo with an antisense compound or ribozyme of 8 to 35 nucleotides in length targeted to a nucleic acid molecule encoding human OAS2 or OAS3 so that expression of the target human OAS is inhibited.

The invention still further provides for identifying target regions of OAS2 or OAS3 polynucleotides. The invention also provides labeled probes for identifying OAS2 or OAS3 polynucleotides by in situ hybridization.

The invention provides for the use of an OAS2 or OAS3 inhibitor according to the invention to prepare a medicament for preventing or inhibiting HCV infection.

The invention further provides for directing an OAS2 or OAS3 inhibitor to specific regions of the target OAS protein or at specific functions of the protein.

The invention also provides a pharmaceutical composition for inhibiting expression of OAS2 or OAS3, comprising an antisense oligonucleotide according to the invention in a mixture with a physiologically acceptable carrier or diluent.

The invention further provides a ribozyme capable of specifically cleaving OAS2 or OAS3 RNA, and a pharmaceutical composition comprising the ribozyme.

The invention also provides small molecule inhibitors of OAS2 or OAS3 wherein the inhibitors are capable of reducing the activity of the target OAS or of reducing or preventing the expression of the target OAS mRNA.

The invention further provides for compounds that alter post-translational modifications of OAS2 or OAS3 including but not limited to myristoylation, glycosylation and phosphorylation.

The invention further provides a human genetic screening method for identifying an oligoadenylate synthetase gene mutation comprising: (a) treating, under amplification conditions, a sample of genomic DNA from a human with a polymerase chain reaction (PCR) primer pair for amplifying a region of human genomic DNA containing nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15, said treating producing an amplification product containing said region; and (b) detecting in the amplification product of step (a) the presence of an nucleotide mutation at nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15, thereby identifying said mutation.

In certain embodiments of this method, the region comprises a nucleotide sequence represented by a sequence selected from the group consisting of SEQUENCE: 153-226. In other embodiments, the region consists essentially of a nucleotide sequence selected from the group consisting of SEQUENCE: 153-226. Also provided is a method of detecting, wherein the detecting comprises treating, under hybridization conditions, the amplification product of step (a) above with an oligonucleotide probe specific for the point mutation, and detecting the formation of a hybridization product. In certain embodiments of the method, the oligonucleotide probe comprises a nucleotide sequence selected from the group consisting of SEQUENCE:80-152 and SEQUENCE:230-239.

The invention also relates to a method for detecting in a human a hepatitis C infection sensitivity or resistance allele containing a mutation at nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15, which method comprises: (a) forming a polymerase chain reaction (PCR) admixture by combining, in a PCR buffer, a sample of genomic DNA from said human and an oligoadenylate synthetase gene-specific PCR primer pair selected from of the group consisting of: SEQUENCE: 16-79 and SEQUENCE:228-229, the amplicon of which will span the nucleotide position of the desired mutation; (b) subjecting the PCR admixture to a plurality of PCR thermocycles to produce an oligoadenylate synthetase gene amplification product; and (c) treating, under hybridization conditions products produced in step (b), with a probe corresponding to the desired mutation selected from the group consisting of SEQUENCE:80-152 and SEQUENCE:230-239, thereby detecting said mutation.

Also provided is an isolated OAS2 or OAS3 inhibitor selected from the group consisting of an antisense oligonucleotide, a ribozyme, a small inhibitory RNA (RNAi), a protein, a polypeptide, an antibody, and a small molecule. The isolated inhibitor may be an antisense molecule or the complement thereof comprising at least 15 consecutive nucleic acids of the sequence of SEQUENCE: 1 (for OAS3) or SEQUENCE:2 (for OAS2). In other embodiments, the isolated OAS inhibitor (antisense molecule or the complement thereof) hybridizes under high stringency conditions to either SEQUENCE: 1 or SEQUENCE:2.

The isolated OAS2 or OAS3 inhibitor may be selected from the group consisting of an antibody and an antibody fragment. Also provided is a composition comprising a therapeutically effective amount of at least one OAS inhibitor in a pharmaceutically acceptable carrier.

The invention also relates to a method of inhibiting the expression of OAS2 or OAS3 in a mammalian cell, comprising administering to the cell an OAS2 or OAS3 inhibitor (as desired) selected from the group consisting of an antisense oligonucleotide, a ribozyme, a protein, an RNAi, a polypeptide, an antibody, and a small molecule.

The invention further relates to a method of inhibiting OAS2 or OAS3 gene expression in a subject, or gene expression of a specific OAS 2 or 3 allele in a subject, comprising administering to the subject, in a pharmaceutically effective vehicle, an amount of an antisense oligonucleotide which is effective to specifically hybridize to all or part of a selected target nucleic acid sequence derived from said OAS gene.

The invention still further relates to a method of preventing infection by a flavivirus in a human subject susceptible to the infection, comprising administering to the human subject an OAS2 or OAS3 inhibitor selected from group consisting of an antisense oligonucleotide, a ribozyme, an RNAi, a protein, a polypeptide, an antibody, and a small molecule, wherein said OAS inhibitor prevents infection by said flavivirus.

The invention still further relates to a method of preventing or curing infection by a flavivirus or other virus in a human subject susceptible to the infection, comprising administering to the human subject an OAS2 or OAS3 inhibitor selected from group consisting of an antisense oligonucleotide, a ribozyme, an RNAi, a protein, a polypeptide, an antibody, and a small molecule, wherein said OAS2 or OAS3 inhibitor prevents infection by said flavivirus or other virus and wherein said OAS2 or OAS3 inhibitor is directed at one or more specific forms of the protein encoded by a gene with a mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15.

The invention still further relates to a method of preventing or curing infection by a flavivirus or any other virus in a human subject susceptible to the infection by administering one of the polypeptides of the sequence: SEQUENCE:6, SEQUENCE:7, SEQUENCE:8, SEQUENCE:9, SEQUENCE:10, SEQUENCE: 11, SEQUENCE: 12, SEQUENCE: 14, SEQUENCE: 15 and/or SEQUENCE:227.

The invention embodies also treatments for infection with the human immunodeficiency virus (HIV).

The invention still further relates to a method of preventing or treating insulin dependent diabetes mellitus (IDDM) in a human subject, comprising administering to the human subject an OAS2 or OAS3 inhibitor selected from group consisting of an antisense oligonucleotide, a ribozyme, an RNAi, a protein, a polypeptide, an antibody, and a small molecule, wherein said OAS2 or OAS3 inhibitor prevents IDDM.

The invention still further relates to a method of preventing or treating IDDM in a human subject, comprising administering to the human subject an OAS2 or OAS3 inhibitor selected from group consisting of an anti sense oligonucleotide, a ribozyme, an RNAi, a protein, a polypeptide, an antibody, and a small molecule, wherein said OAS2 or OAS3 inhibitor prevents IDDM and wherein said OAS2 or OAS3 inhibitor is directed at one or more specific forms of an OAS protein encoded by a gene with a mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of NT_{—}009775.15.

The invention still further relates to a method of treating cancer, such as prostate cancer by increasing expression of the OAS2 or OAS3 gene or by therapeutic administration of polypeptides disclosed herein.

Also provided is a method for inhibiting expression of an OAS2 or OAS3 target gene in a cell in vitro comprising introduction of a ribonucleic acid (RNA) into the cell in an amount sufficient to inhibit expression of the target gene, wherein the RNA is a double-stranded molecule with a first strand consisting essentially of a ribonucleotide sequence which corresponds to a nucleotide sequence of the target gene and a second strand consisting essentially of a ribonucleotide sequence which is complementary to the nucleotide sequence of the target gene, wherein the first and the second ribonucleotide strands are separate complementary strands that hybridize to each other to form said double-stranded molecule, and the double-stranded molecule inhibits expression of the target gene.

In certain embodiments of the method, the first ribonucleotide sequence comprises at least 20 bases which correspond to the OAS target gene and the second ribonucleotide sequence comprises at least 20 bases which are complementary to the nucleotide sequence of the OAS target gene. In still further embodiments, the target gene expression is inhibited by at least 10%.

In still further embodiments of the method, the double-stranded ribonucleic acid structure is at least 20 bases in length and each of the ribonucleic acid strands is able to specifically hybridize to a deoxyribonucleic acid strand of the OAS target gene over the at least 20 bases.

The invention provides a polypeptide or protein capable of restoring function of OAS2 or OAS3 that may be diminished or lost due to gene mutation. In some embodiments the polypeptide or protein has the amino acid sequence of reference OAS3 (encoded by a gene comprising SEQUENCE: 1) or OAS2 (encoded by a gene comprising SEQUENCE:2). In other embodiments, wherein a mutation in the OAS2 or OAS3 gene confers increased activity, stability, and/or half life on the encoded protein, or other change making the encoded protein more suitable for anti-viral activity, the protein or polypeptide encoded by the mutated OAS gene is preferred.

Any of the foregoing proteins and polypeptides can be provided as a component of a therapeutic composition.

Also provided is the use of any of the proteins consisting of SEQUENCE:6, SEQUENCE:7, SEQUENCE: 8, SEQUENCE:9, SEQUENCE: 10, SEQUENCE: 11, SEQUENCE: 12, SEQUENCE: 14, SEQUENCE: 15 and/or SEQUENCE:227 as a component of a therapeutic composition.

In a further embodiment, a nucleic acid encoding any of the OAS2 or OAS3 polypeptides of the present invention can be administered in the form of gene therapy.

### BRIEF DESCRIPTION OF THE FIGURES

FIG. 1 shows SEQUENCE: 1, a polynucleotide sequence consisting of the consecutive nucleotide bases at positions 3,940,021-3,981,000 of NCBI Accession No. NT_{—}009775.15, OAS3.

FIG. 2 shows SEQUENCE:2, a polynucleotide sequence consisting of the consecutive nucleotide bases at positions 3,985,021-4,020,000 of NCBI Accession No. NT_{—}009775.15, OAS2.

FIG. 3 shows SEQUENCE:3-5 and SEQUENCE: 13, polynucleotides of the present invention, and SEQUENCE:6-12, SEQUENCE: 14-15, and SEQUENCE:227, polypeptides of the present invention.

FIG. 4 is a Table showing the locations of the mutations of the present invention in the OAS3 gene, the allelic variants (base substitutions), coordinates of the mutation on the genomic sequence, and NCBI dbSNP ID if any.

FIG. 5 is a Table showing the locations of the mutations of the present invention in the OAS2 gene, the allelic variants (base substitutions), coordinates of the mutation on the genomic sequence, and NCBI dbSNP ID if any.

FIG. 6 is a Table showing the locations of the amino acid mutations of the present invention in the primate OAS3 proteins.

FIG. 7 is a Table showing the locations of the amino acid mutations of the present invention in the primate OAS2 proteins.

FIG. 8 shows a polypeptide sequence alignment of members of the primate OAS2 and OAS3 gene families.

FIG. 9 shows the ability of an oligoadenylate synthetase to enter a cell by protein transduction and become resident in subcellular compartments. The protein remains enzymatically active in these subcellular compartments for up to 72 hours.

FIG. 10 shows the antiviral activity affected when an oligoadenylate synthetase polypeptide is used to contact a cell infected with a virus.

### DETAILED DESCRIPTION OF THE INVENTION

Introduction and Definitions

This invention relates to novel mutations in an oligoadenylate synthetase gene, use of these mutations for diagnosis of susceptibility or resistance to viral infection, to proteins encoded by a gene having a mutation according to the invention, and to prevention or inhibition of viral infection using the proteins, antibodies, and related nucleic acids. These mutations correlate with resistance (as further defined below) of the carrier to infection with flavivirus, particularly hepatitis C virus. The invention also relates to therapeutic treatments for cancer and diabetes using proteins, antibodies, and related nucleic acids of the present invention.

Much of current medical research is focused on identifying mutations and defects that cause or contribute to disease. Such research is designed to lead to compounds and methods of treatment aimed at the disease state. Less attention has been paid to studying the genetic influences that allow people to remain healthy despite exposure to infectious agents and other risk factors. The present invention represents a successful application of a process developed by the inventors by which specific populations of human subjects are ascertained and analyzed in order to discover genetic variations or mutations that confer resistance to disease. The identification of a sub-population segment that has a natural resistance to a particular disease or biological condition further enables the identification of genes and proteins that are suitable targets for pharmaceutical intervention, diagnostic evaluation, or prevention, such as prophylactic vaccination.

The sub-population segment identified herein is comprised of individuals who, despite repeated exposure to hepatitis C virus (HCV) have nonetheless remained sero-negative, while cohorts have become infected (sero-positive). The populations studied included hemophiliac patients subjected to repeated blood transfusions, and intravenous drug users who become exposed through shared needles and other risk factors.

HCV infection involves a complex set of proteins and immune system components that work together to achieve a level of infection that, while it causes disease, can develop into low steady state of virus in infected cells, apparently allowing HCV to escape from the host immuno-surveillance system, while enabling persistent viral infection. (Dansako et al., Virus Research 97:17-30, 2003.) The present invention focuses on one component of this system, an interferon-inducible 2′-5′-oligoadenylate synthetase gene, specifically OAS2 or OAS3. OAS2 and OAS3, each independently play a major role in the antiviral activity of host cells in the human, by activating ribonuclease L (RNase L) to cleave viral RNA. The OAS proteins also activate other components of the innate and adaptive immune responses independently of activation of RNAseL. HCV RNA activates the 2′-5′-OAS/RNase L pathway. As pointed out by Dansako et al., it may appear contradictory for HCV RNA to activate a pathway that leads to cleavage of the viral RNA. However, such activity may serve to retain a balance between the host immune defense and a level of infection that would kill the host.

In view of this complex role of these OAS genes, it is of significant interest that the present invention has identified a strong correlation between mutations in each of the OAS2 and OAS3 genes, and resistance to HCV infection in carriers of these mutations. The presence of such individuals now permits the elucidation of how OAS2 and OAS3 contribute to resistance to HCV infection despite repeated exposure to infectious levels of the virus. This information will then lead to development of methods and compositions for replicating the resistance mechanism by developing therapeutic treatments for individuals lacking natural resistance.

The present invention therefore provides that, regardless of the mechanism, the mutations identified herein are useful for identifying individuals who are resistant to HCV infection. The resistance may come about through a loss of function of either the OAS2 or OAS3 protein, in which case it is predicted that HCV viral levels would be high enough to prevent the virus from escaping from the host immuno-surveillance system, hence facilitating destruction of the virus. The resistance may also come about through gain of function in that either the OAS2 or OAS3 protein level is enhanced, the half life of the respective protein is increased, and/or the protein structure is affected in a way that enhances its ability to activate ribonuclease L to cleave viral RNA. The resistance may also come about through modifications to either the OAS2 or OAS3 protein that prevent inhibition of normal OAS2 or OAS3 protein function by HCV viral proteins or nucleotides. The resistance may also come about through modifications to either the OAS2 or OAS3 protein that prevent interaction of the protein with HCV viral proteins or nucleotides that are necessary for the normal HCV viral lifespan. The invention is not limited to one mechanism. Furthermore, although several different point mutations are disclosed herein, this is not intended to be indicative that each mutation has the same effect on OAS2 or OAS3 protein structure or function.

The present invention also provides that, regardless of mechanism, the polynucleotides, polypeptides, and other envisaged therapeutic applications of the present invention are useful for treating mammalian diseases as discussed in the following. Utility may be achieved by therapeutic treatments of the present invention increasing oligoadenylate synthetase enzymatic activity thereby mediating increased cleavage of viral RNA. Utility may also be achieved by therapeutic treatments of the present invention remaining enzymatically active even in the presence of HCV viral proteins or nucleotides. Utility may also be achieved by therapeutics of the present invention interacting with HCV viral proteins or nucleotides in such a manner as to interfere with or modify the HCV viral cycle. The invention is not limited to one mechanism. Furthermore, as several different therapeutic treatments are disclosed herein, this is not intended to be construed that each treatment has the same mechanistic effect or even the same applicability across different disease modalities.

OAS2 and OAS3 play a role in infection by other viruses of the flavivirus family, of which HCV is a member. The flavivirus family also includes viruses that cause yellow fever, dengue fever, St. Louis encephalitis, Japanese encephalitis, and other viral diseases disclosed herein. The host defense to these viruses includes virus-inducible interferon. The interferon induces 2′-5′-oligoadenylate synthetases, which as discussed above, are involved in the activation of RNaseL. RNaseL in turn cleaves viral RNA. Other viral infections may by amenable to prevention and/or inhibition by the methods disclosed herein, including RSV.

Several novel forms of the OAS 1, OAS2, and OAS3 genes have been cloned by us, and we have developed polypeptide pharmaceutical compositions derived from these and other novel oligoadenylate synthetase forms. We have demonstrated that these polypeptide pharmaceutical compositions are antiviral in vitro. We have further demonstrated that these pharmaceutical compositions promote cellular growth in certain cell lines. We have further demonstrated that these pharmaceutical compositions have a mitogenic effect. We have further demonstrated that these pharmaceutical compositions have the ability to enter a cell and remain enzymatically active in intracellular stores for several days or more. We have further demonstrated that the cell-penetrating property of the polypeptide pharmaceutical compositions can be enhanced through the addition of basic amino acid residues including arginine, lysine, and histidine. We have further demonstrated that these pharmaceutical compositions have broad antiviral activity. We have further demonstrated that these polypeptide pharmaceutical compositions can be derivatized with polyethylene glycol and retain their enzymatic activity. We show that the stability of the pharmaceutical compositions is dependent on the presence of reducing agents, and we propose several modifications to provide more oxidation resistant forms of the protein. We demonstrate that bulk quantities of the pharmaceutical compositions can be manufactured using recombinant DNA technologies by heterologous expression in Escherichia coli. We further demonstrate that these manufactured polypeptide pharmaceutical compositions can be administered to mammals and produce no observable toxic effects. We further demonstrate that these manufactured pharmaceutical compositions have good biodistribution and pharmacokinetic properties when administered to a mammal by injection.

In reference to the detailed description and preferred embodiment, the following definitions are used:

A: adenine; C: cytosine; G: guanine; T: thymine (in DNA); and U: uracil (in RNA)

Allele: A variant of DNA sequence of a specific gene. In diploid cells a maximum of two alleles will be present, each in the same relative position or locus on homologous chromosomes of the chromosome set. When alleles at any one locus are identical the individual is said to be homozygous for that locus, and when they differ the individual is said to be heterozygous for that locus. Since different alleles of any one gene may vary by only a single base, the possible number of alleles for any one gene is very large. When alleles differ, one is often dominant to the other, which is said to be recessive. Dominance is a property of the phenotype and does not imply inactivation of the recessive allele by the dominant. In numerous examples the normally functioning (wild-type) allele is dominant to all mutant alleles of more or less defective function. In such cases the general explanation is that one functional allele out of two is sufficient to produce enough active gene product to support normal development of the organism (i.e., there is normally a two-fold safety margin in quantity of gene product).

Haplotype: The set of alleles across one or more genes or DNA segments carried by one particular homologous chromosome of the chromosome set. The haplotype is often represented by a reduced sequence containing only the particular allelic forms found at a plurality of polymorphic sites spanning the segment or gene(s) of interest.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Base Pair (bp): A partnership of adenine (A) with thymine (T), or of cytosine (C) with guanine (G) in a double stranded DNA molecule. In RNA, uracil (U) is substituted for thymine. When referring to RNA herein, the symbol T may be used interchangeably with U to represent uracil at a particular position in the RNA molecule.

Nucleic Acid: A polymer of nucleotides, either single or double stranded.

Polynucleotide: A polymer of single or double stranded nucleotides. As used herein “polynucleotide” and its grammatical equivalents will include the full range of nucleic acids. A polynucleotide will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention include primers, probes, RNA/DNA segments, oligonucleotides or “oligos” (relatively short polynucleotides), genes, vectors, plasmids, and the like.

Gene: A nucleic acid whose nucleotide sequence codes for an RNA or polypeptide. A gene can be either RNA or DNA.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Complementary Bases: Nucleotides that normally pair up when DNA or RNA adopts a double stranded configuration.

Complementary Nucleotide Sequence: A sequence of nucleotides in a single-stranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Hybridization: The pairing of substantially complementary nucleotide sequences (strands of nucleic acid) to form a duplex or heteroduplex by the establishment of hydrogen bonds between complementary base pairs. It is a specific, i.e. non-random, interaction between two complementary polynucleotides that can be competitively inhibited.

Nucleotide Analog: A purine or pyrimidine nucleotide that differs structurally from A, T, G, C, or U, but is sufficiently similar to substitute for the normal nucleotide in a nucleic acid molecule.

DNA Homolog: A nucleic acid having a preselected conserved nucleotide sequence and a sequence coding for a receptor capable of binding a preselected ligand.

Upstream: In the direction opposite to the direction of DNA transcription, and therefore going from 5′ to 3′ on the non-coding strand, or 3′ to 5′ on the mRNA.

Downstream: Further along a DNA sequence in the direction of sequence transcription or read out, that is traveling in a 3′- to 5′-direction along the non-coding strand of the DNA or 5′- to 3′-direction along the RNA transcript.

Stop Codon: Any of three codons that do not code for an amino acid, but instead cause termination of protein synthesis (i.e. translation). They are UAG, UAA and UGA and are also referred to as a nonsense or termination codon.

Reading Frame: Particular sequence of contiguous nucleotide triplets (codons) employed in translation. The reading frame depends on the location of the translation initiation codon.

Intron: Also referred to as an intervening sequence, a noncoding sequence of DNA that is initially copied into RNA but is cut out of the final RNA transcript.

Resistance: As used herein with regard to viral infection, resistance specifically includes all degrees of enhanced resistance or susceptibility to viral infection as observed in the comparison between two or more groups of individuals.

Protein or polypeptide: The term “protein” or “polypeptide” refers to a polymer of amino acids and does not refer to a specific length of the product. Peptides, oligopeptides, polypeptides, proteins, and polyproteins, as well as fragments of these, are included within this definition. The term may include post expression modifications of the protein, for example, glycosylations, acetylations, phosphorylations and the like. Included within the definition are, for example, proteins containing one or more analogs of an amino acid (including, for example, unnatural amino acids, etc.), proteins with substituted linkages, as well as other modifications known in the art, both naturally occurring and non-naturally occurring.

A “variant” is a polypeptide comprising a sequence which differs in one or more amino acid position(s) from that of a parent polypeptide sequence.

The term “parent polypeptide” is intended to indicate the polypeptide sequence to be modified in accordance with the present invention.

A “fragment” or “subsequence” is any portion of an entire sequence, up to but not including the entire sequence. Thus, a fragment or subsequence refers to a sequence of amino acids or nucleic acids that comprises a part of a longer sequence of amino acids (e.g., polypeptide) or nucleic acids (e.g., polynucleotide).

A polypeptide, nucleic acid, or other component is “isolated” when it is partially or completely separated from components with which it is normally associated (other peptides, polypeptides, proteins (including complexes, e.g., polymerases and ribosomes which may accompany a native sequence), nucleic acids, cells, synthetic reagents, cellular contaminants, cellular components, etc.), e.g., such as from other components with which it is normally associated in the cell from which it was originally derived. A polypeptide, nucleic acid, or other component is isolated when it is partially or completely recovered or separated from other components of its natural environment such that it is the predominant species present in a composition, mixture, or collection of components (i.e., on a molar basis it is more abundant than any other individual species in the composition). In some instances, the preparation consists of more than about 60%, 70% or 75%, typically more than about 80%, or preferably more than about 90% of the isolated species.

A “substantially pure” nucleic acid (e.g., RNA or DNA), polypeptide, protein, or composition also means where the object species (e.g., nucleic acid or polypeptide) comprises at least about 50, 60, or 70 percent by weight (on a molar basis) of all macromolecular species present. A substantially pure composition can also comprise at least about 80, 90, or 95 percent by weight of all macromolecular species present in the composition. An isolated object species can also be purified to essential homogeneity (contaminant species cannot be detected in the composition by conventional detection methods) wherein the composition consists essentially of derivatives of a single macromolecular species. The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or antisense.

A “recombinant polynucleotide” or a “recombinant polypeptide” is a non-naturally occurring polynucleotide or polypeptide which may include nucleic acid or amino acid sequences, respectively, from more than one source nucleic acid or polypeptide, which source nucleic acid or polypeptide can be a naturally occurring nucleic acid or polypeptide, or can itself have been subjected to mutagenesis or other type of modification. A nucleic acid or polypeptide may be deemed “recombinant” when it is synthetic or artificial or engineered, or derived from a synthetic or artificial or engineered polypeptide or nucleic acid. A recombinant nucleic acid (e.g., DNA or RNA) can be made by the combination (e.g., artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. A “recombinant polypeptide” often refers to a polypeptide that results from a cloned or recombinant nucleic acid. The source polynucleotides or polypeptides from which the different nucleic acid or amino acid sequences are derived are sometimes homologous (i.e., have, or encode a polypeptide that encodes, the same or a similar structure and/or function), and are often from different isolates, serotypes, strains, species, of organism or from different disease states, for example.

The term “recombinant” when used with reference, e.g., to a cell, polynucleotide, vector, protein, or polypeptide typically indicates that the cell, polynucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences that are not found in the native (non-recombinant) form of the cell or express native nucleic acid sequences that would otherwise be abnormally expressed, under-expressed, or not expressed at all. The term “recombinant” when used with reference to a cell indicates that the cell replicates a heterologous nucleic acid, or expresses a polypeptide encoded by a heterologous nucleic acid. Recombinant cells can contain coding sequences that are not found within the native (non-recombinant) form of the cell. Recombinant cells can also contain coding sequences found in the native form of the cell wherein the coding sequences are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, recombination, and related techniques.

The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated.

An “immunogen” refers to a substance capable of provoking an immune response, and includes, e.g., antigens, autoantigens that play a role in induction of autoimmune diseases, and tumor-associated antigens expressed on cancer cells. An immune response generally refers to the development of a cellular or antibody-mediated response to an agent, such as an antigen or fragment thereof or nucleic acid encoding such agent. In some instances, such a response comprises a production of at least one or a combination of CTLs, B cells, or various classes of T cells that are directed specifically to antigen-presenting cells expressing the antigen of interest.

An “antigen” refers to a substance that is capable of eliciting the formation of antibodies in a host or generating a specific population of lymphocytes reactive with that substance. Antigens are typically macromolecules (e.g., proteins and polysaccharides) that are foreign to the host.

An “adjuvant” refers to a substance that enhances an antigen's immune-stimulating properties or the pharmacological effect(s) of a drug. An adjuvant may non-specifically enhance the immune response to an antigen. “Freund's Complete Adjuvant,” for example, is an emulsion of oil and water containing an immunogen, an emulsifying agent and mycobacteria. Another example, “Freund's incomplete adjuvant,” is the same, but without mycobacteria.

A “vector” is a component or composition for facilitating cell transduction or transfection by a selected nucleic acid, or expression of the nucleic acid in the cell. Vectors include, e.g., plasmids, cosmids, viruses, YACs, bacteria, poly-lysine, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter. The nucleic acid to be transcribed is typically under the direction or control of the promoter.

The term “subject” as used herein includes, but is not limited to, an organism; a mammal, including, e.g., a human, non-human primate (e.g., baboon, orangutan, monkey), mouse, pig, cow, goat, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition suitable for pharmaceutical use in a subject, including an animal or human. A pharmaceutical composition generally comprises an effective amount of an active agent (“active pharmaceutical ingredient” or API) and a carrier, including, e.g., a pharmaceutically acceptable carrier.

The term “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

“Dialysis” or “Ultrafiltration/Diafiltration” refers to standard methods for exchanging one buffer, e.g. the solubilization solution and/or the purification buffers, into a different buffer, e.g. the formulation solution to stabilize the solubilized protein and/or the final purified product.

“Inclusion” (or refractile) bodies shall mean dense, insoluble (i.e., not easily dissolved) protein aggregates (i.e., clumps) that are produced within the cells of certain microorganisms, generally by high expression levels of heterologous genes during fermentation. The term refractile bodies is used in some instances because their greater density (than the rest of the microorganism's body mass) causes light to be refracted (bent) when it is passed through them. This bending of light causes the appearance of very bright and dark areas around the retractile body and makes them visible under a microscope.

The term “refractile bodies” and “inclusion bodies” encompass insoluble cytoplasmic aggregates produced within a recombinant host organism wherein the aggregates contain, at least in part, a heterologous protein to be recovered.

“Disrupting” or “lysing” the host organism (cell) shall mean the process of breaking the bacterial cells to isolate the inclusion bodies or the recombinant polypeptides or proteins.

“Lysate” shall mean the residue from disruption of the host organism in the present method. A lysate arises, typically, from cytolysis, the dissolution of cells, particularly by destruction of their surface membranes. In some embodiments lysozymes lyse certain kinds of bacteria, by dissolving the polysaccharide components of the bacteria's cell wall. When that cell wall is weakened, the bacteria cell then bursts because osmotic pressure (inside that bacteria cell) is greater than the weakened cell wall can contain. In a particular embodiment cells are lysed by digestion with Lysozyme or disrupted by three cycles of cell dispersion with a Teflon homogenizer followed by centrifugation. In another embodiment, cells are disrupted by several passes in a pressurized homogenizer (e.g., Gaulin) or a microfluidizer. Sonication is also used.

“Chaotropic agent” refers to a compound that, in a suitable concentration in aqueous solution, is capable of changing the spatial configuration or conformation of proteins through alterations at the surface, rendering a protein to be isolated, soluble in the aqueous medium but without biological activity.

A “reducing agent” is the compound in an oxidation-reduction reaction that serves as the electron donor. A reducing agent is also a compound that maintains the sulfhydryl groups of proteins in the reduced state and reduces disulfide intra- or intermolecular bonds. Exemplary reducing agents include: 2-Mercaptoethylamine HCl, 2-mercaptoethanol, dithiothreitol, Ellman's reagent, Tris-(2-carboxyethyl)-phosphine hydrochloride, cysteine, and the like.

A “chelating agent” is a compound capable of forming a coordinate bond with one or more metal ions.

“Stabilizing compounds” shall mean compounds such as sugars, surfactants such as polysorbate-10, polysorbate-80 and PEG, polyols, chelating agents, amino acids and polymers, which in combination will increase the solubility and biological activity of a protein. The structure of a protein is strongly influenced by pH. Thus, in the presence of solutions containing low quantities of OH.sup.− or H.sup.+ ions and stabilizers, ionization of the side chains occurs and solubilization takes place. Unfolding of tangled protein in inclusion bodies, at low concentration of the ions in the non-buffered aqueous solution, releases monomeric protein. Aqueous solutions containing osmolytic stabilizers such as sugars and polyols (polyhydric alcohols) provide protein stability, and thus the maintenance of solubility and biological activity of proteins. Such stability of protein structure by sugars is due to the preferential interaction of proteins with solvent components. The major effects of stabilizing compounds are on the viscosity and surface tension of the water. Many of these compounds include sugars, polyols, polysaccharides, neutral polymers, amino acids (glycine and alanine) and derivatives, and large dipolar molecules (i.e., trimethylamine N-oxide). Sugars such as mannitol and lactose maintain protein stability. Proteins are preferably hydrated in the presence of sugars. There is a positive change in the chemical potential of the protein induced by the addition of lactose and hence the stabilization of a protein. Polyols such as mannitol and glycerol are used also as protein stabilizers. Mannitol induces structure in the water molecules and stabilizes proteins by competing with water. This is believed due to the stronger hydrophobic interaction between pairs of hydrophobic groups in the solutions of mannitol than in pure water. Without being bound by any specific theory, it is believed that Mannitol (and other polyols such as glycerol, sorbitol, arabitol and Xylitol) displace water allowing stabilization of hydrophobic interactions which are the major factor stabilizing the three-dimensional structure of proteins. Glycerol stabilizes proteins in solution, likely due to its ability to enter into and strengthen the water lattice structure. It is believed to prevent formation of precipitates by assisting preferential hydration and leads to the net stabilization of the native structure of proteins. Sorbitol likely competes for the hydration by water of the protein stabilizing the protein from denaturation, and amino acids such as L-arginine, taurine, sarcosine, glycine and serine, likely increase the surface tension of water, stabilizing proteins and suppressing aggregation.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoters include, for example, but are not limited to, IPTG-inducible promoters, bacteriophage T7 promoters and bacteriophage .lamda.p.sub.L. See Sambrook et al., Molecular Cloning: A Laboratory Manual, 3rd ed., Cold Spring Harbor Laboratory Press, Cold Spring Harbor, N.Y., 2001. A typical promoter will have three components, consisting of consensus sequences at −35 and −10 with a sequence of between 16 and 19 nucleotides between them (Lisset, S. and Margalit, H., Nucleic Acids Res. 21: 1512, 1993). Promoters of this sort include the lac, trp, trp-lac (tac) and trp-lac(trc) promoters. If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a eukaryotic regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner. Bacterial promoters have regulatory elements that bind and modulate the activity of the core promoter, such as operator sequences that bind activator or repressor molecules.

A “cloning vector” is a nucleic acid molecule, such as a plasmid, cosmid, or bacteriophage, which has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide resistance to antibiotic.

An “expression vector” is a nucleic acid molecule encoding a gene that is expressed in a host cell. Typically, an expression vector comprises a transcriptional promoter, a gene, an origin of replication, a selectable marker, and a transcriptional terminator. Gene expression is usually placed under the control of a promoter, and such a gene is said to be “operably linked to” the promoter. Similarly, a regulatory element and a core promoter are operably linked if the regulatory element modulates the activity of the core promoter. An expression vector may also be known as an expression construct.

The term “expression” refers to the biosynthesis of a gene product. For example, in the case of a structural gene, expression involves transcription of the structural gene into mRNA and the translation of mRNA into one or more polypeptides.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The terms “amino-terminal” or “N-terminal” and “carboxyl-terminal” or “C-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

A “fusion protein” is a hybrid protein expressed by a nucleic acid molecule comprising nucleotide sequences of at least two genes.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

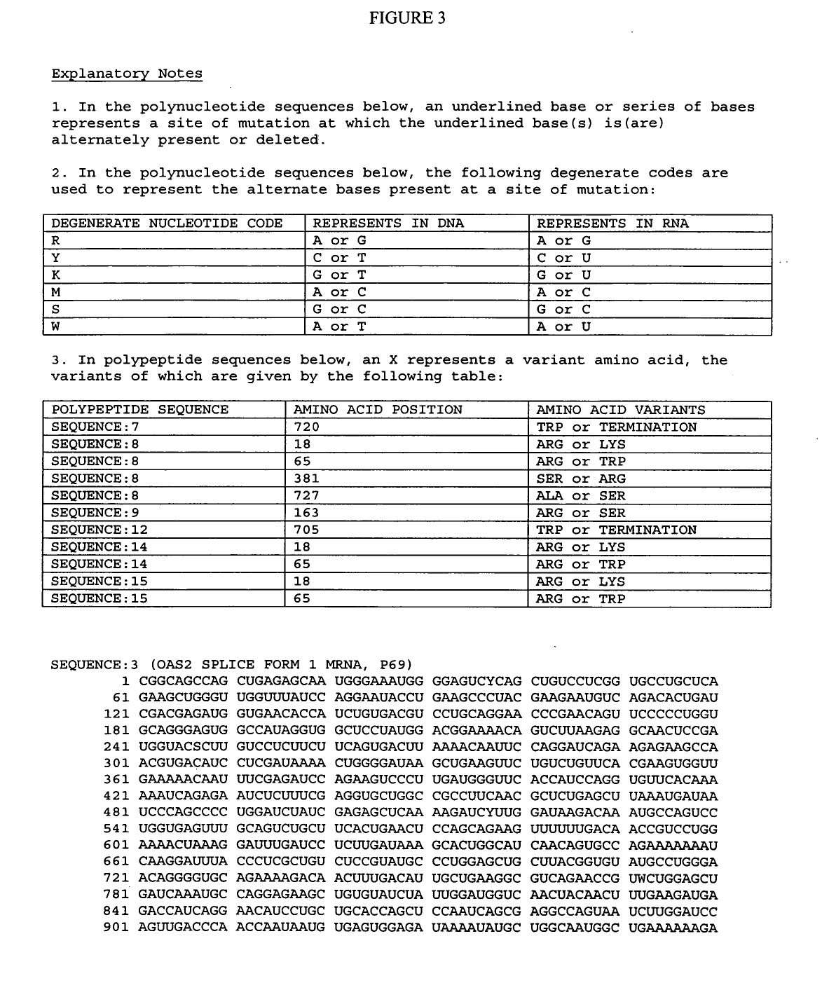

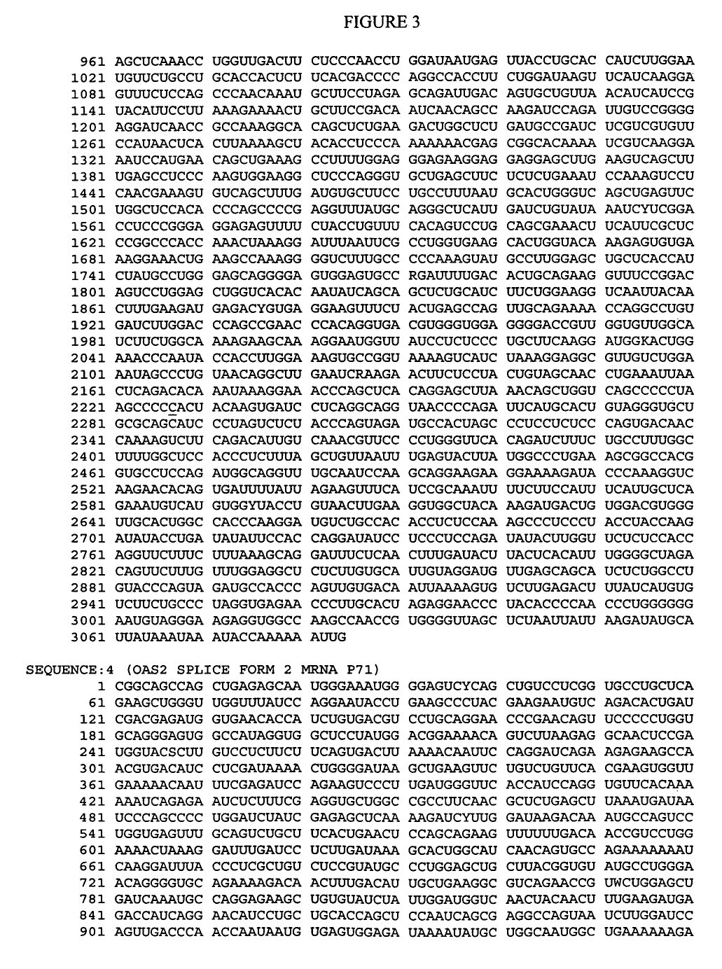

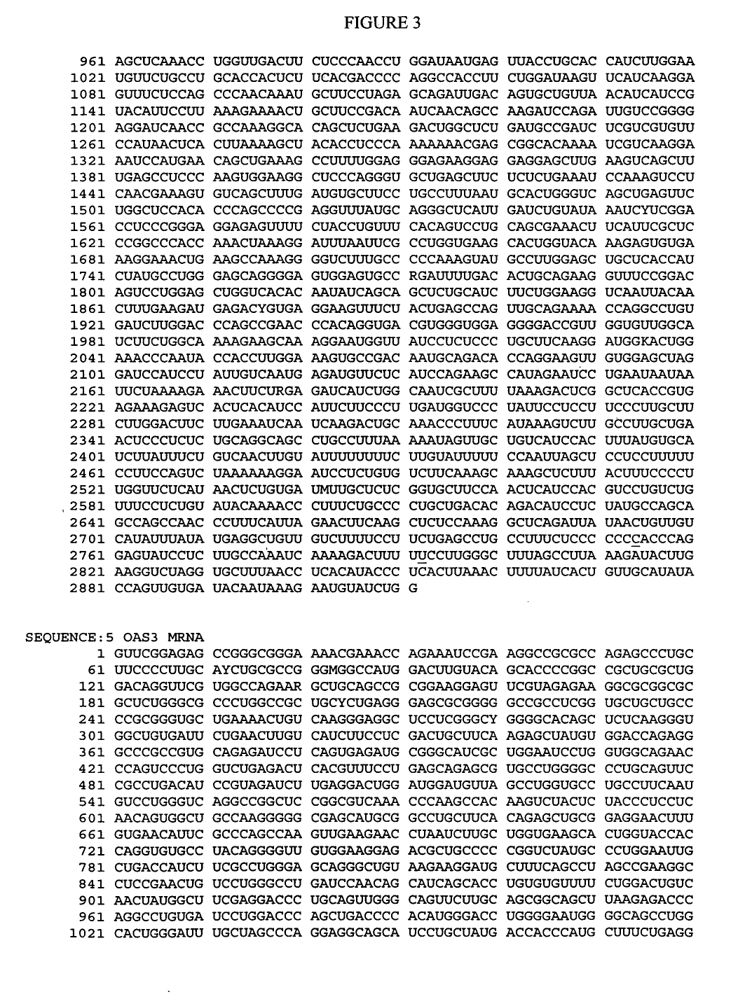

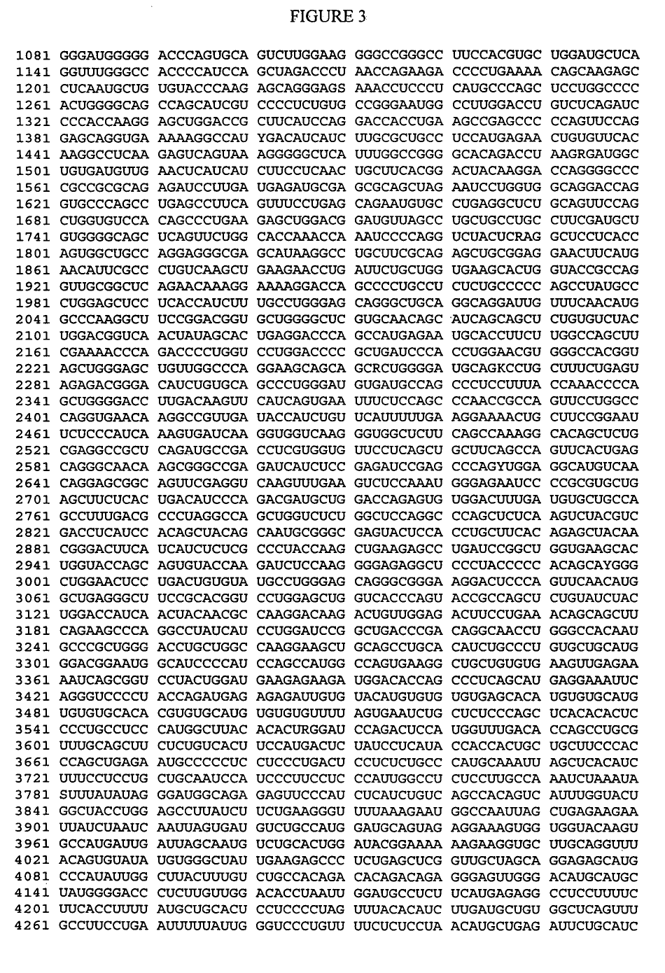

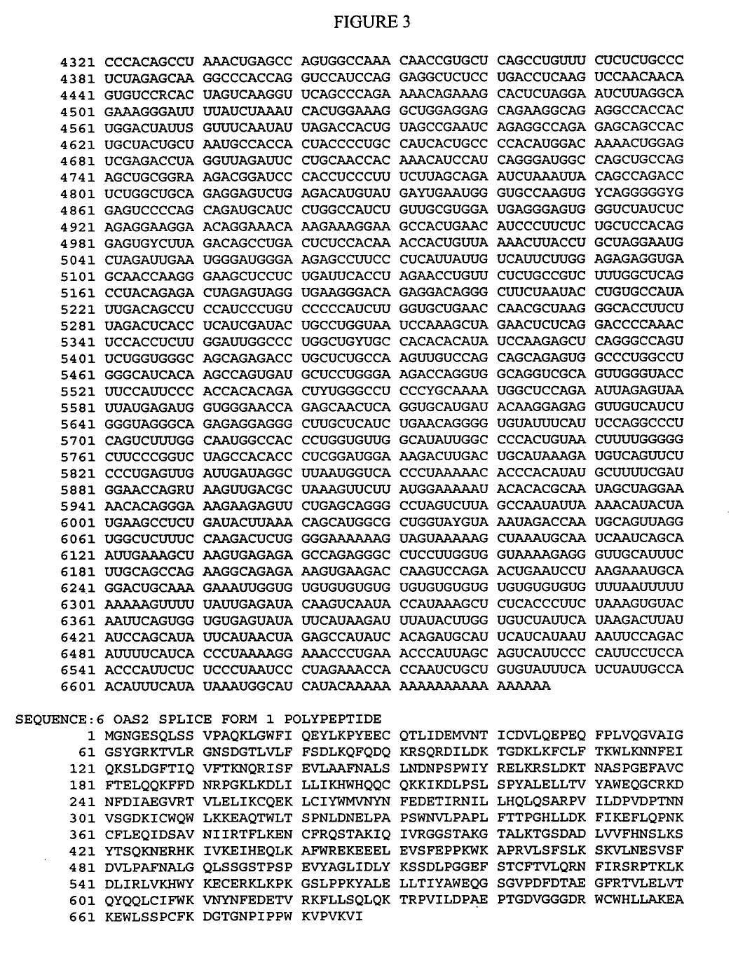

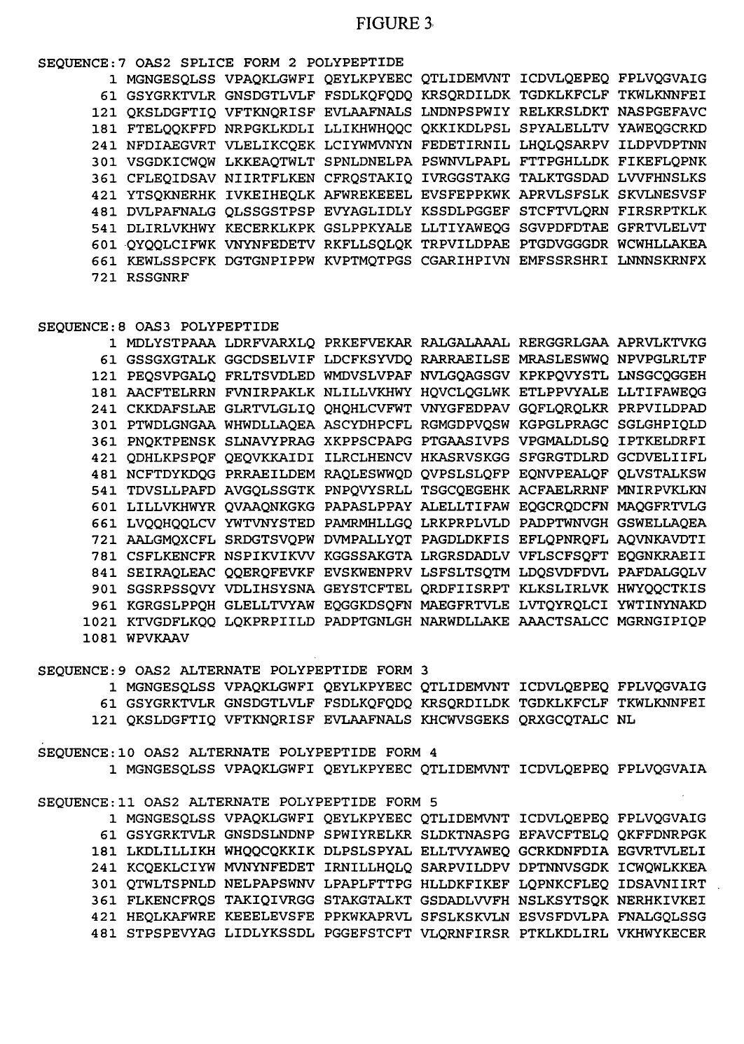

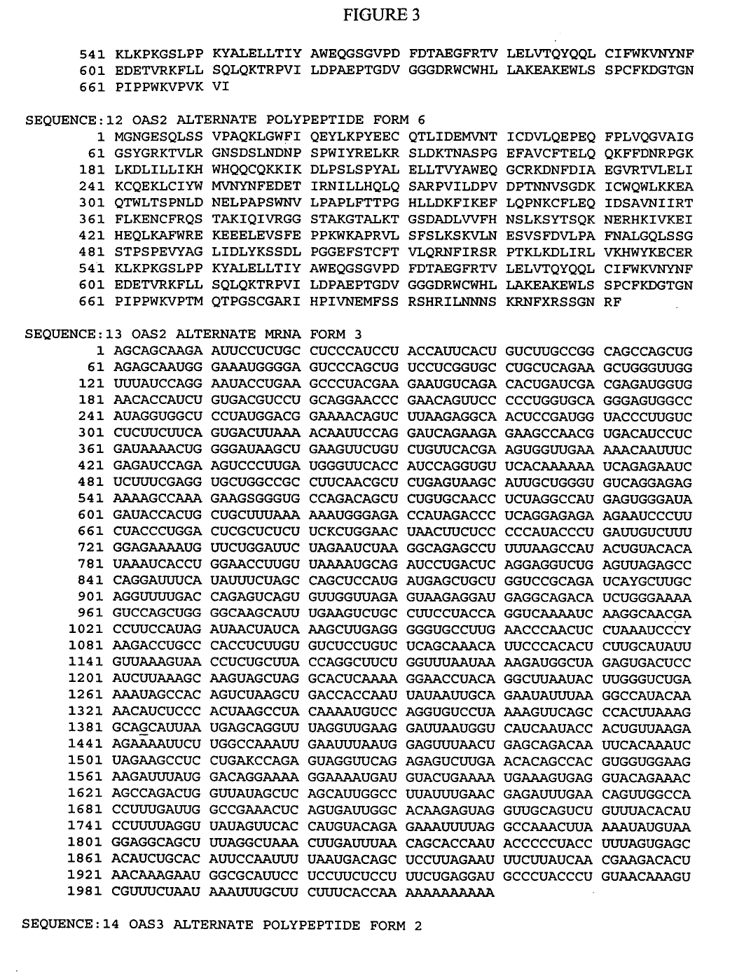

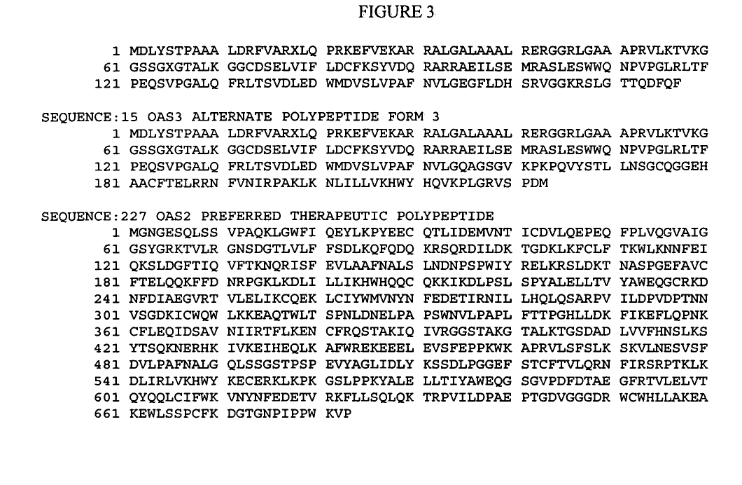

The terms “OAS protein”, “OAS polypeptide”, and “polypeptide expressed by an OAS nucleotide” as utilized in the present invention with regard to producing active pharmaceutical ingredients shall mean any polypeptide having at least 40%, 45%, 50%, 55%, 60%, 65%, 70%, 75%, 80%, 85%, 90%, or 95% homology to known oligoadenylate synthetase polypeptides, including but not limited to the sequences of FIG. 3, regardless of whether said polypeptide has oligoadenylate synthetase activity.

The term “manufacture” or “manufacturing” as utilized in the present invention with regard to OAS proteins or polypeptides means the process of producing milligram or gram quantities of the desired proteins or polypeptides under conditions suitable for use as an active ingredient (i.e. active pharmaceutical ingredient) or active agent in a pharmaceutical composition.

Modes of Practicing the Invention

As known to those skilled in the art, multiple experimental and analytical approaches are applied to the study design of the present invention. Without limiting the scope of the present invention, several preferred modes are presented below and in the examples attached.

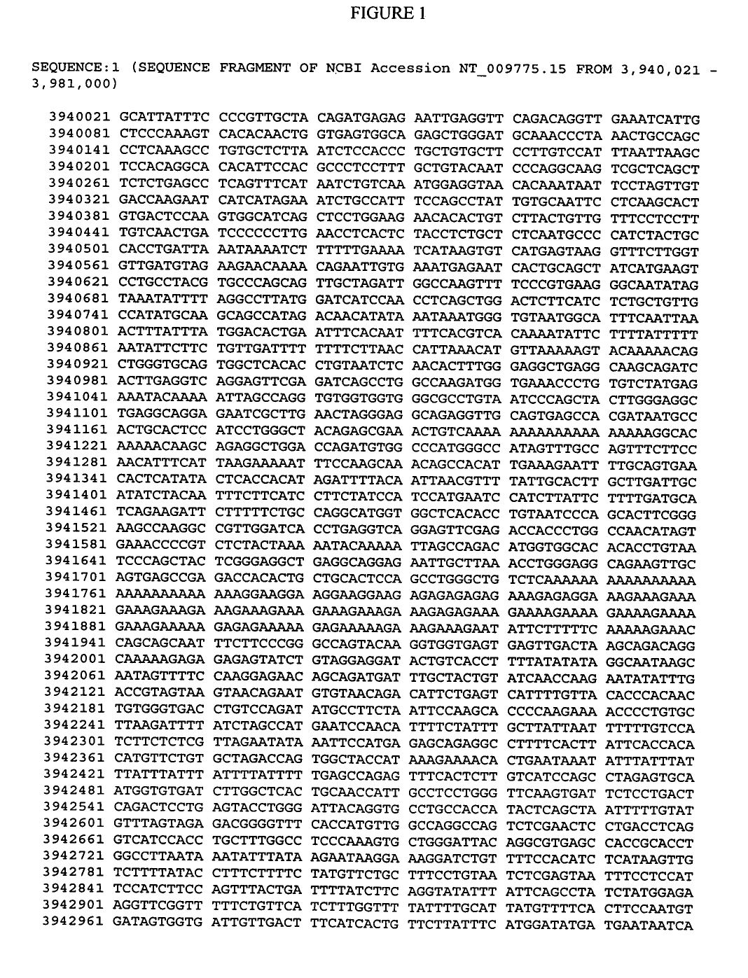

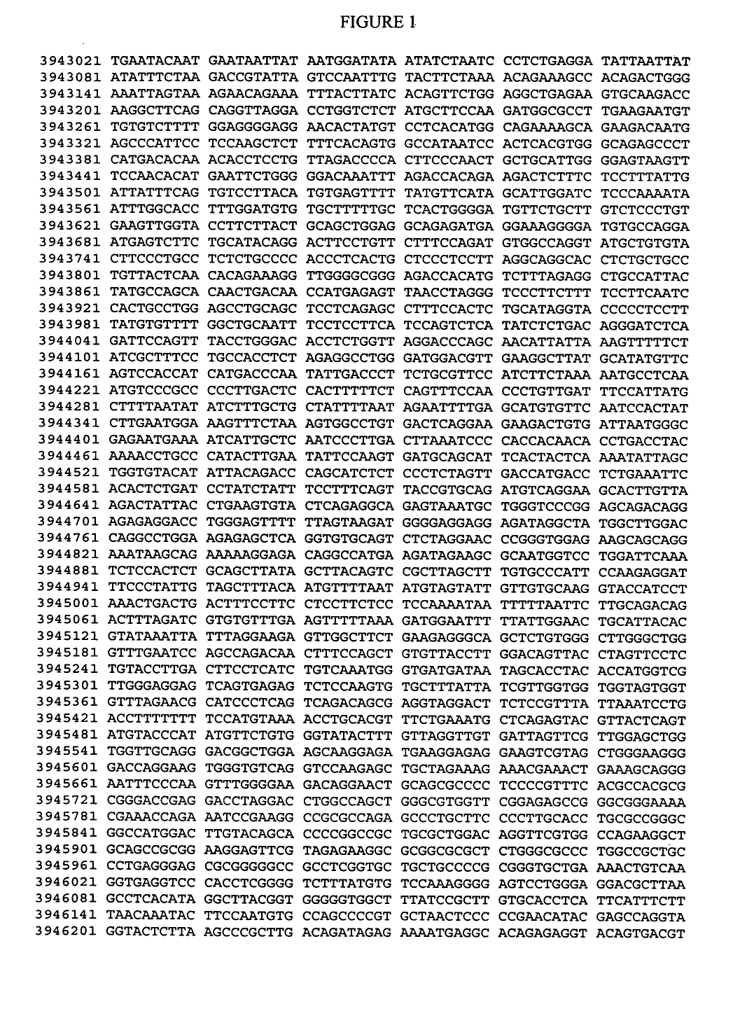

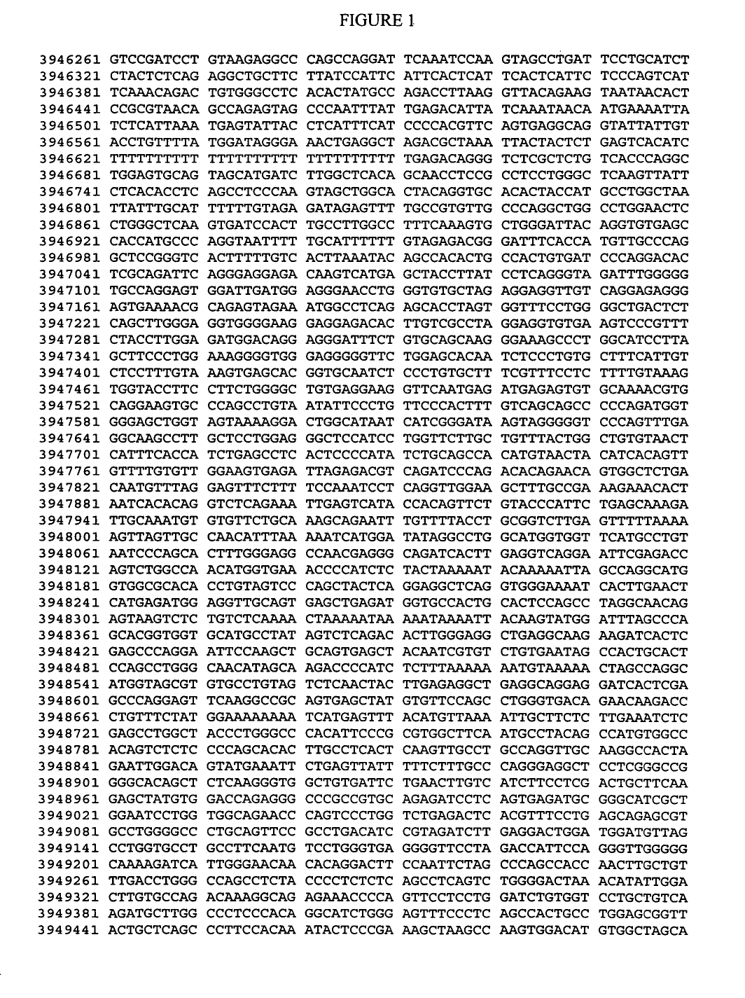

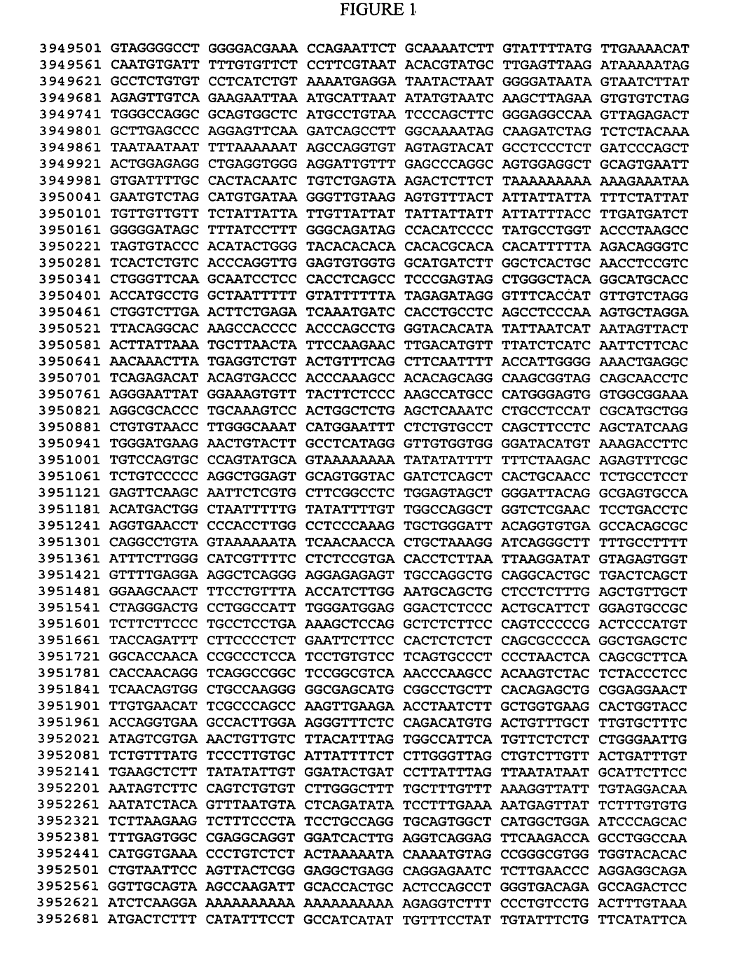

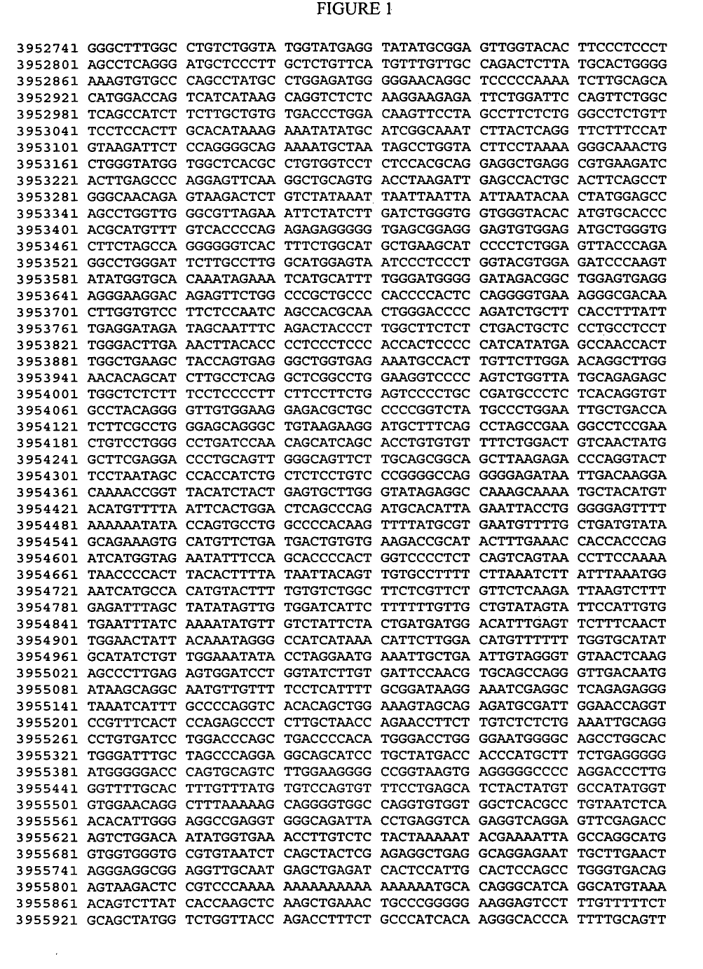

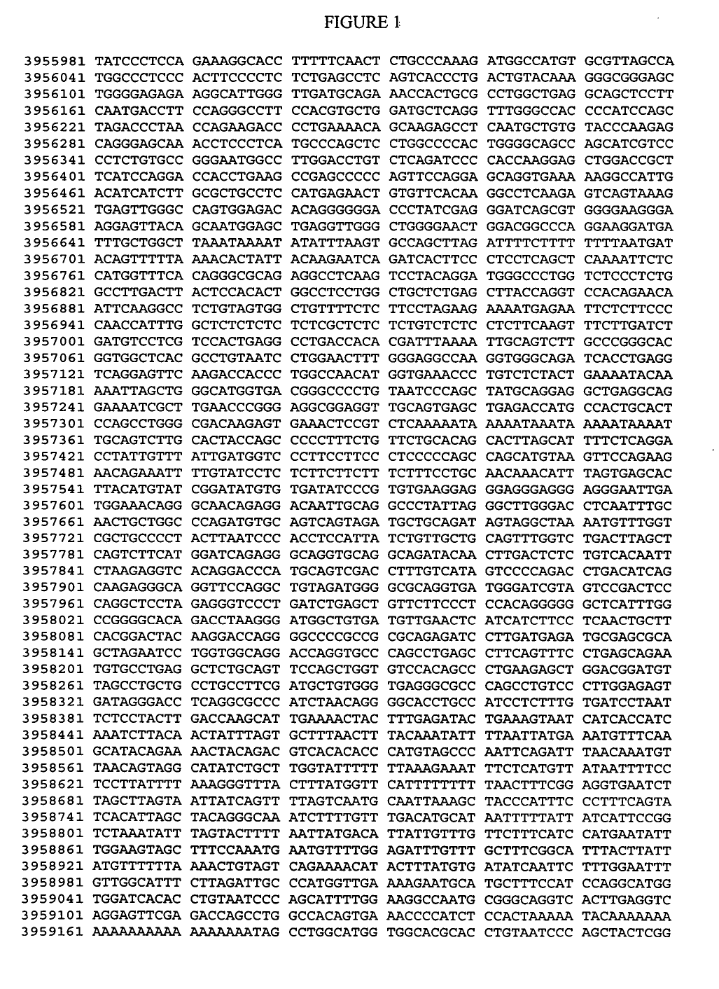

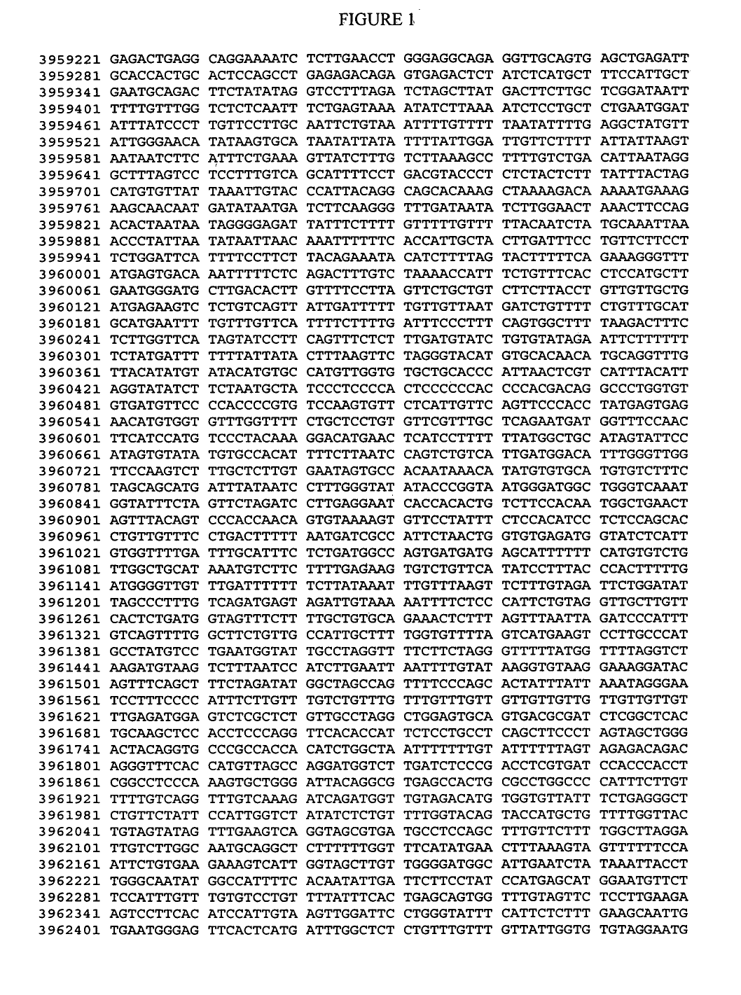

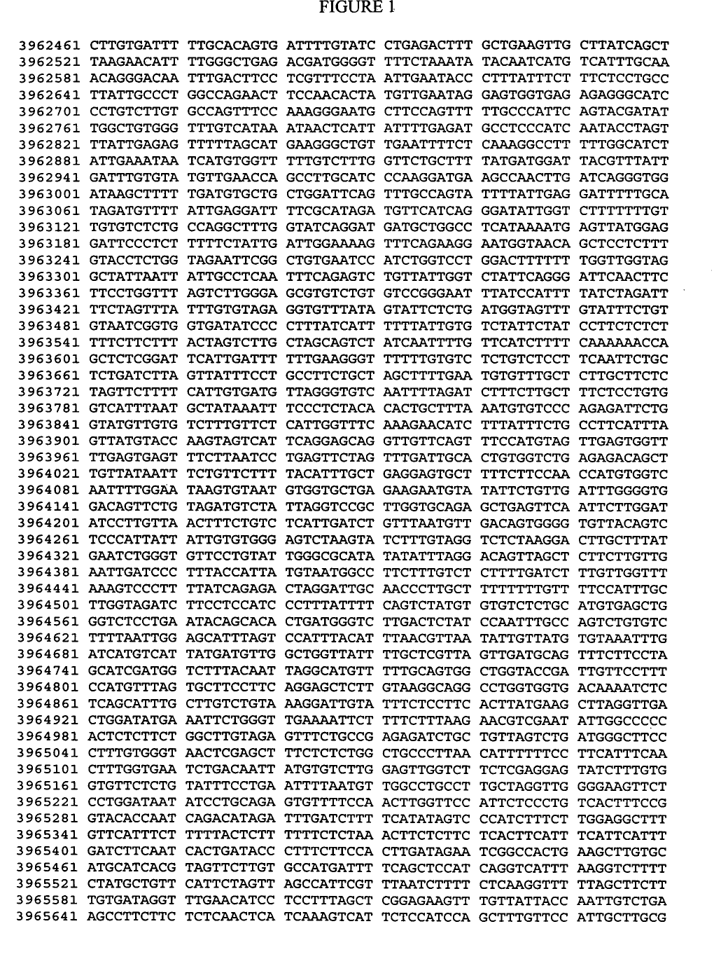

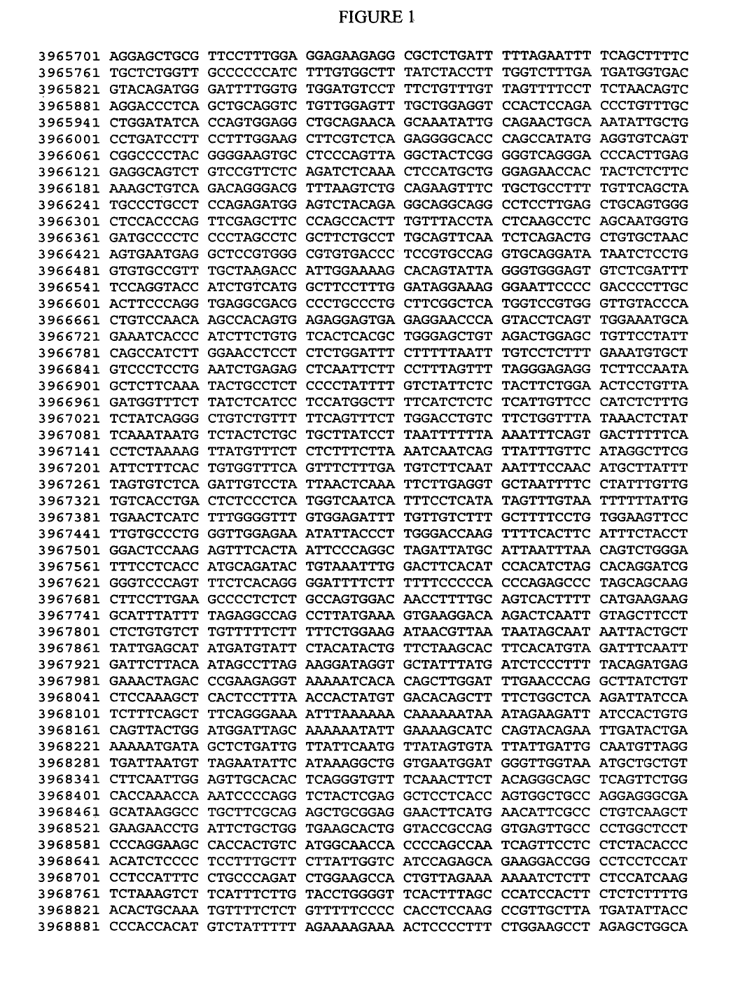

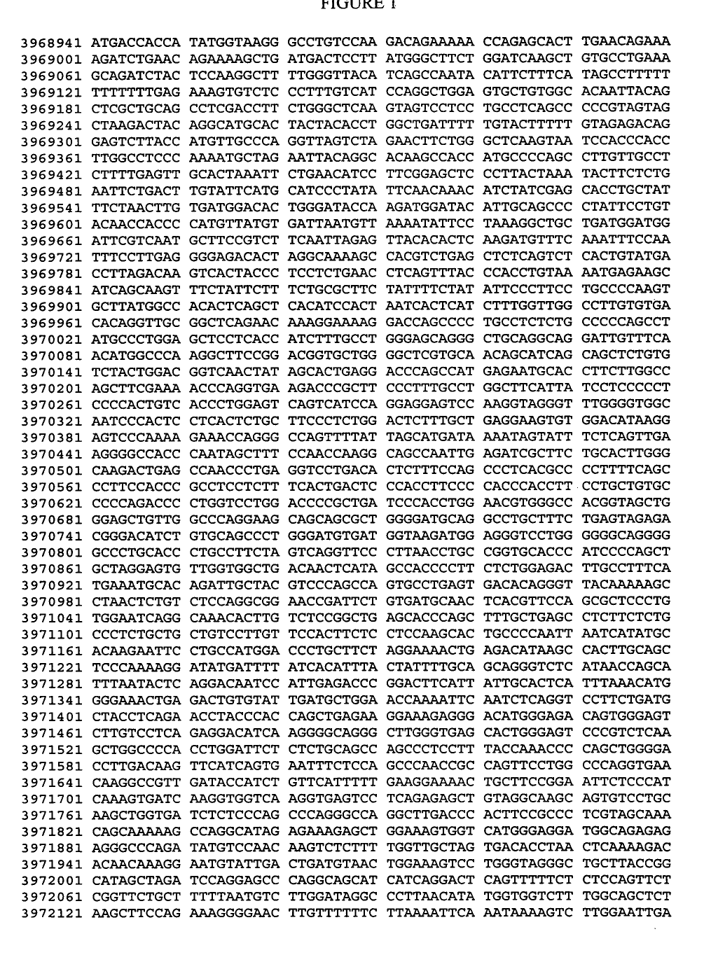

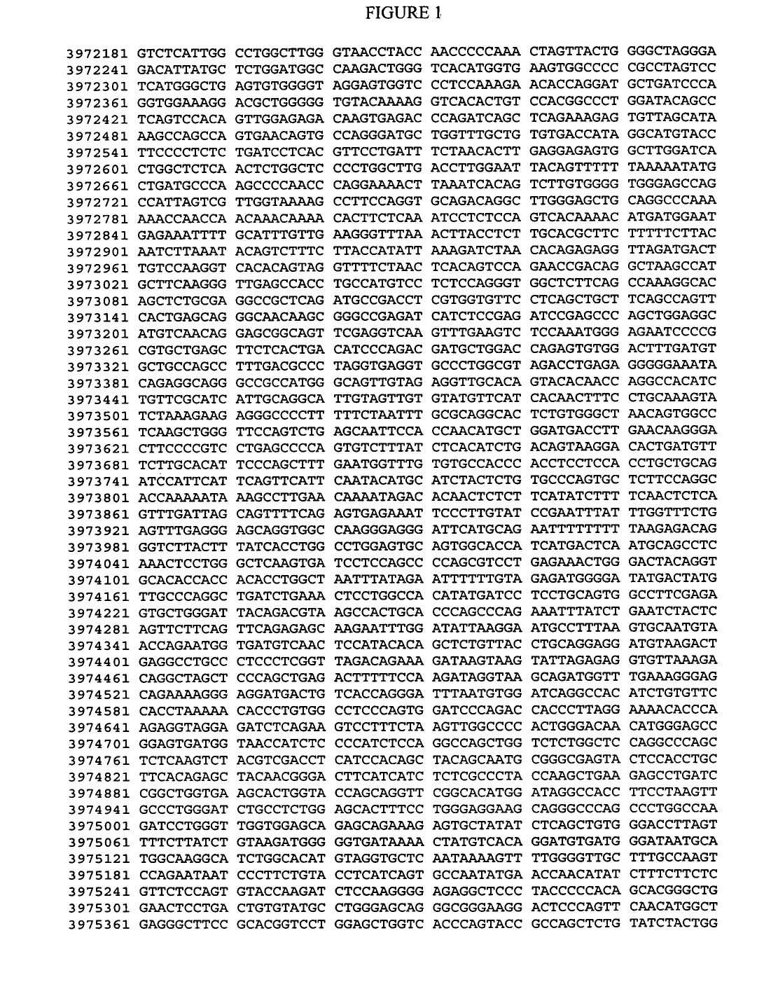

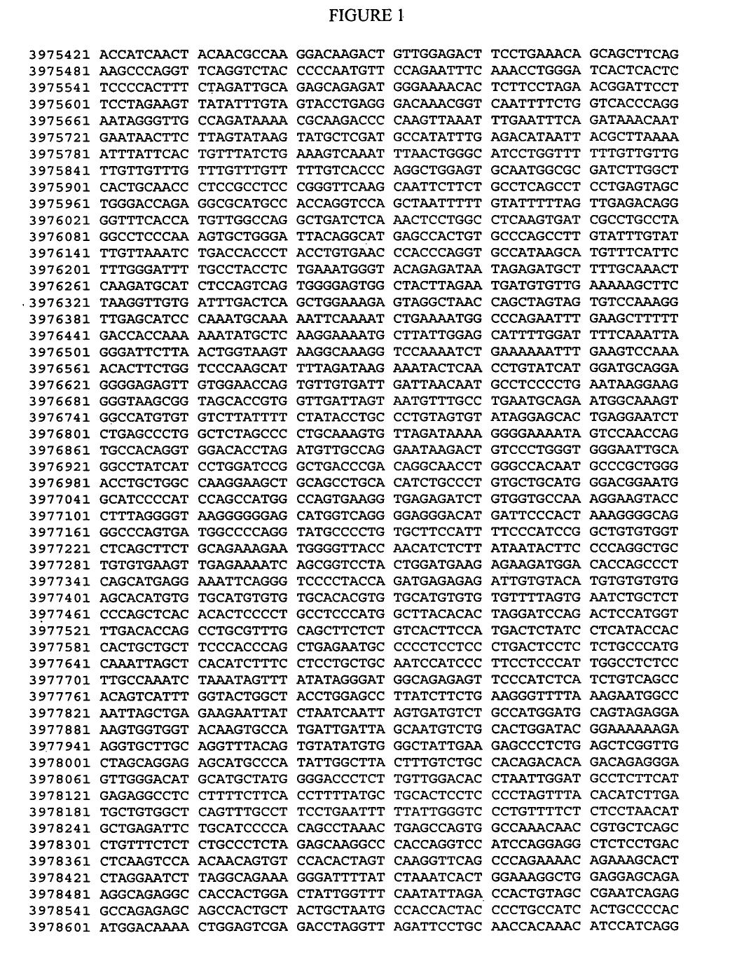

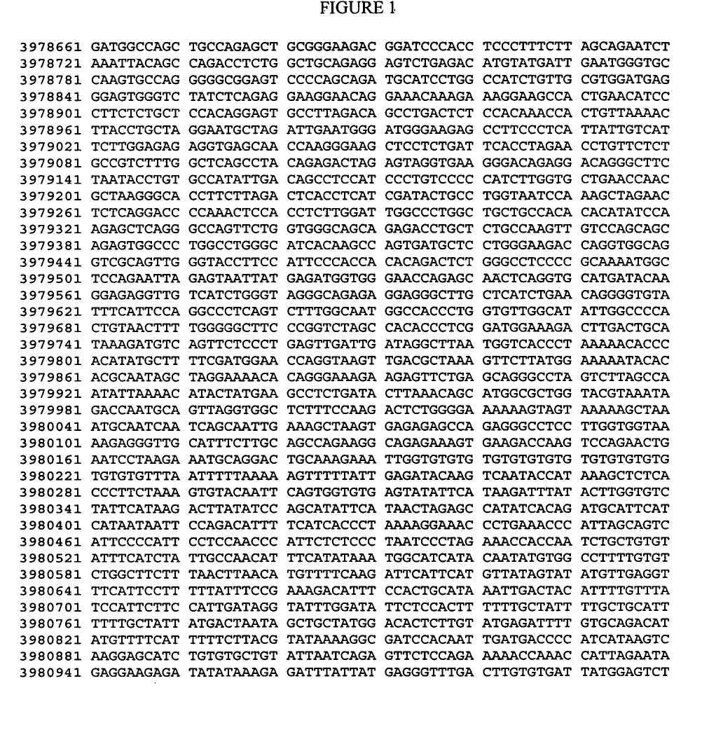

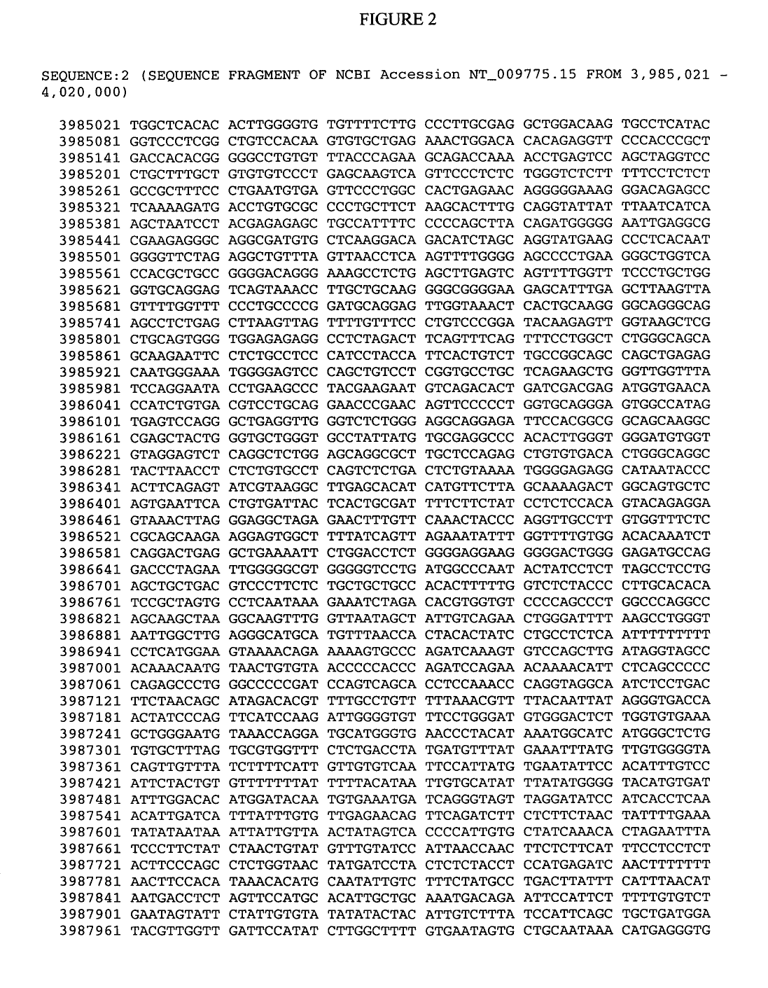

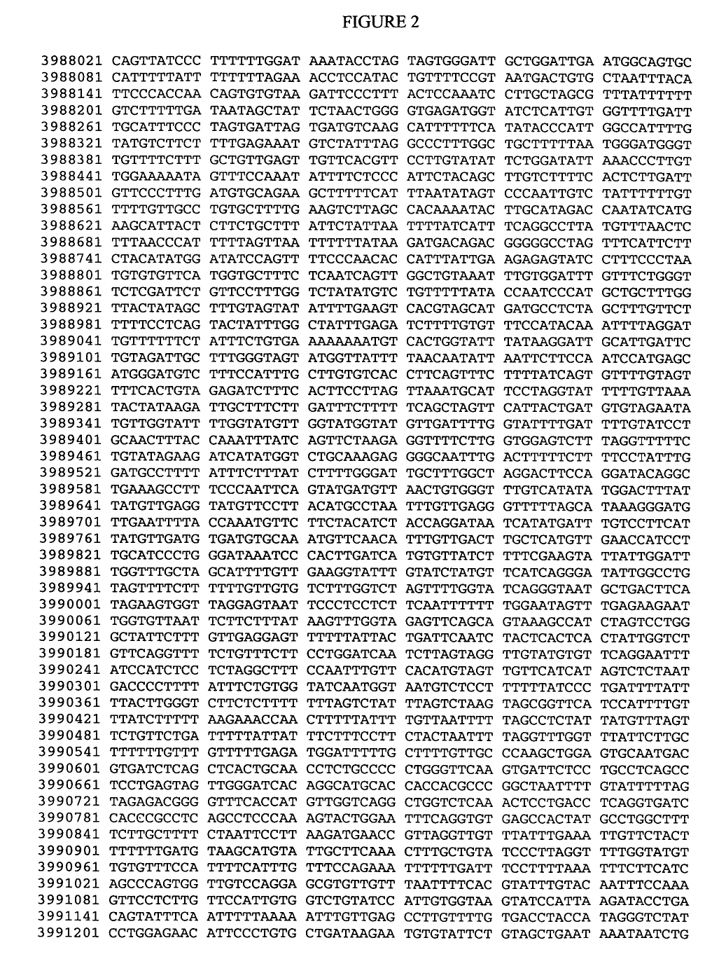

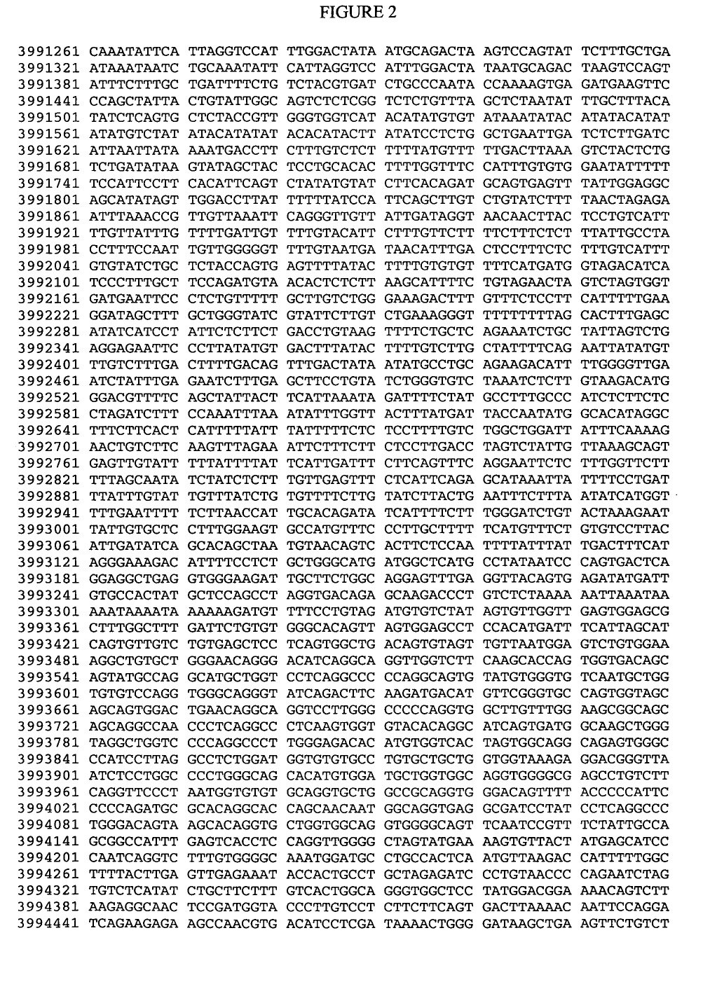

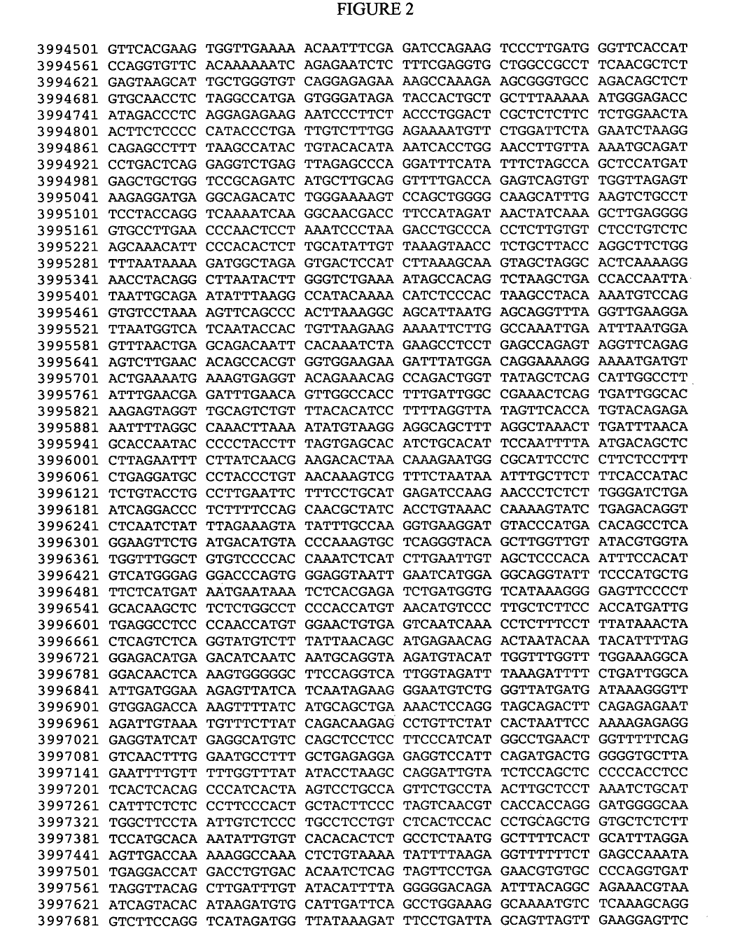

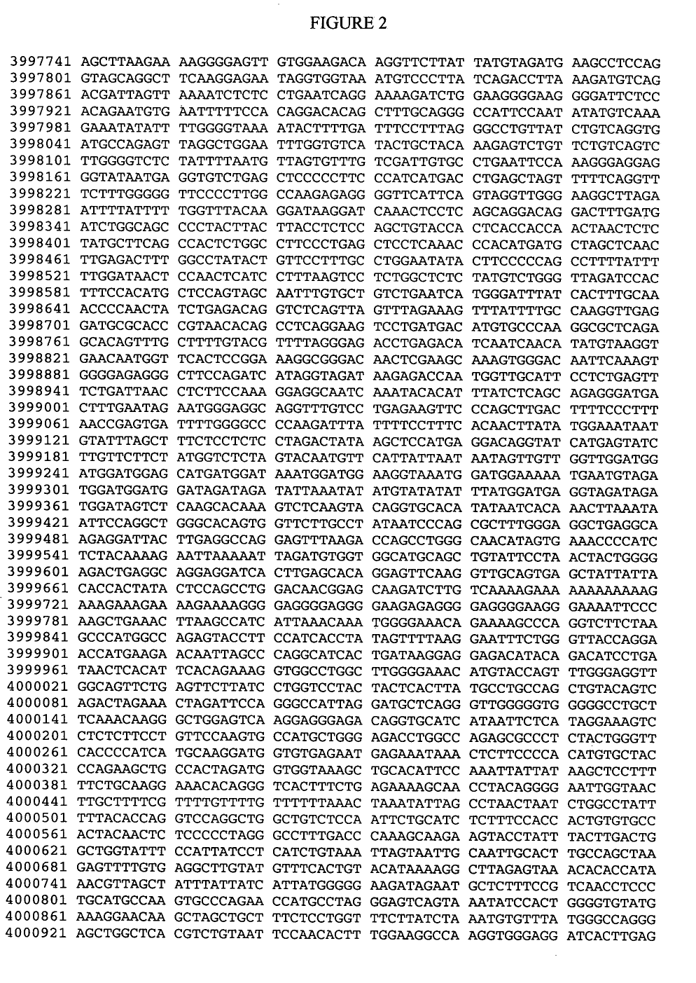

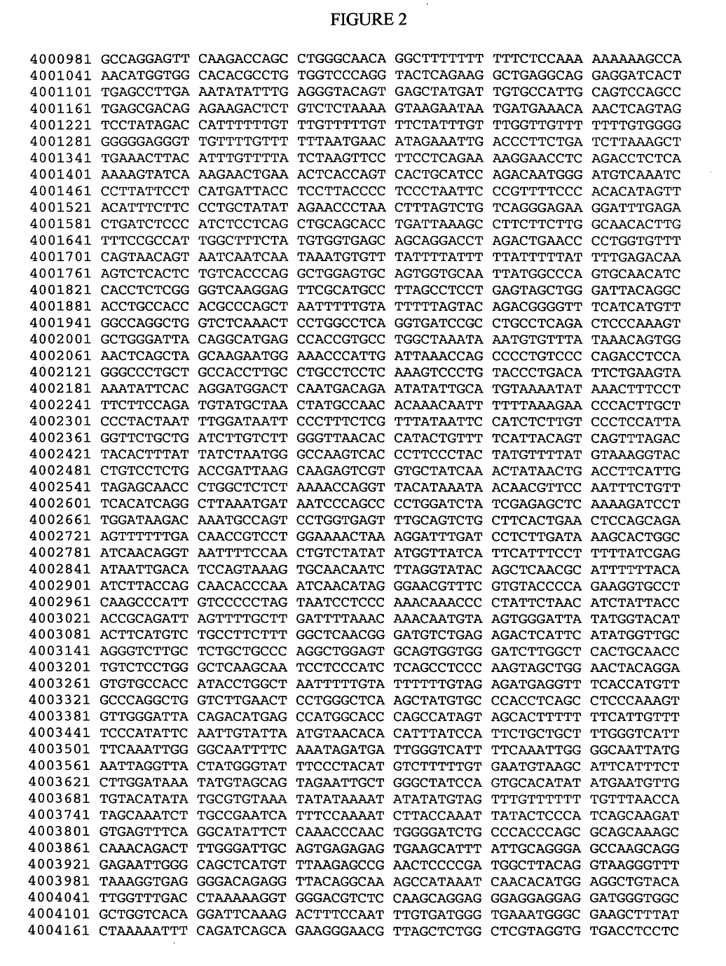

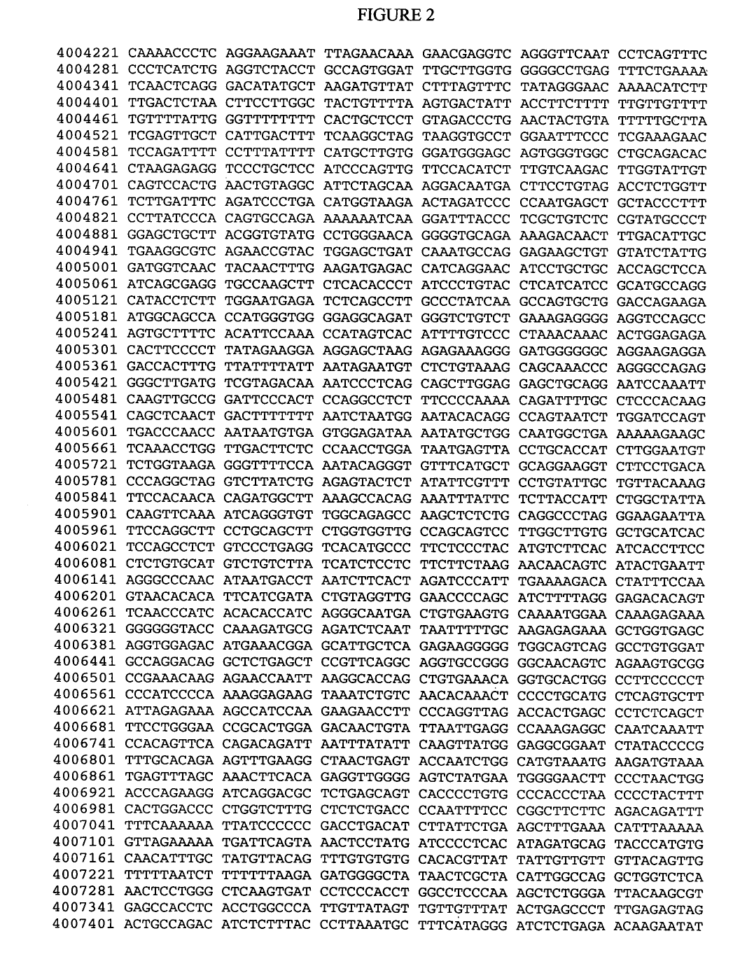

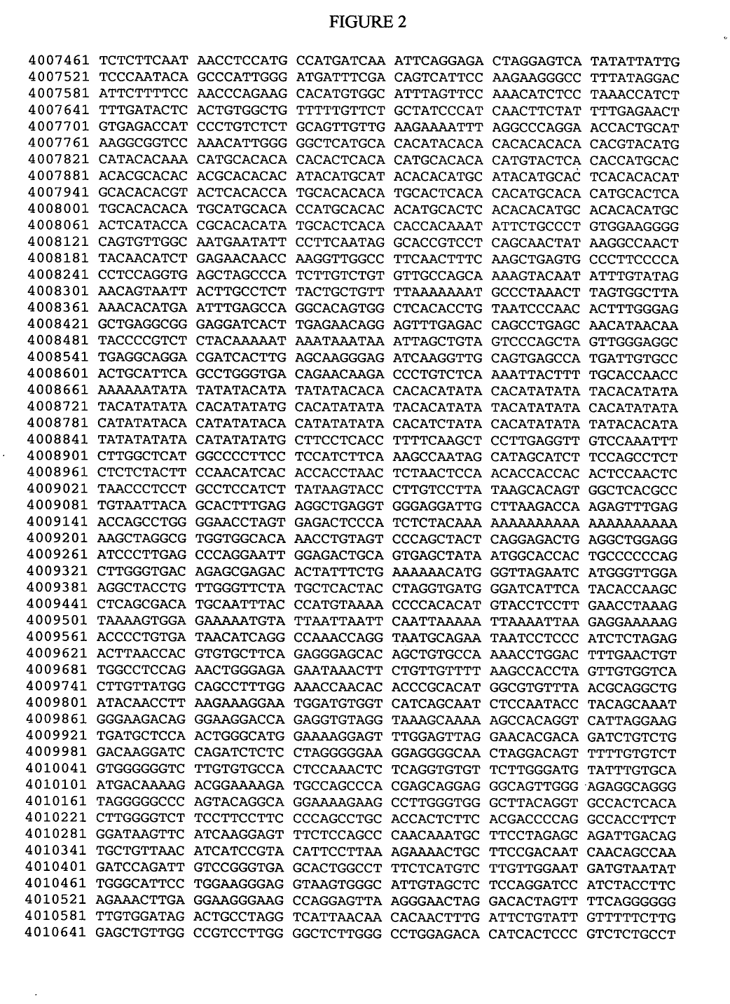

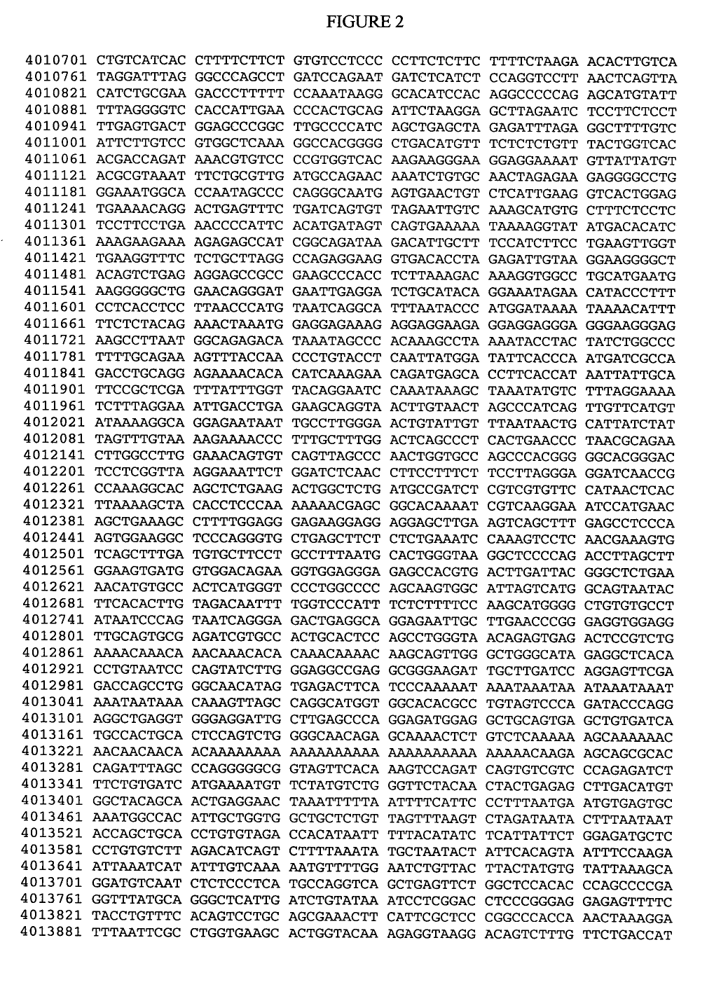

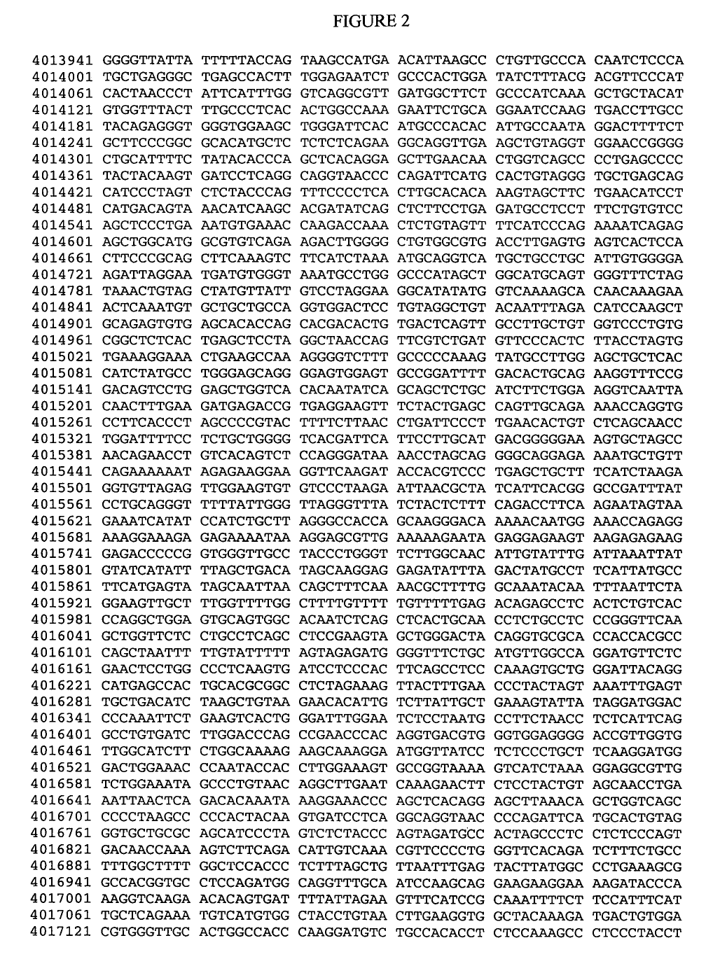

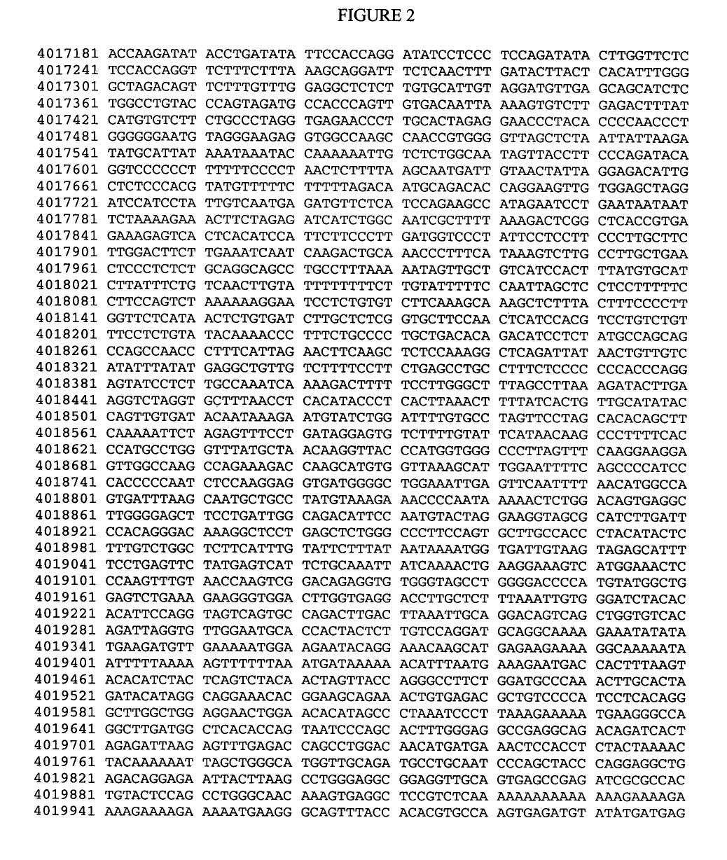

The present invention provides a novel method for screening humans for oligoadenylate synthetase alleles associated with sensitivity or resistance to infection by a virus, particularly, a flavivirus, particularly hepatitis C. The invention is based on the discovery that such resistance is associated with the particular base(s) encoded at one or more sites of mutation (as further described herein) in an oligoadenylate synthetase gene DNA sequence at nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15 (consecutive bases 3,940,021-3,981,000 of which are provided as SEQUENCE: 1 in FIG. 1 and consecutive bases 3,985,021-4,020,000 of which are provided as SEQUENCE:2 in FIG. 2), which encodes the human OAS3 and OAS2 genes. The screening method is not limited by the type of virus infection to be studied. Exemplary viruses include, but are not limited to, viruses of the Flaviviridae family, such as, for example, Hepatitis C Virus, Yellow Fever Virus, West Nile Virus, Japanese Encephalitis Virus, Dengue Virus, and Bovine Viral Diarrhea Virus; viruses of the Hepadnaviridae family, such as, for example, Hepatitis B Virus; viruses of the Picomaviridae family, such as, for example, Encephalomyocarditis Virus, Human Rhinovirus, and Hepatitis A Virus; viruses of the Retroviridae family, such as, for example, Human Immunodeficiency Virus, Simian Immunodeficiency Virus, Human T-Lymphotropic Virus, and Rous Sarcoma Virus; viruses of the Coronaviridae family, such as, for example, SARS coronavirus; viruses of the Rhabdoviridae family, such as, for example, Rabies Virus and Vesicular Stomatitis Virus, viruses of the Paramyxoviridae family, such as, for example, Respiratory Syncytial Virus and Parainfluenza Virus, viruses of the Papillomaviridae family, such as, for example, Human Papillomavirus, and viruses of the Herpesviridae family, such as, for example, Herpes Simplex Virus.

This invention discloses the results of a study that identified populations of subjects resistant or partially resistant to infection with the hepatitis C virus (HCV) and that further identified genetic mutations that confer this beneficial effect. Genetic mutations in the 2′-5′-oligoadenylate synthetase genes are identified, that are significantly associated with resistance to HCV infection. The study design used was a case-control, allele association analysis. Cases assigned as subjects had serially documented or presumed exposure to HCV, but who did not develop infection as documented by the development of antibodies to the virus (i.e. HCV seronegative). Control subjects were serially exposed subjects who did seroconvert to HCV positive. Case and control subjects were recruited from three populations, hemophilia patients from Vancouver, British Columbia, Canada; hemophilia patients from Northwestern France; and injecting drug users from the Seattle metropolitan region.

Case and control definitions differed between the hemophilia and IDU groups and were based upon epidemiological models of infection risk published in the literature and other models developed by the inventors, as described herein. For the hemophilia population, control subjects were documented to be seropositive for antibodies to HCV using commercial diagnostics laboratory testing. Case subjects were documented as being HCV seronegative, having less than 5% of normal clotting factor, and having received concentrated clotting factors before January 1987. Control injecting drug users were defined as documented HCV seropositive. Case injecting drug users were defined as documented HCV seronegative, having injected drugs for more than ten years, and having reported engaging in one or more additional risk behaviors. Additional risk behaviors include the sharing of syringes, cookers, or cottons with another IDU. Forty-seven (47) cases and 106 controls were included in this study.

Selection of case and control subjects was performed essentially as described in U.S. patent application Ser. No. 09/707,576 using the population groups at-risk affected (“controls”) and at-risk unaffected (“cases”).

The present inventive approach to identifying gene mutations associated with resistance to HCV infection involved the selection of candidate genes. Approximately 50 candidate genes involved in viral binding to the cell surface, viral propagation within the cell, the interferon response, and aspects of the innate immune system and the antiviral response, were interrogated. Candidate genes were sequenced in cases and controls by using the polymerase chain reaction to amplify target sequences from the genomic DNA of each subject. PCR products from candidate genes were sequenced directly using automated, fluorescence-based DNA sequencing and an ABI3730 automated sequencer.

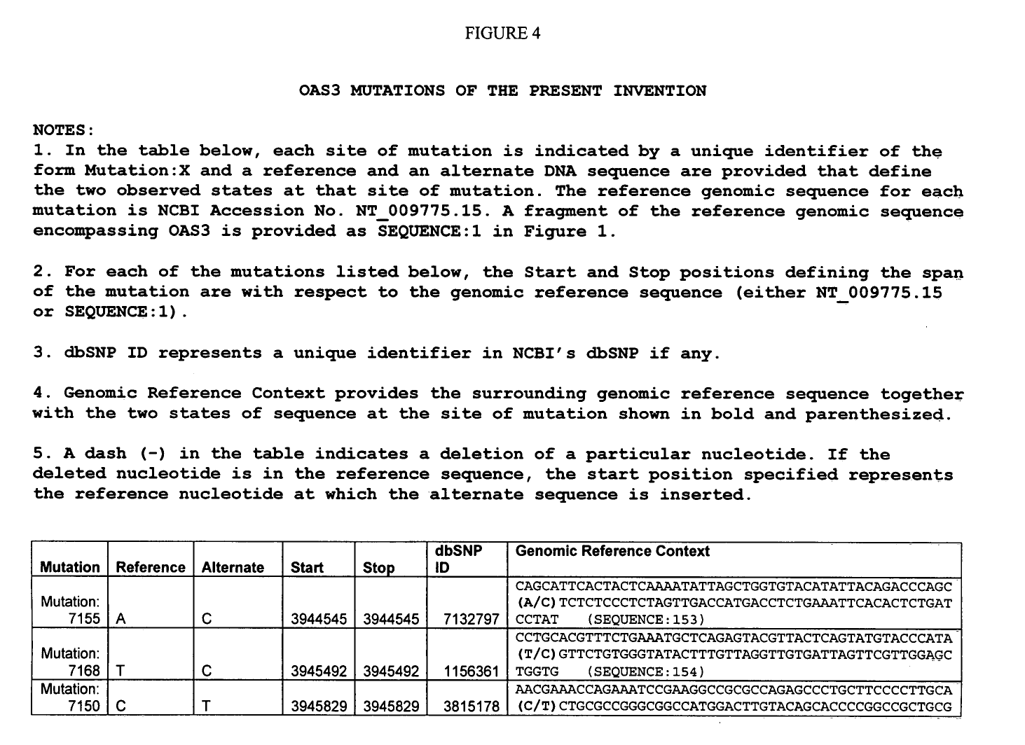

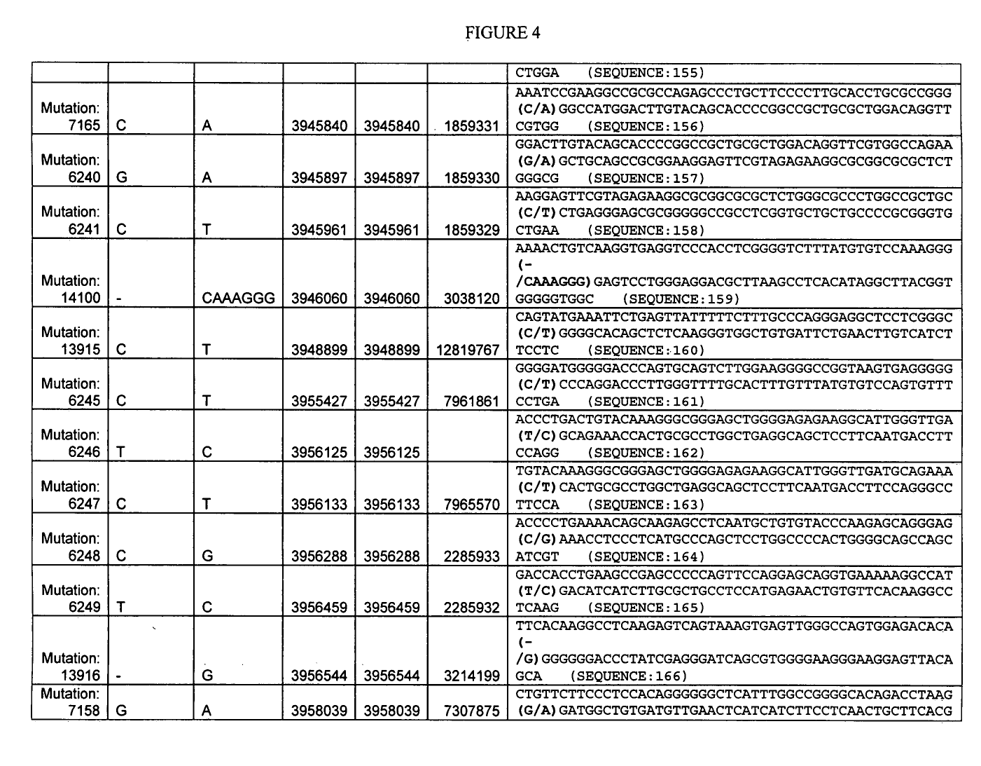

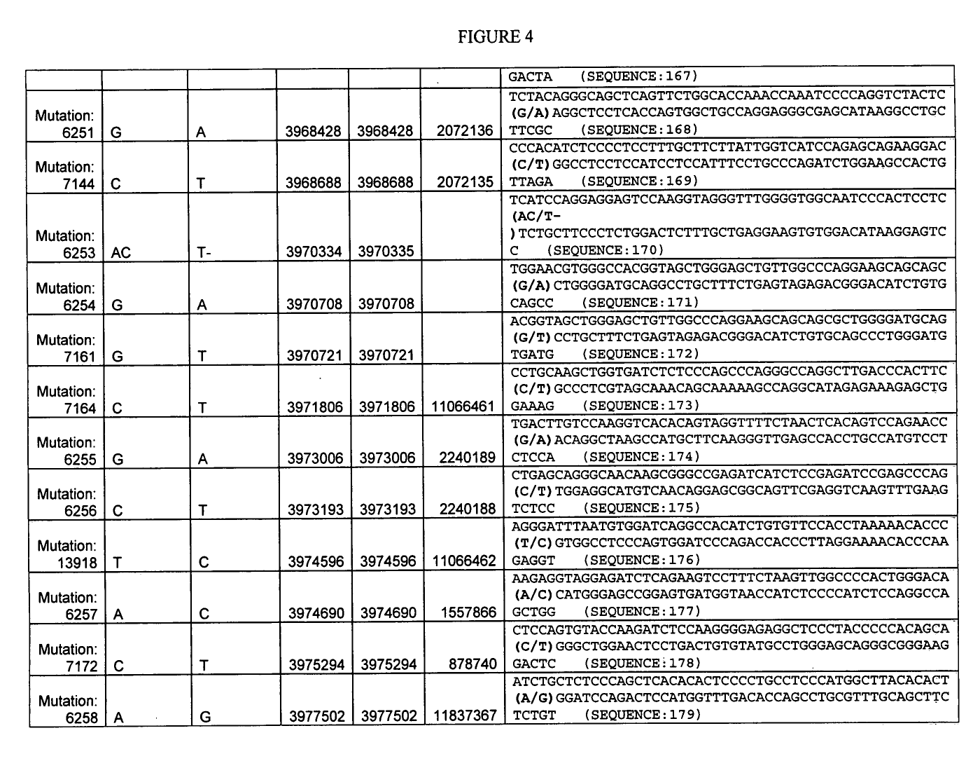

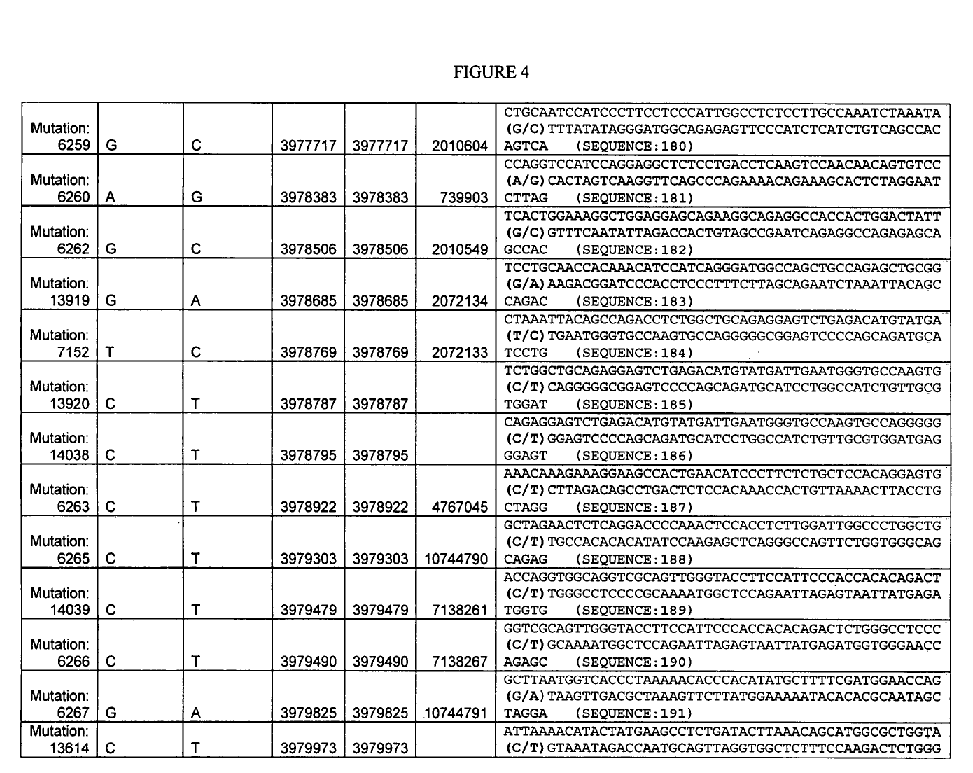

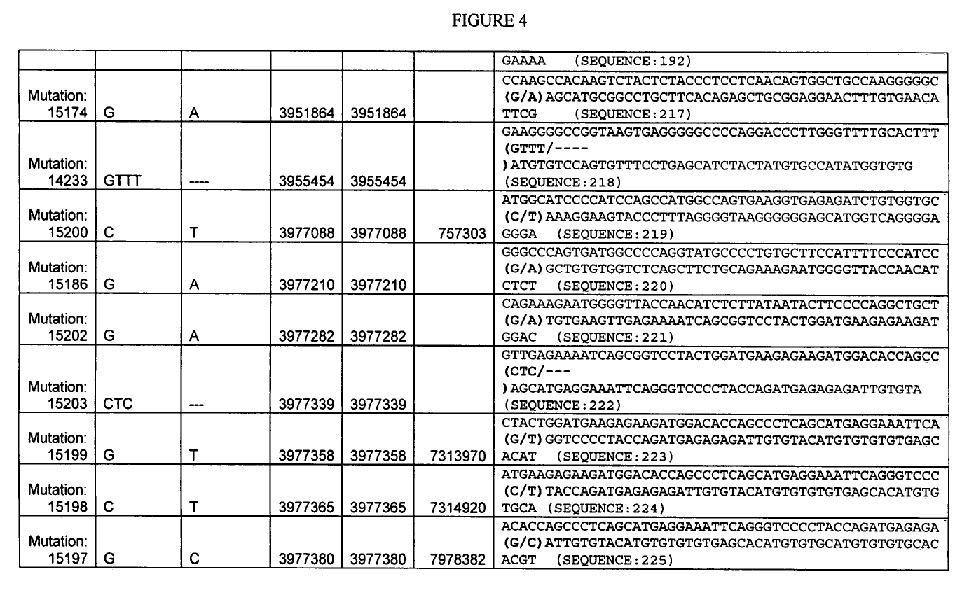

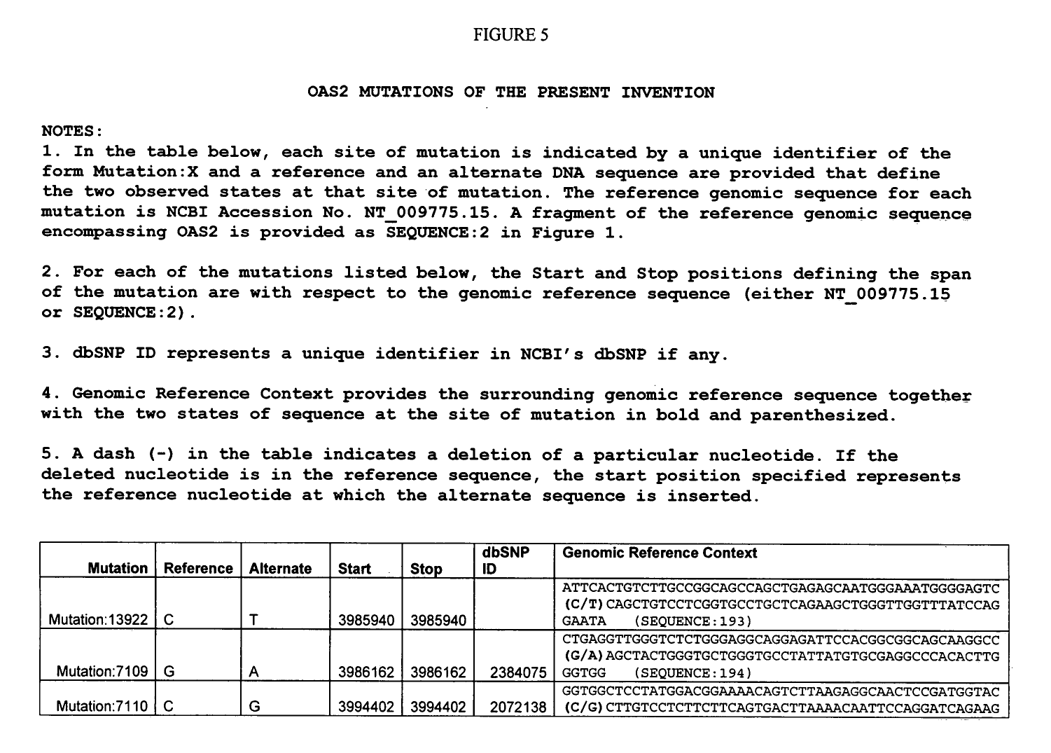

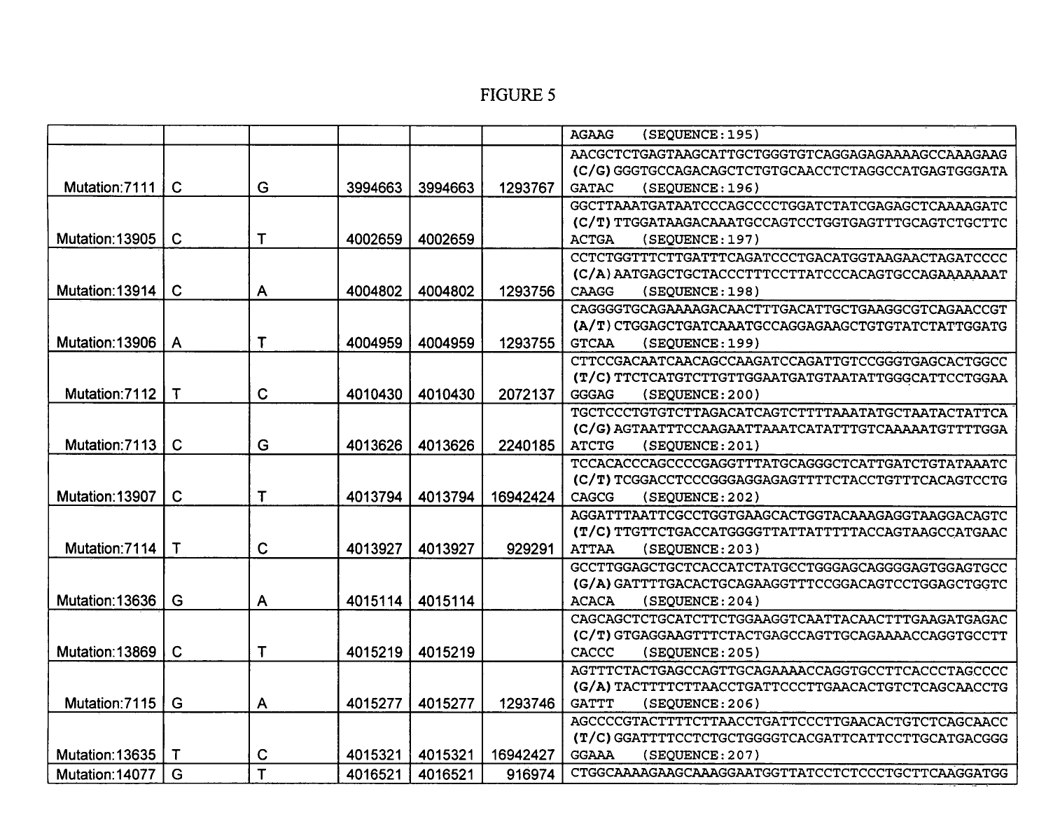

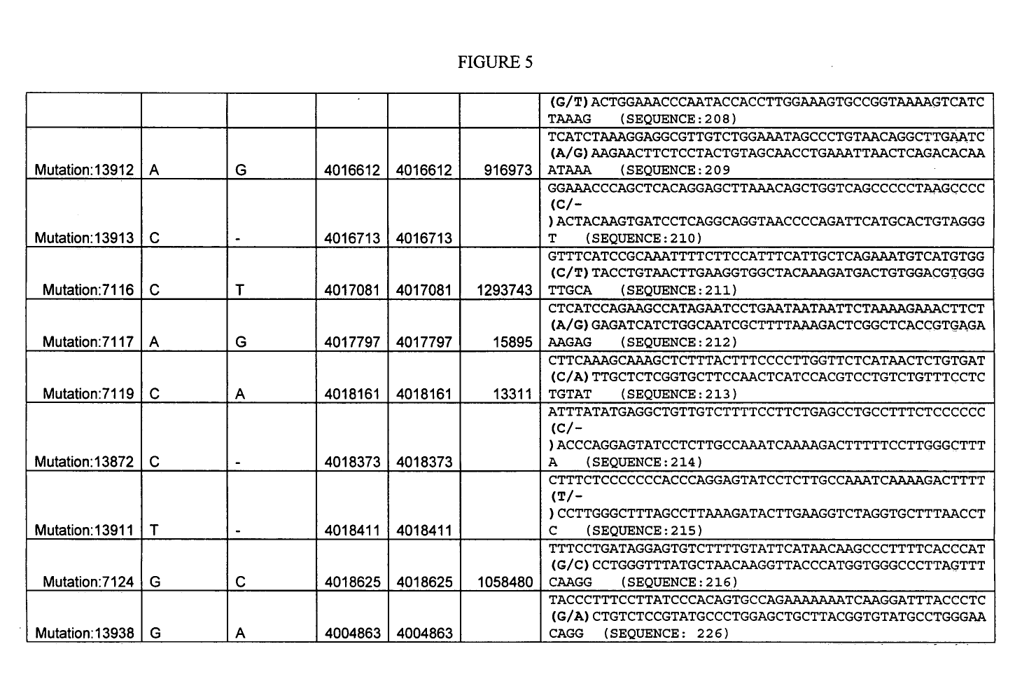

Exhaustive sequencing of the coding and regulatory regions of the oligoadenylate synthetase 2 and 3 genes (OAS2 and OAS3, respectively) in the present population identified 70 polymorphic mutations occurring more than once and 4 singleton mutations. Forty nine of these mutations are characterized and identified in FIG. 4 corresponding to OAS3. Twenty five mutations are characterized and identified in FIG. 5 corresponding to OAS2. These mutations produce variant forms of the OAS3 or OAS2 genes, respectively. As further described below, resistance to HCV infection in the present population was found to be significantly associated (p<0.05) with distinct subsets of this group of mutations. Therefore, variant forms of both the OAS2 and OAS3 gene are believed to confer resistance to viral infection.

In one preferred mode of numerical analysis, allele association analysis is performed to identify bias in the frequency of occurrence of a particular allele at one or more sites of mutation with respect to either the case or control group, thereby identifying one or more mutations associated with resistance to HCV infection. Said association is tested for statistical significance using any of a number of accepted statistical tests known to those skilled in the art, including chi-square analysis.

In another preferred mode of numerical analysis, linkage disequilibrium analysis as known to those skilled in the art is performed to identify predictive relationships between pluralities of mutations in the genotype data. One example is the well-known calculation of a linkage disequilibrium estimate, commonly referred to as D' (Lewontin, Genetics 49:49-67, 1964). Those skilled in the art will recognize that numerous other analytical methods exist for assessing the evolutionary importance of particular mutations in a genetic analysis. Other particularly relevant methods attempt to estimate selective pressures and/or recent evolutionary events within a genetic locus (for example, selective sweeps) by comparing the relative abundance of high-, moderate-, or low-frequency mutations in the locus. Most familiar of these tests is the Tajima D statistic (Tajima, Genetics 123:585-595, 1989). Fu and Li, Genetics 133:693-709 (1993) have also developed a variant to the Tajima and other statistics that also makes use of knowledge regarding the ancestral allele for each mutation. These and other methods are applied to the mutations of the present invention to assess their relative contribution to the observed phenotypic effects with regard to viral infection, IDDM, or cancer.

In another preferred mode of numerical analysis, haplotypes comprising combinatorial subsets of OAS2 or OAS3 mutations are computationally inferred by Expectation Maximation (EM) methods as known to those skilled in the art (Excoffier, L et. al. Mol Biol Evol., 12(5):921-7, 1995). A number of haplotypes are identified in the case and control population by this analysis. Using this method, each subject in the population is assigned two parental haplotypes. Haplotype distributions among case and control subjects are analyzed by known statistical methods (including chi-square analysis) to identify bias toward either other group, thereby identifying particular haplotype that conferring resistance to HCV infection.

In other preferred modes of analysis, specific genetic models of resistance to HCV infection are examined utilizing mutation allele data or inferred haplotype data (as described above). Exemplary genetic models include those that model resistance as dominant, additive, and recessive effects. Models are tested for their ability to significantly predict resistance or sensitivity to HCV infection by any one of a number of accepted statistical approaches, including without limitation, logistic regression.

Specific haplotypes or allelic states at one or more sites of mutation that are shown to be significantly associated with resistance or sensitivity to HCV infection by any of the above analytical approaches are further analyzed to identify biological effectors of said resistance or sensitivity. Such further analysis includes both computational and experimental modes of analysis. In one such further preferred embodiment, the haplotype identified as associated with resistance to HCV infection (a “resistant haplotype” corresponding to a “resistant form” of OAS2 or OAS3) is compared with its nearest “neighbors” in terms of total mutational content. Such comparison identifies particular mutational states at specific sites within the gene that act to confer resistance. In another preferred embodiment, further population genotyping analysis is conducted in other portions of the OAS2 or OAS3 gene and surrounding genomic region, including without limitation the introns, in order to identify additional mutations that are either independently associated with resistance to HCV infection or that contribute to more expansive haplotypes associated with resistance to HCV infection. In another preferred embodiment, a “resistant haplotype” is experimentally analyzed in comparison with closely-related neighbor haplotypes to identify biological differences that confer resistance. Such experimental analysis includes, without limitation, comparative analysis of expression levels, transcription of variant mRNAs, identification of exonic and intronic splice enhancers, mRNA stability, viral and anti-viral interactions, metabolic effects, and cell cycle modifications by methods as described elsewhere herein and as known to those skilled in the art. In one such embodiment, the comparative analyses are performed between samples derived from homozygous individuals carrying the resistant haplotype and one or more samples derived from individuals carrying other haplotypes for comparison.

As further described in Examples 7-8 below, particular haplotypes were determined to be significantly associated with resistance (by definition also specifically including herein all degrees of increased or decreased susceptibility) to HCV infection. Thus the invention provides genetic haplotypes that are resistant to HCV infection. As described further below, the mutations in these haplotypes are used to screen human subjects for resistance to viral infection, particularly flavivirus infection, most particularly hepatitis C infection. The invention further provides one or more specific regions of OAS2 or OAS3 (as described below) that are targets for therapeutic intervention in viral infection, particularly flavivirus infection, most particularly HCV infection. Furthermore, the invention also provides novel forms of OAS2 or OAS3 that may be useful in treating viral infection, particularly flavivirus infection, most particularly HCV infection.

The present invention is not limited by either the foregoing or other illustrative examples. In another illustrative example, Mutation:7117 in OAS2 is a substitution of a G nucleotide for the reference A nucleotide. This mutation only occurs in the terminal exon of the second transcript form of OAS2 (SEQUENCE:4 mRNA, SEQUENCE:7 polypeptide, the so-called p71 form) due to differential splicing that creates an eleventh exon on this second form that is missing from the smaller first transcript form (SEQUENCE:3 mRNA, SEQUENCE:6 polypeptide, so-called p69 form). Importantly, this mutation abolishes the termination codon in favor of a tryptophan at amino acid position 720 and thereby lengthening the polypeptide by eight amino acids. Two of these eight are positively charged arginines that are likely to be exposed on the surface of the protein. The importance of this mutation is seen in the common role played in exemplary haplotypes from Example 8. Therein, the G nucleotide/Tryptophan form is identified in haplotypes showing both increased resistance and increased susceptibility to HCV infection suggesting that factors relating to the extended peptide form of SEQUENCE:7 (e.g. change in relative expression of the extended peptide due to other concomitant mutations within the gene) are important mediators of viral resistance.

As the propensity toward alternative splice variants is a hallmark of the OAS gene family and is consistent with the notion that increased structural variety in immune system genes increases survivability when challenged with pathogens, OAS2 and OAS3 are examined for evidence of additional alternate splice forms. Data sets containing multiply sampled cDNA fragments from clone libraries derived from multiple human tissues, such as NCBI's dbEST (Boguski, M.S. et. al., Nat Genet. 1993 Aug;4(4):332-3), are analyzed for evidence of alternate splice forms of OAS2 or OAS3 other than those previously known in the art. As an illustrative example of this analysis, Examples 9-10 below provide evidence for novel splice forms of OAS2 and OAS3, respectively. Such alternate splice forms are further analyzed (as described elsewhere herein) in human tissue samples of known OAS2 or OAS3 haplotype as appropriate and the presence and relative expression of such alternate splice forms is correlated with OAS haplotype.

These variant forms of the OAS2 or OAS3 genes and corresponding transcript variants are believed to encode one or more of the polypeptides consisting of SEQUENCE:9-12 (for OAS2) and SEQUENCE:14-15 (for OAS3). The foregoing polypeptides, either singly or plurally, and any gene or RNA polynucleotides that encode them, are investigated for their relationship to viral resistance, IDDM, and cancer and their utility in developing treatments thereto, in the same manner as with other polypeptides of the present invention. Several of these polypeptides are extreme truncations of their respective OAS2 or OAS3 canonical forms and may therefore represent defective proteins whose prevalence and/or function (or lack thereof) may play a significant role in any of the above disease indications.

In addition to the simple production (or non-production as the case may be) of such alternative transcripts, resistant forms of the OAS2 or OAS3 gene may also contain or abolish specific sequence contexts (such as Exon Splice Enhancers) that modify the selective preference for such specific transcript variants. This in turn would cause differing relative levels of abundance of the resulting proteins. These variant forms of the OAS2 or OAS3 gene may also modify localization or post-translational modification of the resulting proteins. Those skilled in the art will appreciate that increased abundance or other modifications that improve the activity, stability, or availability of a specific OAS2 or OAS3 protein form may improve the overall anti-viral performance of the 2′-5′-OAS/RNase L pathway. Those skilled in the art can likewise appreciate that depressing the activity or availability of a specific OAS2 or OAS3 form may also improve the overall anti-viral performance of the 2′-5′-OAS/RNase L pathway in cases where said specific protein is not advantaged, or even disadvantaged, over other specific OAS2 or OAS3 forms. Without limitation, one embodiment of a disadvantaged OAS2 or OAS3 protein is one which is specifically targeted by viral protein(s) in such a manner as to preclude the enzymatic activity of said specific OAS2 or OAS3 protein. A further embodiment of a non-advantaged OAS2 or OAS3 protein is one with lower enzymatic activity polymerizing with other active forms thereby lowering, or abolishing, the overall enzymatic activity (and hence decreasing overall anti-viral effect) of the polymerized protein. Despite the foregoing, however, it is recognized that enzymatically inactive forms of the OAS proteins have antiviral activity. One or more of the foregoing mechanisms may contribute to resistance to viral infection. The present invention is not limited, however, by the specific mechanism of action of the disclosed variant polynucleotides or polypeptides. The present invention is also not limited by any particular allele or haplotype disclosed herein and the examples and modes described herein are purely exemplary.

The invention also provides forms of the OAS2 and OAS3 genes that are characterized by the presence in the respective gene of one or more genetic mutations or haplotypes not previously disclosed in the public databases.

The invention therefore provides novel forms of human 2′-5′-oligoadenylate synthetase genes, novel mRNA transcripts, and associated proteins. The invention also discloses utility for the novel mRNA transcripts and novel proteins.

The invention provides OAS2 or OAS3 gene forms that confer on carriers a level of resistance to the hepatitis C virus and associated flaviviruses including but not limited to the West Nile virus, dengue viruses, yellow fever virus, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and the Kyasanur forest disease virus. The OAS proteins have also been shown to be important in attenuating infection in experimental respiratory syncitial virus and picornavirus cell culture infection systems. Failure of human immunodeficiency virus-1 (HIV-1) infected cells to release virus has been correlated with high concentrations of OAS and/or 2-5A. Furthermore, HIV-1 transactivator protein (tat) has been shown to block activation of OAS (Muller et al, J Biol Chem. 1990 Mar 5;265(7):3803-8) thus indicating that novel forms of OAS might evade HIV-1 defense mechanisms and provide an effective therapy. Thus, the OAS2 or OAS3 forms disclosed herein may confer resistance to these non-flavivirus infectious agents as well.

Each OAS2 or OAS3 cDNA is cloned from human subjects who are carriers of these mutations. Cloning is carried out by standard cDNA cloning methods that involve the isolation of RNA from cells or tissue, the conversion of RNA to cDNA, and the conversion of cDNA to double-stranded DNA suitable for cloning. As one skilled in the art will recognize, all of these steps are routine molecular biological analyses. Other methods include the use of reverse transcriptase PCR, 5′RACE (Rapid Amplification of cDNA Ends), or traditional cDNA library construction and screening by Southern hybridization. All OAS2 or OAS3 alleles described herein are recovered from patient carriers. Each newly cloned OAS2 or OAS3 cDNA is sequenced to confirm its identity and to identify any additional sequence differences relative to wild-type.

OAS2 or OAS3 gene mutations may affect resistance to viral infection by modifying the properties of the resulting OAS2 or OAS3 mRNA. Therefore, differences in mRNA stability between carriers of the resistant OAS2 or OAS3 alleles and homozygous non-resistant subjects are evaluated. RNA stability is evaluated and compared using known assays including Taqman® and simple Northern hybridization. These constitute routine methods in molecular biology.

OAS2 or OAS3 mutations may affect infection resistance by modifying the regulation of the corresponding gene. It is known that expression of OAS genes is induced by interferon treatment and during viral infection. The resistant OAS2 or OAS3 alleles may confer resistance to viral infection through constitutive expression, over-expression, or other disregulated expression. Several methods are used to evaluate gene expression with and without interferon or viral stimulation. These methods include expression microarray analysis, Northern hybridization, Taqman®, and others. Samples are collected from tissues known to express the OAS genes such as the peripheral blood mononuclear cells. Gene expression is compared between tissues from carriers of resistant OAS2 or OAS3 and non-carriers. In one embodiment, peripheral blood mononuclear cells are collected from carriers and non carriers, propagated in culture, and stimulated with interferon. The level of expression of OAS2 or OAS3 alleles during interferon induction is compared to wild-type alleles. In another embodiment, human subjects are treated with interferon and the level of induction of the OAS2 or OAS3 gene is evaluated in carriers of the resistant OAS2 or OAS3 forms versus non-carriers. As one skilled in the art can appreciate, numerous combinations of tissues, experimental designs, and methods of analysis are used to evaluate OAS2 or OAS3 gene regulation.

Once the cDNA for each OAS2 or OAS3 form is cloned, it is used to manufacture recombinant OAS2 or OAS3 proteins using any of a number of different known expression cloning systems. In one embodiment of this approach, a resistant OAS2 or OAS3 is cloned by standard molecular biological methods into an Escherichia coli expression vector adjacent to an epitope tag that contains a sequence of DNA coding for a polyhistidine polypeptide. The recombinant protein is then purified from Escherichia coli lysates using immobilized metal affinity chromatography or similar method. One skilled in the art will recognize that there are many different expression vectors and host cells that can be used to purify recombinant proteins, including but not limited to yeast expression systems, baculovirus expression systems, Chinese hamster ovary cells, and others.

Computational methods are used to identify short peptide sequences from resistant OAS2 or OAS3 proteins that uniquely distinguish these proteins from non-resistant forms. Various computational methods and commercially available software packages can be used for peptide selection. These computationally selected peptide sequences can be manufactured using the FMOC peptide synthesis chemistry or similar method. One skilled in the art will recognize that there are numerous chemical methods for synthesizing short polypeptides according to a supplied sequence.

Peptide fragments and the recombinant protein from the resistant OAS2 or OAS3 gene can be used to develop antibodies specific to this gene product. As one skilled in the art will recognize, there are numerous methods for antibody development involving the use of multiple different host organisms, adjuvants, etc. In one classic embodiment, a small amount (150 micrograms) of purified recombinant protein is injected subcutaneously into the backs of New Zealand White Rabbits with subsequent similar quantities injected every several months as boosters. Rabbit serum is then collected by venipuncture and the serum, purified IgG, or affinity purified antibody specific to the immunizing protein can be collected. As one skilled in the art will recognize, similar methods can be used to develop antibodies in rat, mouse, goat, and other organisms. Peptide fragments as described above can also be used to develop antibodies specific to the resistant OAS2 or OAS3 protein. The development of both monoclonal and polyclonal antibodies is suitable for practicing the invention. The generation of mouse hybridoma cell lines secreting specific monoclonal antibodies to the resistant OAS2 or OAS3 proteins can be carried out by standard molecular techniques.

Antibodies prepared as described above can be used to develop diagnostic methods for evaluating the presence or absence of the resistant OAS2 or OAS3 proteins in cells, tissues, and organisms. In one embodiment of this approach, enzyme-linked immunosorbent assays can be developed using purified recombinant, resistant OAS2 or OAS3 proteins and specific antibodies in order to detect these proteins in human serum. These diagnostic methods can be used to validate the presence or absence of OAS proteins in the tissues of carriers and non-carriers of the above-described genetic mutations.

Antibodies prepared as described above can also be used to purify OAS2 or OAS3 proteins from those patients who carry any of the mutational forms of the present invention. Numerous methods are available for using antibodies to purify proteins from human cells and tissues. In one embodiment, antibodies can be used in immunoprecipitation experiments involving homogenized human tissues and antibody capture using protein A. This method enables the concentration and further evaluation of any of the OAS2 or OAS3 proteins of the present invention. Numerous other methods for isolating the forms of OAS2 or OAS3 are available including column chromatography, affinity chromatography, high pressure liquid chromatography, salting-out, dialysis, electrophoresis, isoelectric focusing, differential centrifugation, and others.

Proteomic methods are used to evaluate the effect of OAS2 or OAS3 mutations on secondary, tertiary, and quaternary protein structure. Proteomic methods are also used to evaluate the impact of OAS2 or OAS3 mutations on the post-translational modification of the OAS protein. There are many known possible post-translational modifications to a protein including protease cleavage, myristoylation, glycosylation, phosphorylation, sulfation, the addition of chemical groups or complex molecules, and the like. A common method for evaluating secondary and tertiary protein structure is nuclear magnetic resonance (NMR) spectroscopy. NMR is used to probe differences in secondary and tertiary structure between resistant and non-resistant OAS proteins. Modifications to traditional NMR are also suitable, including methods for evaluating the activity of functional sites including Transfer Nuclear Overhauser Spectroscopy (TrNOESY) and others. As one skilled in the art will recognize, numerous minor modifications to this approach and methods for data interpretation of results can be employed. All of these methods are intended to be included in practicing this invention. Other methods for determining protein structure by crystallization and X-ray diffraction are employed.

Mass spectroscopy can also be used to evaluate differences between resistant and non-resistant OAS proteins. This method can be used to evaluate structural differences as well as differences in the post-translational modifications of proteins. In one typical embodiment of this approach, the resistant and non-resistant OAS proteins are purified from human peripheral blood mononuclear cells using one of the methods described above. These cells can be stimulated with interferon, as described above, in order to increase expression of the OAS proteins. Purified proteins are digested with specific proteases (e.g. trypsin) and evaluated using mass spectrometry. As one skilled in the art will recognize, many alternative methods can also be used. This invention contemplates these additional alternative methods. For instance, either matrix-assisted laser desorption/ionization (MALDI) or electrospray ionization (ESI) mass spectrometric methods can be used. Furthermore, mass spectroscopy can be coupled with the use of two-dimensional gel electrophoretic separation of cellular proteins as an alternative to comprehensive pre-purification. Mass spectrometry can also be coupled with the use of peptide fingerprint database and various searching algorithms. Differences in post-translational modification, such as phosphorylation or glycosylation, can also be probed by coupling mass spectrometry with the use of various pretreatments such as with glycosylases and phosphatases. All of these methods are to be considered as part of this application.

OAS2 is believed to form dimers, and mutations that interfere with self-association may therefore affect enzyme activity. Known methods are used to evaluate the effect of OAS2 mutations on dimer formation. For instance, immunoprecipitation with OAS2 form-specific antibodies is performed in order to isolate OAS2 complexes from patient cells, cell culture, or transfected cells over-expressing the desired OAS2 forms. These complexes can then be evaluated by gel electrophoresis or other chromatographic methods which are well known to those skilled in the art.

The OAS2 and OAS3 proteins are enzymes that catalyze the conversion of ATP into oligoadenylate molecules. Several methods are available to evaluate the activity of OAS enzymes. These methods are employed to determine the effects of OAS2 or OAS3 mutations on the activity of the mutant proteins relative to the wild type enzyme. For example, oligoadenylate synthesis activity can be measured by quantifying the incorporation of ³²P-radiolabeled ATP into polyadenylates. The radiolabeled polyadenylates can be quantified and characterized in terms of length by a number of chromatographic methods including electrophoresis or ion exchange chromatography. These assays also enable characterization of substrate (ATP) binding and enzyme kinetics. OAS2 and OAS3 are activated by dsRNA. The kinetics of this activation is analyzed in OAS2 and OAS3 and compared between resistant and non-resistant forms using the activity assays described herein and synthetic dsRNAs as described in the art.

The polypeptides of the present invention are demonstrated by these and other methods known in the art to possess oligoadenylate synthesis activity. Regardless of their quantitative level of activity, this capacity to produce 2′-5′-oligodenylates is well understood by those skilled in the art to produce anti-viral effects through the activation of RNaseL. As such, the mere fact that the polypeptides of the present invention possess oligoadenylate synthesis activity indicates that said polypeptides have utility, particularly in consideration of therapeutic uses thereof which are disclosed below.

Biological studies are performed to evaluate the degree to which resistant OAS2 or OAS3 genes protect from viral infection. These biological studies generally take the form of introducing the OAS gene or protein in question into cells or whole organisms, and evaluating their biological and antiviral activities relative to wild-type controls. In one typical embodiment of this approach, the OAS genes are introduced into African Green monkey kidney (Vero) cells in culture by cloning the cDNAs isolated as described herein into a mammalian expression vector that drives expression of the cloned cDNA from an SV40 promoter sequence. This vector will also contain SV40 and cytomegalovirus enhancer elements that permit efficient expression of the OAS genes, and a neomycin resistance gene for selection in culture. The biological effects of OAS expression can then be evaluated in Vero cells infected with the dengue virus. In the event that OAS confers broad resistance to multiple flaviviruses, one would expect an attenuation of viral propagation in cell lines expressing these resistant forms of OAS relative to non-resistant forms. As one skilled in the art will recognize, there are multiple different experimental approaches that can be used to evaluate the biological effects of OAS genes and proteins in cells and organisms and in response to different infectious agents. For instance, in the above example, different expression vectors, cell types, and viral species may be used to evaluate the OAS resistance effects. Primary human cells in culture may be evaluated as opposed to cell lines. Cells may be stimulated with double-stranded RNA or interferon before introduction of the virus. Expression vectors containing alternative promoter and enhancer sequences may be evaluated. Viruses other than the flaviviruses (e.g. respiratory syncytial virus and picornavirus) are evaluated.

Transgenic animal models are developed to assess the usefulness of particular OAS gene forms in protecting against whole-organism viral infection. In one embodiment, OAS genes are introduced into the genomes of mice susceptible to flavivirus infection (e.g. the C3H/He inbred laboratory strain). These OAS genes are evaluated for their ability to modify infection or confer resistance to infection in susceptible mice. As one skilled in the art will appreciate, numerous standard methods can be used to introduce transgenic human OAS genes into mice. These methods can be combined with other methods that affect tissue specific expression patterns or that permit regulation of the transgene through the introduction of endogenous chemicals, the use of inducible or tissue specific promoters, etc.

As a model for hepatitis C infection, cell lines expressing OAS genes can be evaluated for susceptibility, resistance, or modification of infection with the bovine diarrheal virus (BVDV). BVDV is a commonly used model for testing the efficacy of potential anti-HCV antiviral drugs (Buckwold et. al., Antiviral Research 60:1-15, 2003). In one embodiment, resistant OAS2 or OAS3 genes can be introduced into KL (calf lung) cells using expression vectors essentially as described above and tested for their ability to modify BVDV infection in this cell line. Furthermore, mouse models of HCV infection (e.g. the transplantation of human livers into mice, the infusion of human hepatocyte into mouse liver, etc.) may also be evaluated for modification of HCV infection in the transgenic setting of resistant OAS2 or OAS3 genes. Experiments can be performed whereby the effects of expression of OAS genes are assessed in HCV viral culture systems.

Cell culture systems can also be used to assess the impact of the resistant OAS gene on promoting apoptosis under varying conditions. In one embodiment, cell culture forms of resistant OAS2 or OAS3 can be assessed relative to non-resistant OAS sequences for their ability to promote apoptosis in cells infected with a number of viruses including BVDV, HCV, and other flaviviruses. As one skilled in the art will recognize, numerous methods for measuring apoptosis are available. The most common method involves the detection of the characteristic genomic “DNA laddering” effect in apoptosing cells using fluorescent conjugation methods coupled to agarose gel electrophoresis.

The ability of defective interfering viruses to potentiate the effects of resistant OAS2 or OAS3 forms can be tested in cell culture and in small animal models.

The degree to which the presence or absence of a particular OAS2 or OAS3 genotype affects other human phenotypes can also be examined. For instance, OAS2 or OAS3 mutations are evaluated for their association with viral titer and spontaneous viral clearance in HCV infected subjects. Similar methods of correlating host OAS2 or OAS3 genotype with the course of other flavivirus infections can also be undertaken. The impact of these OAS mutations on promoting successful outcomes during interferon or interferon with ribavirin treatment in HCV infected patients is also examined. These mutations may not only confer a level of infection resistance, but also promote spontaneous viral clearance in infected subjects with or without interferon-ribavirin treatment. Furthermore, it has been reported that schizophrenia occurs at a higher frequency in geographic areas that are endemic for flavivirus infection, suggesting an association between flavivirus resistance alleles and predisposition to schizophrenia. This link is evaluated by performing additional genetic association studies involving the schizophrenia phenotype and the OAS2 or OAS3 mutations. The impact of OAS2 or OAS3 mutations on susceptibility to IDDM, prostate and other cancers, and schizophrenia will also be evaluated.

The present invention discloses OAS2 and OAS3 variant mRNAs that also have utility. The invention is not limited by the mode of use of the disclosed variant mRNAs. In one preferred embodiment, these variant mRNAs are used in differentially screening human subjects for increased or decreased viral (including HCV) susceptibility. In other preferred embodiments, these variant mRNAs are useful in screening for susceptibility to IDDM, prostate and other cancers, and/or schizophrenia. Such differential screening is performed by expression analyses known to those skilled in the art to determine relative amounts of one or more variant OAS mRNAs present in samples derived from a given human subject. Increased or decreased amounts of one or more OAS mRNA variants in a human subject's sample relative to a control sample is indicative of the subject's degree of susceptibility to viral, IDDM, prostate and other cancers, and/or schizophrenia, as appropriate to the test under consideration.

As discussed herein, 2′,5′-oligoadenylate synthetases (OAS) are a family of IFN-a-inducible, RNA dependent effector molecules enzymes that synthesize short 2′ to 5′ linked oligoadenylate (2-5A) molecules from ATP. OAS enzymes constitute an important part of the nonspecific immune defense against viral infections and have been used as a cellular marker for viral infection. In addition to the role in hepatitis C infection discussed herein, OAS activity is implicated in other disease states, particularly those in which a viral infection plays a role.

While specific pathogenic mechanisms are subjects of current analysis, viral infections are believed to play a role in the development of diseases such as diabetes. Lymphocytic OAS activity is significantly elevated in patients with type 1 diabetes, suggesting that OAS may be an important link between viral infections and disease development. In a study involving diabetic twins from monozygotic twin pairs, Bonnevie-Nielsen et al. (Clin Immunol. 2000 Jul;96(1):11-8) showed that OAS is persistently activated in both recent-onset and long-standing type 1 diabetes. Field et al. (Diabetes. 2005 May;54(5):1588-91) have further shown both elevated basal OAS activity and type 1 diabetes are associated with an allele of at least one polymorphism within the OAS gene cluster. Continuously elevated OAS activity in type 1 diabetes is clearly different from a normal antiviral response and might indicate a chronic stimulation of the enzyme, a failure of down regulatory mechanisms, or an aberrant response to endogenous or exogenous viruses or their products.

A more direct link between a viral infection and the development of diabetes is exemplified by a number of studies showing that between 13 and 33% of patients with chronic hepatitis C have diabetes mellitus (type 2 diabetes), a level that is significantly increased compared with that in matched healthy controls or patients with chronic hepatitis B (Knobler et al. Am J Gastroenterol. 2003 Dec;98(12):2751-6). While OAS has not to date been reported to play a role in the development of diabetes mellitus following hepatitis C infection, it may be a useful marker for the antiviral response system. Furthermore, the results reported according to the present invention illustrate that if hepatitis C infection is causally related to diabetes mellitus, inhibition or abolition of hepatitis C infection using the compositions and methods disclosed herein may be advantageous in preventing or alleviating development of diabetes mellitus.

A further published study has shown that OAS plays an essential role in wound healing and its pathological disorders, particularly in the case of venous ulcers and diabetes-associated poorly-healing wounds (WO 02/090552). In the case of poor wound healing, OAS mRNA levels in the affected tissues were reduced, rather than elevated as in lymphocytes derived from patients suffering from type 1 diabetes. These findings point to OAS as an etiologically important marker of immune reactions in diabetes and diabetes-related wound healing.

OAS may also play an intermediary role in cell processes involved in prostate cancer. A primary biochemical function of OAS is to promote the activity of RNaseL, a uniquely-regulated endoribonuclease that is enzymatically stimulated by 2-5A molecules. RNaseL has a well-established role in mediating the antiviral effects of IFN, and is a strong candidate for the hereditary prostate cancer 1 allele (HPCl). Mutations in RNaseL have been shown to predispose men to an increased incidence of prostate cancer, which in some cases reflect more aggressive disease and/or decreased age of onset compared with non RNase L-linked cases. Xiang et al. (Cancer Res. 2003 Oct 15;63(20):6795-801) demonstrated that biostable phosphorothiolate analogs of 2-5A induced RNaseL activity and caused apoptosis in cultures of late-stage metastatic human prostate cancer cell lines. Their findings suggest that the elevation of OAS activity with a concurrent increase in 2-5A levels may facilitate the destruction of cancer cells through a potent apoptotic pathway. Thus, use of compositions and methods disclosed herein may find utility in the detection, treatment and/or prevention of prostate cancer.

OAS may further play a role in normal cell growth regulation, either through its regulation of RNaseL or through another as yet undiscovered pathway. There is considerable evidence to support the importance of OAS in negatively regulating cell growth. Rysiecki et al. (J. Interferon Res. 1989 Dec;9(6):649-57) demonstrated that stable transfection of human OAS into a glioblastoma cell line results in reduced cellular proliferation. OAS levels have also been shown to be measurable in several studies comparing quiescent versus proliferating cell lines (e.g. Hassel and Ts'O, Mol Carcinog. 1992;5(1):41-51 and Kimchi et al., Eur J Biochem. 1981;1 14(1):5-10) and in each case the OAS levels were greatest in quiescent cells. Other studies have shown a correlation between OAS level and cell cycle phase, with OAS levels rising sharply during late S phase and then dropping abruptly in G2 (Wells and Mallucci, Exp Cell Res. 1985 Jul;159(1):27-36). Several studies have shown a correlation between the induction of OAS and the onset of antiproliferative effects following stimulation with various forms of interferon (see Player and Torrence, Pharmacol Ther. 1998 May;78(2):55-113). Induction of OAS has also been shown during cell differentiation (e.g. Salzberg et al., J Cell Sci. 1996 Jun; 109(Pt 6):1517-26 and Schwartz and Nilson, Mol Cell Biol. 1989 Sep;9(9):3897-903). Other reports of induction of OAS by platelet derived growth factor (PDGF) (Zullo et al. Cell. 1985 Dec;43(3 Pt 2):793-800) and under conditions of heat-shock induced growth (Chousterman et al., J Biol Chem. 1987 Apr 5;262(10):4806-11) lead to the hypothesis that induction of OAS is a normal cell growth control mechanism. Thus, use of compositions and methods disclosed herein may find broad utility in the detection, treatment and/or prevention of cancer.

Polynucleotide Analysis

An oligoadenylate synthetase gene is a nucleic acid whose nucleotide sequence codes for oligoadenylate synthetase, a variant oligoadenylate synthetase, or oligoadenylate synthetase pseudogene. It can be in the form of genomic DNA, an mRNA or cDNA, and in single or double stranded form. Preferably, genomic DNA is used because of its relative stability in biological samples compared to mRNA. Reference genomic sequences for the oligoadenylate synthetase 3 and 2 genes, respectively, are provided in FIGS. 1 and 2 as SEQUENCE: 1 and SEQUENCE:2, respectively. As used in the present invention, these reference sequences may be modified with any one or more of the variant allele states of the mutations described above and detailed in FIGS. 4 and 5.

The nucleic acid sample is obtained from cells, typically peripheral blood leukocytes. Where mRNA is used, the cells are lysed under RNase inhibiting conditions. In one embodiment, the first step is to isolate the total cellular mRNA. Poly A+mRNA can then be selected by hybridization to an oligo-dT cellulose column.

In preferred embodiments, the nucleic acid sample is enriched for a presence of oligoadenylate synthetase allelic material. Enrichment is typically accomplished by subjecting the genomic DNA or mRNA to a primer extension reaction employing a polynucleotide synthesis primer as described herein. Particularly preferred methods for producing a sample to be assayed use preselected polynucleotides as primers in a polymerase chain reaction (PCR) to form an amplified (PCR) product.

Preparation of Polynucleotide Primers

The term “polynucleotide” as used herein in reference to primers, probes and nucleic acid fragments or segments to be synthesized by primer extension is defined as a molecule comprised of two or more deoxyribonucleotides or ribonucleotides, preferably more than three. Its exact size will depend on many factors, which in turn depends on the ultimate conditions of use.

The term “primer” as used herein refers to a polynucleotide whether purified from a nucleic acid restriction digest or produced synthetically, which is capable of acting as a point of initiation of nucleic acid synthesis when placed under conditions in which synthesis of a primer extension product which is complementary to a nucleic acid strand is induced, i.e., in the presence of nucleotides and an agent for polymerization such as DNA polymerase, reverse transcriptase and the like, and at a suitable temperature and pH. The primer is preferably single stranded for maximum efficiency, but may alternatively be in double stranded form. If double stranded, the primer is first treated to separate it from its complementary strand before being used to prepare extension products. Preferably, the primer is a polydeoxyribonucleotide. The primer must be sufficiently long to prime the synthesis of extension products in the presence of the agents for polymerization. The exact lengths of the primers will depend on many factors, including temperature and the source of primer. For example, depending on the complexity of the target sequence, a polynucleotide primer typically contains 15 to 25 or more nucleotides, although it can contain fewer nucleotides. Short primer molecules generally require cooler temperatures to form sufficiently stable hybrid complexes with template.

The primers used herein are selected to be “substantially” complementary to the different strands of each specific sequence to be synthesized or amplified. This means that the primer must be sufficiently complementary to non-randomly hybridize with its respective template strand. Therefore, the primer sequence may or may not reflect the exact sequence of the template. For example, a non-complementary nucleotide fragment can be attached to the 5′ end of the primer, with the remainder of the primer sequence being substantially complementary to the strand. Such non-complementary fragments typically code for an endonuclease restriction site. Alternatively, non-complementary bases or longer sequences can be interspersed into the primer, provided the primer sequence has sufficient complementarity with the sequence of the strand to be synthesized or amplified to non-randomly hybridize therewith and thereby form an extension product under polynucleotide synthesizing conditions.

Primers of the present invention may also contain a DNA-dependent RNA polymerase promoter sequence or its complement. See for example, Krieg, et al., Nucl. Acids Res., 12:7057-70 (1984); Studier, et al., J. Mol. Biol., 189:113-130(1986); and Molecular Cloning: A Laboratory Manual, Second Edition, Maniatis, et al., eds., Cold Spring Harbor, N.Y. (1989).

When a primer containing a DNA-dependent RNA polymerase promoter is used, the primer is hybridized to the polynucleotide strand to be amplified and the second polynucleotide strand of the DNA-dependent RNA polymerase promoter is completed using an inducing agent such as E. coli DNA polymerase I, or the Klenow fragment of E. coli DNA polymerase. The starting polynucleotide is amplified by alternating between the production of an RNA polynucleotide and DNA polynucleotide.

Primers may also contain a template sequence or replication initiation site for a RNA-directed RNA polymerase. Typical RNA-directed RNA polymerase include the QB replicase described by Lizardi, et al., Biotechnology, 6:1197-1202 1988). RNA-directed polymerases produce large numbers of RNA strands from a small number of template RNA strands that contain a template sequence or replication initiation site. These polymerases typically give a one million-fold amplification of the template strand as has been described by Kramer, et al., J. Mol. Biol., 89:719-736 (1974).

The polynucleotide primers can be prepared using any suitable method, such as, for example, the phosphotriester or phosphodiester methods see Narang, et al., Meth. Enzymol., 68:90, (1979); U.S. Pat. Nos. 4,356,270, 4,458,066, 4,416,988, 4,293,652; and Brown, et al., Meth. Enzymol., 68:109 (1979).

The choice of a primer's nucleotide sequence depends on factors such as the distance on the nucleic acid from the hybridization point to the region coding for the mutation to be detected, its hybridization site on the nucleic acid relative to any second primer to be used, and the like.

If the nucleic acid sample is to be enriched for oligoadenylate synthetase gene material by PCR amplification, two primers, i.e., a PCR primer pair, must be used for each coding strand of nucleic acid to be amplified. The first primer becomes part of the non-coding (anti-sense or minus or complementary) strand and hybridizes to a nucleotide sequence on the plus or coding strand. Second primers become part of the coding (sense or plus) strand and hybridize to a nucleotide sequence on the minus or non-coding strand. One or both of the first and second primers can contain a nucleotide sequence defining an endonuclease recognition site. The site can be heterologous to the oligoadenylate synthetase gene being amplified.

In one embodiment, the present invention utilizes a set of polynucleotides that form primers having a priming region located at the 3′-terminus of the primer. The priming region is typically the 3′-most (3′-terminal) 15 to 30 nucleotide bases. The 3′-terminal priming portion of each primer is capable of acting as a primer to catalyze nucleic acid synthesis, i.e., initiate a primer extension reaction off its 3′ terminus. One or both of the primers can additionally contain a 5′-terminal (5′-most) non-priming portion, i.e., a region that does not participate in hybridization to the preferred template.

In PCR, each primer works in combination with a second primer to amplify a target nucleic acid sequence. The choice of PCR primer pairs for use in PCR is governed by considerations as discussed herein for producing oligoadenylate synthetase gene regions. When a primer sequence is chosen to hybridize (anneal) to a target sequence within an oligoadenylate synthetase gene allele intron, the target sequence should be conserved among the alleles in order to insure generation of target sequence to be assayed.

Polymerase Chain Reaction

Oligoadenylate synthetase genes are comprised of polynucleotide coding strands, such as mRNA and/or the sense strand of genomic DNA. If the genetic material to be assayed is in the form of double stranded genomic DNA, it is usually first denatured, typically by melting, into single strands. The nucleic acid is subjected to a PCR reaction by treating (contacting) the sample with a PCR primer pair, each member of the pair having a preselected nucleotide sequence. The PCR primer pair is capable of initiating primer extension reactions by hybridizing to nucleotide sequences, preferably at least about 10 nucleotides in length, more preferably at least about 20 nucleotides in length, conserved within the oligoadenylate synthetase alleles. The first primer of a PCR primer pair is sometimes referred to herein as the “anti-sense primer” because it hybridizes to a non-coding or anti-sense strand of a nucleic acid, i.e., a strand complementary to a coding strand. The second primer of a PCR primer pair is sometimes referred to herein as the “sense primer” because it hybridizes to the coding or sense strand of a nucleic acid.

The PCR reaction is performed by mixing the PCR primer pair, preferably a predetermined amount thereof, with the nucleic acids of the sample, preferably a predetermined amount thereof, in a PCR buffer to form a PCR reaction admixture. The admixture is thermocycled for a number of cycles, which is typically predetermined, sufficient for the formation of a PCR reaction product, thereby enriching the sample to be assayed for oligoadenylate synthetase genetic material.

PCR is typically carried out by thermocycling i.e., repeatedly increasing and decreasing the temperature of a PCR reaction admixture within a temperature range whose lower limit is about 30 degrees Celsius (30° C.) to about 55° C. and whose upper limit is about 90° C. to about 100° C. The increasing and decreasing can be continuous, but is preferably phasic with time periods of relative temperature stability at each of temperatures favoring polynucleotide synthesis, denaturation and hybridization.

A plurality of first primer and/or a plurality of second primers can be used in each amplification, e.g., one species of first primer can be paired with a number of different second primers to form several different primer pairs. Alternatively, an individual pair of first and second primers can be used. In any case, the amplification products of amplifications using the same or different combinations of first and second primers can be combined for assaying for mutations.

The PCR reaction is performed using any suitable method. Generally it occurs in a buffered aqueous solution, i.e., a PCR buffer, preferably at a pH of 7-9, most preferably about 8. Preferably, a molar excess (for genomic nucleic acid, usually about 10⁶:1 primer:template) of the primer is admixed to the buffer containing the template strand. A large molar excess is preferred to improve the efficiency of the process.

The PCR buffer also contains the deoxyribonucleotide triphosphates (polynucleotide synthesis substrates) dATP, dCTP, dGTP, and dTTP and a polymerase, typically thermostable, all in adequate amounts for primer extension (polynucleotide synthesis) reaction. The resulting solution (PCR admixture) is heated to about 90° C. -100° C. for about 1 to 10 minutes, preferably from 1 to 4 minutes. After this heating period the solution is allowed to cool to 54° C., which is preferable for primer hybridization. The synthesis reaction may occur at from room temperature up to a temperature above which the polymerase (inducing agent) no longer functions efficiently. The thermocycling is repeated until the desired amount of PCR product is produced. An exemplary PCR buffer comprises the following: 50 mM KCl; 10 mM Tris-HCl at pH 8.3; 1.5 mM MgCl.; 0.001% (wt/vol) gelatin, 200 μM dATP; 200 μM dTTP; 200 μM dCTP; 200² μM dGTP; and 2.5 units Thermus aquaticus (Taq) DNA polymerase I (U.S. Pat. No. 4,889,818) per 100 microliters of buffer.

The inducing agent may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There may be inducing agents, however, which initiate synthesis at the 5′ end and proceed in the above direction, using the same process as described above.

The inducing agent also may be a compound or system which will function to accomplish the synthesis of RNA primer extension products, including enzymes. In preferred embodiments, the inducing agent may be a DNA-dependent RNA polymerase such as T7 RNA polymerase, T3 RNA polymerase or SP6 RNA polymerase. These polymerases produce a complementary RNA polynucleotide. The high turn-over rate of the RNA polymerase amplifies the starting polynucleotide as has been described by Chamberlin, et al., The Enzymes, ed. P. Boyer, pp. 87-108, Academic Press, New York (1982). Amplification systems based on transcription have been described by Gingeras, et al., in PCR Protocols, A Guide to Methods and Applications, pp. 245-252, Innis, et al., eds, Academic Press, Inc., San Diego, Calif. (1990).

If the inducing agent is a DNA-dependent RNA polymerase and, therefore incorporates ribonucleotide triphosphates, sufficient amounts of ATP, CTP, GTP and UTP are admixed to the primer extension reaction admixture and the resulting solution is treated as described above.

The newly synthesized strand and its complementary nucleic acid strand form a double-stranded molecule which can be used in the succeeding steps of the process.

The PCR reaction can advantageously be used to incorporate into the product a preselected restriction site useful in detecting a mutation in the oligoadenylate synthetase gene.

PCR amplification methods are described in detail in U.S. Pat. Nos. 4,683,192, 4,683,202, 4,800,159, and 4,965,188, and at least in several texts including PCR Technology: Principles and Applications for DNA Amplification, H. Erlich, ed., Stockton Press, New York (1989); and PCR Protocols: A Guide to Methods and Applications, Innis, et al., eds., Academic Press, San Diego, Calif. (1990).

In some embodiments, two pairs of first and second primers are used per amplification reaction. The amplification reaction products obtained from a plurality of different amplifications, each using a plurality of different primer pairs, can be combined or assayed separately.

However, the present invention contemplates amplification using only one pair of first and second primers. Exemplary primers for amplifying the sections of DNA containing the mutations disclosed herein are shown below in Table 1.

TABLE 1
Amplicons Containing Mutations of the Present Invention

| | | |
|---|---|---|
| Amplicon | Primer A | Primer B |

| | | |
|---|---|---|
| Amplicon2001 | 5′-GCAGGAGTTGGTAAACTCAC-3′ | 5′-GAGGTTAAGTAGCCTGCCCA-3′ |
| | (SEQUENCE:16) | (SEQUENCE:17) |
| Amplicon2002 | 5′-GCCTGCCACTCAATGTTAAG-3′ | 5′-ACTCATGGCCTAGAGGTTGC-3′ |
| | (SEQUENCE:18) | (SEQUENCE:19) |
| Amplicon2003 | 5′-ATCTAATGGGCCAAGTCACC-3′ | 5′-GGTACACGAAACGTTCCCTA-3′ |
| | (SEQUENCE:20) | (SEQUENCE:21) |
| Amplicon2006 | 5′- TCTTGTGTGCCACTCCAAAC-3′ | 5′-GAGCTACAATGCCCACTTAC-3′ |
| | (SEQUENCE:22) | (SEQUENCE:23) |
| Amplicon2008 | 5′-TATTCTGGAGATGCTCCCTG-3′ | 5′-TGGGCAGATTCTCCAAAGTG-3′ |
| | (SEQUENCE:24) | (SEQUENCE:25) |
| Amplicon2009 | 5′-GACATCCAAGCTGCAGAGTG-3′ | 5′-CTGTTGGCTAGCACTTTCCC-3′ |
| | (SEQUENCE:26) | (SEQUENCE:27) |
| Amplicon2011 | 5′-ACTACAAGTGATCCTCAGGC-3′ | 5′-GTGCAAGGGTTCTCACCTAG-3′ |
| | (SEQUENCE:28) | (SEQUENCE:29) |
| Amplicon2012 | 5′-ACTCACATTTGGGGCTAGAC-3′ | 5′-GGAGTTCAGCAAGGCAAGAC-3′ |
| (SEQUENCE:30) | (SEQUENCE:31) | |
| Amplicon2013 | 5′-GTTGTGGAGCTAGGATCCAT-3′ | 5′-GAGGTTA~AGCACCTAGACC-3′ |
| | (SEQUENCE:32) | (SEQUENCE:33) |
| Amplicon2014 | 5′-GACATCCTCTATGCCAGCAG-3′ | 5′- CCATGGGTAACCTTGTTAGC-3′ |
| | (SEQUENCE:34) | (SEQUENCE:35) |
| Amplicon2016 | 5′-GTTACTTTGAACCCTACTAGTA-3′ | 5′-GCTTTCAGGGCCATAAGTAC-3′ |
| | (SEQUENCE:36) | (SEQUENCE:37) |
| Amplicon2017 | 5′-TTTCTTGATTTCAGATCCCTGAC-3′ | 5′- TGGAATGTGAAAAGCACTGG-3′ |
| | (SEQUENCE:38) | (SEQUENCE:39) |
| Amplicon3001 | 5′-TGTCAGGTCCAAGAGCTGCT-3′ | 5′-TGAGGTGCACAAGCGGATAA-3′ |
| | (SEQUENCE:40) | (SEQUENCE:41) |
| Amplicon3002 | 5′-CGTGGCTTCAATGCCTACAG-3′ | 5′-CTGGGCTAGAATTGGAAGTC-3′ |
| | (SEQUENCE:42) | (SEQUENCE:43) |
| Amplicon3005 | 5′-GTGCAGCCAGGGTTGACAAT-3′ | 5′-ACCTCAGGTAATCTGCCCAC-3′ |
| | (SEQUENCE:44) | (SEQUENCE:45) |
| Amplicon3006 | 5′-AAGATGGCCATGTGCGTTAG-3′ | 5′-CAGCTCCATTGCTGTAACTC-3′ |
| | (SEQUENCE:46) | (SEQUENCE:47) |
| Amplicon3007 | 5′-TTCTAAGAGGTCACAGGACC-3′ | 5′-ACAAAGAGGATGGCAGGTGC-3′ |
| | (SEQUENCE:48) | (SEQUENCE:49) |
| Amplicon3008 | 5′-TCCAGTACAGAATTGATACTG-3′ | 5′-GCTTCCAGATCTGGGCAG-3′ |
| | (SEQUENCE:50) | (SEQUENCE:51) |
| Amplicon3009 | 5′-CTCTGAACCTCAGTTTACCC-3′ | 5′-TTGGGACTCCTTATGTCCAC-3′ |
| | (SEQUENCE:52) | (SEQUENCE:53) |
| Amplicon3010 | 5′-CAGCCAATTGAGATCGCTTC-3′ | 5′-GCTATGAGTTGTCAGCCACC-3′ |
| | (SEQUENCE: 54) | (SEQUENCE: 55) |
| Amplicon3011 | 5′-CAGGTCCTTCTGATGCTACC-3′ | 5′-CATGACCACTTTCCAGCTCT-3′ |
| | (SEQUENCE:56) | (SEQUENCE:57) |
| Amplicon3012 | 5′-GATGACTTGTCCAAGGTCAC-3′ | 5′-CGAACAGATGTGGCCTGGTT-3′ |
| | (SEQUENCE:58) | (SEQUENCE:59) |
| Amplicon3013 | 5′-GATGACTGTCACCAGGGATT-3′ | 5′-CTCAGCCATGTTGAACTGGG-3′ |
| | (SEQUENCE:60) | (SEQUENCE:61) |
| Amplicon3014 | 5′-TCAGCTGTGGGACCTTAGTT-3′ | 5′-CTATTCCTGGGTGACCAGAA-3′ |
| | (SEQUENCE:62) | (SEQUENCE:63) |
| Amplicon3016 | 5′-ATCAGCGGTCCTACTGGATG-3′ | 5′-AGGGCTCTTCAATAGCCCAC-3′ |
| | (SEQUENCE:64) | (SEQUENCE:65) |
| Ampiicon3017 | 5′-GCCACAGTCATTTGGTACTG-3′ | 5′-CTGATTCGGCTACAGTGGTC-3′ |
| | (SEQUENCE:66) | (SEQUENCE:67) |
| Amplicon3018 | 5′-ACAACCGTGCTCAGCCTGTT-3′ | 5′-ATCAGAGGAGCTTCCCTTGG-3′ |
| | (SEQUENCE:68) | (SEQUENCE:69) |
| Amplicon3019 | 5′-ATTACAGCCAGACCTCTGGC-3′ | 5′-ATGGAAGGTACCCAACTGCG-3′ |
| | (SEQUENCE:70) | (SEQUENCE:71) |
| Amplicon3020 | 5′-TCGATACTGCCTGGTAATCC-3′ | 5′-GCCACCTAACTGCATTGGTC-3′ |
| | (SEQUENCE:72) | (SEQUENCE:73) |
| Amplicon3021 | 5′-CGATGGAACCAGGTAAGTTG-3′ | 5′-CAGGGTTTCCTTTTAGGGTG-3′ |
| | (SEQUENCE:74) | (SEQUENCE:75) |
| Amplicon3025 | 5′-AATAGCACCTACACCATGGTCG-3′ | 5′-TACGAACTCCTTCCGCGGCTGC-3′ |
| | (SEQUENCE:76) | (SEQUENCE:77) |
| Amplicon3026 | 5′-TGAATATTCCAAGTGATGCAGC-3′ | 5′-TCAGTCAGTTTAGGATGGTACC-3′ |
| | (SEQUENCE:78) | (SEQUENCE:79) |
| Amplicon3030 | 5′-TCTAGCCCCTGCAAAGTGTT-3′ | 5′-GCACACATGTGCTCACACAC-3′ |
| | (SEQUENCE:228) | (SEQUENCE:229) |

Table 2 discloses the position in the above Amplicons of the mutations of the invention.

TABLE 2
Position of Mutations of the Invention in Amplicons

| | | Nucleotide Position in Amplicon |
|---|---|---|
| | | (relative to 5′ end of PrimerA |
| Mutation ID | Amplicon | side of Amplicon) |
| Mutation: 7155 | Amplicon3026 | 69 |
| Mutation: 7168 | Amplicon3025 | 214 |
| Mutation: 7150 | Amplicon3025 | 551 |
| Mutation: 7165 | Amplicon3025 | 562 |
| Mutation: 7142 | Amplicon3025 | 619 |
| Mutation: 6240 | Amplicon3001 | 283 |
| Mutation: 6241 | Amplicon3001 | 347 |
| Mutation: 14100 | Amplicon3001 | 446 |
| Mutation: 13915 | Amplicon3002 | 149 |
| Mutation: 6245 | Amplicon3005 | 368 |
| Mutation: 6246 | Amplicon3006 | 108 |
| Mutation: 6247 | Amplicon3006 | 116 |
| Mutation: 6248 | Amplicon3006 | 271 |
| Mutation: 6249 | Amplicon3006 | 442 |
| Mutation: 13916 | Amplicon3006 | 527 |
| Mutation: 7158 | Amplicon3007 | 201 |
| Mutation: 6251 | Amplicon3008 | 230 |
| Mutation: 7144 | Amplicon3008 | 490 |
| Mutation: 6253 | Amplicon3009 | 532-533 |
| Mutation: 6254 | Amplicon3010 | 238 |
| Mutation: 7161 | Amplicon3010 | 251 |
| Mutation: 7164 | Amplicon3011 | 421 |
| Mutation: 6255 | Amplicon3012 | 53 |
| Mutation: 6256 | Amplicon3012 | 240 |
| Mutation: 13918 | Amplicon3013 | 64 |
| Mutation: 6257 | Amplicon3013 | 158 |
| Mutation: 7172 | Amplicon3013 | 762 |
| Mutation: 6258 | Amplicon3016 | 205 |
| Mutation: 6259 | Amplicon3016 | 420 |
| Mutation: 6260 | Amplicon3017 | 626 |
| Mutation: 6262 | Amplicon3018 | 221 |
| Mutation: 13919 | Amplicon3018 | 400 |
| Mutation: 7152 | Amplicon3018 | 484 |
| Mutation: 13920 | Amplicon3018 | 502 |
| Mutation: 14038 | Amplicon3018 | 510 |
| Mutation: 6263 | Amplicon3018 | 637 |
| Mutation: 6265 | Amplicon3019 | 581 |
| Mutation: 7153 | Amplicon3020 | 74 |
| Mutation: 14039 | Amplicon3020 | 250 |
| Mutation: 6266 | Amplicon3020 | 261 |
| Mutation: 6267 | Amplicon3020 | 596 |
| Mutation: 13614 | Amplicon3021 | 161 |
| Mutation: 13922 | Amplicon2001 | 237 |
| Mutation: 7109 | Amplicon2001 | 459 |
| Mutation: 7110 | Amplicon2002 | 174 |
| Mutation: 7111 | Amplicon2002 | 435 |
| Mutation: 13905 | Amplicon2003 | 228 |
| Mutation: 13914 | Amplicon2017 | 44 |
| Mutation: 13906 | Amplicon2017 | 201 |
| Mutation: 7112 | Amplicon2006 | 382 |
| Mutation: 7113 | Amplicon2008 | 62 |
| Mutation: 13907 | Amplicon2008 | 230 |
| Mutation: 7114 | Amplicon2008 | 363 |
| Mutation: 13636 | Amplicon2009 | 226 |
| Mutation: 13869 | Amplicon2009 | 331 |
| Mutation: 7115 | Amplicon2009 | 389 |
| Mutation: 13635 | Amplicon2009 | 433 |
| Mutation: 14077 | Amplicon2016 | 272 |
| Mutation: 13912 | Amplicon2016 | 363 |
| Mutation: 13913 | Amplicon2016 | 464 |
| Mutation: 7116 | Amplicon2011 | 368 |
| Mutation: 7117 | Amplicon2012 | 510 |
| Mutation: 7119 | Amplicon2013 | 455 |
| Mutation: 13872 | Amplicon2014 | 133 |
| Mutation: 13911 | Amplicon2014 | 171 |
| Mutation: 7124 | Amplicon2014 | 385 |
| Mutation: 15174 | Amplicon3003 | 216 |
| Mutation: 14233 | Amplicon3005 | 398 |
| Mutation: 15200 | Amplicon3030 | 276 |
| Mutation: 15186 | Amplicon3030 | 398 |
| Mutation: 15202 | Amplicon3030 | 470 |
| Mutation: 15203 | Amplicon3030 | 529 |
| Mutation: 15199 | Amplicon3030 | 546 |
| Mutation: 15198 | Amplicon3030 | 553 |
| Mutation: 15197 | Amplicon3030 | 568 |
| Mutation: 13938 | Amplicon2017 | 105 |

Nucleic Acid Sequence Analysis

Nucleic acid sequence analysis is approached by a combination of (a) physiochemical techniques, based on the hybridization or denaturation of a probe strand plus its complementary target, and (b) enzymatic reactions with endonucleases, ligases, and polymerases. Nucleic acid can be assayed at the DNA or RNA level. The former analyzes the genetic potential of individual humans and the latter the expressed information of particular cells.

In assays using nucleic acid hybridization, detecting the presence of a DNA duplex in a process of the present invention can be accomplished by a variety of means.

In one approach for detecting the presence of a DNA duplex, an oligonucleotide that is hybridized in the DNA duplex includes a label or indicating group that will render the duplex detectable. Typically such labels include radioactive atoms, chemically modified nucleotide bases, and the like.

The oligonucleotide can be labeled, i.e., operatively linked to an indicating means or group, and used to detect the presence of a specific nucleotide sequence in a target template.

Radioactive elements operatively linked to or present as part of an oligonucleotide probe (labeled oligonucleotide) provide a useful means to facilitate the detection of a DNA duplex. A typical radioactive element is one that produces beta ray emissions. Elements that emit beta rays, such as ³H, ¹²C, ³²P and ³⁵S represent a class of beta ray emission-producing radioactive element labels. A radioactive polynucleotide probe is typically prepared by enzymatic incorporation of radioactively labeled nucleotides into a nucleic acid using DNA kinase.

Alternatives to radioactively labeled oligonucleotides are oligonucleotides that are chemically modified to contain metal complexing agents, biotin-containing groups, fluorescent compounds, and the like.

One useful metal complexing agent is a lanthanide chelate formed by a lanthanide and an aromatic beta-diketone, the lanthanide being bound to the nucleic acid or oligonucleotide via a chelate-forming compound such as an EDTA-analogue so that a fluorescent lanthanide complex is formed. See U.S. Pat. Nos. 4,374,120, 4,569,790 and published Patent Application EP0139675 and W087/02708.

Biotin or acridine ester-labeled oligonucleotides and their use to label polynucleotides have been described. See U.S. Pat. No. 4,707,404, published Patent Application EP0212951 and European Patent No. 0087636. Useful fluorescent marker compounds include fluorescein, rhodamine, Texas Red, NBD and the like.

A labeled oligonucleotide present in a DNA duplex renders the duplex itself labeled and therefore distinguishable over other nucleic acids present in a sample to be assayed. Detecting the presence of the label in the duplex and thereby the presence of the duplex, typically involves separating the DNA duplex from any labeled oligonucleotide probe that is not hybridized to a DNA duplex.

Techniques for the separation of single stranded oligonucleotide, such as non-hybridized labeled oligonucleotide probe, from DNA duplex are well known, and typically involve the separation of single stranded from double stranded nucleic acids on the basis of their chemical properties. More often separation techniques involve the use of a heterogeneous hybridization format in which the non-hybridized probe is separated, typically by washing, from the DNA duplex that is bound to an insoluble matrix. Exemplary is the Southern blot technique, in which the matrix is a nitrocellulose sheet and the label is ³²P. Southern, J. Mol. Biol., 98:503 (1975).

The oligonucleotides can also be advantageously linked, typically at or near their 5′-terminus, to a solid matrix, i.e., aqueous insoluble solid support. Useful solid matrices are well known in the art and include cross-linked dextran such as that available under the tradename SEPHADEX from Pharmacia Fine Chemicals (Piscataway, N.J.); agarose, polystyrene or latex beads about 1 micron to about 5 millimeters in diameter, polyvinyl chloride, polystyrene, cross-linked polyacrylamide, nitrocellulose or nylon-based webs such as sheets, strips, paddles, plates microtiter plate wells and the like.

It is also possible to add “linking” nucleotides to the 5′ or 3′ end of the member oligonucleotide, and use the linking oligonucleotide to operatively link the member to the solid support.

In nucleotide hybridizing assays, the hybridization reaction mixture is maintained in the contemplated method under hybridizing conditions for a time period sufficient for the oligonucleotides having complementarity to the predetermined sequence on the template to hybridize to complementary nucleic acid sequences present in the template to form a hybridization product, i.e., a complex containing oligonucleotide and target nucleic acid.

The phrase “hybridizing conditions” and its grammatical equivalents, when used with a maintenance time period, indicates subjecting the hybridization reaction admixture, in the context of the concentrations of reactants and accompanying reagents in the admixture, to time, temperature and pH conditions sufficient to allow one or more oligonucleotides to anneal with the target sequence, to form a nucleic acid duplex. Such time, temperature and pH conditions required to accomplish hybridization depend, as is well known in the art, on the length of the oligonucleotide to be hybridized, the degree of complementarity between the oligonucleotide and the target, the guanine and cytosine content of the oligonucleotide, the stringency of hybridization desired, and the presence of salts or additional reagents in the hybridization reaction admixture as may affect the kinetics of hybridization. Methods for optimizing hybridization conditions for a given hybridization reaction admixture are well known in the art.

Typical hybridizing conditions include the use of solutions buffered to pH values between 4 and 9, and are carried out at temperatures from 4° C. to 37° C., preferably about 12° C. to about 30° C., more preferably about 22° C., and for time periods from 0.5 seconds to 24 hours, preferably 2 minutes (min) to 1 hour.

Hybridization can be carried out in a homogeneous or heterogeneous format as is well known. The homogeneous hybridization reaction occurs entirely in solution, in which both the oligonucleotide and the nucleic acid sequences to be hybridized (target) are present in soluble forms in solution. A heterogeneous reaction involves the use of a matrix that is insoluble in the reaction medium to which either the oligonucleotide, polynucleotide probe or target nucleic acid is bound.

Where the nucleic acid containing a target sequence is in a double stranded (ds) form, it is preferred to first denature the dsDNA, as by heating or alkali treatment, prior to conducting the hybridization reaction. The denaturation of the dsDNA can be carried out prior to admixture with an oligonucleotide to be hybridized, or can be carried out after the admixture of the dsDNA with the oligonucleotide.

Predetermined complementarity between the oligonucleotide and the template is achieved in two alternative manners. A sequence in the template DNA may be known, such as where the primer to be formed can hybridize to known oligoadenylate synthetase sequences and can initiate primer extension into a region of DNA for sequencing purposes, as well as subsequent assaying purposes as described herein, or where previous sequencing has determined a region of nucleotide sequence and the primer is designed to extend from the recently sequenced region into a region of unknown sequence. This latter process has been referred to a “directed sequencing” because each round of sequencing is directed by a primer designed based on the previously determined sequence.

Effective amounts of the oligonucleotide present in the hybridization reaction admixture are generally well known and are typically expressed in terms of molar ratios between the oligonucleotide to be hybridized and the template. Preferred ratios are hybridization reaction mixtures containing equimolar amounts of the target sequence and the oligonucleotide. As is well known, deviations from equal molarity will produce hybridization reaction products, although at lower efficiency. Thus, although ratios where one component can be in as much as 100 fold molar excess relative to the other component, excesses of less than 50 fold, preferably less than 10 fold, and more preferably less than two fold are desirable in practicing the invention.

Detection of Membrane-Immobilized Target Sequences

In the DNA (Southern) blot technique, DNA is prepared by PCR amplification as previously discussed. The PCR products (DNA fragments) are separated according to size in an agarose gel and transferred (blotted) onto a nitrocellulose or nylon membrane. Conventional electrophoresis separates fragments ranging from 100 to 30,000 base pairs while pulsed field gel electrophoresis resolves fragments up to 20 million base pairs in length. The location on the membrane a containing particular PCR product is determined by hybridization with a specific, labeled nucleic acid probe.

In preferred embodiments, PCR products are directly immobilized onto a solid-matrix (nitrocellulose membrane) using a dot-blot (slot-blot) apparatus, and analyzed by probe-hybridization. See U.S. Pat. Nos. 4,582,789 and 4,617,261.

Immobilized DNA sequences may be analyzed by probing with allele-specific oligonucleotide (ASO) probes, which are synthetic DNAn oligomers of approximately 15, 17, 20, 25 or up to about 30 nucleotides in length. These probes are long enough to represent unique sequences in the genome, but sufficiently short to be destabilized by an internal mismatch in their hybridization to a target molecule. Thus, any sequences differing at single nucleotides may be distinguished by the different denaturation behaviors of hybrids between the ASO probe and normal or mutant targets under carefully controlled hybridization conditions. Exemplary probes are disclosed herein as SEQUENCE:80-152 and SEQUENCE:230-239 (Table 3), but any probes are suitable as long as they hybridize specifically to the region of the OAS gene carrying the mutation of choice, and are capable of specifically distinguishing between polynucleotides carrying the alternate states at the site of mutation.

Detection of Target Sequences in Solution

Several rapid techniques that do not require nucleic acid purification or immobilization have been developed. For example, probe/target hybrids may be selectively isolated on a solid matrix, such as hydroxylapatite, which preferentially binds double-stranded nucleic acids. Alternatively, probe nucleic acids may be immobilized on a solid support and used to capture target sequences from solution. Detection of the target sequences can be accomplished with the aid of a second, labeled probe that is either displaced from the support by the target sequence in a competition-type assay or joined to the support via the bridging action of the target sequence in a sandwich-type format.

In the oligonucleotide ligation assay (OLA), the enzyme DNA ligase is used to covalently join two synthetic oligonucleotide sequences selected so that they can base pair with a target sequence in exact head-to-tail juxtaposition. Ligation of the two oligomers is prevented by the presence of mismatched nucleotides at the junction region. This procedure allows for the distinction between known sequence variants in samples of cells without the need for DNA purification. The joining of the two oligonucleotides may be monitored by immobilizing one of the two oligonucleotides and observing whether the second, labeled oligonucleotide is also captured.

Scanning Techniques for Detection of Base Substitutions

Three techniques permit the analysis of probe/target duplexes several hundred base pairs in length for unknown single-nucleotide substitutions or other sequence differences. In the ribonuclease (RNase) A technique, the enzyme cleaves a labeled RNA probe at positions where it is mismatched to a target RNA or DNA sequence. The fragments may be separated according to size allowing for the determination of the approximate position of the mutation. See U.S. Pat. No. 4,946,773.

In the denaturing gradient gel technique, a probe-target DNA duplex is analyzed by electrophoresis in a denaturing gradient of increasing strength. Denaturation is accompanied by a decrease in migration rate. A duplex with a mismatched base pair denatures more rapidly than a perfectly matched duplex.

A third method relies on chemical cleavage of mismatched base pairs. A mismatch between T and C, G, or T, as well as mismatches between C and T, A, or C, can be detected in heteroduplexes. Reaction with osmium tetroxide (T and C mismatches) or hydroxylamine (C mismatches) followed by treatment with piperidine cleaves the probe at the appropriate mismatch.

Therapeutic agents for restoring and/or enhancing OAS function

Where a mutation in the OAS2 or OAS3 gene leads to defective OAS function and this defective function is associated with increased susceptibility of a patient to pathogenic infection, whether through lower levels of OAS protein, mutation in the protein affecting its function, or other mechanisms, it may be advantageous to treat the patient with wild type OAS protein. Furthermore, if the mutation gives rise in infection-resistant carriers to a form of the protein that differs from the non-resistant protein, and that has an advantage in terms of inhibiting HCV infection, it may be advantageous to administer a protein encoded by the mutated gene. As described previously, administration of either native or mutant forms of OAS proteins or polypeptides may also be advantageous in the treatment of other indications including but not limited to cancer, diabetes mellitus, and wound healing. The discussion below pertains to administration of any of the foregoing proteins or polypeptides.

The polypeptides of the present invention, including those encoded by resistant OAS2 or OAS3 genes, may be a naturally purified product, or a product of chemical synthetic procedures, or produced by recombinant techniques from a prokaryotic or eukaryotic host (for example, by bacterial, yeast, higher plant, insect and mammalian cells in culture) of a polynucleotide sequence of the present invention. Depending upon the host employed in a recombinant production procedure, the polypeptides of the present invention may be glycosylated with mammalian or other eukaryotic carbohydrates or may be non-glycosylated. Polypeptides of the invention may also include an initial methionine amino acid residue (at position minus 1).

The polypeptides of the present invention also include the protein sequences defined in SEQUENCE:6-12, SEQUENCE: 14-15, and SEQUENCE:227 and derivatives thereof.

In addition to naturally occurring allelic forms of the polypeptide(s) the present invention also embraces analogs and fragments thereof, which function similarly to the naturally occurring allelic forms. Thus, for example, one or more of the amino acid residues of the polypeptide may be replaced by conserved amino acid residues, as long as the function of the resistant OAS2 or OAS3 protein is maintained. Similarly, truncated forms of the polypeptides of the present invention may also be demonstrated to be enzymatically active. Such truncated forms are characterized using methods disclosed herein to establish their enzymatic and antiviral properties. As those skilled in the art will appreciate, therapeutic use of truncated but functional forms of OAS2 or OAS3 polypeptides can preclude the development of antibody response which would otherwise hinder the therapeutic efficacy of the polypeptide. Such truncated polypeptides that can be envisioned by one skilled in the art, maintain function but remove non-ubiquitous portions of the polypeptide that could induce antibody response in individuals not possessing the full length OAS2 or OAS3 polypeptide endogenously. Those skilled in the art will also appreciate that smaller polypeptides, in general, are more amenable to the complexities of manufacturing, delivery, and clearance typically encountered in therapeutic development. The invention is not limited by the form of the fragment and specifically includes amino-terminus truncations and internal amino acid deletions that retain enzymatic function.

The polypeptides may also be employed in accordance with the present invention by expression of such polypeptides in vivo, which is often referred to as gene therapy. Thus, for example, cells may be transduced with a polynucleotide (DNA or RNA) encoding the polypeptides ex vivo with those transduced cells then being provided to a patient to be treated with the polypeptide. Such methods are well known in the art. For example, cells may be transduced by procedures known in the art by use of a retroviral particle containing RNA encoding the polypeptide of the present invention.

Similarly, transduction of cells may be accomplished in vivo for expression of the polypeptide in vivo, for example, by procedures known in the art. As known in the art, a producer cell for producing a retroviral particle containing RNA encoding the polypeptides of the present invention may be administered to a patient for transduction in vivo and expression of the polypeptides in vivo.

These and other methods for administering the polypeptides of the present invention by such methods should be apparent to those skilled in the art from the teachings of the present invention. For example, the expression vehicle for transducing cells may be other than a retrovirus, for example, an adenovirus which may be used to transduce cells in vivo after combination with a suitable delivery vehicle.

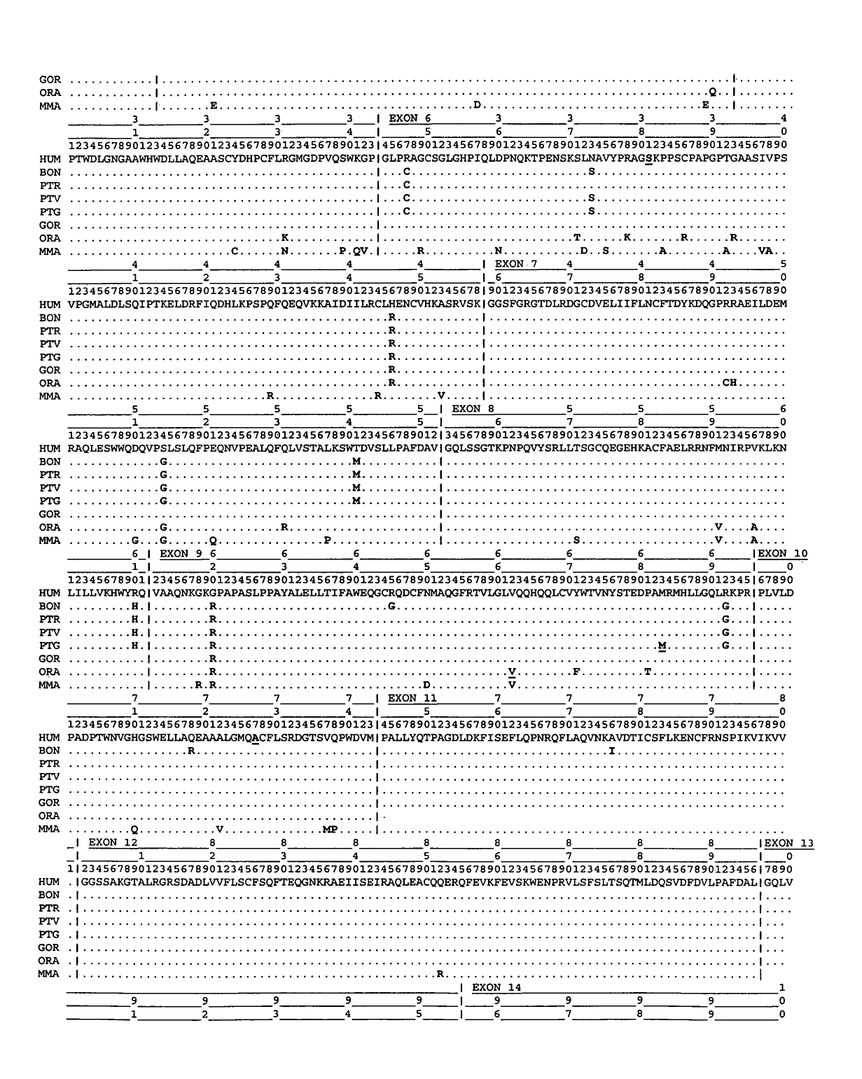

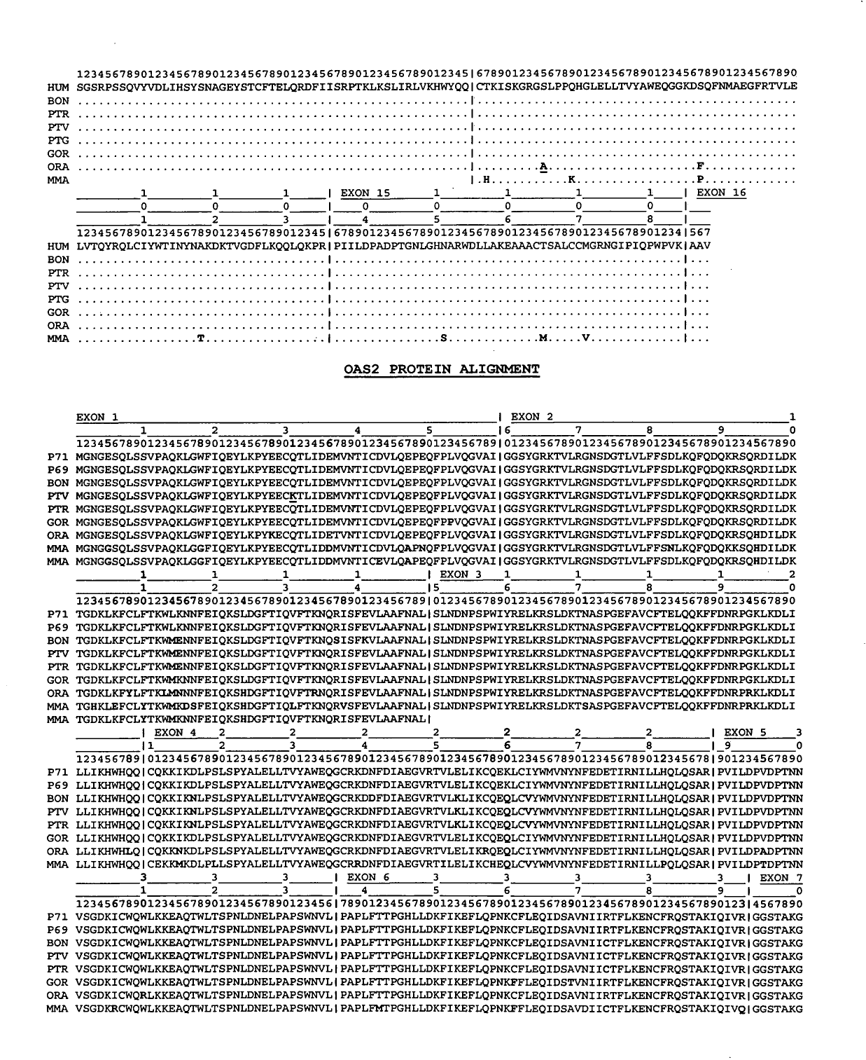

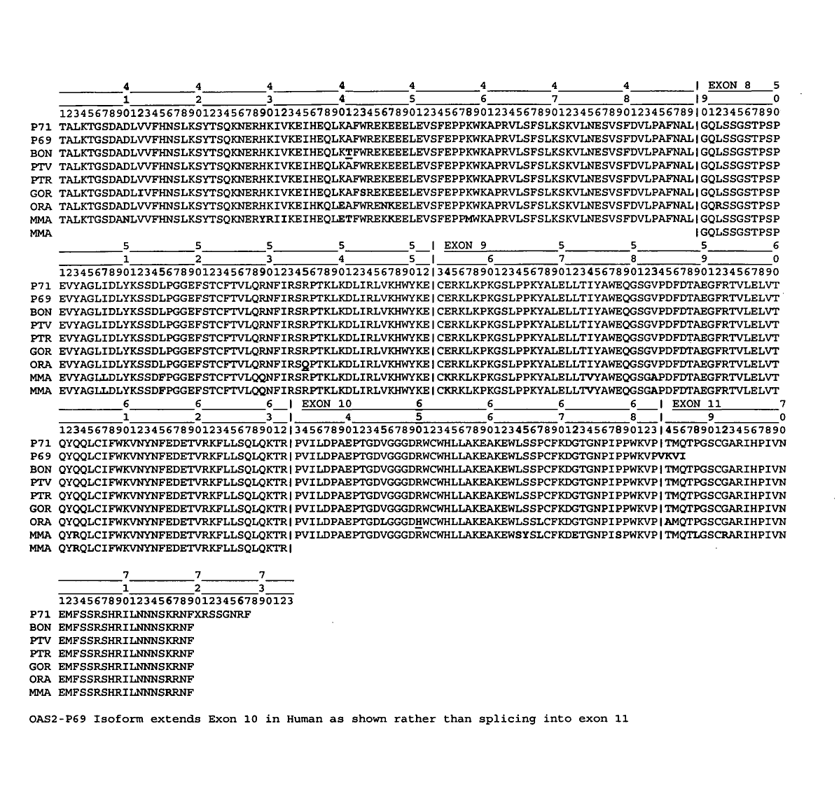

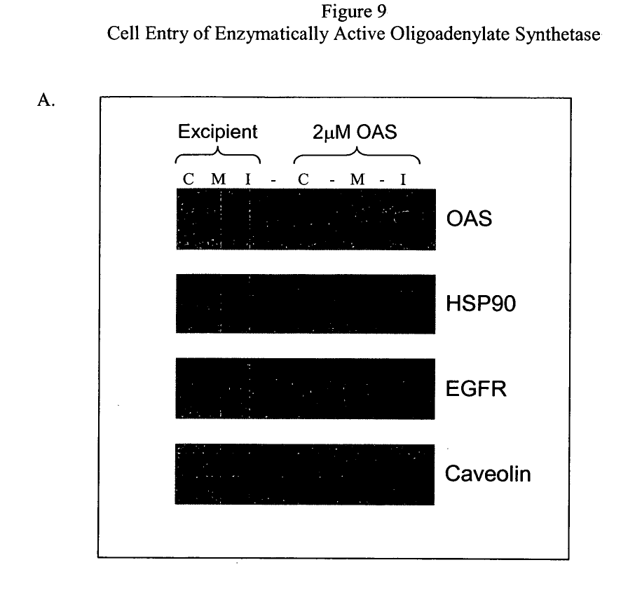

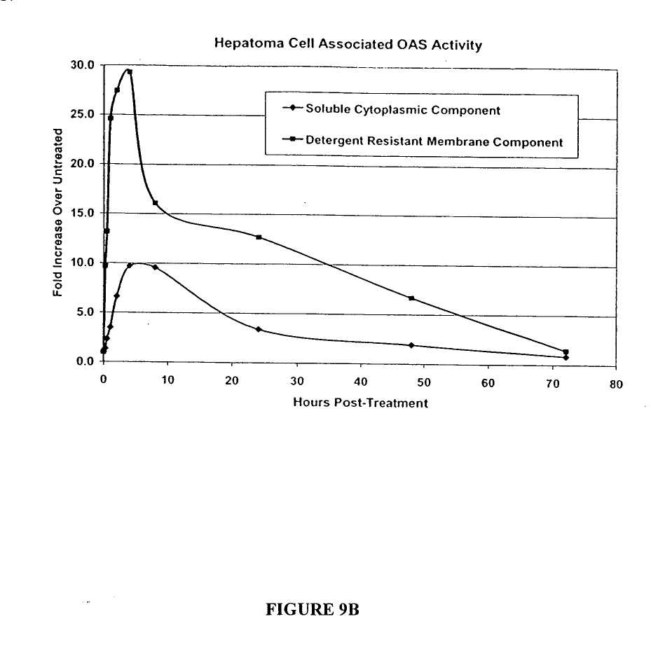

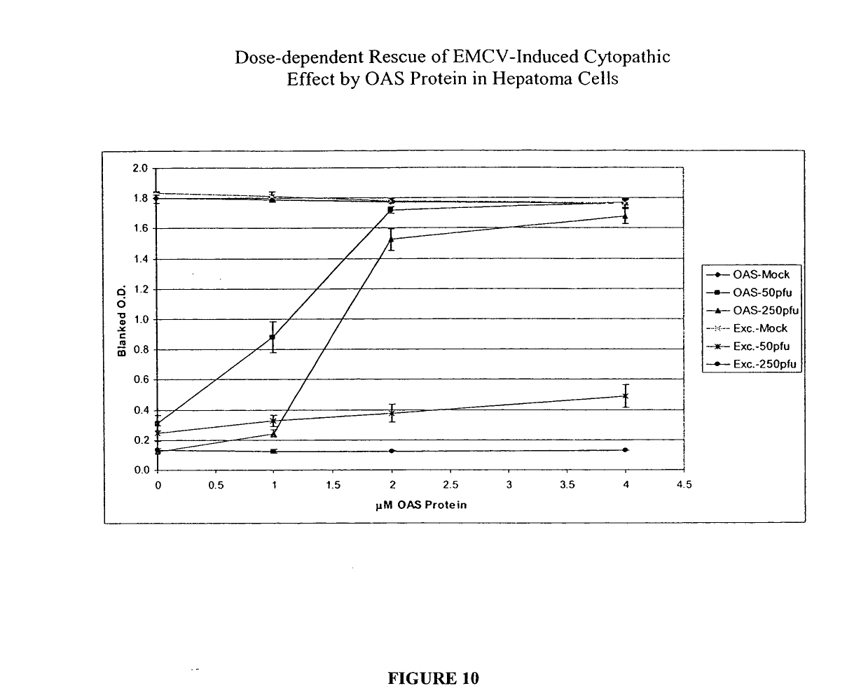

Furthermore, oligoadenylate synthetase polypeptides are able, as part of their native function, to transduce across a cell membrane and mediate their antiviral effects in the absence of a delivery vector or expression vehicle. The mechanism of polypeptide transduction is likely absorptive endocytosis or lipid raft-mediated macropinocytosis, with significant amounts of the active polypeptide present in the cytoplasm and in detergent insoluble membrane fractions of treated cells as demonstrated in FIG. 9. The essentially basic and positively charged character of the proteins (the OAS2 pI=8.3 and OAS3 pI=8.7) likely mediates this unusual characteristic, making the polypeptides themselves effective pharmaceutical compositions without the need for carriers to increase cell permeability. The cell transduction properties of basic, positively charged proteins has been previously described and is well known to those skilled in the art (Ryser and Hancock, Science. 1965 Oct 22;150(695):501-3). It is clear from FIG. 10 of the present invention that oligoadenylate synthetase polypeptides can affect an antiviral function in cell culture that can only be mediated by transduction of the polypeptides into the cell.

In the case where the polypeptides are prepared as a liquid formulation and administered by injection, preferably the solution is an isotonic salt solution containing 140 millimolar sodium chloride and 10 millimolar calcium at pH 7.4. The injection may be administered, for example, in a therapeutically effective amount, preferably in a dose of about 1 μg/kg body weight to about 5 mg/kg body weight daily, taking into account the routes of administration, health of the patient, etc.

The polypeptide(s) of the present invention may be employed in combination with a suitable pharmaceutical carrier. Such compositions comprise a therapeutically effective amount of the protein, and a pharmaceutically acceptable carrier or excipient. Such a carrier includes but is not limited to saline, buffered saline, dextrose, water, glycerol, ethanol, and combinations thereof. The formulation should suit the mode of administration.

The polypeptide(s) of the present invention can also be modified by chemically linking the polypeptide to one or more moieties or conjugates to enhance the activity, cellular distribution, or cellular uptake of the polypeptide(s). Such moieties or conjugates include lipids such as cholesterol, cholic acid, thioether, aliphatic chains, phospholipids and their derivatives, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

The polypeptide(s) of the present invention may also be modified to target specific cell types for a particular disease indication, including but not limited to liver cells in the case of hepatitis C infection. As can be appreciated by those skilled in the art, suitable methods have been described that achieve the described targeting goals and include, without limitation, liposomal targeting, receptor-mediated endocytosis, and antibody-antigen binding. In one embodiment, the asiaglycoprotein receptor may be used to target liver cells by the addition of a galactose moiety to the polypeptide(s). In another embodiment, mannose moieties may be conjugated to the polypeptide(s) in order to target the mannose receptor found on macrophages and liver cells. The polypeptide(s) of the present invention may also be modified for cytosolic delivery by methods known to those skilled in the art, including, but not limited to, endosome escape mechanisms or protein transduction domain (PTD) systems. Known endosome escape systems include the use of ph-responsive polymeric carriers such as poly(propylacrylic acid). Known PTD systems range from natural peptides such as HIV-1 TAT, HSV-1 VP22, Drosophila Antennapedia, or diphtheria toxin to synthetic peptide carriers (Wadia and Dowdy, Cur. Opin. Biotech. 13:52-56, 2002; Becker-Hapak et. al., Methods 24:247-256, 2001). FIG. 10 provides detailed description of several of these exemplary PTDs. As one skilled in the art will recognize, multiple delivery and targeting methods may be combined. For example, the polypeptide(s) of the present invention may be targeted to liver cells by encapsulation within liposomes, such liposomes being conjugated to galactose for targeting to the asialoglycoprotein receptor.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptide of the present invention may be employed in conjunction with other therapeutic compounds.

When the OAS forms of the present invention are used as a pharmaceutical, they can be given to mammals, in a suitable vehicle. When the polypeptides of the present invention are used as a pharmaceutical as described above, they are given, for example, in therapeutically effective doses of about 10 μg/kg body weight to about 4 mg/kg body weight daily, taking into account the routes of administration, health of the patient, etc. The amount given is preferably adequate to achieve prevention or inhibition of infection by a virus, preferably a flavivirus, most preferably HCV, thus replicating the natural resistance found in humans carrying a resistant OAS allele as disclosed herein.

Inhibitor-based drug therapies that mimic the beneficial effects of at least one mutation at position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15 are also envisioned, as discussed in detail below. As discussed previously, one exemplary rationale for developing such inhibitors is the case where the beneficial mutation diminishes or eradicates expression, translation, or function of one or more particular isoforms of OAS2 or OAS3. The present invention is not limited by the precise form or effect of the beneficial mutation nor the biological activity of the particular isoforms thereby affected. In such case, one skilled in the art will appreciate the utility of therapeutically inhibiting said particular isoform(s) of OAS2 or OAS3. These inhibitor-based therapies can take the form of chemical entities, peptides or proteins, antisense oligonucleotides, small interference RNAs, and antibodies.

The proteins, their fragments or other derivatives, or analogs thereof, or cells expressing them can be used as an immunogen to produce antibodies thereto. These antibodies can be, for example, polyclonal, monoclonal, chimeric, single chain, Fab fragments, or the product of an Fab expression library. Various procedures known in the art may be used for the production of polyclonal antibodies.

Antibodies generated against the polypeptide encoded by an OAS2 or OAS3 form of the present invention can be obtained by direct injection of the polypeptide into an animal or by administering the polypeptide to an animal, preferably a nonhuman. The antibody so obtained will then bind the polypeptide itself. In this manner, even a sequence encoding only a fragment of the polypeptide can be used to generate antibodies binding the whole native polypeptide. Moreover, a panel of such antibodies, specific to a large number of polypeptides, can be used to identify and differentiate such tissue.

For preparation of monoclonal antibodies, any technique which provides antibodies produced by continuous cell line cultures can be used. Examples include the hybridoma technique (Kohler and Milstein, 1975, Nature, 256:495-597), the trioma technique, the human B-cell hybridoma technique (Kozbor, et al., 1983, Immunology Today 4:72), and the EBV-hybridoma technique to produce human monoclonal antibodies (Coe, et al., 1985, Monoclonal Antibodies and Cancer Therapy, Alan R. Liss, Inc. pp. 77-96).

Techniques described for the production of single chain antibodies (U.S. Pat. No. 4,946,778) can be adapted to produce single chain antibodies to immunogenic polypeptide products of this invention.

The antibodies can be used in methods relating to the localization and activity of the protein sequences of the invention, e.g., for imaging these proteins, measuring levels thereof in appropriate physiological samples, and the like.

The present invention provides detectably labeled oligonucleotides for imaging OAS2 or OAS3 polynucleotides within a cell. Such oligonucleotides are useful for determining if gene amplification has occurred, and for assaying the expression levels in a cell or tissue using, for example, in situ hybridization as is known in the art.

Therapeutic Agents for Inhibition of OAS Function

The present invention also relates to antisense oligonucleotides designed to interfere with the normal function of OAS2 or OAS3 polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Preferred antisense oligonucleotides can be selected by routine experimentation using, for example, assays described in the Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles et al., Nuc. Acids Res. 23:954-61, 1995; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

As disclosed in U.S. Pat. No. 5,998,383, incorporated herein by reference, the oligonucleotide is selected such that the sequence exhibits suitable energy related characteristics important for oligonucleotide duplex formation with their complementary templates, and shows a low potential for self-dimerization or self-complementation (Anazodo et al., Biochem. Biophys. Res. Commun. 229:305-09, 1996). The computer program OLIGO (Primer Analysis Software, Version 3.4), is used to determined antisense sequence melting temperature, free energy properties, and to estimate potential self-dimer formation and self-complimentarity properties. The program allows the determination of a qualitative estimation of these two parameters (potential self-dimer formation and self-complimentary) and provides an indication of “no potential” or “some potential” or “essentially complete potential.” Segments of OAS polynucleotides are generally selected that have estimates of no potential in these parameters. However, segments can be used that have “some potential” in one of the categories. A balance of the parameters is used in the selection.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in the Examples. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.) According to the present invention, cultures of cells are transfected with two different antisense oligonucleotides designed to target OAS2 or OAS3. The levels of mRNA corresponding to OAS2 or OAS3 as appropriate are measured in treated and control cells.

Additional inhibitors include ribozymes, proteins or polypeptides, antibodies or fragments thereof as well as small molecules. Each of these OAS inhibitors share the common feature in that they reduce the expression and/or biological activity of OAS2 or OAS3. In addition to the exemplary OAS2 or OAS3 inhibitors disclosed herein, alternative inhibitors may be obtained through routine experimentation utilizing methodology either specifically disclosed herein or as otherwise readily available to and within the expertise of the skilled artisan.

Ribozymes

OAS2 or OAS3 inhibitors may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell 48:211-20, 1987; Haseloff and Gerlach, Nature 328:596-600, 1988; Walbot and Bruening, Nature 334:196, 1988; Haseloff and Gerlach, Nature 334:585, 1988); the hairpin ribozyme (for example, as described by Haseloffet al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such OAS2 or OAS3 mRNA-specific ribozyme, or a nucleic acid encoding such a ribozyme, may be delivered to a host cell to effect inhibition of the corresponding OAS2 or OAS3 gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed.

RNAi

The invention also provides for the introduction of RNA with partial or fully double-stranded character into the cell or into the extracellular environment. Inhibition is specific to the OAS2 or OAS3 expression in that a nucleotide sequence from a portion of the target OAS gene is chosen to produce inhibitory RNA. This process is (1) effective in producing inhibition of gene expression, and (2) specific to the targeted OAS gene. The procedure may provide partial or complete loss of function for the target OAS gene. A reduction or loss of gene expression in at least 99% of targeted cells has been shown using comparable techniques with other target genes. Lower doses of injected material and longer times after administration of dsRNA may result in inhibition in a smaller fraction of cells. Quantitation of gene expression in a cell may show similar amounts of inhibition at the level of accumulation of target mRNA or translation of target protein. Methods of preparing and using RNAi are generally disclosed in U.S. Pat. No. 6,506,559, incorporated herein by reference.

The RNA may comprise one or more strands of polymerized ribonucleotide; it may include modifications to either the phosphate-sugar backbone or the nucleoside. The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequence identical to a portion of the OAS target gene is preferred for inhibition. RNA sequences with insertions, deletions, and single point mutations relative to the target sequence have also been found to be effective for inhibition. Thus, sequence identity may optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For RNAi, the RNA may be directly introduced into the cell (i.e., intracellularly), or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected. Physical methods of introducing nucleic acids include injection directly into the cell or extracellular injection into the organism of an RNA solution.

The advantages of the method include the ease of introducing double-stranded RNA into cells, the low concentration of RNA which can be used, the stability of double-stranded RNA, and the effectiveness of the inhibition.

Inhibition of gene expression refers to the absence (or observable decrease) in the level of protein and/or mRNA product from a OAS target gene. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of dsRNA may result in inhibition in a smaller fraction of cells (e.g., at least 10%, 20%, 50%, 75%, 90%, or 95% of targeted cells). Quantitation of target OAS gene expression in a cell may show similar amounts of inhibition at the level of accumulation of OAS target mRNA or translation of OAS target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

The RNA may comprise one or more strands of polymerized ribonucleotide. It may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

RNA containing a nucleotide sequences identical to a portion of the OAS target gene are preferred for inhibition. RNA sequences with insertions, deletions, and single point mutations relative to the target sequence may be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the OAS target gene is preferred. Alternatively, the duplex region of the RNA may be defined functionally as a nucleotide sequence that is capable of hybridizing with a portion of the OAS target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 25, 50, 100, 200, 300 or 400 bases.

100% sequence identity between the RNA and the OAS target gene is not required to practice the present invention. Thus the methods have the advantage of being able to tolerate sequence variations that might be expected due to genetic mutation, strain polymorphism, or evolutionary divergence.

OAS2 or OAS3 RNA may be synthesized either in vivo or in vitro. Endogenous RNA polymerase of the cell may mediate transcription in vivo, or cloned RNA polymerase can be used for. transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in (see WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

RNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The present invention may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶ molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

Proteins and Polypeptides

In addition to the antisense molecules and ribozymes disclosed herein, OAS inhibitors of the present invention also include proteins or polypeptides that are effective in either reducing OAS2 or OAS3 gene expression or in decreasing one or more of OAS2 or OAS3's biological activities, including but not limited to enzymatic activity; interaction with single stranded RNA, configurations; and binding to other proteins such as viral proteins or a fragment thereof. A variety of methods are readily available in the art by which the skilled artisan may, through routine experimentation, rapidly identify such OAS inhibitors. The present invention is not limited by the following exemplary methodologies.

Literature is available to the skilled artisan that describes methods for detecting and analyzing protein-protein interactions. Reviewed in Phizicky et al., Microbiological Reviews 59:94-123, 1995, incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive OAS inhibitors may be identified through biological screening assays that rely on the direct interaction between the OAS2 or OAS3 protein and a panel or library of potential inhibitor proteins. Biological screening methodologies, including the various “n-hybrid technologies,” are described in, for example, Vidal et al., Nucl. Acids Res. 27(4):919-29, 1999; Frederickson, R. M., Curr. Opin. Biotechnol. 9(1):90-96, 1998; Brachmann et al., Curr. Opin. Biotechnol. 8(5):561-68, 1997; and White, M. A., Proc. Natl. Acad. Sci. U.S.A. 93:10001-03, 1996, each of which is incorporated herein by reference.

The two-hybrid screening methodology may be employed to search new or existing target cDNA libraries for OAS2 or OAS3 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien et al., Proc. Natl. Acad. Sci. U.S.A. 88:9578-82, 1991; Dalton et al., Cell 68:597-612, 1992; Durfee et al., Genes Dev. 7:555-69, 1993; Vojtek et al., Cell 74:205-14, 1993; and Zervos et al., Cell 72:223-32, 1993. Commonly used reporter genes include the E. coli lacZ gene as well as selectable yeast genes such as HIS3 and LEU2. Fields et al., Nature (London) 340:245-46, 1989; Durfee, T. K., supra; and Zervos, A. S., supra. A wide variety of activation domain libraries is readily available in the art such that the screening for interacting proteins may be performed through routine experimentation.

Suitable bait proteins for the identification of OAS2 or OAS3 interacting proteins may be designed based on the OAS2 or OAS3 DNA sequence presented herein as SEQUENCE:2 or SEQUENCE: 1, respectively. Such bait proteins include either the full-length OAS protein or fragments thereof.

Plasmid vectors, such as, e.g., pBTM116 and pAS2-1, for preparing OAS bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

OAS inhibitors of the present invention may alternatively be identified through one of the physical or biochemical methods available in the art for detecting protein-protein interactions.

Through the protein affinity chromatography methodology, lead compounds to be tested as potential OAS inhibitors may be identified by virtue of their specific retention to OAS2 or OAS3 when either covalently or non-covalently coupled to a solid matrix such as, e.g., Sepharose beads. The preparation of protein affinity columns is described in, for example, Beeckmans et al., Eur. J. Biochem. 117:527-35, 1981, and Formosa et al., Methods Enzymol. 208:24-45, 1991. Cell lysates containing the full complement of cellular proteins may be passed through the OAS2 or OAS3 affinity column. Proteins having a high affinity for OAS2 or OAS3 will be specifically retained under low-salt conditions while the majority of cellular proteins will pass through the column. Such high affinity proteins may be eluted from 20 the immobilized OAS under conditions of high-salt, with chaotropic solvents or with sodium dodecyl sulfate (SDS). In some embodiments, it may be preferred to radiolabel the cells prior to preparing the lysate as an aid in identifying the OAS2- or OAS3-specific binding proteins. Methods for radiolabeling mammalian cells are well known in the art and are provided, e.g., in Sopta et al., J. Biol. Chem. 260:10353-60, 1985.

Suitable OAS2 or OAS3 proteins for affinity chromatography may be fused to a protein or polypeptide to permit rapid purification on an appropriate affinity resin. For example, the OAS2 or OAS3 cDNA may be fused to the coding region for glutathione S-transferase (GST) which facilitates the adsorption of fusion proteins to glutathione-agarose columns. Smith et al., Gene 67:31-40, 1988. Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of OAS2 or OAS3 fusion proteins that is presented herein is the epitope for the influenza virus hemagglutinin (HA) against which monoclonal antibodies are readily available and from which antibodies an affinity column may be prepared.

Proteins that are specifically retained on a OAS2 or OAS3 affinity column may be identified after subjecting to SDS polyacrylamide gel electrophoresis (SDS-PAGE). Thus, where cells are radiolabeled prior to the preparation of cell lysates and passage through the OAS2 or OAS3 affinity column, proteins having high affinity for the said OAS2 or OAS3 may be detected by autoradiography. The identity of OAS2 or OAS3 specific binding proteins may be determined by protein sequencing techniques that are readily available to the skilled artisan, such as Mathews, C. K. et al., Biochemistry, The Benjamin/Cummings Publishing Company, Inc., 1990, pp.166-70.

Small Molecules

The present invention also provides small molecule OAS2 or OAS3 inhibitors that may be readily identified through routine application of high-throughput screening (HTS) methodologies. Reviewed by Persidis, A., Nature Biotechnology 16:488-89, 1998. HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. See, e.g., Gonzalez et al., Curr. Opin. Biotechnol. 9(6):624-31, 1998, incorporated herein by reference.

HTS methodology may be employed, e.g., to screen for lead compounds that block one of OAS2 or OAS3's biological activities. By this method, OAS protein may be immunoprecipitated from cells expressing the protein and applied to wells on an assay plate suitable for robotic screening. Individual test compounds may then be contacted with the immunoprecipitated protein and the effect of each test compound on the target OAS.

Methods for Assessing the Efficacy of OAS Inhibitors

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit OAS gene expression or biological activity. As discussed in further detail in the Examples provided below, OAS inhibitors of the present invention are effective in reducing OAS2 or OAS3 expression levels. Thus, the present invention further discloses methods that permit the skilled artisan to assess the effect of candidate inhibitors.

Candidate OAS inhibitors may be tested by administration to cells that either express endogenous target OAS or that are made to express the target OAS by transfection of a mammalian cell with a recombinant target OAS plasmid construct.

Effective OAS inhibitory molecules will be effective in reducing the enzymatic activity of OAS2 or OAS3 or the ability of OAS2 or OAS3 to respond to IFN induction. Methods of measuring OAS enzymatic activity and IFN induction are known in the art, for example, as described in Eskildsen et al., Nuc. Acids Res. 31:3166-3173, 2003; and Justesen et al., Nuc. Acids Res. 8:3073-3085, 1980, incorporated herein by reference. The effectiveness of a given candidate antisense molecule may be assessed by comparison with a control “antisense” molecule known to have no substantial effect on target OAS expression when administered to a mammalian cell.

OAS inhibitors effective in reducing target OAS gene expression by one or more of the methods discussed above may be further characterized in vitro for efficacy in one of the readily available established cell culture or primary cell culture model systems as described herein, in reference to use of Vero cells challenged by infection with a flavivirus, such as dengue virus.

Nucleic Acid Pharmaceutical Compositions

The antisense oligonucleotides and ribozymes of the present invention can be synthesized by any method known in the art for ribonucleic or deoxyribonucleic nucleotides. For example, the oligonucleotides can be prepared using solid-phase synthesis such as in an Applied Biosystems 380B DNA synthesizer. Final purity of the oligonucleotides is determined as is known in the art.

The antisense oligonucleotides identified using the methods of the invention modulate tumor cell proliferation. Therefore, pharmaceutical compositions and methods are provided for interfering with virus infection, preferably flavivirus, most preferably HCV infection, comprising contacting tissues or cells with one or more of antisense oligonucleotides identified using the methods of the invention.

The invention provides pharmaceutical compositions of antisense oligonucleotides and ribozymes complementary to the OAS2 or OAS3 mRNA gene sequence as active ingredients for therapeutic application. These compositions can also be used in the method of the present invention. When required, the compounds are nuclease resistant. In general the pharmaceutical composition for inhibiting virus infection in a mammal includes an effective amount of at least one antisense oligonucleotide as described above needed for the practice of the invention, or a fragment thereof shown to have the same effect, and a pharmaceutically physiologically acceptable carrier or diluent.

The compositions can be administered orally, subcutaneously, or parenterally including intravenous, intraarterial, intramuscular, intraperitoneally, and intranasal administration, as well as intrathecal and infusion techniques as required. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate oligonucleotide uptake. Implants of the compounds are also useful. In general, the pharmaceutical compositions are sterile.

By bioactive (expressible) is meant that the oligonucleotide is biologically active in the cell when delivered directly to the cell and/or is expressed by an appropriate promotor and active when delivered to the cell in a vector as described below. Nuclease resistance is provided by any method known in the art that does not substantially interfere with biological activity as described herein.

“Contacting the cell” refers to methods of exposing or delivering to a cell antisense oligonucleotides whether directly or by viral or non-viral vectors and where the antisense oligonucleotide is bioactive upon delivery.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promotor to allow the sequence to be expressed in the targeted cell.

Once the oligonucleotide sequences are ready for delivery they can be introduced into cells as is known in the art. Transfection, electroporation, fusion, liposomes, colloidal polymeric particles, and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

The present invention provides vectors comprising an expression control sequence operatively linked to the oligonucleotide sequences of the invention. The present invention further provides host cells, selected from suitable eukaryotic and prokaryotic cells, which are transformed with these vectors as necessary.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either procaryotic or eucaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York, 1989, 1992; in Ausubel et al., Current Protocols in Molecular Biology, John Wiley and Sons, Baltimore, Md., 1989; Chang et al., Somatic Gene Therapy, CRC Press, Ann Arbor, Mich., 1995; Vega et al., Gene Targeting, CRC Press, Ann Arbor, Mich., 1995; Vectors: A Survey of Molecular Cloning Vectors and Their Uses, Butterworths, Boston, Mass., 1988; and Gilboa et al., BioTechniques 4:504-12, 1986, and include, for example, stable or transient transfection, lipofection, electroporation and infection with recombinant viral vectors.

Recombinant methods known in the art can also be used to achieve the antisense inhibition of a target nucleic acid. For example, vectors containing antisense nucleic acids can be employed to express an antisense message to reduce the expression of the target nucleic acid and therefore its activity.

The present invention also provides a method of evaluating if a compound inhibits transcription or translation of an OAS gene and thereby modulates (i.e., reduces) the ability of the cell to activate RNaseL, comprising transfecting a cell with an expression vector comprising a nucleic acid sequence encoding a desired OAS, the necessary elements for the transcription or translation of the nucleic acid; administering a test compound; and comparing the level of expression of the desired OAS with the level obtained with a control in the absence of the test compound.

Polypeptide Pharmaceutical Compositions

The invention provides pharmaceutical compositions of the polypeptides as active ingredients for a therapeutic application. These compositions can also be used in the method of the present invention. In general the pharmaceutical composition for inhibiting virus infection, cancer, neoplasm, inflammation, or other disease in a mammal or subject includes an effective amount of at least one polypeptide as described above needed for the practice of the invention, or a fragment thereof shown to have the same effect, and a pharmaceutically physiologically acceptable carrier or diluent. According to the present invention, a pharmaceutical composition can be composed of two or more of the polypeptides of of the invention in combination. The pharmaceutical composition may further be composed of a single polypeptide that contains one or more of the modifications of described herein within a contiguous molecule.

The compositions can be administered orally, subcutaneously, or parenterally including intravenous, intraarterial, intramuscular, intraperitoneally, and intranasal administration, as well as intrathecal and infusion techniques as required. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate polypeptide uptake. Implants of the compounds are also useful. In general, the pharmaceutical compositions are sterile.

The present invention relates to compositions of the polypeptides to which a detectable label is attached, such as a fluorescent, chemiluminescent or radioactive molecule.

Another example is a pharmaceutical composition which may be formulated by known techniques using known materials, see, Remington's Pharmaceutical Sciences, 18th Ed. (1990, Mack Publishing Co., Easton, Pa. 18042) pp. 1435-1712, which are herein incorporated by reference. Generally, the formulation will depend on a variety of factors such as administration, stability, production concerns and other factors. The polypeptides of FIG. 3 and derivatives thereof may be administered by injection or by pulmonary administration via inhalation. Enteric dosage forms may also be available, and therefore oral administration may be effective. The polypeptides of the invention may be inserted into liposomes or other microcarriers for delivery, and may be formulated in gels or other compositions for sustained release. Although preferred compositions will vary depending on the use to which the composition will be put, generally, for the polypeptides of the present invention, preferred pharmaceutical compositions are those prepared for subcutaneous injection or for pulmonary administration via inhalation, although the particular formulations for each type of administration will depend on the characteristics of the specific polypeptide.

Therapeutic formulations of the polypeptides or polypeptide conjugates of the invention are typically administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals.

The polypeptides or polypeptide conjugates of the invention can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

“Pharmaceutically acceptable” means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3rd edition, A. Kibbe, Ed., Pharmaceutical Press (2000)).

The composition of the invention may be administered alone or in conjunction with other therapeutic agents. Ribavirin and interferon alpha, for example, have been shown to be an effective treatment for HCV infection when used in combination. Their efficacy in combination exceeds the efficacy of either drug product when used alone. The compositions of the invention may be administered alone or in combination with interferon, ribavirin and/or a variety of small molecules that are being developed against both viral targets (viral proteases, viral polymerase, assembly of viral replication complexes) and host targets (host proteases required for viral processing, host kinases required for phosphorylation of viral targets such as NS5A and inhibitors of host factors required to efficiently utilize the viral IRES). Cytokines may be co-administered, such as for example IL-2, IL-12, IL-23, IL-27, or IFN-gamma. These agents may be incorporated as part of the same pharmaceutical composition or may be administered separately from the polypeptides or conjugates of the invention, either concurrently or in accordance with another treatment schedule. In addition, the polypeptides, polypeptide conjugates or compositions of the invention may be used as an adjuvant to other therapies.

A “patient” for the purposes of the present invention includes both humans and other mammals. Thus the methods are applicable to both human therapy and veterinary applications

The pharmaceutical composition comprising the polypeptide or conjugate of the invention may be formulated in a variety of forms, e.g. as a liquid, gel, lyophilized, or as a compressed solid. The preferred form will depend upon the particular indication being treated and will be apparent to one skilled in the art.

The administration of the formulations of the present invention can be performed in a variety of ways, including, but not limited to, orally, subcutaneously, intravenously, intracerebrally, intranasally, transdermally, intraperitoneally, intramuscularly, intrapulmonary, intrathecally, vaginally, rectally, intraocularly, or in any other acceptable manner. The formulations can be administered continuously by infusion, although bolus injection is acceptable, using techniques well known in the art, such as pumps (e.g., subcutaneous osmotic pumps) or implantation. In some instances the formulations may be directly applied as a solution or spray.

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

Parenterals may be prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, alpha-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

In one aspect of the invention the composition is a liquid composition, such as an aqueous composition, and comprises a sulfoalkyl ether cyclodextrin derivative.

Parenteral formulations to be used for in vivo administration must be sterile. This is readily accomplished, for example, by filtration through sterile filtration membranes.

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S--S bond formation through thio-disulfide interchange, stabilization may be achieved by modifying sulfbydryl residues, lyophilizing from acidic solutions, controlling moisture content, using appropriate additives, and developing specific polymer matrix compositions.

Oral administration of the peptides and peptide conjugates is an intended practice of the invention. For oral administration, the pharmaceutical composition may be in solid or liquid form, e.g. in the form of a capsule, tablet, suspension, emulsion or solution. The pharmaceutical composition is preferably made in the form of a dosage unit containing a given amount of the active ingredient. A suitable daily dose for a human or other mammal may vary widely depending on the condition of the patient and other factors, but can be determined by persons skilled in the art using routine methods.

Solid dosage forms for oral administration may include capsules, tablets, suppositories, powders and granules. In such solid dosage forms, the active compound may be admixed with at least one inert diluent such as sucrose, lactose, or starch. Such dosage forms may also comprise, as is normal practice, additional substances, e.g. lubricating agents such as magnesium stearate. In the case of capsules, tablets and pills, the dosage forms may also comprise buffering agents. Tablets and pills can additionally be prepared with enteric coatings.

The polypeptides or conjugates may be admixed with adjuvants such as lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they may be dissolved in saline, water, polyethylene glycol, propylene glycol, ethanol, oils (such as corn oil, peanut oil, cottonseed oil or sesame oil), tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be subjected to conventional pharmaceutical operations such as sterilization and/or may contain conventional adjuvants such as preservatives, stabilizers, wetting agents, emulsifiers, buffers, fillers, etc., e.g. as disclosed elsewhere herein.

Liquid dosage forms for oral administration may include pharmaceutically acceptable emulsions, solutions, suspensions, syrups and elixirs containing inert diluents commonly used in the art, such as water. Such compositions may also comprise adjuvants such as wetting agents, sweeteners, flavoring agents and perfuming agents.

Formulations suitable for pulmonary administration are intended as part of the invention. Formulations suitable for use with a nebulizer, either jet or ultrasonic, will typically comprise the polypeptide or conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 94/20069, U.S. Pat. No.5,915,378, U.S. Pat. No.5,960,792, U.S. Pat. No. 5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385 which are hereby incorporated by reference.

Formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C., USA.

Formulations for powder inhalers will comprise a finely divided dry powder containing polypeptides or polypeptide conjugates and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers. An example of a powder inhaler suitable for use in accordance with the teachings herein is the Spinhaler powder inhaler, manufactured by Fisons Corp., Bedford, Mass., USA. The powders for these devices may be generated and/or delivered by methods disclosed in U.S. Pat. No. 5,997,848, U.S. Pat. No. 5,993,783, U.S. Pat. No. 5,985,248, U.S. Pat. No. 5,976,574, U.S. Pat. No. 5,922,354, U.S. Pat. No. 5,785,049 and U.S. Pat. No. 5,654,007.

Mechanical devices designed for pulmonary delivery of therapeutic products, include but are not limited to nebulizers, metered dose inhalers, and powder inhalers, all of which are familiar to those of skill in the art. Specific examples of commercially available devices suitable for the practice of this invention are the Ultravent nebulizer, manufactured by Mallinckrodt, Inc., St. Louis, Mo., USA; the Acorn II nebulizer, manufactured by Marquest Medical Products, Englewood, Colo., USA; the Ventolin metered dose inhaler, manufactured by Glaxo Inc., Research Triangle Park, N.C., USA; the Spinhaler powder inhaler, manufactured by Fisons Corp., Bedford, Mass., USA the “standing cloud” device of Nektar Therapeutics, Inc., San Carlos, Calif., USA; the AIR inhaler manufactured by Alkermes, Cambridge, Mass., USA; and the AERx pulmonary drug delivery system manufactured by Aradigm Corporation, Hayward, Calif., USA.

The present invention also provides kits including the polypeptides, conjugates, polynucleotides, expression vectors, cells, methods, compositions, and systems, and apparatuses of the invention. Kits of the invention optionally comprise at least one of the following of the invention: (1) an apparatus, system, system component, or apparatus component as described herein; (2) at least one kit component comprising a polypeptide or conjugate or polynucleotide of the invention; a plasmid expression vector encoding a polypeptide of the invention; a cell expressing a polypeptide of the invention; or a composition comprising at least one of any such component; (3) instructions for practicing any method described herein, including a therapeutic or prophylactic method, instructions for using any component identified in (2) or any composition of any such component; and/or instructions for operating any apparatus, system or component described herein; (4) a container for holding said at least one such component or composition, and (5) packaging materials.

In a further aspect, the present invention provides for the use of any apparatus, component, composition, or kit described above and herein, for the practice of any method or assay described herein, and/or for the use of any apparatus, component, composition, or kit to practice any assay or method described herein.

Chemical Modifications, Conjugates, and Fusions of OAS2 and OAS3

The present invention relates to novel pharmaceutical compositions composed of engineered forms of the oligoadenylate synthetases. These pharmaceutical compositions include mutant forms designed to have enhanced cell permeability, reduced oxidative potential, enhanced antiviral activity, enhanced enzymatic activity, or absent enzymatic activity. These pharmaceutical compositions further embody oligoadenylate synthetases chemically modified with polyethylene glycol. The present invention further relates to any possible combination of mutant forms or chemical modifications in a single polypeptide.

The present invention relates to mutant oligoadenylate synthetase forms that have no enzymatic activity, but that retain their antiviral activity. These forms have one or two mutations of aspartic acid to alanine in the magnesium binding site of the polypeptide, rendering the resulting OAS forms enzymatically inactive. These enzymatically inactive OAS polypeptides retain antiviral activity, demonstrated using an encephalomyocarditis virus replication assay.

The present invention further relates to mutant oligoadenylate synthetase forms that have reduced oxidative potential. These forms have one or more cysteine amino acid residues deleted or replaced with an alternative residue of the form: alanine, serine, threonine, methionine, or glycine. Deletion or modification of these residues reduces the oxidative potential of the resulting polypeptide drug product, thereby improving manufacturability and in vivo serum stability of the drug. Manufacturability is improved by obviating the need for a reducing environment during drug manufacture while reducing the propensity of drug aggregation during manufacture, transport, and drug delivery.

The present invention further relates to mutant oligoadenylate synthetase forms that have enhanced cell permeability. Cell permeability is enhanced by the addition of basic amino acids, histidine, arginine, and lysine, to the amino terminus of the polypeptide. Addition of basic or positively-charged amino acids increases cell permeability through an absorptive endocytic process, thereby increasing the antiviral activity of the pharmaceutical compositions. Enhancement of absorptive endocytosis of the polypeptide drug through the addition of basic amino acids results in the significant accumulation of active drug in intracellular, detergent insoluble stores thereby enhancing in vivo therapeutic effect. However, given their native basic nature, OAS proteins have an innate ability to enter cells.

The present invention further relates to chemical modifications of the polypeptide drug to contain a polyethylene glycol moiety. Chemical modification of cysteine residues results in retention of full enzyme activity, improved in vitro bulk drug product stability, enhanced serum elimination half life, reduced in vivo drug immunogenicity, and reduced in vivo proteolytic cleavage of the drug polypeptide.

The present invention further relates to any combination of one or more of the mutations or modifications above within a single polypeptide or pharmaceutical composition.

The invention provides for increasing the cell permeability of a drug by conjugation to the polypeptides of the invention. The invention further provides for increasing the cell permeability of a drug by conjugation to five or more consecutive amino acids of the polypeptides of the invention.

The invention provides a method for delivering a drug into a cell by conjugation to the polypeptides of the present invention or five or more consecutive amino acids of the polypeptides of the present invention. In a further embodiment, conjugation may be affected using chemical methods and may be through covalent or non-covalent interaction. In a still further embodiment, nucleic acids encoding the polypeptides of the present invention may be joined with other nucleic acids in order to make heterologous polypeptides with increased cell permeability, said increased permeability being derived from five or more amino acids of the polypeptides of the present invention.

Any polypeptide of the invention may be present as part of a larger polypeptide sequence, e.g. a fusion protein, such as occurs upon the addition of one or more domains or subsequences for stabilization or detection or purification of the polypeptide. A polypeptide purification subsequence may include, e.g., an epitope tag, a FLAG tag, a polyhistidine sequence, a GST fusion, or any other detection/purification subsequence or “tag” known in the art. These additional domains or subsequences either have little or no effect on the activity of the polypeptide of the invention, or can be removed by post synthesis processing steps such as by treatment with a protease, inclusion of an intein, or the like.

The invention includes fusion proteins comprising a polypeptide of the invention, e.g., as described herein, fused to an Ig molecule, e.g., a human IgG Fc (“fragment crystallizable,” or fragment complement binding) hinge, CH2 domain and CH3 domain, and nucleotide sequences encoding such fusion protein. Fc is the portion of the antibody responsible for binding to antibody receptors on cells and the C1q component of complement. These fusion proteins and their encoding nucleic acids are useful as prophylactic and/or therapeutic drugs or as diagnostic tools (see also, e.g., Challita-Eid, P. et al. (1998) J. Immunol 160:3419-3426; Sturmhoefel, K. et al. (1999) Cancer Res 59:4964-4972). The invention also includes fusion proteins comprising a polypeptide of the invention, fused to an albumin molecule, such as human serum albumin (HSA), as described, for example, in U.S. Pat. No. 5,876,969, and nucleotide sequences encoding the fusion protein. The Ig and albumin fusion proteins may exhibit increased polypeptide serum half-life and/or functional in vivo half-life, reduced polypeptide antigenicity, increased polypeptide storage stability, or increasing bioavailability, e.g. increased AUC_{sc}, and are thus may be useful as prophylactic and/or therapeutic drugs.

All of the polypeptides of the invention have an inherent ability to transduce across cellular membranes and affect therapeutic functions within cells (FIG. 9 and FIG. 10). The invention therefore provides for the use of the polypeptides of the invention to enhance the cell permeability or transducibility of any other molecule. The invention further provides for the use of any fragment or subfragment of the polypeptides of the invention to enhance the cell permeability of any other molecule, such fragments or subfragments being of about 5 amino acids in length, of about 10 amino acids in length, such as 15 amino acids in length, e.g. about 20 amino acids in length, of about 25 amino acids in length, of about 30 amino acids in length, such as 35 amino acids in length, of about 35-50 amino acids in length, of about 50-100 amino acids in length, such as 75 amino acids in length, e.g. 100-125 amino acids in length.

Any polypeptide of the invention may also comprise one or more modified amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. The presence of modified amino acids may be advantageous in, for example, (a) increasing polypeptide serum half-life and/or finctional in vivo half-life, (b) reducing polypeptide antigenicity, (c) increasing polypeptide storage stability, or (d) increasing bioavailability, e.g. increasing the AUC_{sc}. Amino acid(s) are modified, for example, co-translationally or post-translationally during recombinant production (e.g., N-linked glycosylation at N-X-S/T motifs during expression in mammalian cells) or modified by synthetic means.

In another aspect, the invention relates to a conjugate comprising a polypeptide of the invention and at least one non-polypeptide moiety attached to the polypeptide.

The invention provides for polypeptides that differ from the polypeptides of FIG. 3 by 1 to 34 amino acid substitutions or insertions where such substitutions or insertions introduce one or more attachment groups for the non-polypeptide moiety (e.g., by substitution of an amino acid residue for a different residue which comprises an attachment group for the non-polypeptide moiety, or by insertion of an additional amino acid residue which comprises an attachment group for the non-polypeptide moiety).

The term “conjugate” (or interchangeably “polypeptide conjugate” or “conjugated polypeptide”) is intended to indicate a heterogeneous (in the sense of composite) molecule formed by the covalent attachment of one or more polypeptides of the invention to one or more non-polypeptide moieties. The term “covalent attachment” means that the polypeptide and the non-polypeptide moiety are either directly covalently joined to one another, or else are indirectly covalently joined to one another through an intervening moiety or moieties, such as a bridge, spacer, or linkage moiety or moieties. Preferably, a conjugated polypeptide is soluble at relevant concentrations and conditions, i.e. soluble in physiological fluids such as blood. Examples of conjugated polypeptides of the invention include glycosylated and/or PEGylated polypeptides. The term “non-conjugated polypeptide” may be used to refer to the polypeptide part of the conjugated polypeptide.

The term “non-polypeptide moiety” is intended to mean a molecule that is capable of conjugating to an attachment group of the polypeptide. Preferred examples of non-polypeptide moieties include polymer molecules, sugar moieties, lipophilic compounds, or organic derivatizing agents, in particular polymer molecules or sugar moieties. It will be understood that the non-polypeptide moiety is linked to the polypeptide through an attachment group of the polypeptide. Except where the number of non-polupeptide moieties, such as polymer molecule(s), attached to the polypeptide is expressly indicated, every reference to “a non-polypeptide moiety” attached to the polypeptide or otherwise used in the present invention shall be a reference to one or more non-polypeptide moieties attached to the polypeptide.

The term “polymer molecule” is defined as a molecule formed by covalent linkage of two or more monomers, wherein none of the monomers is an amino acid residue. The term “polymer” may be used interchangeably with the term “polymer molecule”.

The term “sugar moiety” is intended to indicate a carbohydrate molecule attached by in vivo or in vitro glycosylation, such as N- or O-glycosylation. An “N-glycosylation site” has the sequence N-X-S/T/C, wherein X is any amino acid residue except proline, N is asparagine and S/T/C is either serine, threonine or cysteine, preferably serine or threonine, and most preferably threonine. An “O-glycosylation site” comprises the OH-group of a serine or threonine residue.

The term “attachment group” is intended to indicate an amino acid residue group capable of coupling to the relevant non-polypeptide moiety such as a polymer molecule or a sugar moiety.

For in vivo N-glycosylation, the term “attachment group” is used in an unconventional way to indicate the amino acid residues constituting an N-glycosylation site (with the sequence N-X-S/T/C, wherein X is any amino acid residue except proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is a sugar moiety and the conjugation is to be achieved by N-glycosylation, the term “amino acid residue comprising an attachment group for the non-polypeptide moiety” as used in connection with alterations of the amino acid sequence of the polypeptide of the invention is to be understood as one, two or all of the amino acid residues constituting an N-glycosylation site is/are to be altered in such a manner that either a functional N-glycosylation site is introduced into the amino acid sequence, removed from said sequence, or a functional N-glycosylation site is retained in the amino acid sequence (e.g. by substituting a serine residue, which already constitutes part of an N-glycosylation site, with a threonine residue and vice versa).

The term “introduce” (i.e., an “introduced” amino acid residue, “introduction” of an amino acid residue) is primarily intended to mean substitution of an existing amino acid residue for another amino acid residue, but may also mean insertion of an additional amino acid residue.

The term “remove” (i.e., a “removed” amino acid residue, “removal” of an amino acid residue) is primarily intended to mean substitution of the amino acid residue to be removed for another amino acid residue, but may also mean deletion (without substitution) of the amino acid residue to be removed.

The term “amino acid residue comprising an attachment group for the non-polypeptide moiety” is intended to indicate that the amino acid residue is one to which the non-polypeptide moiety binds (in the case of an introduced amino acid residue) or would have bound (in the case of a removed amino acid residue).

The term “functional in vivo half-life” is used in its normal meaning, i.e. the time at which 50% of the biological activity of the polypeptide is still present in the body/target organ, or the time at which the activity of the polypeptide is 50% of the initial value. The functional in vivo half-life may be determined in an experimental animal, such as rat, mouse, rabbit, dog or monkey. Preferably, the functional in vivo half-life is determined in a non-human primate, such as a monkey. Furthermore, the functional in vivo half-life may be determined for a sample that has been administered intravenously or subcutaneously.

As an alternative to determining functional in vivo half-life, “serum half-life” may be determined, i.e. the time at which 50% of the polypeptide circulates in the plasma or bloodstream prior to being cleared. Determination of serum half-life is often more simple than determining the functional in vivo half-life and the magnitude of serum half-life is usually a good indication of the magnitude of finctional in vivo half-life. Alternatively terms to serum half-life include “plasma half-life”, “circulating half-life”, “serum clearance”, “plasma clearance” and “clearance half-life”.

The term “serum” is used in its normal meaning, i.e. as blood plasma without fibrinogen and other clotting factors.

The term “increased” as used about the functional in vivo half-life or serum half-life is used to indicate that the relevant half-life of the conjugate of the invention is statistically significantly increased relative to that of a reference molecule or the corresponding non-conjugated polypeptide. Thus, interesting conjugates of the invention include those which have an increased functional in vivo half-life or an increased serum half-life as compared to a reference molecule mentioned above.

The term “AUC_{sc}” or “Area Under the Curve when administered subcutaneously” is used in its normal meaning, i.e. as the area under the drug concentration vs. time curve, where the conjugated molecule has been administered subcutaneously to an experimental animal. Once the experimental drug concentration time points have been determined, the AUC_{sc} may conveniently be calculated by a computer program, such as GraphPad Prism 3.01.

The term “increased” as used about the AUC_{sc} is used to indicate that the Area Under the Curve for a conjugate of the invention, when administered subcutaneously, is statistically significantly increased relative to that of a reference molecule or the corresponding non-conjugated polypeptide, when determined under comparable conditions.

The term “T_{max,sc}” is used about the time point in the drug concentration vs. time curve where the highest drug concentration in serum is observed.

By removing and/or introducing amino acid residues comprising an attachment group for the non-polypeptide moiety it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the oliagoadenylate synthetase molecule and thereby, e.g., effectively shield epitopes and other surface parts of the polypeptide without significantly impairing the function thereof). For instance, by introduction of attachment groups, the oligoadenylate synthetase polypeptide is altered in the content of the specific amino acid residues to which the relevant non-polypeptide moiety binds, whereby a more efficient, specific and/or extensive conjugation is achieved. By removal of one or more attachment groups it is possible to avoid conjugation to the non-polypeptide moiety in parts of the polypeptide in which such conjugation is disadvantageous, e.g. to an amino acid residue located at or near a functional site of the polypeptide (since conjugation at such a site may result in inactivation or reduced therapeutic or prophylactic activity of the resulting conjugate). Further, it may be advantageous to remove an attachment group located close to another attachment group.

It will be understood that the amino acid residue comprising an attachment group for a non-polypeptide moiety, whether it be removed or introduced, is selected on the basis of the nature of the non-polypeptide moiety and, in some instances, on the basis of the conjugation method to be used. For instance, when the non-polypeptide moiety is a polymer molecule, such as a polyethylene glycol or polyalkylene oxide derived molecule, amino acid residues capable of functioning as an attachment group may be selected from the group consisting of cysteine, lysine (and/or the N-terminal amino group of the polypeptide), aspartic acid, glutamic acid, histidine and arginine. When the non-polypeptide moiety is a sugar moiety, the attachment group is an in vivo or in vitro N- or O-glycosylation site, preferably an N-glycosylation site.

In case of removal of an attachment group, the relevant amino acid residue comprising such group and occupying a position as defined above may be substituted with a different amino acid residue that does not comprise an attachment group for the non-polypeptide moiety in question, or may be deleted. Removal of an N-glycosylation group, may also be accomplished by insertion or removal of an amino acid reside within the motif N-X-S/T/C. In case of introduction of an attachment group, an amino acid residue comprising such group is introduced into the position, such as by substitution of the amino acid residue occupying such position.

The exact number of attachment groups available for conjugation is dependent on the effect desired to be achieved by conjugation. The effect to be obtained is, e.g., dependent on the nature and degree of conjugation (e.g the identity of the non-polypeptide moiety, the number of non-polypeptide moieties desirable or possible to conjugate to the polypeptide, where they should be conjugated or where conjugation should be avoided, etc.). For instance, if reduced immunogenicity is desired, the number (and location of) attachment groups should be sufficient to shield most or all epitopes. This is normally obtained when a greater proportion of the polypeptide is shielded. Effective shielding of epitopes is normally achieved when the total number of attachment groups available for conjugation is in the range of 1-6 attachment groups, e.g., 1-5, such as in the range of 1-3, such as 1, 2, or 3 attachment groups.

Functional in vivo half-life is i.a. dependent on the molecular weight of the conjugate, and the number of attachment groups needed for providing increased half-life thus depends on the molecular weight of the non-polypeptide moiety in question. Some such conjugates comprise 1-6, e.g., 1-5, such as 1-3, e.g. 1, 2, or 3 non-polypeptide moieties each having a MW of about 2-40 kDa, such as about 2 kDa, about 5 kDa, about 12 kDa, about 15 kDa, about 20 kDa, about 30 kDa, or about 40 kDa.

In the conjugate of the invention, some, most, or substantially all conjugatable attachment groups are occupied by the relevant non-polypeptide moiety.

The conjugate of the invention may exhibit one or more of the following improved properties. For example, the conjugate may exhibit a reduced immunogenicity as compared to the corresponding non-conjugated polypeptide, e.g. a reduction of at least 10%, such as a reduction of at least of 25%, such as a reduction of at least of 50%, e.g. a reduction of at least 75% compared to the non-conjugated polypeptide. In another aspect the conjugate may exhibit a reduced reaction or no reaction with neutralizing antibodies from patients treated with the parent polypeptide as compared to the corresponding non-conjugated polypeptide, e.g. a reduction of neutralization of at least 10%, such as at least 25%, such as of at least 50%, e.g., at least 75%.

In another aspect of the invention the conjugate may exhibit an increased functional in vivo half-life and/or increased serum half-life as compared to a reference molecule or as compared to the corresponding non-conjugated polypeptide. Particular preferred conjugates are such conjugates where the ratio between the functional in vivo half-life (or serum half-life) of said conjugate and the functional in vivo half-life (or serum half-life) of said reference molecule is at least 1.25, such as at least 1.50, such as at least 1.75, such as at least 2, such as at least 3, such as at least 4, such as at least 5, such as at least 6, such as at least 7, such as at least 8, such as at least 9, e.g. 10-100. As mentioned above, the half-life is conveniently determined in an experimental animal, such as rat or monkey, and may be based on intravenous, subcutaneous, or other route of administration.

In a further aspect the conjugate may exhibit an increased bioavailability as compared to a reference molecule or the corresponding non-conjugated polypeptide. For example, the conjugate may exhibit an increased AUC_{sc} as compared to a reference molecule or the corresponding non-conjugated polypeptide. Thus, exemplary conjugates are such conjugates where the ratio between the AUC_{sc} of said conjugate and the AUC_{sc} of said reference molecule is at least 1.25, such as at least 1.5, such as at least 2, such as at least 3, such as at least 4, such as at least 5 or at least 6, such as at least 7, such as at least 8, such as at least 9 or at least 10, such as at least 12, such as at least 14, e.g. at least 16, at least 18 or at least 20 when administered subcutaneously, intravenously, intrathecally, intramuscularly, or intraperitoneally, or by ingestion or inhalation, in particular when administered subcutaneously in an experimental animal such as rat or monkey. Analogously, some conjugates of the invention are such conjugates wherein the ratio between Tₘₐₓ for said conjugate and Tₘₐₓ for said reference molecule, or the corresponding non-conjugated polypeptide, is at least 1.2, such as at least 1.4, e.g. at least 1.6, such as at least 1.8, such as at least 2, e.g. at least 2.5, such as at least 3, such as at least 4, e.g. at least 5, such as at least 6, such as at least 7, e.g. at least 8, such as at least 9, such as at least 10, when administered subcutaneously, intravenously, intrathecally, intramuscularly, or intraperitoneally, or by ingestion or inhalation, in particular when administered subcutaneously in an experimental animal such as rat or monkey.

In some instances, the magnitude of the antiviral, anticancer, anti-neoplastic, anti-inflammatory, pro-regenerative or other therapeutic activity of a conjugate of the invention may be reduced (e.g. by at least about 75%, at least about 50%, at least about 25%, at least about 10%) or increased (e.g. by at least about 10%) or is about equal (e.g. within about +/−10% or about +/−5%) to that of the corresponding non-conjugated polypeptide.

In one aspect, the invention relates to a conjugate comprising at least one non-polypeptide moiety conjugated to at least one lysine residue and/or to the N-terminal amino group of a polypeptide of the invention most particularly the polypeptides described in FIG. 3.

In another aspect, the invention relates to a conjugate comprising at least one non-polypeptide moiety conjugated to at least one lysine residue, or to the N-terminal amino group, of a polypeptide comprising a sequence which differs in 1 to 34 amino acid positions from a sequence of FIG. 3.

Some conjugates of the invention comprise a polypeptide sequence comprising a substitution of an amino acid residue for a different amino acid residue, or a deletion of an amino acid residue, which removes one or more lysines from a polypeptide of the invention. The one or more lysine residue(s) to be removed may be substituted with any other amino acid, may be substituted with an Arg (R), His(H) or Gln (Q), or may be deleted.

In instances where amine-reactive conjugation chemistries are employed, it may be advantageous to avoid or to minimize the potential for conjugation to histidine residues. Therefore, some conjugates of the invention comprise a polypeptide sequence comprising a substitution or a deletion which removes one or more histidines from any polypeptide sequence of the invention. The one or more histidine residue(s) to be removed may be substituted with any other amino acid, may be substituted with an Arg (R), Lys(L) or Gln (Q), or may be deleted.

Alternatively, or in addition, some conjugates of the invention comprise a polypeptide sequence comprising a modification which introduces a lysine into a position that is occupied in the parent sequence by an amino acid residue that is exposed to the surface of the molecule, e.g., one that has at least 25%, such as at least 50% of its side chain exposed to the surface.

Non-polypeptide moieties contemplated for this aspect of the invention include polymer molecules, such as PEG or mPEG or mPEG2. The conjugation between the lysine-containing polypeptide and the polymer molecule may be achieved in any suitable manner as known in the art. An exemplary method for PEGylating the polypeptide is to covalently attach PEG to lysine residues using lysine-reactive PEGs. A number of highly specific, lysine-reactive PEGs (such as for example, succinimidyl propionate (SPA), succinimidyl butanoate (SBA), N-hydroxylsuccinimide (NHS), and aldehyde (e.g., ButyrALD)) and different size linear or branched PEGs (e.g., 2-40 kDa, such as 2 kDa, 5 kDa, 12 kDa, 15 kDa, 20 kDa, 30 kDa, or 40 kDa) are commercially available, e.g. from Nektar Therapeutics Inc., Huntsville, Ala., USA, or SunBio, Anyang City, South Korea.

In another aspect, the invention includes a composition comprising a population of conjugates wherein the majority of the conjugates of said population each contain a single non-polypeptide moiety (such as, a single polymer molecule, e.g., a single PEG, such as a linear PEG or a branched PEG) covalently attached to a single lysine residue or N-terminal amino group of the polypeptide. For example, a “monoconjugated” (such as, a “monoPEGylated”) composition of the invention comprises one or more “positional isomers” of said conjugate, wherein each positional isomer contains a single non-polypeptide moiety (e.g., a single PEG molecule) covalently attached to a single lysine residue of the polypeptide.

The invention includes a monoPEGylated composition comprising a population of conjugates, wherein the majority of the conjugates of said population are positional isomers each containing a single PEG molecule (such as, a linear or branched PEG, such as a 2 kDa, 5 kDa, 12 kDa, 15 kDa, 20 kDa, 30 kDa, or 40 kDa mPEG or mPEG2 molecule) covalently attached to a single lysine residue of a polypeptide of the invention.

In one aspect, the invention relates to a conjugate comprising at least one non-polypeptide moiety conjugated to at least one cysteine residue of a polypeptide of the invention or a polypeptide comprising a sequence which differs in 1 to 34 amino acid positions from a sequence of FIG. 3. Some conjugates according to this aspect comprise at least one introduced cysteine residue.

In another aspect, the invention relates to conjugation of the non-polypeptide moiety to one or more cysteine residues of the polypeptides of the invention.

In another aspect, the invention relates to the addition of one or more cysteine residues to the polypeptides of the invention to enable conjugation of a non-polypeptide moiety at a novel location.

In some instances, only a single cysteine residue is introduced in order to avoid formation of disulfide bridges between two or more introduced cysteine residues.

Non-polypeptide moieties contemplated in this aspect of the invention include polymer molecules, such as PEG or mPEG and others as known to those skilled in the art and as described herein. The conjugation between the cysteine-containing polypeptide and the polymer molecule may be achieved in any suitable manner as known to those skilled in the art. An exemplary method for PEGylating the polypeptides of the invention is to covalently attach PEG to cysteine residues using cysteine-reactive PEGs. A number of highly specific, cysteine-reactive PEGs with different groups (e.g. orthopyridyl-disulfide (OPSS), maleimide (MAL) and vinylsulfone (VS)) and different size linear or branched PEGs (e.g., 2-40 kDa, such as 2 kDa, 5 kDa, 12 kDa, 15 kDa, 20 kDa, 30 kDa, or 40 kDa) are commercially available, e.g. from Nektar Therapeutics Inc., Huntsville, Ala., USA, or SunBio, Anyang City, South Korea.

As indicated above, the non-polypeptide moiety of the conjugate of the invention is generally selected from the group consisting of a polymer molecule, a lipophilic compound, a sugar moiety (e.g., by way of in vivo N-glycosylation) and an organic derivatizing agent. All of these agents may confer desirable properties to the polypeptide part of the conjugate, such as reduced immunogenicity, increased functional in vivo half-life, increased serum half-life, increased bioavailability and/or increased AUC_{sc}. The polypeptide part of the conjugate is often conjugated to only one type of non-polypeptide moiety, but may also be conjugated to two or more different types of non-polypeptide moieties, e.g. to a polymer molecule and a sugar moiety, etc. The conjugation to two or more different non-polypeptide moieties may be done simultaneously or sequentially. The choice of non-polypeptide moiety/moieties, depends especially on the effect desired to be achieved by the conjugation. For instance, sugar moieties have been found particularly useful for reducing immunogenicity, whereas polymer molecules such as PEG are of particular use for increasing functional in vivo half-life and/or serum half-life. Using a combination of a polymer molecule and a sugar moiety may enhance the reduction in immunogenicity and the increase in functional in vivo or serum half-life.

For conjugation to a lipophilic compound, the following polypeptide groups may function as attachment groups: the N-terminus or C-terminus of the polypeptide, the hydroxy groups of the amino acid residues Ser, Thr or Tyr, the epsilon-amino group of Lys, the SH group of Cys or the carboxyl group of Asp and Glu. The polypeptide and the lipophilic compound may be conjugated to each other either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine and sulphonic acid with one or more alkyl, aryl, alkenyl or other multiple unsaturated compounds. The conjugation between the polypeptide and the lipophilic compound, optionally through a linker may be done according to methods known in the art, e.g. as described by Bodanszky in Peptide Synthesis, John Wiley, New York, 1976 and in WO 96/12505.

The polymer molecule to be coupled to the polypeptide may be any suitable polymer molecule, such as a natural or synthetic homo-polymer or heteropolymer, typically with a molecular weight in the range of about 300-100,000 Da, such as about 1000-50,000 Da, e.g. in the range of about 1000-40,000 Da. More particularly, the polymer molecule, such as PEG, in particular mPEG, will typically have a molecular weight of about 2, 5, 10, 12, 15, 20, 30, 40 or 50 kDa, in particular a molecular weight of about 5 kDa, about 10 kDa, about 12 kDa, about 15 kDa, about 20 kDa, about 30 kDa or about 40 kDa. The PEG molecule may be branched (e.g., mPEG2), or may be unbranched (i.e., linear).

When used about polymer molecules herein, the word “about” indicates an approximate average molecular weight and reflects the fact that there will normally be a certain molecular weight distribution in a given polymer preparation.

Examples of homo-polymers include a polyol (i.e. poly-OH), a polyamine (i.e. poly-NH₂) and a polycarboxylic acid (i.e. poly-COOH). A hetero-polymer is a polymer which comprises one or more different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs (PEG2), poly-vinyl alcohol (PVA), poly-carboxylate, poly-(vinylpyrolidone), polyethylene-co-maleic acid anhydride, polystyrene-co-malic acid anhydride, dextran including carboxymethyl-dextran, or any other biopolymer suitable for reducing immunogenicity and/or increasing functional in vivo half-life and/or serum half-life. Generally, polyalkylene glycol-derived polymers are biocompatible, non-toxic, non-antigenic, non-immunogenic, have various water solubility properties, and are easily excreted from living organisms.

PEG is the preferred polymer molecule to be used, since it has only few reactive groups capable of cross-linking compared to e.g. polysaccharides such as dextran. In particular, monofunctional PEG, e.g. monomethoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butanoate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitably activated polymer molecules are commercially available, e.g. from Nektar Therapeutics, Inc., Huntsville, Ala., USA; PolyMASC Pharmaceuticals plc, UK; or SunBio Corporation, Anyang City, South Korea. Alternatively, the polymer molecules can be activated by conventional methods known in the art, e.g. as disclosed in WO 90/13540. Specific examples of activated linear or branched polymer molecules suitable for use in the present invention are described in the Nektar Therapeutics, Inc. 2003 Catalog (“Nektar Molecule Engineering: Polyethylene Glycol and Derivatives for Advanced Pegylation, Catalog 2003”), incorporated by reference herein. Specific examples of activated PEG polymers include the following linear PEGs: NHS-PEG, SPA-PEG, SSPA-PEG, SBA-PEG, SS-PEG, SSA-PEG, SC-PEG, SG-PEG, SCM-PEG, NOR-PEG, BTC-PEG, EPOX-PEG, NCO-PEG, NPC-PEG, CDI-PEG, ALD-PEG, TRES-PEG, VS-PEG, OPSS-PEG, IODO-PEG, and MAL-PEG, and branched PEGs, such as PEG2-NHS, PEG2-MAL, and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): Harris and Zalipsky, eds., Poly(ethylene glycol) Chemistry and Biological Applications, AZC, Washington; R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, New York.

For PEGylation of cysteine residues, the polypeptide is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to about 16 hours. Examples of activated PEG polymers for coupling to cysteine residues include the following linear and branched PEGs: vinylsulfone-PEG (PEG-VS), such as vinylsulfone-mPEG (mPEG-VS); orthopyridyl-disulfide-PEG (PEG-OPSS), such as orthopyridyl-disulfide-mPE-G (mPEG-OPSS); and maleimide-PEG (PEG-MAL), such as maleimide-mPEG (mPEG-MAL) and branched maleimide-mPEG2 (mPEG2-MAL).

Pegylation of lysines often employs PEG-N-hydroxylsuccinimide (e.g., mPEG-NHS or mPEG2-NHS), or esters such as PEG succinimidyl propionate (e.g., mPEG-SPA) or PEG succinimidyl butanoate (e.g., mPEG-SBA). One or more PEGs can be attached to a protein within 30 minutes at pH 8-9.5 at room temperature if about equimolar amounts of PEG and protein are mixed. A molar ratio of PEG to protein amino groups of 1-5 to 1 will usually suffice. Increasing pH increases the rate of reaction, while lowering pH reduces the rate of reaction. These highly reactive active esters can couple at physiological pH, but less reactive derivatives typically require higher pH. Low temperatures may also be employed if a labile protein is being used. Under low temperature conditions, a longer reaction time may be used.

N-terminal PEGylation is facilitated by the difference between the pKa values of the alpha-amino group of the N-terminal amino acid (about 6 to 8.0) and the epsilon-amino group of lysine (about 10). PEGylation of the N-terminal amino group often employs PEG-aldehydes (such as mPEG-propionaldehyde or mPEG-butylaldehyde), which are more selective for amines and thus are less likely to react with the imidazole group of histidine; in addition, PEG reagents used for lysine conjugation (such as mPEG-SPA, mPEG-SBA, or mPEG-NHS) may also be used for conjugation of the N-terminal amine. Conjugation of a PEG-aldehyde to the N-terminal amino group typically takes place in a suitable buffer (such as, 100 mM sodium acetate or 100 mM sodium bisphosphate buffer with 20 mM sodium cyanoborohydride) at pH about 5.0 overnight at temperatures varying from about 4° C. to 25° C. Useful N-terminal PEGylation methods and chemistries are also described in U.S. Pat. No. 5,985,265 and U.S. Pat. No. 6,077,939, both incorporated herein by reference.

Typically, linear PEG or mPEG polymers will have a molecular weight of about 5 kDa, about 10 kDa, about 12 kDa, about 15 kDa, about 20 kDa, or about 30 kDa. Branched PEG (PEG2 or mPEG2) polymers will typically have a molecular weight of about 10 kDa, about 20 kDa, or about 40 kDa. In some instances, the higher-molecular weight branched PEG2 reagents, such as 20 kDa or 40 kDa PEG2, including e.g. mPEG2-NHS for lysine PEGylation, mPEG2-MAL for cysteine PEGylation, or MPEG2-aldehyde for N-terminal PEGylation (all available from Nektar Therapeutics, Inc, Huntsville Ala.), may be used. The branched structure of the PEG2 compound results in a relatively large molecular volume, so fewer attached molecules (or, one attached molecule) may impart the desired characteristics of the PEGylated molecule.

The skilled person will be aware that the activation method and/or conjugation chemistry to be used depends on the attachment group(s) of the oligoadenylate synthetase polypeptide as well as the functional groups of the polymer (e.g., being amino, hydroxyl, carboxyl, aldehyde or sulfhydryl). The PEGylation may be directed towards conjugation to all available attachment groups on the polypeptide (i.e. such attachment groups that are exposed at the surface of the polypeptide) or may be directed towards specific attachment groups, e.g. cysteine residues, lysine residues, or the N-terminal amino group. Furthermore, the conjugation may be achieved in one step or in a stepwise manner (e.g. as described in WO 99/55377).

In some instances, the polymer conjugation is performed under conditions aiming at reacting as many of the available polymer attachment groups as possible with polymer molecules. This is achieved by means of a suitable molar excess of the polymer in relation to the polypeptide. Typical molar ratios of activated polymer molecules to polypeptide are up to about 1000-1, such as up to about 200-1 or up to about 100-1. In some cases, the ratio may be somewhat lower, however, such as up to about 50-1, 10-1 or 5-1. Also equimolar ratios may be used.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation residual activated polymer molecules are blocked according to methods known in the art, e.g. by addition of primary amine to the reaction mixture, and the resulting inactivated polymer molecules removed by a suitable method.

Covalent in vitro coupling of a sugar moiety to amino acid residues of the polypeptides of the invention may be used to modify or increase the number or profile of sugar substituents. Depending on the coupling mode used, the carbohydrate(s) may be attached to: a) arginine and histidine (Lundblad and Noyes, Chemical Reagents for Protein Modification, CRC Press Inc. Boca Raton, Fla.), b) free carboxyl groups (e.g. of the C-terminal amino acid residue, asparagine or glutamine), c) free sulfhydryl groups such as that of cysteine, d) free hydroxyl groups such as those of serine, threonine, tyrosine or hydroxyproline, e) aromatic residues such as those of phenylalanine or tryptophan or f) the amide group of glutamine. These amino acid residues constitute examples of attachment groups for a sugar moiety, which may be introduced and/or removed in the polypeptides of the invention. Suitable methods of in vitro coupling are described in WO 87/05330 and in Aplin et al., CRC Crit Rev. Biochem., pp. 259-306, 1981. The in vitro coupling of sugar moieties or PEG to protein- and peptide-bound Gln-residues can also be carried out by transglutaminases (TGases), e.g. as described by Sato et al., 1996 Biochemistry 35, 13072-13080 or in EP 725145.

In order to achieve in vivo glycosylation of an oligoadenylate synthetase polypeptide that has been modified by introduction of one or more glycosylation sites, the nucleotide sequence encoding the polypeptide part of the conjugate is inserted in a glycosylating, eukaryotic expression host. The expression host cell may be selected from fungal (filamentous fungal or yeast), insect, mammalian animal cells, from transgenic plant cells or from transgenic animals. Furthermore, the glycosylation may be achieved in the human body when using a nucleotide sequence encoding the polypeptide part of a conjugate of the invention or a polypeptide of the invention in gene therapy. In one aspect the host cell is a mammalian cell, such as a CHO cell, a COS cell, a BHK or HEK cell, e.g. HEK293, or an insect cell, such as an SF9 cell, or a yeast cell, e.g. Saccharomyces cerevisiae, Pichia pastoris or any other suitable glycosylating host, e.g. as described further below. Optionally, sugar moieties attached to the oligoadenylate synthetase polypeptide by in vivo glycosylation are further modified by use of glycosyltransferases, e.g. using the GlycoAdvance™ technology marketed by Neose, Horsham, Pa., USA. Thereby, it is possible to, e.g., increase the sialyation of the glycosylated oligoadenylate synthetase polypeptide following expression and in vivo glycosylation by CHO cells.

Covalent modification of the polypeptides of the invention may be performed by reacting (an) attachment group(s) of the polypeptide with an organic derivatizing agent. Suitable derivatizing agents and methods are well known in the art. For example, cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(4-imidozoyl- )propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide is also useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine guanidino group. Carboxyl side groups (aspartyl or glutamyl or C-terminal amino acid residue) are selectively modified by reaction with carbodiimides (R—N—double bond—C—double bond —N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Since excessive polymer conjugation may lead to a loss of activity of the oligoadenylate synthetase polypeptides to which the polymer is conjugated, it may be advantageous to remove attachment groups located at the functional site or to block the functional site prior to conjugation. These latter strategies constitute further aspects of the invention (the first strategy being exemplified further above, e.g. by removal of lysine residues which may be located close to a functional site). More specifically, according to the second strategy the conjugation between the oligoadenylate synthetase polypeptide and the non-polypeptide moiety is conducted under conditions where the functional site of the polypeptide is blocked by a helper molecule capable of binding to the functional site of the polypeptide. Preferably, the helper molecule is one which specifically recognizes a functional site of the polypeptide. Alternatively, the helper molecule may be an antibody, in particular a monoclonal antibody recognizing the polypeptide. In particular, the helper molecule may be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such as a polymer. Following its elution from the helper molecule, the conjugate between the non-polypeptide moiety and the polypeptide can be recovered with at least a partially preserved functional site. The subsequent conjugation of the polypeptide having a blocked functional site to a polymer, a lipophilic compound, an organic derivatizing agent or any other compound is conducted in the normal way.

Irrespective of the nature of the helper molecule to be used to shield the functional site of the polypeptide from conjugation, it is desirable that the helper molecule is free from or comprises only a few attachment groups for the non-polypeptide moiety of choice in parts of the molecule where the conjugation to such groups would hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in some instances the helper molecule is a protein or peptide capable of binding to the functional site of the polypeptide, which protein or peptide is free from any conjugatable attachment groups for the non-polypeptide moiety of choice.

In a further aspect the helper molecule is first covalently linked to a solid phase such as column packing materials, for instance Sephadex or agarose beads, or a surface, e.g. reaction vessel. Subsequently, the polypeptide is loaded onto the column material carrying the helper molecule and conjugation carried out according to methods known in the art. This procedure allows the polypeptide conjugate to be separated from the helper molecule by elution. The polypeptide conjugate is eluted by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatized with a second molecule (e.g. biotin) that can be recognized by a specific binder (e.g. streptavidin). The specific binder may be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. De-protection may be achieved by providing conditions in which the helper molecule dissociates from the functional site of the polypeptide to which it is bound; for instance, a complex between an antibody to which a polymer is conjugated and an anti-idiotypic antibody can be dissociated by adjusting the pH to an acid or alkaline pH.

In another aspect the oligoadenylate synthetase polypeptide is expressed as a fusion protein with a tag, i.e. an amino acid sequence or peptide made up of typically 1-30, such as 1-20 or 1-15 or 1-10 or 1-5 amino acid residues, e.g. added to the N-terminus or to the C-terminus of the polypeptide. Besides allowing for fast and easy purification, the tag is a convenient tool for achieving conjugation between the tagged polypeptide and the non-polypeptide moiety. In particular, the tag may be used for achieving conjugation in microtiter plates or other carriers, such as paramagnetic beads, to which the tagged polypeptide can be immobilised via the tag. The conjugation to the tagged polypeptide in, e.g., microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag may function as a spacer molecule ensuring an improved accessibility to the immobilised polypeptide to be conjugated. The conjugation using a tagged polypeptide may be to any of the non-polypeptide moieties disclosed herein, e.g. to a polymer molecule such as PEG.

The identity of the specific tag to be used is not critical as long as the tag is capable of being expressed with the polypeptide and is capable of being immunobilised on a suitable surface or carrier material. A number of suitable tags are commercially available, e.g. from Unizyme Laboratories, Denmark. Antibodies against such tags are commercially available, e.g. from ADI, Aves Lab and Research Diagnostics.

The polypeptides of the invention include modified or mutant oligoadenylate synthetases with increased cell permeability, such increased cell permeability being affected by the addition of one or more basic amino acids residues (e.g. arginine, lysine, histidine), such as the addition of one basic residue, such as two basic residues, e.g. three basic residues, such as about four basic residues, e.g. five basic residues, such as about six basic residues, e.g. about 10 basic residues, e.g. 1-10 basic residues, such as about 5-10 basic residues, such as about 10-15 basic residues, e.g. 5-20 basic residues, said residues being added anywhere within the polypeptides of the invention, including but not limited to at the N-terminus or C-terminus.

Antiviral Treatments Using OAS Polypeptides

The polynucleotides and polypeptides of the invention may be used therapeutically or prophylactically to treat or prevent virus infection. Exemplary viruses include, but are not limited to, viruses of the Flaviviridae family, such as, for example, Hepatitis C Virus, Yellow Fever Virus, West Nile Virus, Japanese Encephalitis Virus, Dengue Virus, and Bovine Viral Diarrhea Virus; viruses of the Hepadnaviridae family, such as, for example, Hepatitis B Virus; viruses of the Picomaviridae family, such as, for example, Encephalomyocarditis Virus, Human Rhinovirus, and Hepatitis A Virus; viruses of the Retroviridae family, such as, for example, Human Immunodeficiency Virus, Simian Immunodeficiency Virus, Human T-Lymphotropic Virus, and Rous Sarcoma Virus; viruses of the Coronaviridae family, such as, for example, SARS coronavirus; viruses of the Rhabdoviridae family, such as, for example, Rabies Virus and Vesicular Stomatitis Virus, viruses of the Paramyxoviridae family, such as, for example, Respiratory Syncytial Virus and Parainfluenza Virus, viruses of the Papillomaviridae family, such as, for example, Human Papillomavirus, and viruses of the Herpesviridae family, such as, for example, Herpes Simplex Virus.

Anticancer and Inflammation Treatments Using OAS Polypeptides

It has been demonstrated that oligoadenylate synthetase polypeptides and 2-prime, 5-prime-oligoadenylates can cause certain cell types and cell lines to undergo apoptosis or to affect growth retardation of said cell lines or cell types. Such cell lines or cell types include in an exemplary embodiment those derived from the prostate and breast.

The invention provides a method of inhibiting proliferation of a cell population, comprising contacting the cell population with a polypeptide of the invention in an amount effective to decrease proliferation of the cell population. The cell population may be in culture or otherwise isolated from a mammal (i.e., in vitro or ex vivo), or may be in vivo, e.g., in a subject, in a mammal, a primate, or man.

The invention provides for treating cancers and neoplastic diseases using the polypeptides and polynucleotides of the invention. Exemplary cancers and neoplastic diseases include but are not limited to: adrenocortical carcinoma, AIDS related cancers, such as for example, Kaposi's sarcoma, AIDS-related lymphoma, anal cancer, astrocytoma, basal cell carcinoma, bile duct cancers, such as for example those of an extrahepatic nature, bladder cancer, bone cancers, such as for example osteosarcomas and malignant fibrous histiocytomas, brain stem glioma, brain tumors, such as for example gliomas, astrocytomas, malignant gliomas, ependymomas, medulloblastomas, and neuroblastomas, supratentorial primitive neuroectodermal tumor, visual pathway and hypothalamic glioma, breast cancer, bronchial adenoma, Burkitt's lymphoma, carcinoid tumors, central nervous system lymphoma, cervical cancer, leukemias, such as for example, hairy cell leukemia, acute lymphoblastic leukemia, acute myeloid leukemia, chronic lymphocytic leukemia and chronic myelogenous leukemia, chronic myeloproliferative disorders, colorectal cancer, cutaneous T-cell lymphoma, endometrial cancer, esophageal cancer, Ewing's family of tumors, extracranial germ cell tumor, extragonadal germ cell tumor, eye cancers, such as for example, intraocular melanoma and retinoblastoma, gallbladder cancer, stomach cancer, gestational trophoblastic tumor, head and neck cancer, hepatocellular carcinoma, Hodgkin's lymphoma, Non-Hodgkin's lymphoma, primary CNS lymphoma, nasopharyngeal cancer, islet cell carcinoma, kidney (renal cell) cancer, laryngeal cancer, lip and oral cancer, liver cancer, lung cancer, such as for example non-small cell and small cell lung cancers, Waldenstrom's macroglobulinemia, Merkel cell carcinoma, mesothelioma, metastatic squamous neck cancer, multiple endocrine neoplasia, multiple myeloma, plasma cell neoplasm, mycosis fungoides, myelodysplastic syndromes, myeloproliferative diseases, nasal cavity and paranasal sinus cancer, ovarian cancer, such as germ cell and epithelial, low-malignant potential ovarian tumor, pancreatic cancer, parathyroid cancer, penile cancer, pheochromocytoma, pituitary tumor, pleuropulmonary blastoma, prostate cancer, rhabdomyosarcoma, salivary gland cancer, sarcomas, Sezary syndrome, skin cancer, such as for example melanoma and squamous cell carcinoma, testicular cancer, thymoma, thymic carcinoma, thyroid cancer, transitional cell cancer, trophoblastic tumor, urethral cancer, uterine cancer, vaginal cancer, vulvar cancer, and Wilms' tumor.

The invention further provides for treating autoimmune diseases and inflammation using the polypeptides and polynucleotides of the invention, said autoimmune and inflammatory diseases include but are not limited to: asthma, Crohn's disease, Guillain-Barre syndrome, multiple sclerosis, myasthenia gravis, optic neuritis, psoriasis, rheumatoid arthritis, Grave's disease, Hashimoto's (thyroiditis) disease, Ord's thyroiditis, diabetes, diabetes mellitus, Reiter's syndrome, autoimmune hepatitis, primary biliary cirrhosis, liver cirrhosis, liver fibrosis, antiphospholipd antibody syndrome, opsoclonus myoclonus syndrome, temporal arteritis, acute disseminated encephalomyelitis, Goodpasture's syndrome, Wegener's granulomatosis, coeliac disease, pemphigus, polyarthritis, warm autoimmune hemolytic anemia, Takayasu's arteritis, coronary artery disease, endometriosis, interstitial cystitis, neuromyotonia, scleroderma, vitiligo, vulvodynia, Chagas' disease, sarcoidosis, chronic fatigue syndrome, acute respiratory distress syndrome, tendonitis, bursitis, polymyalgia rheumatica, inflammatory bowel disease, chronic obstructive pulmonary disease, allergic rhinitis, cardiovascular disease, chronic cholecystitis, bronchiectasis, pneumoconiosis, such as for example, silicosis, osteoarthritis, atherosclerosis, dysautonomia, ankylosing spondylitis, acute anterior uvelitis, systemic lupus erythematosus, insulin-dependent diabetes mellitus, pemphigus vulgaris, experimental allergic encephalomyelitis, experimental autoimmune uveorenitis, mixed connective tissue disease, Sjorgen's syndrome, autoimmune hemolytic anemia, autoimmune thrombocytopenic purpura, acute rheumatic fever, mixed essential cryoglobulinemia, juvenile rheumatoid arthritis, degenerative joint disease, ankylosing spondylitis, psoriatic arthritis, neuralgia, synoviitis, glomerulonephritis, vasculitis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a subject.

Cell Growth and Tissue Regeneration Treatments using OAS Polvpeptide

Oligoadenylate synthetase polypeptides have been shown to stimulate a mitogenic, cell growth-promoting program in specific cell types and cell lines, such as for example, Huh7 hepatoma cells and MRC5 fetal lung fibroblast cells. This mitogenic program is identified using expression microarray analysis and cell viability assays of cells and cell lines treated with OAS polypeptides. The invention provides for uses of the polypeptides of the invention to stimulate cell growth and tissue regeneration in vitro, in vivo, and ex vivo using tissues and cells derived from subjects or mammals.

Recombinant Expression and Purification of OAS Proteins

Recombinant methods for producing and isolating OAS polypeptides or proteins are described herein. One such method comprises introducing into a population of cells any nucleic acid, which is operatively linked to a regulatory sequence effective to produce the encoded OAS polypeptide, culturing the cells in a culture medium to express the polypeptide, and isolating the polypeptide from the cells or from the culture medium. An amount of OAS encoding nucleic acid sufficient to facilitate uptake by the cells (transfection) and/or expression of the OAS polypeptide is utilized. The nucleic acid is introduced into such cells by any delivery method as is known in the art, including, e.g., injection, gene gun, passive uptake, etc. As one skilled in the art will recognize, the nucleic acid may be part of a vector, such as a recombinant expression vector, including a DNA plasmid vector, or any vector as known in the art. The nucleic acid or vector comprising a nucleic acid encoding an OAS polypeptide may be prepared and formulated by standard recombinant DNA technologies and isolation methods as known in the art. Such a nucleic acid or expression vector may be introduced into a population of cells of a mammal in vivo, or selected cells of the mammal (e.g., tumor cells) may be removed from the mammal and the nucleic acid expression vector introduced ex vivo into the population of such cells in an amount sufficient such that uptake and expression of the encoded polypeptide results. Or, a nucleic acid or vector comprising a nucleic acid encoding an OAS polypeptide is produced using cultured cells in vitro. In one aspect, the method of producing an OAS polypeptide comprises introducing into a population of cells a recombinant expression vector comprising any nucleic acid encoding an OAS polypeptide in an amount and formula such that uptake of the vector and expression of the encoded polypeptide will result; administering the expression vector into a mammal by any introduction/delivery format described herein; and isolating the polypeptide from the mammal or from a byproduct of the mammal.

The invention provides isolated or recombinant nucleic acids (also referred to herein as polynucleotides), collectively referred to as “nucleic acids (or polynucleotides) of the invention”, which encode OAS polypeptides. The polynucleotides of the invention are useful in a variety of applications. As discussed above, the polynucleotides are useful in producing OAS polypeptides. Exemplary polynucleotides of the invention include those of FIG. 1, FIG. 2, and FIG. 3.

Any of the polynucleotides of the invention (which includes those described above) may encode a fusion protein comprising at least one additional amino acid sequence, such as, for example, a secretion/localization sequence, a sequence useful for solubilization or immobilization (e.g., for cell surface display) of the OAS polypeptide, a sequence useful for detection and/or purification of the OAS polypeptide (e.g., a polypeptide purification subsequence, such as an epitope tag, a polyhistidine sequence, and the like), or a sequence for increasing cellular uptake. In another aspect, the invention provides cells comprising one or more of the polynucleotides of the invention. Such cells may express one or more OAS polypeptides encoded by the polynucleotides of the invention.

The invention also provides vectors comprising any of the polynucleotides of the invention. Such vectors may comprise a plasmid, a cosmid, a phage, a virus, or a fragment of a virus. Such vectors may comprise an expression vector, and, if desired, the nucleic acid is operably linked to a promoter, including those discussed herein and below.

The present invention also includes recombinant constructs comprising one or more of the nucleic acid sequences as broadly described above. The constructs comprise a vector, such as, a plasmid, a cosmid, a phage, a virus, a bacterial artificial chromosome (BAC), a yeast artificial chromosome (YAC), and the like, into which a nucleic acid sequence of the invention has been inserted, in a forward or reverse orientation. In some instances, the construct further comprises regulatory sequences, including, for example, a promoter, operably linked to the nucleic acid sequence. Large numbers of suitable vectors and promoters are known to those of skill in the art, and are commercially available.

General texts that describe molecular biological techniques useful herein, including the use of vectors, promoters and many other relevant topics, include Berger, supra; Sambrook (1989), supra, and Ausubel, supra. Examples of techniques sufficient to direct persons of skill through in vitro amplification methods, including the polymerase chain reaction (PCR) the ligase chain reaction (LCR), Q beta-replicase amplification and other RNA polymerase mediated techniques (e.g., NASBA), e.g., for the production of the homologous nucleic acids of the invention are found in Berger, Sambrook, and Ausubel, all supra, as well as Mullis et al. (1987) U.S. Pat. No. 4,683,202; PCR Protocols: A Guide to Methods and Applications (Innis et al., eds.) Academic Press Inc. San Diego, Calif. (1990) (“Innis”); Arnheim & Levinson (Oct. 1, 1990) C&EN 36-47; The Journal Of NIH Research (1991) 3:81-94; (Kwoh et al. (1989) Proc Natl Acad Sci USA 86:1173-1177; Guatelli et al. (1990) Proc Natl Acad Sci USA 87:1874-1878; Lomeli et al. (1989) J Clin Chem 35:1826-1831; Landegren et al. (1988) Science 241:1077-1080; Van Brunt (1990) Biotechnology 8:291-294; Wu and Wallace (1989) Gene 4:560-569; Barringer et al. (1990) Gene 89:117-122, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kilobases (kb) are generated. One of skill will appreciate that essentially any RNA can be converted into a double stranded DNA suitable for restriction digestion, PCR expansion and sequencing using reverse transcriptase and a polymerase. See Ausubel, Sambrook and Berger, all supra.

The present invention also provides host cells that are transduced with vectors of the invention, and the production of OAS polypeptides of the invention by recombinant techniques. Host cells are genetically engineered (e.g., transduced, transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants, or amplifying genes. The culture conditions, such as temperature, pH, and the like, are those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein.

OAS polypeptides can also be produced in non-animal cells such as plants, yeast, fungi, bacteria and the like. In addition to Sambrook, Berger and Ausubel, details regarding cell culture are found in, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York); Atlas & Parks (eds.) The Handbook of Microbiological Media (1993) CRC Press, Boca Raton, Fla.

The polynucleotides of the present invention and fragments thereof may be included in any one of a variety of expression vectors for expressing an OAS polypeptide. Such vectors include chromosomal, nonchromosomal and synthetic DNA sequences, e.g., derivatives of SV40, bacterial plasmids, phage DNA, baculovirus, yeast plasmids, vectors derived from combinations of plasmids and phage DNA. Any vector that transduces genetic material into a cell, and, if replication is desired, which is replicable and viable in the relevant host can be used.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda PL promoter, CMV promoter, and other promoters known to control expression of genes in prokaryotic or eukaryotic ceils. The expression vector also contains a ribosome binding site for translation initiation, and a transcription terminator. The vector optionally includes appropriate sequences for amplifying expression, e.g., an enhancer. In addition, the expression vectors optionally comprise one or more selectable marker genes to provide a phenotypic trait for selection of transformed host cells, such as dihydrofolate reductase or neomycin resistance for eukaryotic cell culture, or such as tetracycline, kanamycin or ampicillin resistance in E. coli.

The vector containing the appropriate DNA sequence encoding an OAS polypeptide of the invention, as well as an appropriate promoter or control sequence, may be employed to transform an appropriate host to permit the host to express the polypeptide. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 or Bowes melanoma; plant cells, etc. It is understood that not all cells or cell lines need to be capable of producing fully functional OAS polypeptides or fragments thereof; for example, antigenic fragments of the polypeptide may be produced in a bacterial or other expression system. The invention is not limited by the host cells employed.

In bacterial systems, a number of expression vectors may be selected depending upon the use intended for the OAS polypeptide or fragment thereof. For example, when large quantities of a polypeptide or fragments thereof are needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the nucleotide coding sequence may be ligated into the vector in-frame with sequences for the amino-terminal Met and the subsequent 7 residues of beta-galactosidase so that a hybrid protein is produced; pIN vectors (Van Heeke & Schuster (1989) J Biol Chem 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH may be used for production of the polypeptides of the invention. For reviews, see Ausubel, supra, Berger, supra, and Grant et al. (1987) Methods in Enzymology 153:516-544.

In mammalian host cells, a number of expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome results in a viable virus capable of expressing an OAS polypeptide in infected host cells (Logan and Shenk (1984) Proc Natl Acad Sci USA 81:3655-3659). In addition, transcription enhancers, such as the rous sarcoma virus (RSV) enhancer, are used to increase expression in mammalian host cells. Host cells, media, expression systems, and methods of production include those known for cloning and expression of various mammalian proteins.

Specific initiation signals can aid in efficient translation of a polynucleotide coding sequence of the invention and/or fragments thereof. These signals can include, e.g., the ATG initiation codon and adjacent sequences. In cases where a coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous nucleic acid transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (see, e.g., Scharf D. et al. (1994) Results Probl Cell Differ 20:125-62; and Bittner et al. (1987) Methods in Enzymol 153:516-544).

Polynucleotides encoding OAS polypeptides can also be fused, for example, in-frame to nucleic acids encoding a secretion/localization sequence, to target polypeptide expression to a desired cellular compartment, membrane, or organelle, or to direct polypeptide secretion to the periplasmic space or into the cell culture media. Such sequences are known to those of skill, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

In a further aspect, the present invention relates to host cells containing any of the above-described nucleic acids, vectors, or other constructs of the invention. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-dextran mediated transfection, electroporation, gene or vaccine gun, injection, or other common techniques (see, e.g., Davis, L., Dibner, M., and Battey, I. (1986) Basic Methods in Molecular Biology) for in vivo, ex vivo or in vitro methods.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre” or a “prepro” form of the protein may also be important for correct insertion, folding and/or function. Different host cells such as E. coli, Bacillus sp., yeast or mammalian cells such as CHO, HeLa, BHK, MDCK, HEK 293, W138, etc. have specific cellular machinery and characteristic mechanisms for such post-translational activities and may be chosen to ensure the correct modification and processing of the introduced foreign protein.

Stable expression can be used for long-term, high-yield production of recombinant OAS proteins. For example, cell lines which stably express a polypeptide of the invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

Host cells transformed with a nucleotide sequence encoding an OAS polypeptide are optionally cultured under conditions suitable for the expression and recovery of the encoded protein from cell culture. The polypeptide produced by a recombinant cell may be secreted, membrane-bound, or contained intracellularly, depending on the sequence and/or the vector used. As will be understood by those of skill in the art, expression vectors containing polynucleotides encoding polypeptides of the invention can be designed with signal sequences which direct secretion of the mature polypeptides through a prokaryotic or eukaryotic cell membrane.

The polynucleotides of the present invention optionally comprise a coding sequence fused in-frame to a marker sequence which, e.g., facilitates purification and/or detection of the encoded polypeptide. Such purification subsequences include, but are not limited to, metal chelating peptides such as histidine-tryptophan modules that allow purification on immobilized metals, a sequence which binds glutathione (e.g., GST), a hemagglutinin (HA) tag (corresponding to an epitope derived from the influenza hemagglutinin protein; Wilson, I. et al. (1984) Cell 37:767), maltose binding protein sequences, the FLAG epitope utilized in the FLAGS extension/affinity purification system, and the like. The inclusion of a protease-cleavable polypeptide linker sequence between the purification domain and the polypeptide sequence is useful to facilitate purification.

For example, one expression vector possible to use in the compositions and methods described herein provides for expression of a fusion protein comprising an OAS polypeptide fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281) while the enterokinase cleavage site provides a method for separating the desired polypeptide from the polyhistidine region. pGEX vectors (Promega; Madison, Wis.) are optionally used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Eukaryotic or microbial cells employed in expression of the proteins can be disrupted by any convenient method, including freeze-thaw cycling, sonication, mechanical disruption, or use of cell lysing agents, or other methods, which are well know to those skilled in the art.

As noted, many references are available for the culture and production of many cells, including cells of bacterial, plant, animal (especially mammalian) and archebacterial origin. See, e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, New York; Humason (1979) Animal Tissue Techniques, fourth edition W. H. Freeman and Company; and Ricciardelli et al. (1989) In vitro Cell Dev Biol 25:1016-1024. For plant cell culture and regeneration see, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy (ed.) Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue from Sigma-Aldrich, Inc (St Louis, Mo.) (“Sigma-LSRCCC”) and, e.g., the Plant Culture Catalogue and supplement also from Sigma-Aldrich, Inc (St Louis, Mo.) (“Sigma-PCCS”).

OAS polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature OAS protein or fragments thereof. Finally, high performance liquid chromatography (HPLC) can be employed in the final purification steps. In addition to the references noted, supra, a variety of purification methods are well known in the art, including, e.g., those set forth in Sandana (1997) Bioseparation of Proteins, Academic Press, Inc.; Bollag et al. (1996) Protein Methods, 2.sup.nd Edition Wiley-Liss, New York; Walker (1996) The Protein Protocols Handbook Humana Press, New Jersey; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3.sup.rd Edition Springer Verlag, New York; Janson and Ryden (1998) Protein Purification: Principles, High Resolution Methods and Applications, Second Edition Wiley-VCH, New York; and Walker (1998) Protein Protocols on CD-ROM Humana Press, New Jersey.

### EMBODIMENTS

OAS Protein Active Pharmaceutical Ingredient (API) Expression and Fermentation

In an exemplary embodiment, an E. coli strain containing a lysogen of λDE3, and therefore carrying a chromosomal copy of the T7 RNA polymerase gene under the control of the lacUV5 promoter, is transformed with a bacterial expression vector containing an isopropyl beta-D-1-thiogalactopyranoside (IPTG)-inducible promoter encoding a nucleic acid sequence corresponding to one or more OAS proteins or polypeptides. Cultures are grown in Luria broth medium supplemented with 15 μg/mL kanamycin at 37° C. When the OD600 reaches >0.6, the temperature is reduced to 18° C. and the cells are induced with 0.5 mM IPTG for 17 hours. The above low temperature induction favors the expression of primarily full-length, soluble OAS proteins outside of inclusion bodies. The bacterial cells are then resuspended in buffer containing 50 mM NaH₂PO₄, pH 8, 300 mM NaCl, 20 mM imidazole, 10% glycerol, 0.1% NP40,2 mM DTT and protease inhibitors, lysed in a Gaulin homogenizer, and centrifuged to remove cell debris before protein purification.

In another exemplary embodiment, OAS proteins are expressed by cloning into the pET9d expression vector and transformed into the BL2 1 (DE3) host E. coli strain. Recombinant bacterial cultures are grown in Luria broth to an OD(600 nm) of about 0.6 and induced to express OAS proteins by the addition of IPTG to a final concentration of 1 mM for 3-4 hours at 37° C. Under these induction conditions, a majority of full length OAS proteins are found in an insoluble form in inclusion bodies. Bacterial cell cultures are centrifuged to collect the cell pellet at 9000× g. Cell pellets are resuspended in 50 mM NaH₂PO₄, 0.5% Triton X-100, 100 mM NaCl, 1 mM EDTA, pH 7.4. Lysozyme is added to 1 mg/mL and sonication is used to disrupt the cell membrane. DNAse and RNAse are added to a final concentration of 50 ug/mL each to reduce the viscosity of the cell lysate. An equal volume of a solution of 50 mM NaH₂PO₄, 5% Triton X-100, 2 M urea, 100 mM NaCl, 1 mM EDTA, pH 7.4 is added and the mixture is stirred for 30 minutes at room temperature. The lysate is snap-frozen, thawed, and centrifuged at 9000× g to recover inclusion bodies. Inclusion bodies are washed one time in a solution of 50 mM NaH₂PO₄, 5% Triton X-100, 2 M urea, 100 mM NaCl, 1 mM EDTA, pH 7.4 followed by centrifugation at 9000× g for 30 minutes. Additional inclusion body washes are performed using phosphate buffered saline (PBS) pH 7.4 followed by centrifugation as above. Inclusion body pellets are solubilized by the addition of 50 mL of a solution of 50 mM NaH₂PO₄, 6 M guanidine HCl, pH 8.0 for every 2.5 grams of wet inclusion body pellet. Dithiothreitol (DTT) is added to a final concentration of 50 mM. The mixture is stirred at room temperature for at least two hours or until clear. Sonication is used to improve the clarity and solubilization of inclusion bodies. Bacterial expression of OAS proteins can be evaluated by SDS-PAGE of solubilized inclusion body preparations. Approximately 50% or more of solubilized inclusion body protein is found to be OAS protein.

In one embodiment, the bacterial strain used is a derivative of BL21. In another embodiment, bacteria are grown in terrific broth or a synthetic media. In a still further embodiment, media are supplemented with buffers, amino acids, sugars, or other carbon sources. In a still further embodiment, bacteria are grown in shaker flasks, seed cultures, or fermenters. Bacterial cultures are grown to a variety of cell densities before induction of OAS protein expression, depending on culture conditions. Cell density at the time of induction—as measured by optical density at a wavelength of 600 nm—is for example about 0.2, 0.3, 0.4, 0.5, 0.6, 0.7, 0.8, 0.9, 1.0, 1.2, 1.5, 2.0, or 2.5. Bacteria are grown under a variety of selective conditions, depending on the recombinant protein expression vector used and the host E. coli strain. In preferred embodiments, bacteria are grown in the presence of about, for example, 1 ug/mL, 5 ug/mL, 10 ug/mL, 15 ug/mL, 20 ug/mL, 50 ug/mL, or 100 ug/mL kanamycin. In a still further embodiment, bacterial cultures can be grown at any temperature between 30° C. and 40° C.

Induction is performed under a variety of concentrations of the IPTG inducer, such as for example, about 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.5 mM, 2.0 mM, 2.5 mM, 3.0 mM, 4.0 mM, or 5.0 mM. In a still further embodiment, OAS protein induction can be performed at temperatures between 4° C. and 40° C. and at times between 30 minutes and 48 hours. In a still further embodiment, bacterial cultures at appropriate densities are induced for OAS protein expression for 3-4 hours at 37° C. with a final concentration of 1 mM IPTG. A variety of induction temperatures and times are appropriate for OAS protein expression. Shorter induction times and higher temperatures favor the expression of full-length insoluble OAS proteins into inclusion bodies. Longer induction periods and lower temperatures favor expression of soluble OAS protein outside of inclusions bodies. At the end of induction, cells are collected by a variety of methods including centrifugation and filtration. As one skilled in the art will recognize, a variety of cell collection methods are envisioned by the specification. OAS proteins exceed 10% of total cellular protein.

Bacterial cells and inclusion bodies containing recombinant OAS proteins are collected, washed, lysed, and solubilized under a variety of buffer and solution conditions. A variety of buffers over a range of pKₐ values are used for buffering solutions including N-(2-acetamido)-2-aminoethane sulfonic acid (ACES), imidazole, phosphate, N-morpholinopropane sulfonic acid (MOPS) N-tris(hydroxymethyl)methyl-2-aminoethane sulfonic acid (TES), triethanolamine, Tris(hydroxymethyl)aminomethane (TRIS), N-Tris(hydroxymethyl)methyl-glycine (Tricine), Tris(hydroxymethyl)aminopropoane (TAPS), N-(2-Hydroxyethyl)piperazine-N′-(2-ethanesulfonic acid) (HEPES), 2-amino-2-methyl-1,3-propanediol, diethanolamine, boric acid, and ethanolamine. Buffers are used at a variety of concentrations, such as for example, 1 mM, 5 mM, 10 mM, about 25mM, about 50 mM, about 100 mM, about 200 mM and at a variety of pH values, such as for example, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, and sodium thiocyanate, at concentrations of about 10 mM, about 25 mM, about 50 mM, about 75 mM, about 100 mM, about 200 mM, about 300 mM, about 500 mM, about 700 mM, about 1M. Chaotropic agents are used to enhance washing and solubilization of OAS protein containing inclusion bodies, such chaotropic agents include: urea, guanidine HCl, thiourea, and the like, at concentrations such as for example about 0.25M, 0.5M, 1.0M, 2.0M, 3.0M, 4.0M, 5.0M, 6.0M, 7.0M, such as for example 8.0M and above including near saturation solutions. A variety of detergents are added to facilitate bacterial cell lysis and inclusion body washing and solubilization, such as Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), aminosulfobetaine-16 (ASB-16), 3-(1-pyridinio)-1-propanesulfonate (NDSB 201), and dodecyl sulfate, at concentrations of for example, 0.1% w/v, 0.2% w/v, 0.3% w/v, 0.4% w/v, 0.5% w/v, about 1% w/v, about 5% w/v, about 10% w/v, more than 10% w/v. In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Stabilizing agents including surfactants, sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added during cell lysis to stabilize OAS proteins. Thiol-protective or reducing agents are added to prevent errant disulfide bond formation, such thiol-protective groups including dithiothreitol (DTT), dithioerythritol (DTE), 2-mercaptoethanol, 2-3-dimercaptopropanol, tributylphosphine (TBP), tris-carboxyethylphosphine (TCEP), thioglycolate, glutathione, and cysteine at concentrations of between 0.5 and 100 mM, such as for example, 1 mM, about 5 mM, about 10 mM, about 25 mM, about 50 mM, about 100 mM.

Enzymes are added to aid in cell lysis, such as for example lysozyme at concentrations of 1 mg/mL, about 2 mg/mL, about 5 mg/mL, or about 10 mg/mL. Mechanical disruption is used to lyse bacterial cells and to clarify and solubilize inclusion bodies, such appropriate mechanical methods include: sonication, Gaulin homogenization, use of blenders, use of French pressure cells, Dounce homogenization, polytron homogenization, Potter-Elvehjem homogenization, and freeze/thaw methods as those skilled in the art will recognize. Heat, different pH buffers, different reductants and high pressure are also used to enhance solubilization of inclusion bodies. Numerous techniques are used to separate inclusion bodies from other cellular proteins and debris including: centrifugation, membrane filtration, tangential flow filtration, hollow fiber filtration, and expanded bed absorption. Inclusion bodies can also be washed in water.

OAS Protein API Refolding of Insoluble Preparations

In an exemplary embodiment, solubilized inclusion bodies are adjusted to a final protein concentration of 10 to 15 mg/mL prior to pulse dilution into an appropriate refolding buffer. Solubilized inclusion bodies with final protein concentrations greater than 30 mg/mL demonstrate poor refolding potential. OAS protein refolding is performed by pulse dilution at 4° C. and at a flow-rate of 0.2 mL/minute over a 16 hour period into a stirred solution composed of 50 mM NaH₂PO₄, 300 mM guanidine HCl, 0.5% Tween-20®, 10% glycerol, 5 mM β-mercaptoethanol, pH 8.0. Both the solubilized inclusion bodies and the refolding solution are precooled to 4° C. The final total dilution of solubilized inclusion bodies into refolding solution is approximately 1:20. In exemplary embodiments, detergent is used to facilitate proper refolding of the OAS protein API. CHAPS at 0.1%, 0.5% and 1% w/v is used, as well as Tween-20® at a final concentration of between 0.1% and 1.0% w/v. 1% Tween-20® is shown to reduce aggregation of the OAS protein API during refolding. Refolding solutions at pH 8.0 perform better than refolding solutions at pH 6.8. Likewise, refolding solutions containing 2-mercaptoethanol as a reducing agent perform better than refolding solutions containing DTT. Refolding performed at 4° C. is more efficient than a refolding process performed at room temperature. The presence of chaotropic agents and high salt also enhance OAS protein refolding; for example the addition of 300 mM NaCl and 300 mM guanidine HCl enhances protein refolding efficiency. In an exemplary embodiment, fold-dilutions of solubilized inclusion bodies between 10 and 120 produce large amounts of properly folded and highly active OAS protein API. Refolding efficiencies of greater than 40% are achieved.

In another embodiment, immediate dilution is used for refolding of insoluble OAS proteins. In a still further embodiment, buffer exchange through dialysis, tangential flow filtration and gel filtration are used to mediate OAS protein refolding.

In another embodiment alternative buffers over a range of pKₐ values are used for buffering refolding solutions including ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 M, about 200 mM and at a variety of pH values, such as for example, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 mM, about 700 mM, about 1M. Chaotropic agents are used to enhance refolding of OAS proteins, such chaotropic agents include: urea, guanidine HCl, thiourea, and the like, at concentrations such as for example about 0.05M, 0.1M,0.25M, 0.5M, 1.0M, 2.0M, 3.0M,4.0M, 5.0M, 6.0M, 7.0M, such as for example 8.0M and above including near saturation solutions. A variety of detergents are added to improve OAS protein refolding efficiency, such as Nonidet P-40, Tween-80®, Tween-20®, Triton-X100®, Triton-X 114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.1%w/v, 0.2%w/v, 0.3%w/v, 0.4%w/v, 0.5%w/v, about 1%w/v, about 5%w/v, about 10% w/v, more than 10%w/v. In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Chelating agents increase the half-life of thiol-reductants. Stabilizing agents including surfactants, sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added during refolding to stabilize OAS proteins. Thiol-protective or reducing agents are added to prevent errant disulfide bond formation and to cleave inappropriate disulfide bond within the inclusion body, such thiol-protective groups including dithiothreitol (DTT), dithioerythritol (DTE), 2-mercaptoethanol, 2-3-dimercaptopropanol, tributylphosphine (TBP), tris-carboxyethylphosphine (TCEP), thioglycolate, glutathione, and cysteine at concentrations of between 0.5 and 150 mM, such as for example, 1 mM, about 5 mM, about 10 mM, about 25 mM, about 50 mM, about 100 mM, about 150 mM.

OAS Protein API Purification, Concentration and Sterilization

In an exemplary embodiment, the properly refolded OAS protein-containing inclusion body preparations are filtered through a 0.45 micrometer membrane for clarification and loaded onto HiTrap Heparin HP® FPLC columns for initial capture and purification. Heparin columns bind approximately 4-5 mg of OAS protein per milliliter of resin. Heparin columns are pre-equilibrated with 50 mM NaH₂PO₄, 25 mM NaCl, 5% glycerol, 1 mM EDTA, 0.01% Tween-20®, 2 mM DTT, pH 6.8 before the application of refolded inclusion body preparations. OAS proteins bind efficiently to heparin columns. Once bound, immobilized OAS proteins are washed with two column volumes of 50 mM NaH₂PO₄, 25 mM NaCl, 5% glycerol, 1 mM EDTA, 0.01% Tween-20®, 2 mM DTT, pH 6.8 and eluted in a step gradient with 50 mM NaH₂PO₄, 1 M NaCl, 30% glycerol, 1 mM EDTA, 2 mM DTT, pH 6.8. Column chromatography is performed using fast protein liquid chromatography (FPLC) with commercially supplied columns or resins.

In another exemplary embodiment, HiTrap SP Fast Flow ® columns are used when the conductance of the refolded protein preparation is below 6 mS/cm. In a still further embodiment, Cibacron Blue F3G-A (Blue Sepharose) resins are used that demonstrate a lower binding capacity for OAS proteins—approximately 1 mg/mL. In a still further exemplary embodiment, mixed mode resins (e.g. GE Healthcare's Capto MMC®) are used that bind OAS proteins at low affinity (e.g. <1 mg/mL resin). In a still further exemplary embodiment, Capto S and Phenyl HP columns are used for OAS protein capture from refolded inclusion body preparations.

As one skilled in the art will recognize, numerous cation exchange resins are appropriate for the initial capture of OAS proteins from refolded, solubilized inclusion body preparations. Embodiments of appropriate cation exchange resin functional groups include: methyl sulfonate, sulfopropyl, carboxymethyl, sulfonic acid, carbonic acid, and carboxylic acid. Affinity resins that are derivatized with deoxyribonucleic acid or ribonucleic acid finctional groups can also be used to practice the invention. Nicotinamide dye columns are also used to practice the invention. As one skilled in the art will recognize, a variety of column loading conditions and flow-rates are appropriate for a variety of industrial scales and applications.

Other embodiments include the use tangential flow filtration, diafiltration, dialysis, or gel filtration to allow for buffer exchange and concentration. One or more column steps may be substituted by selective precipitation with, for example, ammonium sulfate.

In one embodiment, buffers and buffer conditions, including buffer pH, can be altered to improve column binding capacities and efficiency. Buffer pH can also be altered to improve elution dynamics from the capture column. The following buffer components are used in column loading, wash and elution solutions: ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 mM, about 200 mM and at a variety of pH values, such as for example, lower than 5.0, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher.

Detergents are added to prevent OAS protein aggregation and to limit non-specific protein interactions with the column matrix. A variety of detergent additives are envisioned as components of column loading, wash, and elution solutions including without limitation: Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.001%w/v, about 0.01% w/v, 0.02% w/v, about 0.05% w/v, 0.1%w/v, 0.2%w/v, 0.3%w/v, 0.4%w/v, 0.5%w/v, about 1%w/v, about 2%w/v, more than 2%w/v.

Reductants are used to prevent the formation of non-specific disulfide bonds. Column loading, wash, and elution buffers contain any of a number of reductants including without limitation: DTT, DTE, 2-mercaptoethanol, 2-3-dimercaptopropanol, TBP, TCEP, thioglycolate, glutathione, and cysteine at concentrations of between 0.5 and 150 mM, such as for example, 0.5 mM, 1 mM, 2 mM, 3 mM, 4 mM, about 5 mM, about 10 mM, about 25 mM, about 50 mM, about 100 mM, about 150 mM.

Salts are used to limit non-specific protein-protein interaction, to prevent protein aggregation, and to effect column elution from the cation exchange resin; typically used salts include: sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 M, about 700 mM, about 1M, about 2M, about 3M. Low concentrations of chaotropic agents serve a similar role. Chelating and stabilizing agents are also included in column wash, and elution buffers as described elsewhere in the specification. All manner, combination and concentration of chelating and stabilizing agents are envisioned as components of column wash and elution buffers.

Hydrophobic interaction chromatography (HIC) is next used to purify OAS proteins away from E. coli host cell contaminants. HIC is an effective method for removing bacterial endotoxin and other pyrogens. In an exemplary embodiment, following elution of OAS protein-containing fractions from the initial cation exchange capture column, fractions are pooled and diluted 1:1 with 50 mM NaH₂PO₄, 300 mM NaCl, 20% glycerol, 1 mM EDTA, 2 mM DTT, pH 6.8 and adjusted to a final concentration of 1 M ammonium sulfate. The OAS fractions are loaded onto a Phenyl HP HIC column at a protein density no greater than 7.5 mg/mL of resin. Columns are washed with three column volumes of a solution of 50 mM NaH₂PO₄, 300 mM NaCl, 1 M (NH₄)₂SO₄, 1 mM EDTA, 20% glycerol, 2 mM DTT, pH 6.8, followed by a step gradient to 40% of the following buffer: 50 mM NaH₂PO₄, 300 mM NaCl, 20% glycerol, 1 mM EDTA, 2 mM DTT, pH 6.8 for three column volumes. OAS protein containing fractions are eluted by a step gradient to 85% of the following buffer: 50 mM NaH₂PO₄, 300 mM NaCl, 20% glycerol, 1 mM EDTA, 2 mM DTT, pH 6.8 for three column volumes.

As one skilled in the art will recognize, a variety of column volumes and gradient functions will affect the same level of purity of OAS proteins following HIC. As one skilled in the art will further recognize, a number of salts and salt concentration are appropriate for column loading, wash and elution, with importance given to decreasing conductivity throughout the washing and elution steps.

In one embodiment, butyl, butyl S, octyl, or phenyl derivatized HIC columns are used for OAS capture and elution. Column loading, wash, and elution buffers are effectively formulated with one or more salts, buffers, stabilizing agents, detergents, reductants, and chelating agents at a variety of appropriate concentrations and pH's as described elsewhere in the specification.

Following HIC capture and elution, fractions containing OAS proteins are subjected to anion exchange chromatography to remove E. coli host cell contaminating pyrogens and nucleic acids. OAS containing fractions are diluted 1:5 with a solution composed of: 10 mM NaH₂PO₄, 20% glycerol, 1 mM EDTA, 2 mM DTT, pH 8. The pH of the resulting solution is adjusted to 8.0 by addition of HCl. The pH-adjusted sample is loaded onto a diethylaminoethyl (DEAE) FF column at a rate of 1.5 mL/minute and then washed with five column volumes of a solution composed of: 10 mM NaH₂PO₄, 20% glycerol, 1 mM EDTA, 2 mM DTT, pH 8. OAS proteins are found in the flow through. This step reduces endotoxin contamination to below 1 EU/mL.

As one skilled in the art will recognize, other anion exchange resins can be substituted for DEAE, including but not limited to those derivatized with quaternary ammonium and diethylaminopropyl groups. Other embodiments specifically for removing endotoxin contamination can be employed, including but not limited to the use of polymixin B columns.

Column loading and wash buffers are effectively formulated with one or more buffers, stabilizing agents, detergents, reductants, and chelating agents at a variety of appropriate concentrations and pH's as described elsewhere in the specification.

Following anion exchange chromatography to remove endotoxins, purified OAS proteins are concentrated by one of a variety of methods including cation exchange chromatography, ultrafiltration, or tangential flow filtration. Buffer exchanges are affected by gel filtration, tangential flow filtration/diafiltration, or ultrafiltration/diafiltration. Buffer exchange, protein concentration and terilization result in an API suitable for inclusion into a pharmaceutical composition.

In one exemplary embodiment, the purified OAS protein is diluted to a conductivity of less than 6 mS/cm and the pH is adjusted to 6.8. The OAS protein is then bound to a cation exchange column, such as for example a HiTrap SP FF column, pre-equilibrated in a solution composed of 50 mM NaH₂PO₄, 25 mM NaCl, 20% glycerol, 1 mM EDTA, 2 mM DTT, pH 6.8. The bound OAS protein is washed with three column volumes of a solution composed of 50 mM NaH₂PO₄, 25 mM NaCl, 20% glycerol, 1 mM EDTA, 2 mM DTT, pH 6.8, and eluted with a step gradient to 70% of a solution composed of 50 mM NaH₂PO₄, 1 M NaCl, 30% glycerol, 2 mM DTT, 1 mM EDTA, pH 6.8. Purified OAS fractions are pooled and subjected to gel filtration for buffer exchange using a 2 mL/minute flow rate and a HiTrap desalting column. As one skilled in the art will recognize, any of a number of cation exchange and gel filtration columns will perform adequately for protein concentration and buffer exchange as described elsewhere in the specification. In other embodiments, purified OAS preparations are concentrated via ultrafiltration on Amicon polyethersulfone 10,000 membranes. Buffer exchange can be carried out by diafiltration. Final buffers are chosen based upon the required pharmaceutical composition for the API.

Exemplary Excipient Components for Purified OAS Proteins

OAS proteins are stabilized by excipients containing salts; solutions stable at 300 mM NaCl can begin to precipitate at 150 mM NaCl. For this reason excipient mixtures will favor these stabilizing salt concentrations, which could include but are not limited to sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate.

The addition of amino acid-based excipients such as arginine or glutamine has proven to be stabilizing to purified OAS proteins. The addition of 2% w/v arginine allows OAS proteins to be stable at 3 mg/mL. The addition of excipients such as glycerol is stabilizing to OAS polypeptides. For example, in one embodiment, a polypeptide has a maximum concentration with 10% glycerol (v/v) of 1 mg/mL; while at 40% glycerol, the OAS polypeptides are stable up to 12 mg/mL. Disaccharides such as sucrose have been found to be stabilizing at 10% w/v; other disaccharides including but not limited to maltose and trehalose are also used. Numerous stabilizing agents are appropriate for use as excipients components, including but not limited to: sugars and polyols (e.g. glycerol, sucrose, trehalose, glucose, lactose, inositol, mannitol, xylitol, ethylene glycol), surfactants (e.g. Tween-20®, Tween-80®), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules.

Antioxidants and preservatives are also used to ensure stability of purified OAS proteins during storage. Antioxidants, including but not limited to sodium citrate, may be stabilizing for long term storage of the OAS proteins. Preservatives, including but not limited to, benzyl alcohol may also be stabilizing to the polypeptides during storage and may be used in final excipient mixtures.

Buffer Components for OAS Polypeptides

Bacterial cells and inclusion bodies containing recombinant OAS proteins are collected, washed, lysed, and solubilized under a variety of buffer and solution conditions. Furthermore, a variety of buffer and solution conditions are appropriate for each purification step in the entire manufacturing process leading to the production of a purified API. Without limiting the generality of the methods of the present invention, the methods disclosed herein include but are not limited to the use of alternate buffers, additives, and reagents as is known to one skilled in the art or as further exemplified in the following.

Buffers over a range of pKa values are used for buffering solutions including ACES, imidazole, phosphate, MOPS, TES, triethanolamine, HEPES, TRIS, Tricine, TAPS, 2-amino-2-methyl-1,3-propanediol, diethanolamine, boric acid, and ethanolamine. Buffers are used at a variety of concentrations, such as for example, 1 mM, 5 mM, 10 mM, about 25 mM, about 50 mM, about 100 mM, about 200 mM and at a variety of pH values, such as for example, around 5.0, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, such as about 8.2, about 8.5, about 8.7, about 9.0, about 9.2, about 9.5, about 10, about 10.5, about 11, about 11.5, about 12, about 12.5 and higher. Salts are added to stabilize OAS proteins, such as for example, sodium chloride, potassium chloride, magnesium chloride, calcium chloride, manganese chloride, magnesium sulfate, sodium sulfate, sodium bromide, sodium acetate, calcium sulfate, lithium chloride, sodium iodide, sodium perchlorate, sodium thiocyanate, and ammonium sulfate at concentrations of about 10 mM, about 25 mM, about 50 mM, about 75 mM, about 100 mM, about 200 mM, about 300 mM, about 500 mM, about 700 mM, about 1M. Chaotropic agents are used to enhance refolding and stabilize OAS proteins, such chaotropic agents include: urea, guanidine HCl, thiourea, and the like, at concentrations such as for example about 0.05M, 0.1M,0.25M, 0.5M, 1.0M, 2.0M, 3.0M, 4.0M, 5.0M, 6.0M, 7.0M, such as for example 8.0M and above including near saturation solutions. A variety of detergents are added to improve OAS protein refolding efficiency, to reduce protein aggregation, and to reduce the non-specific interaction of OAS proteins with solid supports, resins, tubes, containers, etc. Detergents also improve the stability of OAS proteins in solution. Exemplary detergent additives include Nonidet P-40, Tween-80®, Tween-20®, Triton-X100®, Triton-X114®, Emulgens, Lubrol, Digitonin, octyl glucoside, lysolecithin, CHAPS®, CHAPSO®, zwittergents, cholate, deoxycholate, cetyl trimethylammonium bromide, N-lauryl sarcosine, polysorbate 20, polysorbate 80, pluronic F-68, saponin, polysorbate 40, lauryldimethylamine oxide, 3-(docecyldimethyl-ammonio) propanesulfonate inner salt (SB3-10), hexadecyltrimethyl ammonium bromide (CTAB), 3-(1-pyridinio)-1-propanesulfonate (NDSB 201), aminosulfobetaine-16 (ASB-16), and dodecyl sulfate, at concentrations of for example, about 0.01%, about 0.02%, about 0.05%, about 0.07%, 0.1% w/v, 0.2% w/v, 0.3% w/v, 0.4% w/v, 0.5% w/v, about 1% w/v, about 5% w/v, about 10% w/v, more than 10% w/v. In further specific embodiments, chelating agents are added, such as for example, citrate, ethylene diamine tetraacetic acid (EDTA) and ethylene glycol tetraacetic acid (EGTA) at concentrations between 1 mM and 20 mM, for example 2 mM, about 3 mM, about 4 mM, about 5 mM, about 10 mM, about 15 mM, such as for example about 17 mM. Chelating agents increase the half-life of thiol-reductants. Stabilizing agents including sugars and polyols (e.g. glycerol, sucrose, trehalose, glucose, lactose, inositol, mannitol, xylitol, ethylene glycol), polysaccharides (e.g. cyclodextrin), neutral polymers (e.g. polyethylene glycol (PEG)-400, PEG-4000, PEG-8000) amino acids and derivatives (e.g. arginine, glycine, glutamate, aspartate, betaine, trimethylamine-N-oxide (TAMO), phenylalanine, threonine, cysteine, histidine), albumins (e.g. bovine or human serum albumins), and large dipolar molecules can be added throughout the manufacturing process to stabilize OAS proteins. Thiol-protective or reducing agents are added to prevent errant disulfide bond formation and to cleave inappropriate disulfide bonds within inclusion bodies, such thiol-protective groups include dithiothreitol (DTT), dithioerythritol (DTE), 2-mercaptoethanol, 2-3-dimercaptopropanol, tributylphosphine (TBP), tris-carboxyethylphosphine (TCEP), thioglycolate, glutathione, and cysteine at concentrations of between 0.5 and 100 mM, such as for example, 1 mM, about 5 mM, about 10 mM, about 25 mM, about 50 mM, about 100 mM.

Exemplary Manufacturing Process Validation Methods for OAS Polypeptides

A number of biochemical methods are available to validate the purity and activity of in-process and final-stage purified OAS proteins manufactured according to the specification. The analytical methods include, but are not limited to, the following: quantification of protein concentration, measurement of protein purity, measurement of contaminants such as endotoxin, measurement of enzymatic activity (specific activity), and measurement of antiviral potency.

Quantification of OAS Polypeptide Concentration

The concentration of in-process and purified OAS proteins is measured by various assays known to one skilled in the art. One exemplary embodiment is a commercially available bicinchoninic acid (BCA) protein concentration assay kit such as the Reducing Agent Compatible BCA Protein Assay Kit from Pierce Biochemicals. A second exemplary embodiment is ultraviolet (UV) spectroscopy at a wavelength of 280 nm. In-process and purified proteins and their appropriate buffers are diluted in 6M Guanidine Hydrochloride (GuHCl) and the absorbance at 280 nm is recorded. The concentration in mg/ml is calculated by multiplying the corrected absorbance (A280 sample-A280 background) by the appropriate extinction coefficient.

Measurement of OAS Protein Purity

The purity of in-process and purified OAS proteins is measured by various analytical methods. One exemplary embodiment is Sodium Dodecyl Sulfate Polyacrylamide Gel Electrophoresis (SDS-PAGE) as shown in FIG. 3. In-process or purified OAS proteins are separated via SDS-PAGE and visualized using any appropriate method, including but not limited to Coomassie Brilliant Blue staining, silver staining, or western blot analysis with antibodies specific for OAS or contaminating proteins. The intensity of the specific OAS band and contaminating bands is compared by standard densitometry techniques known to one skilled in the arts. A second exemplary embodiment is size exclusion chromatography (SEC) using an appropriate chromatographic system. For example, in-process and purified OAS proteins are separated on size exclusion columns using either FPLC or HPLC chromatographic systems to ensure that the purified proteins are monomeric. A third exemplary embodiment is electrospray ionization mass spectrometry (ESI-MS), in which the sample is separated over an analytical column, ionized, and the mass to charge ratio detected by a mass spectrometer. Impurities in the protein preparation are detected as different mass to charge signals.

Measurement of OAS Polypeptide Contaminants

Assessment of in-process and final purified protein purity includes a measure of contaminants, including but not limited to host cell proteins and pyrogens such as endotoxin. Contamination with other proteins, including host cell proteins, can be assessed using the same techniques described in the section above (Measurement of Protein Purity). One exemplary embodiment of pyrogen testing is the Limulus Amoebocyte Lysate (LAL) endotoxin assay. Various commercially available assay kits are available that utilize a modified LAL and synthetic color-producing substrate to detect endotoxin presence.

Measurement of OAS Enzymatic Activity

The oligoadenylate synthetase activities of the in-process samples and final purified OAS proteins manufactured as per this invention are measured according to previously published methods (Justesen, J., et al. Nuc Acids Res. 8:3073-3085, 1980). Briefly, protein is activated with 200 μg/ml polyinosinic:polycytidylic acid (polyI:C) in buffer containing 20 mM Tris-HCl, pH 7.8, 50 mM Mg(OAc)₂, 1 mM DTT, 0.2 mM EDTA, 2.5 mM ATP, α[³²P]ATP, 0.5 mg/ml BSA, and 10% glycerol. The reaction proceeds at 37° C. for 30 minutes to 24 hours and is terminated by heating to 90° C. for 3 minutes. 2-4 μl of the reaction mixture is spotted onto a polyethylenimine PEI-cellulose thin layer plate (TLC). After drying, the plate is developed with 0.4 M Tris-HCl, 30 mM MgCl₂, pH 8.7. The plate is dried and visualized by phosphorimager analysis. Alternatively, the reaction mixture can be further incubated with 0.05 U/μl calf intestinal phosphatase to remove the terminal phosphate. Thin layer chromatographic separation is achieved using a 0.76 M KH₂PO₄, pH 3.6 developing buffer system. The plate is then dried and visualized by phosphorimager analysis. In another embodiment, cell associated OAS activity can be measured as described in FIG. 9.

A second exemplary embodiment of a method to assess enzymatic activity is to measure the catalysis of NAD-AMP by OAS proteins from the substrates β-Nicotinamide adenine dinucleotide (NAD) and dATP. Different concentrations of protein are mixed with 2 mM NAD, 2 mM dATP, 4 mM Tris pH 7.8, 4 mM Mg(OAc)₂, 0.2 mM DTT, 0.04 mM EDTA, 0.1 mg/ml BSA and 0.05 mg/ml polyl:C. The sample is incubated at 37° C. for 20 min, and the reaction is stopped by heating at 80° C. for 2 min. The sample is spun down and an aliquot taken and diluted 1:1 with an appropriate mobile phase buffer. The analytes are separated via C18 column chromatography on an HPLC. Area under the curve analysis of the peaks is used to calculate the percent conversion NAD and dATP to the NAD-AMP product.

Measurement of Antiviral Activity of OAS Polypeptides

Potency of in-process and final purified OAS proteins are demonstrated using a variety of cell culture antiviral assays. One exemplary embodiment of antiviral activity is the ability of the manufactured proteins to protect cultured cells from cytotoxicity induced by the murine encephalomyocarditis virus (EMCV, ATCC strain VR-129B). Human Huh7 hepatoma cells are seeded at a density of 1×10⁴ cells/well in 96 well culture plates and incubated overnight in complete medium (DMEM containing 10% fetal bovine serum). The following morning, the media is replaced with complete medium containing 0-10 μM protein or equivalent amounts of protein dilution buffer. When desired, alpha-interferon is added at a concentration of 100 IU/ml. Cells are pretreated for 2-8 hours preceding viral infection. After pretreatment, an equal volume of medium containing dilutions of EMC virus in complete medium is added to the wells. In the experiments described herein, a range of 50-250 plaque forming units (pfu) is added per well. Viral infection is allowed to proceed overnight (approximately 18 hours), and the proportion of viable cells is calculated using any available cell viability or cytotoxicity reagents. The results described herein are obtained using a cell viability assay that measures conversion of a tetrazolium compound [3-(4,5-dimethyl-2-yl)-5-(3-carboxymethoxyphenyl)-2-(4-sulfophenyl)-2H-tetrazolium, inner salt; MTS] to a colored formazan compound in viable cells. The conversion of MTS to formazan is detected in a 96-well plate reader at an absorbance of 492 nm. The resulting optical densities either are plotted directly (e.g. FIG. 10) to estimate cell viability or are normalized by control-treated samples to calculate a percentage of viable cells after treatment.

Other in vitro virus infection models include but are not limited to flaviviruses such as bovine diarrheal virus, West Nile Virus, and GBV-C virus, and other RNA viruses such as respiratory syncytial virus, and the HCV replicon systems (e.g. Blight, K. J., et al. 2002. J. Virology, 76:13001-13014). Any appropriate cultured cell competent for viral replication can be utilized in the antiviral assays.

Diagnostic and Screening Methods for OAS2 and OAS3 Mutations

Utilizing methods described above and others known in the art, the present invention contemplates a screening method comprising treating, under amplification conditions, a sample of genomic DNA, isolated from a human, with a PCR primer pair for amplifying a region of human genomic DNA containing any of nucleotide (nt) positions 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15 corresponding to sites of mutation in the OAS2 and OAS3 genes as provided in FIGS. 4 and 5. Amplification conditions include, in an amount effective for DNA synthesis, the presence of PCR buffer and a thermocycling temperature. The PCR product thus produced is assayed for the presence of a mutation at the relevant nucleotide position. In one embodiment, the amplicons as described above in Tables 1 and 2 are exemplary of the PCR products and corresponding primers.

In one preferred embodiment, the PCR product is assayed for the corresponding mutation by treating the amplification product, under hybridization conditions, with an oligonucleotide probe specific for the corresponding mutation, and detecting the formation of any hybridization product. Preferred oligonucleotide probes comprise a nucleotide sequence indicated in Table 3 below, wherein either of the nucleotide sequences enclosed in parentheses and separated by “/” may be used in the construction of the probe. Oligonucleotide hybridization to target nucleic acid is described in U.S. Pat. No. 4,530,901.

TABLE 3

| | |
|---|---|
| Mutation | Probe |

| | | |
|---|---|---|
| Mutation:7155 | TACAGACCCAGC(A/C)TCTCTCCCTCTA | (SEQUENCE:80) |
| Mutation:7168 | TATGTACCCATA(T/C)GTTCTGTGGGTA | (SEQUENCE:81) |
| Mutation:7150 | CTTCCCCTTGCA(C/T)CTGCGCCGGGCG | (SEQUENCE:82) |
| Mutation:6238 | CTTCCCCTTGCA(C/T)CTGCGCCGGGCG | (SEQUENCE:83) |
| Mutation:6239 | ACCTGCGCCGGG(C/A)GGCCATGGACTT | (SEQUENCE:84) |
| Mutation:7165 | ACCTGCGCCGGG(C/A)GGCCATGGACTT | (SEQUENCE:85) |
| Mutation:7142 | TCGTGGCCAGAA(G/A)GCTGCAGCCGCG | (SEQUENCE:86) |
| Mutation:6240 | TCGTGGCCAGAA(G/A)GCTGCAGCCGCG | (SEQUENCE:87) |
| Mutation:6241 | CCTGGCCGCTGC(C/T)CTGAGGGAGCGC | (SEQUENCE:88) |
| Mutation:14100 | GTGTCCAAAGGG(-/CAAAGGG)GAGTCCTGGGAG | (SEQUENCE:89) |
| Mutation:13915 | GGCTCCTCGGGC(C/T)GGGGCACAGCTC | (SEQUENCE:90) |
| Mutation:6245 | TAAGTGAGGGGG(C/T)CCCAGGACCCTT | (SEQUENCE:91) |
| Mutation:6246 | GCATTGGGTTGA(T/C)GCAGAAACCACT | (SEQUENCE:92) |
| Mutation:6247 | TTGATGCAGAAA(C/T)CACTGCGCCTGG | (SEQUENCE:93) |
| Mutation:6248 | AAGAGCAGGGAG(C/G)AAACCTCCCTCA | (SEQUENCE:94) |
| Mutation:6249 | GAAAAAGGCCAT(T/C)GACATCATCTTG | (SEQUENCE:95) |
| Mutation:13916 | AGTGGAGACACA(-/G)GGGGGGACCCTA | (SEQUENCE:96) |
| Mutation:7158 | CACAGACCTAAG(G/A)GATGGCTGTGAT | (SEQUENCE:97) |
| Mutation:6251 | CCAGGTCTACTC(G/A)AGGCTCCTCACC | (SEQUENCE:98) |
| Mutation:7144 | GAGCAGAAGGAC(C/T)GGCCTCCTCCAT | (SEQUENCE:99) |
| Mutation:6253 | ATCCCACTCCTC(AC/T-)TCTGCTTCCCTC | (SEQUENCE:100) |
| Mutation:6254 | GGAAGCAGCAGC(G/A)CTGGGGATGCAG | (SEQUENCE:101) |
| Mutation:7161 | CTGGGGATGCAG(G/T)CCTGCTTTCTGA | (SEQUENCE:102) |
| Mutation:7164 | TTGACCCACTTC(C/T)GCCCTCGTAGCA | (SEQUENCE:103) |
| Mutation:6255 | CAGTCCAGAACC(G/A)ACAGGCTAAGCC | (SEQUENCE:104) |
| Mutation:6256 | ATCCGAGCCCAG(C/T)TGGAGGCATGTC | (SEQUENCE:105) |
| Mutation:13918 | CTAAAAACACCC(T/C)GTGGCCTCCCAG | (SEQUENCE:106) |
| Mutation:6257 | CCCACTGGGACA(A/C)CATGGGAGCCGG | (SEQUENCE:107) |
| Mutation:7172 | ACCCCCACAGCA(C/T)GGGCTGGAACTC | (SEQUENCE:108) |
| Mutation:7143 | ACCCCCACAGCA(C/T)GGGCTGGAACTC | (SEQUENCE:109) |
| Mutation:6258 | GGCTTACACACT(A/G)GGATCCAGACTC | (SEQUENCE:110) |
| Mutation:6259 | CAAATCTAAATA(G/C)TTTATATAGGGA | (SEQUENCE:111) |
| Mutation:6260 | ACAACAGTGTCC(A/G)CACTAGTCAAGG | (SEQUENCE:112) |
| Mutation:6261 | ACAACAGTGTCC(A/G)CACTAGTCAAGG | (SEQUENCE:113) |
| Mutation:6262 | CACTGGACTATT(G/C)GTTTCAATATTA | (SEQUENCE:114) |
| Mutation:7157 | CACTGGACTATT(G/C)GTTTCAATATTA | (SEQUENCE:115) |
| Mutation:13919 | CCAGAGCTGCGG(G/A)AAGACGGATCCC | (SEQUENCE:116) |
| Mutation:7152 | AGACATGTATGA(T/C)TGAATGGGTGCC | (SEQUENCE:117) |
| Mutation:13920 | GGGTGCCAAGTG(C/T)CAGGGGGCGGAG | (SEQUENCE:118) |
| Mutation:14038 | AGTGCCAGGGGG(C/T)GGAGTCCCCAGC | (SEQUENCE:119) |
| Mutation:6263 | TCCACAGGAGTG(C/T)CTTAGACAGCCT | (SEQUENCE:120) |
| Mutation:6264 | TCCACAGGAGTG(C/T)CTTAGACAGCCT | (SEQUENCE:121) |
| Mutation:6265 | TGGCCCTGGCTG(C/T)TGCCACACACAT | (SEQUENCE:122) |
| Mutation:7153 | TGGCCCTGGCTG(C/T)TGCCACACACAT | (SEQUENCE:123) |
| Mutation:14039 | ACCACACAGACT(C/T)TGGGCCTCCCCG | (SEQUENCE:124) |
| Mutation:6266 | TCTGGGCCTCCC(C/T)GCAAAATGGCTC | (SEQUENCE:125) |
| Mutation:6267 | CGATGGAACCAG(G/A)TAAGTTGACGCT | (SEQUENCE:126) |
| Mutation:13614 | ATGGCGCTGGTA(C/T)GTAAATAGACCA | (SEQUENCE:127) |
| Mutation:13922 | AAATGGGGAGTC(C/T)CAGCTGTCCTCG | (SEQUENCE:128) |
| Mutation:7109 | GGCAGCAAGGCC(G/A)AGCTACTGGGTG | (SEQUENCE:129) |
| Mutation:7110 | CTCCGATGGTAC(C/G)CTTGTCCTCTTC | (SEQUENCE:130) |
| Mutation:7111 | AAGCCAAAGAAG(C/G)GGGTGCCAGACA | (SEQUENCE:131) |
| Mutation:13905 | GCTCAAAAGATC(C/T)TTGGATAAGACA | (SEQUENCE:132) |
| Mutation:13914 | AACTAGATCCCC(C/A)AATGAGCTGCTA | (SEQUENCE:133) |
| Mutation:13906 | CGTCAGAACCGT(A/T)CTGGAGCTGATC | (SEQUENCE:134) |
| Mutation:7112 | TGAGCACTGGCC(T/C)TTCTCATGTCTT | (SEQUENCE:135) |
| Mutation:7113 | TAATACTATTCA(C/G)AGTAATTTCCAA | (SEQUENCE:136) |
| Mutation:13907 | TCTGTATAAATC(C/T)TCGGACCTCCCG | (SEQUENCE:137) |
| Mutation:7114 | GTAAGGACAGTC(T/C)TTGTTCTGACCA | (SEQUENCE:138) |
| Mutation:13636 | GAGTGGAGTGCC(G/A)GATTTTGACACT | (SEQUENCE:139) |
| Mutation:13869 | TGAAGATGAGAC(C/T)GTGAGGAAGTTT | (SEQUENCE:140) |
| Mutation:7115 | CACCCTAGCCCC(G/A)TACTTTTCTTAA | (SEQUENCE:141) |
| Mutation:13635 | GTCTCAGCAACC(T/C)GGATTTTCCTCT | (SEQUENCE:142) |
| Mutation:14077 | CTTCAAGGATGG(G/T)ACTGGAAACCCA | (SEQUENCE:143) |
| Mutation:13912 | CAGGCTTGAATC(A/G)AAGAACTTCTCC | (SEQUENCE:144) |
| Mutation:13913 | CCCCTAAGCCCC(C/-)ACTACAAGTGAT | (SEQUENCE:145) |
| Mutation:7116 | AATGTCATGTGG(C/T)TACCTGTAACTT | (SEQUENCE:146) |
| Mutation:7117 | AAAGAAACTTCT(A/G)GAGATCATCTGG | (SEQUENCE:147) |
| Mutation:7118 | AAAGAAACTTCT(A/G)GAGATCATCTGG | (SEQUENCE:148) |
| Mutation:7119 | TAACTCTGTGAT(C/A)TTGCTCTCGGTG | (SEQUENCE:149) |
| Mutation:13872 | CTTTCTCCCCCC(C/-)ACCCAGGAGTAT | (SEQUENCE:150) |
| Mutation:13911 | CAAAAGAC1TTT(T/-)CCTTGGGCTTTA | (SEQUENCE:151) |
| Mutation:7124 | CTTTTCACCCAT(G/C)CCTGGGTTTATG | (SEQUENCE:152) |
| Mutation:15174 | TGCCAAGGGGGC(G/A)AGCATGCGGCCT | (SEQUENCE:230) |
| Mutation:14233 | GTTTTGCACTTT(GTTT/---)ATGTGTCCA | (SEQUENCE:231) |
| Mutation:15200 | GATCTGTGGTGC(C/T)AAAGGAAGTACC | (SEQUENCE:232) |
| Mutation:15186 | ATTTTCCCATCC(G/A)GCTGTGTGGTCT | (SEQUENCE:233) |
| Mutation:15202 | CCCCAGGCTGCT(G/A)TGTGAAGTTGAG | (SEQUENCE:234) |
| Mutation:15203 | TGGACACCAGCC(CTC/---)AGCATGAGGA | (SEQUENCE:235) |
| Mutation:15199 | TGAGGAAATTCA(G/T)GGTCCCCTACCA | (SEQUENCE:236) |
| Mutation:15198 | ATTCAGGGTCCC(C/T)TACCAGATGAGA | (SEQUENCE:237) |
| Mutation:15197 | CCAGATGAGAGA(G/C)ATTGTGTACATG | (SEQUENCE:238) |
| Mutation:13938 | GGATTTACCCTC(G/A)CTGTCTCCGTAT | (SEQUENCE:239) |

The PCR admixture thus formed is subjected to a plurality of PCR thermocycles to produce OAS2 or OAS3 gene amplification products. The amplification products are then treated, under hybridization conditions, with an oligonucleotide probe specific for each mutation. Any hybridization products are then detected.

The following examples are intended to illustrate but are not to be construed as limiting of the specification and claims in any way.

### EXAMPLES

### Example 1

### Preparation and Preliminary Screening of Genoic DNA

This example relates to screening of DNA from two specific populations of patients, but is equally applicable to other patient groups in which repeated exposure to HCV is documented, wherein the exposure does not result in infection. The example also relates to screening patients who have been exposed to other flaviviruses as discussed above, wherein the exposure did not result in infection.

Here, two populations are studied: (1) a hemophiliac population, chosen with the criteria of moderate to severe hemophilia, and receipt of concentrated clotting factor before January, 1987; and (2) an intravenous drug user population, with a history of injection for over 10 years, and evidence of other risk behaviors such as sharing needles. The study involves exposed but HCV negative patients, and exposed and HCV positive patients.

High molecular weight DNA is extracted from the white blood cells from IV drug users, hemophiliac patients, and other populations at risk of hepatitis C infection, or infection by other flaviviruses. For the initial screening of genomic DNA, blood is collected after informed consent from the patients of the groups described above and anticoagulated with a mixture of 0.14 M citric acid, 0.2 M trisodium citrate, and 0.22 M dextrose. The anticoagulated blood is centrifuged at 800×g for 15 minutes at room temperature and the platelet-rich plasma supernatant is discarded. The pelleted erythrocytes, mononuclear and polynuclear cells are resuspended and diluted with a volume equal to the starting blood volume with chilled 0.14M phosphate buffered saline (PBS), pH 7.4. The peripheral blood white blood cells are recovered from the diluted cell suspension by centrifugation on low endotoxin Ficoll-Hypaque (Sigma Chem. Corp. St. Louis, Mo.) at 400×g for 10 minutes at 18° C. (18° C.). The pelleted white blood cells are then resuspended and used for the source of high molecular weight DNA.

The high molecular weight DNA is purified from the isolated white blood cells using methods well known to one skilled in the art and described by Maniatis, et al., Molecular Cloning: A Laboratory Manual, 2nd ed. Cold Spring Harbor Laboratory, Sections 9.16-9.23, (1989) and U.S. Pat. No. 4,683,195.

Each sample of DNA is then examined for a mutation of any one of the nucleotides at position 3944545, 3945492, 3945829,3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15, corresponding to the oligoadenylate synthetase 3 and 2 genes (OAS3 and OAS2).

### Example 2

### Mutation in an OAS Gene Associated with Resistance to HCV Infection

Using methods described in Example 1, a population of unrelated hemophiliac patients and intravenous drug users was studied, and the presence or absence of a mutation in OAS3 or OAS2 as disclosed in the mutations in FIGS. 4 and 5, respectively, was determined.

In a study of 24 cases and 62 controls in a Caucasian population, these mutations were found in the context of resistance to hepatitis C infection. There was a statistically significant correlation between resistance to HCV infection and presence of a mutation in OAS2 or OAS3.

### Example 3

### Preparation and Sequencing of cDNA

Total cellular RNA is purified from cultured lymphoblasts or fibroblasts from the patients having the hepatitis C resistance phenotype. The purification procedure is performed as described by Chomczynski, et al., Anal. Biochem., 162:156-159 (1987). Briefly, the cells are prepared as described in Example 1. The cells are then homogenized in 10 milliliters (ml) of a denaturing solution containing 4.0M guanidine thiocyanate, 0.1M Tris-HCl at pH 7.5, and 0.1M beta-mercaptoethanol to form a cell lysate. Sodium lauryl sarcosinate is then admixed to a final concentration of 0.5% to the cell lysate after which the admixture was centrifuged at 5000×g for 10 minutes at room temperature. The resultant supernatant containing the total RNA is layered onto a cushion of 5.7M cesium chloride and 0.01M EDTA at pH 7.5 and is pelleted by centrifugation. The resultant RNA pellet is dissolved in a solution of 10 mM Tris-HCl at pH 7.6 and 1 mM EDTA (TE) containing 0.1% sodium docecyl sulfate (SDS). After phenolchloroform extraction and ethanol precipitation, the purified total cellular RNA concentration is estimated by measuring the optical density at 260 nm.

Total RNA prepared above is used as a template for cDNA synthesis using reverse transcriptase for first strand synthesis and PCR with oligonucleotide primers designed so as to amplify the cDNA in two overlapping fragments designated the 5′ and the 3′ fragment. The oligonucleotides used in practicing this invention are synthesized on an Applied Biosystems 381A DNA Synthesizer following the manufacturer's instructions. PCR is conducted using methods known in the art. PCR amplification methods are described in detail in U.S. Pat. Nos. 4,683,192, 4,683,202, 4,800,159, and 4,965,188, and at least in several texts including PCR Technology: Principles and Applications for DNA Amplification, H. Erlich, ed., Stockton Press, New York (1989); and PCR Protocols: A Guide to Methods and Applications, Innis, et al., eds., Academic Press, San Diego, Calif. (1990) and primers as described in Table 1 herein.

The sequences determined directly from the PCR-amplified DNAs from the patients with and without HCV infection, are analyzed. The presence of a mutation upstream from the coding region of the OAS gene can be detected in patients who are seronegative for HCV despite repeated exposures to the virus.

### Example 4

### Preparatoin of PCR Amplified Genomic DNA Containing a Mutation and Detecton by Allele Specific Oligonucleotide Hydridization

The mutation in an oligoadenylate synthetase (either OAS2 or OAS3) gene at one of nucleotide positions 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334-3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959, 4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15 can be determined by an approach in which PCR amplified genomic DNA containing the mutation is detected by hybridization with oligonucleotide probes that hybridized to that region. To amplify the region having the mutation for hybridization with oligonucleotide specific probes, PCR amplifications are performed as essentially described in Example 3 with, for example, 180 ng of each of the primers shown in Table 1.

Following the PCR amplification, 2 μl of the amplified oligoadenylate synthetase DNA products are spotted onto separate sheets of nitrocellulose. After the spotted amplified DNA has dried, the nitrocellulose is treated with 0.5N NaOH for 2 minutes, 1M Tris-HCl at pH 7.5 for 2 minutes, followed by 0.5M Tris-HCl at pH 7.5 containing 1.5M NaCl for 2 minutes to denature and then neutralize the DNA. The resultant filters are baked under a vacuum for 1 hour at 80° C., are prehybridized for at least 20 minutes at 42° C. with a prehybridization solution consisting of 6× SSC (1×=0.15M NaCl, 0.15M sodium citrate), 5× Denhardt's solution (5×=0.1% polyvinylpyrrolidone, 0.1% ficoll, and 0.1% bovine serum albumin), 5 mM sodium phosphate buffer at pH 7.0, 0.5 mg/ml salmon testis DNA and 1% SDS.

After the prehybridization step, the nitrocellulose filters are separately exposed to ³²P-labeled oligonucleotide probes diluted in prehybridization buffer. Labeling of the probes with ³²p is performed by admixing 2.5 μl of 10× concentrate of kinase buffer (10×=0.5M Tris[hydroxymethyl] aminomethane hydrochloride (Tris-HCl) at pH 7.6, 0.1M MgCl₂, 50 mM dithiothreitol (DTT), 1 mM spermidine-HCl, and 1 mM ethylenediaminetetraacetic acid (EDTA)), 1.1 μl of 60 μg/μl of a selected oligonucleotide, 18.4 μl water, 2 μl of 6000 Ci/mM of gamma ³²p ATP at a concentration of 150 mCi/μl , and 1 μl of 10 U/μl polynucleotide kinase. The labeling admixture is maintained for 20 minutes at 37° C. followed by 2 minutes at 68° C. The maintained admixture is then applied to a Sephadex G50 (Pharmacia, Inc., Piscataway, N.J.) spin column to remove unincorporated ³²P-labeled ATP.

The oligonucleotide probes used to hybridize to the region containing the mutation are shown in Table 3 above. The underlined nucleotide corresponds to the mutation nucleotide. In probes for detecting wild type (normal), the underlined nucleotide is replaced with the wild-type nucleotide.

Ten×10⁶ cpm of the normal and mutant labeled probes are separately admixed with each filter. The nitrocellulose filters are then maintained overnight at 42° C. to allow for the formation of hybridization products. The nitrocellulose filters exposed to the normal probe are washed with 6× SSC containing 0.1% SDS at 46° C. whereas the filters exposed to the mutant probe are washed with the same solution at a more stringent temperature of 52° C. The nitrocellulose filters are then dried and subjected to radioautography.

Only those products having the mutation hybridize with the mutant probe. Positive and negative controls are included in each assay to determine whether the PCR amplification is successful. Thus, the patients' genomic DNA prepared in Example 1 are determined by this approach to have the unique mutational form in question at the indicated position.

### Example 5

### Antisense Inhibition of Target RNA

A. Preparation of Oligonucleotides for Transfection

A carrier molecule, comprising either a lipitoid or cholesteroid, is prepared for transfection by diluting to 0.5 mM in water, followed by sonication to produce a uniform solution, and filtration through a 0.45 μm PVDF membrane. The lipitoid or cholesteroid is then diluted into an appropriate volume of OptiMEM™ (Gibco/BRL) such that the final concentration would be approximately 1.5-2 nmol lipitoid per μg oligonucleotide.

Antisense and control oligonucleotides are prepared by first diluting to a working concentration of 100 μM in sterile Millipore water, then diluting to 2 μM (approximately 20 mg/mL) in OptiMEM™. The diluted oligonucleotides are then immediately added to the diluted lipitoid and mixed by pipetting up and down.

B. Transfection

Human PH5CH8 hepatocytes, which are susceptible to HCV infection and supportive of HCV replication, are used (Dansako et al., Virus Res. 97:17-30, 2003; Ikeda et al., Virus Res. 56:157-167, 1998; Noguchi and Hirohashi, In Vitro Cell Dev. Biol Anim. 32:135-137, 1996.) The cells are transfected by adding the oligonucleotide/lipitoid mixture, immediately after mixing, to a final concentration of 300 nM oligonucleotide. The cells are then incubated with the transfection mixture overnight at 37° C., 5% CO₂ and the transfection mixture remains on the cells for 3-4 days.

C. Total RNA Extraction and Reverse Transcription

Total RNA is extracted from the transfected cells using the RNeasy™ kit (Qiagen Corporation, Chatsworth, Calif.), following protocols provided by the manufacturer. Following extraction, the RNA is reverse-transcribed for use as a PCR template. Generally 0.2-1 μg of total extracted RNA is placed into a sterile microfuge tube, and water is added to bring the total volume to 3 μL. 7 μL of a buffer/enzyme mixture is added to each tube. The buffer/enzyme mixture is prepared by mixing, in the order listed:
4 μL 25 mM MgCl₂
2 μL 10× reaction buffer
8 μL 2.5 mM dNTPs
1 μL MuLV reverse transcriptase (50 u) (Applied Biosystems)
1 μL RNase inhibitor (20 u)
1 μL oligo dT (50 pmol)

The contents of the microfuge tube are mixed by pipetting up and down, and the reaction is incubated for 1 hour at 42° C.

D. PCR Amplification and Quantification of Target Sequences

Following reverse transcription, target genes are amplified using the Roche Light Cycler™ real-time PCR machine. 20 μL aliquots of PCR amplification mixture are prepared by mixing the following components in the order listed: 2 μL 10× PCR buffer II (containing 10 mM Tris pH 8.3 and 50 mM KCl, Perkin-Elmer, Norwalk, Conn.) 3 mM MgCl₂, 140 μM each dNTP, 0.175 pmol of each OAS2 or OAS3 oligo, 1:50,000 dilution of SYBR® Green, 0.25 mg/mL BSA, 1 unit Taq polymerase, and H₂O to 20 μL. SYBR® Green (Molecular Probes, Eugene, Oreg.) is a dye that fluoresces when bound to double-stranded DNA, allowing the amount of PCR product produced in each reaction to be measured directly. 2 μL of completed reverse transcription reaction is added to each 20 μL aliquot of PCR amplification mixture, and amplification is carried out according to standard protocols.

### Example 6

### Treatment of Cells with OAS RNAi

Using the methods of Example 5, for antisense treatment, cells are treated with an oligonucleotide based on the OAS2 or OAS3 sequence (SEQUENCE:2 or SEQUENCE: 1, respectively). Two complementary ribonucleotide monomers with deoxy-TT extensions at the 3′ end are synthesized and annealed. Cells of the PH3CH8 hepatocyte cell line are treated with 50-200 nM RNAi with 1:3 L2 lipitoid. Cells are harvested on day 1, 2, 3 and 4, and analyzed for target OAS protein by Western analysis, as described by Dansako et al., Virus Res. 97:17-30, 2003.

### Example 7

### Analysis of Resistant Haplotypes in OAS3

Using the methods described herein, a study of caucasian injecting drug users was conducted on 27 cases and 58 controls to identify OAS3 haplotypes associated with resistance to HCV infection. Cases were persistently HCV-seronegative and cases were HCV seropositive as described elsewhere. In one study of ten mutations spanning OAS3, two haplotype patterns shown in Table 4 below were particularly indicated as associated with resistance to HCV infection. In the table, for each mutation, the particular nucleotide composing each haplotype is provided; for haplotype positions that are insensitive to the nucleotide an N (for any nucleotide) is listed. The first haplotype is seen to impute resistance as demonstrated by the much higher percentage of cases compared to controls that possess the haplotype. In contrast, the second haplotype is seen to impute susceptibility due to the counter prevalence of the haplotype observed in controls. This is but one example of OAS3 haplotype mapping. This and finer mapping across the gene are used to delineate regions (of the gene, RNA, or protein) of specific import in relation to infection resistance.

TABLE 4
Inferred

| Haplotype | Mutation: ID | | | | | | | | | | % | % | P |
|---|---|---|---|---|---|---|---|---|---|---|---|---|---|
| Effect | 6240 | 13916 | 6254 | 7164 | 13917 | 6260 | 7157 | 6262 | 13920 | 6265 | Cases | Controls | value |
| Resistance | A | G | G | C | G | A | N | N | N | N | 37 | 18 | 0.007 |
| Susceptibility | N | G | G | N | G | G | G | G | C | C | 20 | 40 | 0.0099 |

### Example 8

### Analysis of Resistant Haplotypes in OAS2

Using the methods described herein, a study of injecting drug users was conducted on 34 cases and 71 controls to identify OAS2 haplotypes associated with resistance to HCV infection. Cases were persistently HCV-seronegative and cases were HCV seropositive as described elsewhere. In one study of eleven mutations spanning OAS2, two haplotype patterns shown in Table 5 below were particularly indicated as associated with resistance to HCV infection. In the table, for each mutation, the particular nucleotide composing each haplotype is provided; for haplotype positions that are insensitive to the nucleotide an N (for any nucleotide) is listed. The first haplotype is seen to impute resistance as demonstrated by the much higher percentage of cases compared to controls that possess the haplotype. In contrast, the second haplotype is seen to impute susceptibility due to the counter prevalence of the haplotype observed in controls. This is but one example of OAS2 haplotype mapping. This and other mapping across the gene are used to delineate regions (of the gene, RNA, or protein) of specific import in relation to infection resistance.

TABLE 5
Inferred

| Haplotype | Mutation: ID | | | | | | | | | | | % | % | P |
|---|---|---|---|---|---|---|---|---|---|---|---|---|---|---|
| Effect | 7114 | 13636 | 7115 | 13635 | 13912 | 13913 | 7116 | 7117 | 7119 | 13872 | 7124 | Cases | Controls | value |
| Resistance | N | G | A | T | A | — | T | G | C | — | N | 26 | 13 | .028 |
| Susceptibility | C | N | G | N | N | N | N | G | N | — | C | 33 | 51 | .015 |

### Example 9

### Identification of Alternate Splice Forms of OAS2

As discussed above, sequence data from multiply-sampled, multi-tissue human clone libraries are analyzed to identify novel splice forms of OAS2. One hundred forty one cDNA sequence entries from NCBI's dBEST that were clustered with OAS2 mRNAs by Unigene analysis (Wheeler, D. L., et al., Nucl Acids Res 31:28-33 ;2003) were collected for processing. Each candidate cDNA was independently aligned with the genomic reference sequence for OAS2, SEQUENCE:2 using the Spidey algorithm (Wheelan, S., http://www.ncbi.nlm.nih.gov/IEB/Research/Ostell/Spidey/index.html.) The resulting alignment was automatically analyzed to identify anomalous splicing patterns. Among those sequences that were identified and determined to be high quality evidence for alternative splicing were the following NCBI Accession numbers: BC010625.1, a colon adenocarcinoma-derived cDNA with a polyA tail that overruns exon 2 into intron 2 and terminates; DN996203.1, a breast cancer-derived cDNA that skips exon 2 causing a frameshift and subsequent termination; and CR990139, a T-lymphocyte cDNA that excises a portion of exon 2 by an in-frame alternate 5′ splice site.

### Example 10

### Identification of Alternate Splice Forms of OAS3

As discussed above, sequence data from multiply-sampled, multi-tissue human clone libraries are analyzed to identify novel splice forms of OAS3. Two hundred thirty nine cDNA sequence entries from NCBI's dBEST that were clustered with OAS2 mRNAs by Unigene analysis (Wheeler, D. L., et al., Nucl Acids Res 31 :28-33;2003) were collected for further processing. Each candidate cDNA was independently aligned with the genomic reference sequence for OAS3, SEQUENCE: 1 using the Spidey algorithm (Wheelan, S., http://www.ncbi.nlm.nih.gov/IEB/Research/Ostell/Spidey/index.html.) The resulting alignment was automatically analyzed to identify anomalous splicing patterns. Among those sequences that were identified and determined to be high quality evidence for alternative splicing were the following NCBI Accession numbers: AK000608.1, a signet ring carcinoma cDNA that contains a polyA tail and overruns the canonical 3′ splice site at the end of exon 2; BC012015.1 a bladder transitional papilloma cDNA that contains a polyA tail and overruns the canonical 3′ splice site at the end of exon 3; and AW505430.1, a lymph germ B cell cDNA that contains a premature stop mutation at amino acid Y 1005 and further demonstrates a run on of exon 14 containing said amino acid suggesting proper further processing of this transcript.

### Example 11

### Utility of Non-Human Primate Mutations in OAS2 and OAS3 Therapeutic Proteins

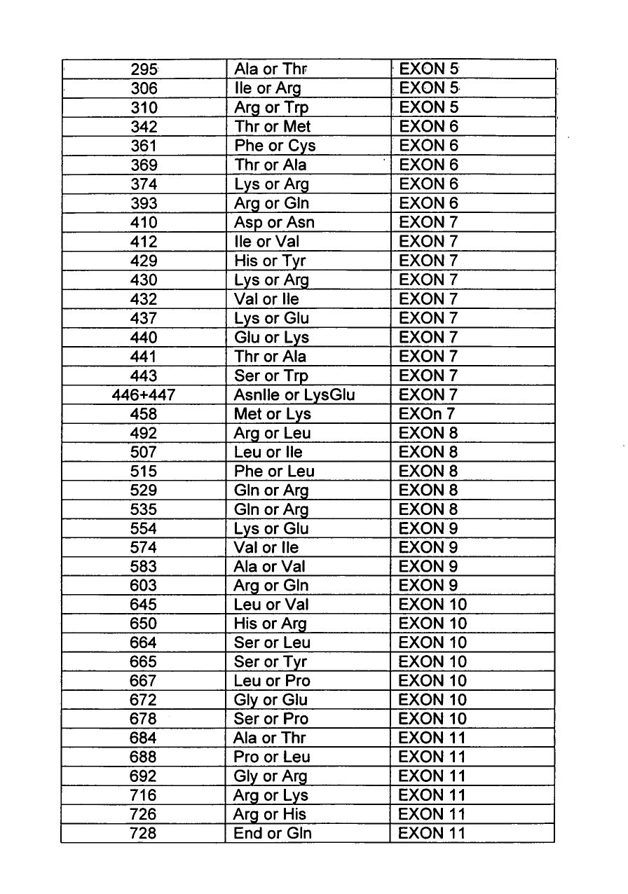

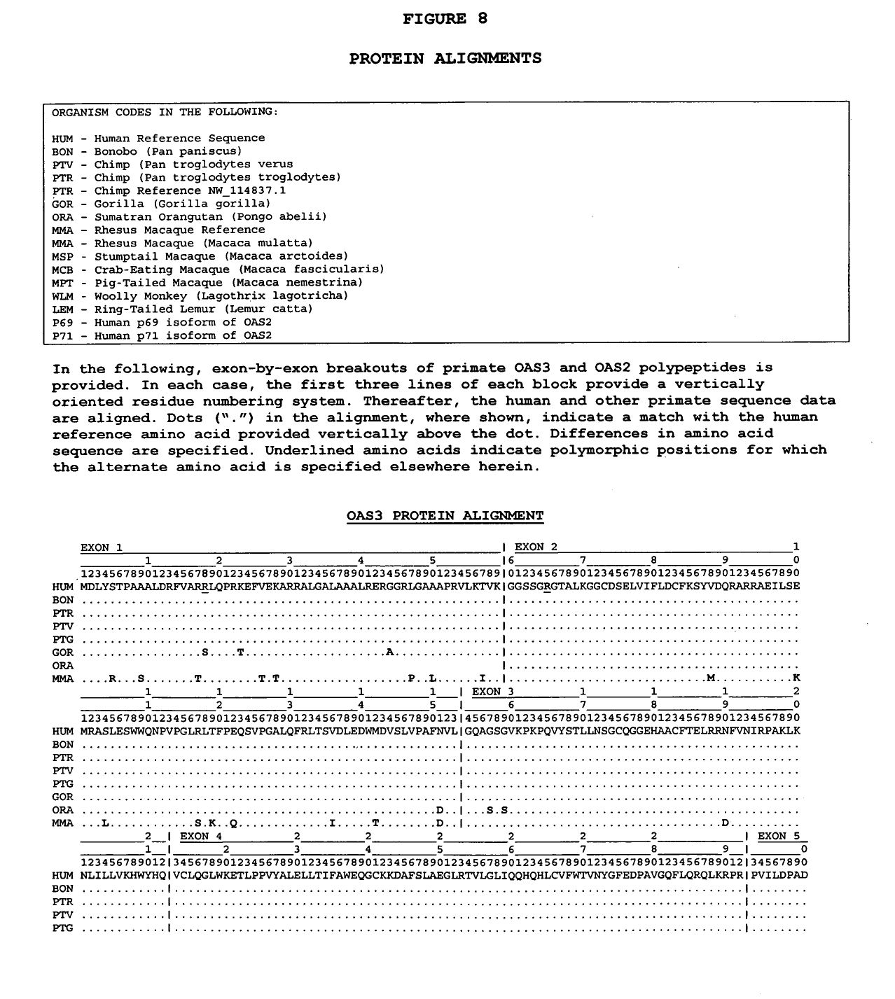

OAS2 and OAS3 genes from non-human primates (NHP) were sequenced using the methods of the present invention and compared with the respective human gene to identify NHP mutations. Exemplary amino acid modifications resulting from mutations identified in gorilla, bonobo, chimpanzee, orangutan, and macaque are depicted depicted in alignment with the respective human sequence in FIG. 8. The foregoing NHP mutations are also useful for the diagnostic and therapeutic purposes of the present invention. Such mutations provide additional insight into evolution of each of the OAS2 and OAS3 genes and their respective proteins. Evolutionarily conserved amino acids suggest sites important, or critical, for protein function or enzymatic activity. Conversely, amino acid residues that have recently mutated, for example in humans only, or show a plurality of amino acid substitutions across primates, indicate sites less critical to finction or enzymatic activity. The abundance of mutated sites within a particular motif of a particular OAS protein is correlated with the tolerance of that functional domain to modification. Such sites and motifs are optimized to improve protein function or specific activity. Similarly, mutations in genes and proteins with immune or viral defense functions like OAS2 and OAS3 are hypothesized to result from historical challenge by viral infection. Mutations in non-human primate OAS2 or OAS3 proteins are hypothesized to improve anti-viral efficacy on this basis and are opportunities for optimization of a human therapeutic OAS2 or OAS3 protein, respectively. The present invention is not limited by any evidence, or the lack thereof, for or against improved protein specific activity or anti-viral efficacy caused by the NHP mutations of the present invention, but rather all such non-human primate mutations represent opportunities for optimization of human OAS protein isoforms.

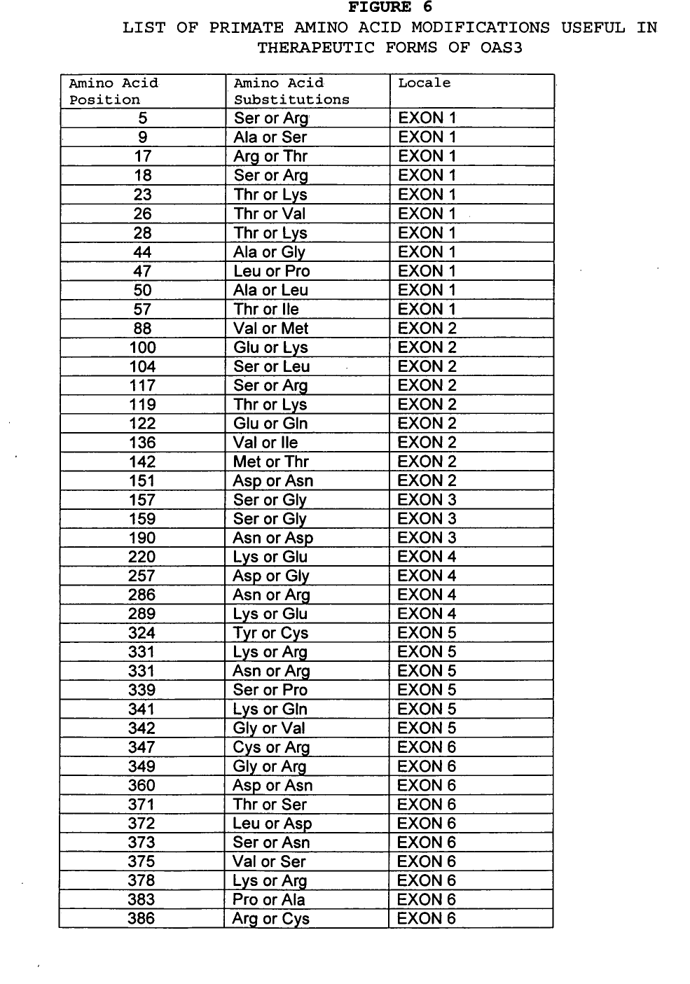

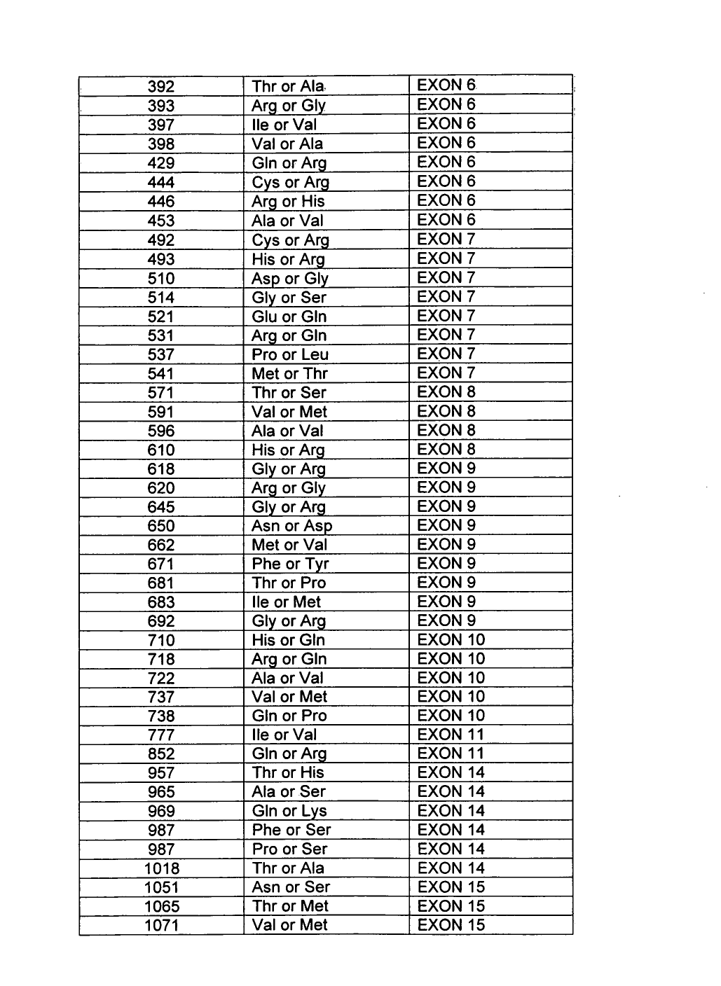

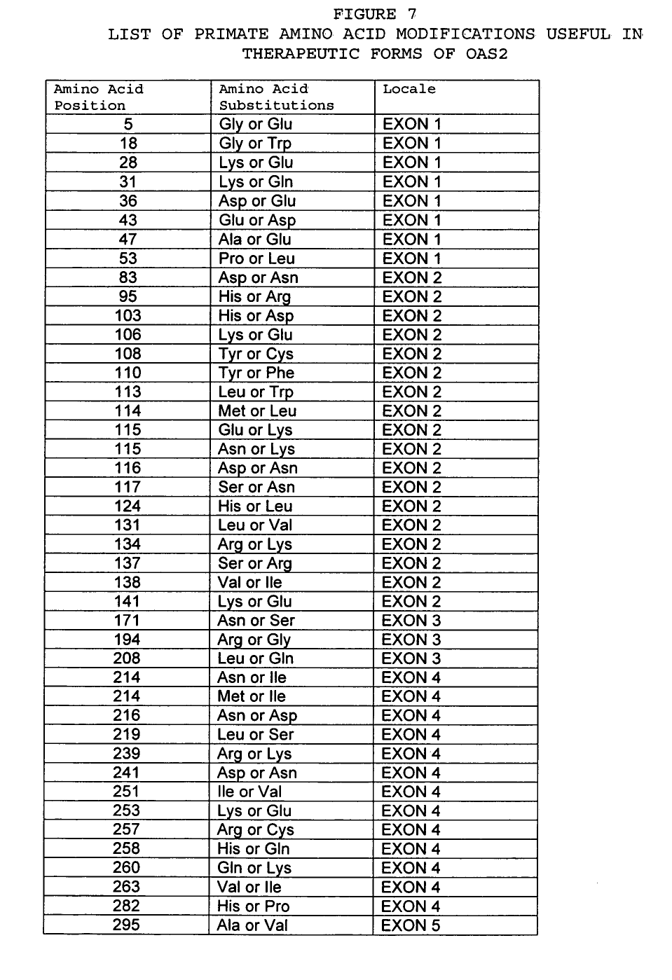

In an exemplary embodiment, the ancestral primate amino acid for a specific site within OAS2 or OAS3 is restored to a human therapeutic form of the corresponding OAS protein to optimize protein specific activity or anti-viral efficacy. In other embodiments, alternative amino acids identified in non-human primate OASs, but not necessarily ancestrally conserved, are substituted into their respective human therapeutic form of OAS2 or OAS3 in order to improve protein specific activity or anti-viral efficacy. FIG. 3 provides isoforms of OAS2 (SEQUENCE:6, SEQUENCE:7, or SEQUENCE:227) and OAS3 (SEQUENCE:8). Modifications to these base protein isoforms in order to develop optimized therapeutic isoforms (or for other purposes of the present invention) is performed using at least one amino acid modification as provided in FIG. 6 or FIG. 7. Additional modifications are made as indicated in FIG. 3. Any of the foregoing modifications described in FIGS. 6 and 7 are also applied in combination with other modifications of the present invention or to alternate therapeutic OAS2 or OAS3 isoforms envisioned by the present invention. Such derived primate-human recombinant proteins are useful for the diagnostic and therapeutic purposes of the present invention.

DNA and mRNA sequences that code for both the native primate proteins as well as such derived primate-human recombinant forms are also novel and have utility and are expressly envisioned by the present invention. Several examples of their utility are: as agents to detect their respective DNA or mRNA counterparts; in expression vectors used in the manufacture of therapeutic proteins; and in the detection of novel compounds that bind the respective mRNA.

### Example 12

### Preferred Therapeutic OAS2 Iosform

As more than one isoform of OAS2 is produced in humans, certain isoforms may elicit a non-self immunogenic reaction to a therapeutically administered isoform in those individuals that produce little or none of the administered isoform. An OAS2 preferred therapeutic polypeptide overcoming this limitation is developed by truncating the divergent carboxyl terminus of the protein as provided by SEQUENCE:227 of FIG. 3. A similar such truncation, as demonstrated in the case of OAS 1 (see WO 2005/040428), does not eliminate protein specific activity since the truncation does not impinge on the conserved OAS1-like motif found in the OAS family of proteins. OAS2 has two such tandem copies of the OAS1-like motif with one copy running from exons 1 through 5 and the other running from exons 6 through 10. In the case of SEQUENCE:227, the truncation omits the final non-conserved exon 11 that does not impinge on the OAS1-like motif portion of OAS2. Said preferred OAS2 therapeutic polypeptide is further optimized according to any of the methods or specifications of the present invention. Said preferred OAS2 therapeutic polypeptide and any of its optimized forms are tested according to the present invention by methods including, but not limited to, tests of specific activity, cellular entry, and antiviral activity.

The foregoing specification, including the specific embodiments and examples, is intended to be illustrative of the present invention and is not to be taken as limiting. Numerous other variations and modifications can be effected without departing from the true spirit and scope of the invention. All patents, patent publications, and non-patent publications cited are incorporated by reference herein.

1. A human genetic screening method for identifying an oligoadenylate synthetase gene (OAS2) mutation comprising detecting in a nucleic acid sample the presence of an OAS2 mutation selected from the group consisting of:
substitution of a non-reference nucleotide for a reference nucleotide at nucleotide position 3985940, 3986162, 3994402, 3994663, 4002659, 4004802, 4004959, 4010430, 4013626, 4013794, 4013927, 4015114, 4015219, 4015277, 4015321, 4016521, 4016612, 4017081, 4017797, 4018161, or 4018625 of reference sequence SEQ ID NO:2; and deletion of the reference nucleotide at position 4016713, 4018373, 4018411 of reference sequence SEQ ID NO:2; thereby identifying said mutation.

2. A human genetic screening method for identifying an oligoadenylate synthetase gene (OAS2 or OAS3) mutation comprising detecting in a nucleic acid sample the presence of an OAS2 or OAS3 mutation selected from the group consisting of: substitution of a non-reference nucleotide for a reference nucleotide at nucleotide position 3944545, 3945492, 3945829, 3945840, 3945897, 3945961, 3946060, 3948899, 39511864, 3955427, 3955454, 3956125, 3956133, 3956288, 3956459, 3956544, 3958039, 3968428, 3968688, 3970334, 3970335, 3970708, 3970721, 3971806, 3973006, 3973193, 3974596, 3974690, 3975294, 3977088, 3977210, 3977282, 3977339, 3977358, 3977365, 3977380, 3977502, 3977717, 3978383, 3978506, 3978685, 3978769, 3978787, 3978795, 3978922, 3979303, 3979479, 3979490, 3979825, 3979973, 3985940, 3986162, 3994402, 3994663, 4002659, 4004802, 4004863, 4004959,4010430, 4013626, 4013794, 4013927, 4015114, 4015219, 4015277, 4015321, 4016521, 4016612, 4016713, 4017081, 4017797, 4018161, 4018373, 4018411, or 4018625 of Genbank Accession No. NT_{—}009775.15.

3. The screening method of claim 1 or 2, wherein said nucleic acid sample is contacted with a probe selected from the group consisting of polynucleotides comprising at least one of SEQ ID NO:80-255.

4. An isolated polypeptide consisting of an amino acid sequence selected from the group consisting of for OAS2, SEQ ID NO:6, 7, 9, 10, 11, 12 and 227, and for OAS3, SEQ ID NO:8, 14 and 15.

5. The polypeptide of claim 4 covalently attached to polyethylene glycol.

6. The polypeptide of claim 4 encapsulated in a liposome.

7. The polypeptide of claim 4 attached to an endosome disrupting agent.

8. The polypeptide of claim 4 attached to an amino acid sequence or peptide to form a fusion protein.

9. The polypeptide of claim 4 covalently conjugated to a sugar moiety.

10. An isolated polypeptide produced by the method comprising:
(a) expressing the polypeptide of claim 4 by a cell; and
(b) recovering said polypeptide.

11. An isolated polynucleotide comprising a nucleotide sequence that encodes the polypeptide sequence of claim 4.

12. The isolated polynucleotide of claim 11, comprising a nucleotide sequence selected from the group consisting of SEQ ID NO:3, 4, 5 and 13.

13. An expression vector comprising the isolated polynucleotide of claim 11, operably linked to an expression control sequence.

14. A host cell transformed or transfected with an expression vector according to claim 13.

15. A method of treating viral infection in a mammal comprising administering to a mammal in need of such treatment a composition comprising a polypeptide of claim 4.

16. The method of claim 15 wherein said viral infection is an infection with a flavivirus.

17. The method of claim 16 wherein said flavivirus is the hepatitis C virus.

18. The method of claim 15 wherein said viral infection is an infection with a virus selected from the group consisting of HIV, respiratory syncytial virus, influenza, coronavirus, parainfluenza, hepatitis A, West Nile, dengue, yellow fever, herpes, and human papilloma virus.

19. A method of treating cancer in a mammal comprising administering to a mammal in need of such treatment a composition comprising a polypeptide of claim 4.

20. A monoclonal or polyclonal antibody directed against an epitope on a polypeptide of claim 4.


SEQUENCE LISTING (272 sequences)

SEQ ID NO: 1 - DNA, 40980 bp, Homo sapiens
gcattatttccccgttgctacagatgagagaattgaggttcagacaggttgaaatcattgctcccaaagtcacacaactggtgagtggcagagctgggatgcaaaccctaaactgccagccctcaaagcctgtgctcttaatctccaccctgctgtgcttccttgtccatttaattaagctccacaggcacacattccacgccctcctttgctgtacaatcccaggcaagtcgctcagcttctctgagcctcagtttcataatctgtcaaatggaggtaacacaaataattcctagttgtgaccaagaatcatcatagaaatctgccatttccagcctattgtgcaattcctcaagcactgtgactccaagtggcatcagctcctggaagaacacactgtcttactgttgtttcctcctttgtcaactgatccccccttgaacctcactctacctctgctctcaatgccccatctactgccacctgattaaataaaatcttttttgaaaatcataagtgtcatgagtaaggtttcttggtgttgatgtagaagaacaaaacagaattgtgaaatgagaatcactgcagctatcatgaagtcctgcctacgtgcccagcagttgctagattggccaagttttcccgtgaagggcaatatagtaaatattttaggccttatggatcatccaacctcagctggactcttcatctctgctgttgccatatgcaagcagccatagacaacatataaataaatgggtgtaatggcatttcaattaaactttatttatggacactgaatttcacaattttcacgtcacaaaatattcttttatttttaatattcttctgttgattttttttcttaaccattaaacatgttaaaaagtacaaaaacagctgggtgcagtggctcacacctgtaatctcaacactttgggaggctgaggcaagcagatcacttgaggtcaggagttcgagatcagcctggccaagatggtgaaaccctgtgtctatgagaaatacaaaaattagccaggtgtggtggtgggcgcctgtaatcccagctacttgggaggctgaggcaggagaatcgcttgaactagggaggcagaggttgcagtgagccacgataatgccactgcactccatcctgggctacagagcgaaactgtcaaaaaaaaaaaaaaaaaaaggcacaaaaacaagcagaggctggaccagatgtggcccatgggccatagtttgccagtttcttccaacatttcattaagaaaaatttccaagcaaacagccacattgaaagaattttgcagtgaacactcatatactcaccacatagattttacaattaacgttttattgcacttgcttgattgcatatctacaatttcttcatccttctatccatccatgaatccatcttattcttttgatgcatcagaagattctttttctgccaggcatggtggctcacacctgtaatcccagcacttcgggaagccaaggccgttggatcacctgaggtcaggagttcgagaccaccctggccaacatagtgaaaccccgtctctactaaaaatacaaaaattagccagacatggtggcacacacctgtaatcccagctactcgggaggctgaggcaggagaattgcttaaacctgggaggcagaagttgcagtgagccgagaccacactgctgcactccagcctgggctgtctcaaaaaaaaaaaaaaaaaaaaaaaaaaaaaggaaggaaggaaggaagagagagagagaaagagaggaaagaaagaaagaaagaaagaaagaaagaaagaaagaaagaaagagagaaagaaaagaaaagaaaagaaaagaaagaaaaagagagaaaaagagaaaaagaaagaaagaatattctttttcaaaaagaaaccagcagcaatttcttcccgggccagtacaaggtggtgagtgagttgactaagcagacaggcaaaaagagagagagtatctgtaggaggatactgtcaccttttatatataggcaataagcaatagttttccaaggagaacagcagatgatttgctactgtatcaaccaagaatatatttgaccgtagtaagtaacagaatgtgtaacagacattctgagtcattttgttacacccacaactgtgggtgacctgtccagatatgccttctaattccaagcaccccaagaaaacccctgtgcttaagattttatctagccatgaatccaacattttctatttgcttattaattttttgtccatcttctctcgttagaatataaattccatgagagcagaggccttttcacttattcaccacacatgttctgtgctagaccagtggctaccataaagaaaacactgaataaatatttatttatttatttatttattttatttttgagccagagtttcactcttgtcatccagcctagagtgcaatggtgtgatcttggctcactgcaaccattgcctcctgggttcaagtgattctcctgactcagactcctgagtacctgggattacaggtgcctgccaccatactcagctaatttttgtatgtttagtagagacggggtttcaccatgttggccaggccagtctcgaactcctgacctcaggtcatccacctgctttggcctcccaaagtgctgggattacaggcgtgagccaccgcacctggccttaataaatatttataagaataaggaaaggatctgttttccacatctcataagttgtcttttatacctttcttttctatgttctgctttcctgtaatctcgagtaatttcctccattccatcttccagtttactgattttatcttcaggtatatttattcagcctatctatggagaaggttcggtttttctgttcatctttggttttattttgcattatgttttcacttccaatgtgatagtggtgattgttgactttcatcactgttcttatttcatggatatgatgaataatcatgaatacaatgaataattataatggatataatatctaatccctctgaggatattaattatatatttctaagaccgtattagtccaatttgtacttctaaaacagaaagccacagactgggaaattagtaaagaacagaaatttacttatcacagttctggaggctgagaagtgcaagaccaaggcttcagcaggttaggacctggtctctatgcttccaagatggcgccttgaagaatgttgtgtcttttggaggggaggaacactatgtcctcacatggcagaaaagcagaagacaatgagcccattcctccaagctcttttcacagtggccataatccactcacgtgggcagagccctcatgacacaaacacctcctgttagaccccacttcccaactgctgcattggggagtaagtttccaacacatgaattctgggggacaaatttagaccacagaagactctttctcctttattgattatttcagtgtccttacatgtgagtttttatgttcatagcattggatctcccaaaataatttggcacctttggatgtgtgctttttgctcactggggatgttctgcttgtctccctgtgaagttggtaccttcttactgcagctggaggcagagatgaggaaaggggatgtgccaggaatgagtcttctgcatacaggacttcctgttctttccagatgtggccaggtatgctgtgtacttccctgcctctctgccccaccctcactgctccctccttaggcaggcacctctgctgcctgttactcaacacagaaaggttggggcgggagaccacatgtctttagaggctgccattactatgccagcacaactgacaaccatgagagttaacctagggtcccttcttttccttcaatccactgcctggagcctgcagctcctcagagcctttccactctgcataggtaccccctcctttatgtgttttggctgcaatttcctccttcatccagtctcatatctctgacagggatctcagattccagtttacctgggacacctctggttaggacccagcaacattattaaagtttttctatcgctttcctgccacctctagaggcctgggatggacgttgaaggcttatgcatatgttcagtccaccatcatgacccaatattgacccttctgcgttccatcttctaaaaatgcctcaaatgtcccgcccccttgactccactttttctcagtttccaaccctgttgatttccattatgcttttaatatatctttgctgctattttaatagaattttgagcatgtgttcaatccactatcttgaatggaaagtttctaaagtggcctgtgactcaggaagaagactgtgattaatgggcgagaatgaaaatcattgctcaatcccttgacttaaatccccaccacaacacctgacctacaaaacctgcccatacttgaatattccaagtgatgcagcattcactactcaaaatattagctggtgtacatattacagacccagcatctctccctctagttgaccatgacctctgaaattcacactctgatcctatctatttcctttcagttaccgtgcagatgtcaggaagcacttgttaagactattacctgaagtgtactcagaggcagagtaaatgctgggtcccggagcagacaggagagaggacctgggagttttttagtaagatggggaggaggagataggctatggcttggaccaggcctggaagagagctcaggtgtgcagtctctaggaacccgggtggagaagcagcaggaaataagcagaaaaaggagacaggccatgaagatagaagcgcaatggtcctggattcaaatctccactctgcagcttatagcttacagtccgcttagctttgtgcccattccaagaggatttccctattgtagctttacaatgttttaatatgtagtattgttgtgcaaggtaccatcctaaactgactgactttccttcctccttctcctccaaaataatttttaattcttgcagacagactttagatcgtgtgtttgaagtttttaaagatggaatttttattggaactgcattacacgtataaattatttaggaagagttggcttctgaagagggcagctctgtgggcttgggctgggtttgaatccagccagacaactttccagctgtgttaccttggacagttacctagttcctctgtaccttgacttcctcatctgtcaaatgggtgatgataatagcacctacaccatggtcgttgggaggagtcagtgagagtctccaagtgtgctttattatcgttggtggtggtagtggtgtttagaacgcatccctcagtcagacagcgaggtaggacttctccgtttattaaatcctgaccttttttttccatgtaaaacctgcacgtttctgaaatgctcagagtacgttactcagtatgtacccatatgttctgtgggtatactttgttaggttgtgattagttcgttggagctggtggttgcagggacggctggaagcaaggagatgaaggagaggaagtcgtagctgggaaggggaccaggaagtgggtgtcaggtccaagagctgctagaaagaaacgaaactgaaagcagggaatttcccaagtttggggaagacaggaactgcagcgcccctccccgtttcacgccacgcgcgggaccgaggacctaggacctggccagctgggcgtggttcggagagccgggcgggaaaacgaaaccagaaatccgaaggccgcgccagagccctgcttccccttgcacctgcgccgggcggccatggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaaggtgaggtcccacctcggggtctttatgtgtccaaaggggagtcctgggaggacgcttaagcctcacataggcttacggtgggggtggctttatccgcttgtgcacctcattcatttctttaacaaatacttccaatgtgccagccccgtgctaactcccccgaacatacgagccaggtaggtactcttaagcccgcttgacagatagagaaaatgaggcacagagaggtacagtgacgtgtccgatcctgtaagaggcccagccaggattcaaatccaagtagcctgattcctgcatctctactctcagaggctgcttcttatccattcattcactcattcactcattctcccagtcattcaaacagactgtgggcctcacactatgccagaccttaaggttacagaagtaataacactccgcgtaacagccagagtagcccaatttattgagacattatcaaataacaatgaaaattatctcattaaatgagtattacctcatttcatccccacgttcagtgaggcaggtattattgtacctgttttatggatagggaaactgaggctagacgctaaattactactctgagtcacatctttttttttttttttttttttttttttttttgagacagggtctcgctctgtcacccaggctggagtgcagtagcatgatcttggctcacagcaacctccgcctcctgggctcaagttattctcacacctcagcctcccaagtagctggcactacaggtgcacactaccatgcctggctaattatttgcattttttgtagagatagagttttgccgtgttgcccaggctggcctggaactcctgggctcaagtgatccacttgccttggcctttcaaagtgctgggattacaggtgtgagccaccatgcccaggtaatttttgcattttttgtagagacgggatttcaccatgttgcccaggctccgggtcactttttgtcacttaaatacagccacactgccactgtgatcccaggacactcgcagattcagggaggagacaagtcatgagctaccttatcctcagggtagatttgggggtgccaggagtggattgatggagggaacctgggtgtgctagaggaggttgtcaggagagggagtgaaaacgcagagtagaaatggcctcagagcacctagtggtttcctggggctgactctcagcttgggaggtggggaaggaggagacacttgtcgcctaggaggtgtgaagtcccgtttctaccttggagatggacaggagggatttctgtgcagcaagggaaagccctggcatccttagcttccctggaaaggggtgggagggggttctggagcacaatctccctgtgctttcattgtctcctttgtaaagtgagcacggtgcaatctccctgtgctttcgtttcctcttttgtaaagtggtaccttccttctggggctgtgaggaaggttcaatgagatgagagtgtgcaaaacgtgcaggaagtgcccagcctgtaatattccctgttcccactttgtcagcagcccccagatggtgggagctggtagtaaaaggactggcataatcatcgggataagtagggggtcccagtttgaggcaagccttgctcctggagggctccatcctggttcttgctgtttactggctgtgtaactcatttcaccatctgagcctcactccccatatctgcagccacatgtaactacatcacagttgttttgtgttggaagtgagattagagacgtcagatcccagacacagaacagtggctctgacaatgtttaggagtttcttttccaaatcctcaggttggaagctttgccgaaagaaacactaatcacacaggtctcagaaattgagtcataccacagttctgtacccattctgagcaaagattgcaaatgtgtgttctgcaaagcagaatttgttttacctgcggtcttgagtttttaaaaagttagttgccaacatttaaaaatcatggatataggcctggcatggtggttcatgcctgtaatcccagcactttgggaggccaacgagggcagatcacttgaggtcaggaattcgagaccagtctggccaacatggtgaaaccccatctctactaaaaatacaaaaattagccaggcatggtggcgcacacctgtagtcccagctactcaggaggctcaggtgggaaaatcacttgaactcatgagatggaggttgcagtgagctgagatggtgccactgcactccagcctaggcaacagagtaagtctctgtctcaaaactaaaaataaaaataaaattacaagtatggatttagcccagcacggtggtgcatgcctatagtctcagacacttgggaggctgaggcaagaagatcactcgagcccaggaattccaagctgcagtgagctacaatcgtgtctgtgaatagccactgcactccagcctgggcaacatagcaagaccccatctctttaaaaaaatgtaaaaactagccaggcatggtagcgtgtgcctgtagtctcaactacttgagaggctgaggcaggaggatcactcgagcccaggagttcaaggccgcagtgagctatgtgttccagcctgggtgacagaacaagaccctgtttctatggaaaaaaaatcatgagtttacatgttaaaattgcttctcttgaaatctcgagcctggctaccctgggcccacattcccgcgtggcttcaatgcctacagccatgtggccacagtctctccccagcacacttgcctcactcaagttgcctgccaggttgcaaggccactagaattggacagtatgaaattctgagttatttttctttgcccagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtgaggggttcctagaccattccagggttgggggcaaaagatcattgggaacaacacaggacttccaattctagcccagccaccaacttgctgtttgacctgggccagcctctacccctctctcagcctcagtctggggactaaacatattggacttgtgccagacaaaggcagagaaaccccagttcctcctggatctgtggtcctgctgtcaagatgcttggccctcccacaggcatctgggagtttccctcagccactgcctggagcggttactgctcagcccttccacaaatactcccgaaagctaagccaagtggacatgtggctagcagtaggggcctggggacgaaaccagaattctgcaaaatcttgtattttatgttgaaaacatcaatgtgatttttgtgttctccttcgtaatacacgtatgcttgagttaagataaaaataggcctctgtgtcctcatctgtaaaatgaggataatactaatggggataatagtaatcttatagagttgtcagaagaattaaatgcattaatatatgtaatcaagcttagaagtgtgtctagtgggccaggcgcagtggctcatgcctgtaatcccagcttcgggaggccaagttagagactgcttgagcccaggagttcaagatcagccttggcaaaatagcaagatctagtctctacaaataataataattttaaaaaatagccaggtgtagtagtacatgcctccctctgatcccagctactggagaggctgaggtgggaggattgtttgagcccaggcagtggaggctgcagtgaattgtgattttgccactacaatctgtctgagtaagactcttcttaaaaaaaaaaaagaaataagaatgtctagcatgtgataagggttgtaagagtgtttactattattattatttctattattgttgttgtttctattattattgttattattattattattattatttaccttgatgatctgggggatagctttatcctttgggcagatagccacatcccctatgcctggtaccctaagcctagtgtacccacatactgggtacacacacacacacgcacacacatttttaagacagggtctcactctgtcacccaggttggagtgtggtggcatgatcttggctcactgcaacctccgtcctgggttcaagcaatcctcccacctcagcctcccgagtagctgggctacaggcatgcaccaccatgcctggctaatttttgtattttttatagagatagggtttcaccatgttgtctaggctggtcttgaacttctgagatcaaatgatccacctgcctcagcctcccaaagtgctaggattacaggcacaagccaccccacccagcctgggtacacatatattaatcataatagttactacttattaaatgcttaactattccaagaacttgacatgttttatctcatcaattcttcacaacaaacttatgaggtctgtactgtttcagcttcaattttaccattggggaaactgaggctcagagacatacagtgacccacccaaagccacacagcaggcaagcggtagcagcaacctcagggaattatggaaagtgtttacttctcccaagccatgcccatgggagtggtggcggaaaaggcgcaccctgcaaagtccactggctctgagctcaaatcctgcctccatcgcatgctggctgtgtaaccttgggcaaatcatggaatttctctgtgcctcagcttcctcagctatcaagtgggatgaagaactgtacttgcctcatagggttgtggtggggatacatgtaaagaccttctgtccagtgcccagtatgcagtaaaaaaaatatatatttttttctaagacagagtttcgctctgtcccccaggctggagtgcagtggtacgatctcagctcactgcaacctctgcctcctgagttcaagcaattctcgtgcttcggcctctggagtagctgggattacaggcgagtgccaacatgactggctaatttttgtatattttgttggccaggctggtctcgaactcctgacctcaggtgaacctcccaccttggcctcccaaagtgctgggattacaggtgtgagccacagcgccaggcctgtagtaaaaaatatcaacaaccactgctaaaggatcagggctttttgccttttatttcttgggcatcgttttcctctccgtgacacctcttaattaaggatatgtagagtggtgttttgaggaaggctcagggaggagagagttgccaggctgcaggcactgctgactcagctggaagcaactttcctgtttaaccatcttggaatgcagctgctcctctttgagctgttgctctagggactgcctggccatttgggatggagggactctcccactgcattctggagtgccgctcttcttccctgcctcctgaaaagctccaggctctcttcccagtcccccgactcccatgttaccagatttcttcccctctgaattcttcccactctctctcagcgccccaggctgagctcggcaccaacaccgccctccatcctgtgtcctcagtgccctccctaactcacagcgcttcacaccaacaggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgaagccacttggaagggtttctccagacatgtgactgtttgctttgtgctttcatagtcgtgaaactgttgtcttacatttagtggccattcatgttctctctctgggaattgtctgtttatgtcccttgtgcattattttctcttgggttagctgtcttgttactgatttgttgaagctctttatatattgtggatactgatccttatttagttaatataatgcattcttccaatagtcttccagtctgtgtcttgggcttttgctttgtttaaaggttatttgtaggacaaaatatctacagtttaatgtactcagatatatcctttgaaaaatgagttattctttgtgtgtcttaagaagtctttccctatcctgccaggtgcagtggctcatggctggaatcccagcactttgagtggccgaggcaggtggatcacttgaggtcaggagttcaagaccagcctggccaacatggtgaaaccctgtctctactaaaaatacaaaatgtagccgggcgtggtggtacacacctgtaattccagttactcgggaggctgaggcaggagaatctcttgaacccaggaggcagaggttgcagtaagccaagattgcaccactgcactccagcctgggtgacagagccagactccatctcaaggaaaaaaaaaaaaaaaaaaaaaagaggtctttccctgtcctgactttgtaaaatgactctttcatatttcctgccatcatattgtttcctattgtatttctgttcatattcagggctttggcctgtctggtatggtatgaggtatatgcggagttggtacacttccctccctagcctcagggatgctcccttgctctgttcatgtttgttgccagactcttatgcactggggaaagtgtgcccagcctatgcctggagatgggggaacaggctcccccaaaatcttgcagcacatggaccagtcatcataagcaggtctctcaaggaagagattctggattccagttctggctcagccatcttcttgctgtgtgaccctggacaagttcctagccttctctgggcctctgtttcctccacttgcacataaagaaatatatgcatcggcaaatcttactcaggttctttccatgtaagattctccaggggcagaaaatgctaatagcctggtacttcctaaaagggcaaactgctgggtatggtggctcacgcctgtggtcctctccacgcaggaggctgaggcgtgaagatcacttgagcccaggagttcaaggctgcagtgacctaagattgagccactgcacttcagcctgggcaacagagtaagactctgtctataaattaattaattaattaatacaactatggagccagcctggttgggcgttagaaattctatcttgatctgggtggtgggtacacatgtgcacccacgcatgtttgtcaccccagagagagggggtgagcggagggagtgtggagatgctgggtgcttctagccaggggggtcactttctggcatgctgaagcatcccctctggagttacccagaggcctgggattcttgccttggcatggagtaatccctccctggtacgtggagatcccaagtatatggtgcacaaatagaaatcatgcattttgggatgggggatagacggctggagtgaggagggaaggacagagttctggcccgctgccccaccccactccaggggtgaaagggcgacaacttggtgtccttctccaatcagccacgcaactgggaccccagatctgcttcacctttatttgaggatagatagcaatttcagactacccttggcttctctctgactgctccctgcctccttgggacttgaaacttacacccctccctcccaccactcccccatcatatgagccaaccacttggctgaagctaccagtgagggctggtgagaaatgccacttgttcttggaacaggcttggaacacagcatcttgcctcaggctcggcctggaaggtccccagtctggttatgcagagagctggctctctttcctccccttcttccttctgagtcccctgccgatgccctctcacaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggtacttcctaatagcccaccatctgctctcctgtcccggggccagggggagataattgacaaggacaaaaccggttacatctactgagtgcttgggtatagaggccaaagcaaaatgctacatgtacatgttttaattcactggactcagcccagatgcacattagaattacctgggggagttttaaaaaatataccagtgcctggccccacaagttttatgcgtgaatgttttgctgatgtatagcagaaagtgcatgttctgatgactgtgtgaagaccgcatactttgaaaccaccacccagatcatggtagaatatttccagcaccccactggtcccctctcagtcagtaaccttccaaaataaccccacttacacttttataattacagttgtgccttttcttaaatcttatttaaatggaatcatgccacatgtacttttgtgtctggcttctcgttctgttctcaagattaagtctttgagatttagctatatagttgtggatcattcttttttgttgctgtatagtattccattgtgtgaatttatcaaaatatgttgtctattctactgatgatggacatttgagttctttcaacttggaactattacaaatagggccatcataaacattcttggacatgtttttttggtgcatatgcatatctgttggaaatatacctaggaatgaaattgctgaattgtagggtgtaactcaagagcccttgagagtggatcctggtatcttgtgattccaacgtgcagccagggttgacaatgataagcaggcaatgttgttttcctcattttgcggataaggaaatcgaggctcagagagggtaaatcatttgccccaggtcacacagctggaaagtagcagagatgcgattggaaccaggtccgtttcactccagagccctcttgctaaccagaaccttcttgtctctctgaaattgcaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccggtaagtgagggggccccaggacccttgggttttgcactttgtttatgtgtccagtgtttcctgagcatctactatgtgccatatggtgtggaacaggctttaaaaagcaggggtggccaggtgtggtggctcacgcctgtaatctcaacacattgggaggccgaggtgggcagattacctgaggtcagaggtcaggagttcgagaccagtctggacaatatggtgaaaccttgtctctactaaaaatacgaaaattagccaggcatggtggtgggtgcgtgtaatctcagctactcgagaggctgaggcaggagaattgcttgaactagggaggcggaggttgcaatgagctgagatcactccattgcactccagcctgggtgacagagtaagactccgtcccaaaaaaaaaaaaaaaaaaaatgcacagggcatcaggcatgtaaaacagtcttatcaccaagctcaagctgaaactgcccgggggaaggagtcctttgtttttctgcagctatggtctggttaccagacctttctgcccatcacaagggcacccattttgcagtttatccctccagaaaggcacctttttcaactctgcccaaagatggccatgtgcgttagccatggccctcccacttcccctctctgagcctcagtcaccctgactgtacaaagggcgggagctggggagagaaggcattgggttgatgcagaaaccactgcgcctggctgaggcagctccttcaatgaccttccagggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccattgacatcatcttgcgctgcctccatgagaactgtgttcacaaggcctcaagagtcagtaaagtgagttgggccagtggagacacaggggggaccctatcgagggatcagcgtggggaagggaaggagttacagcaatggagctgaggttgggctggggaactggacggcccaggaaggatgatttgctggcttaaataaaatatatttaagtgccagcttagattttcttttttttaatgatacagtttttaaaacactattacaagaatcagatcacttccctcctcagctcaaaattctccatggtttcacagggcgcagaggcctcaagtcctacaggatgggccctggtctccctctggccttgacttactccacactggcctcctggctgctctgagcttaccaggtccacagaacaattcaaggcctctgtagtggctgttttctcttcctagaagaaaatgagaattctcttccccaaccatttggctctctctctctcgctctctctgtctctcctcttcaagtttcttgatctgatgtcctcgtccactgaggcctgaccacacgatttaaaattgcagtcttgcccgggcacggtggctcacgcctgtaatcctggaactttgggaggccaaggtgggcagatcacctgaggtcaggagttcaagaccaccctggccaacatggtgaaaccctgtctctactgaaaatacaaaaattagctgggcatggtgacgggcccctgtaatcccagctatgcaggaggctgaggcaggaaaatcgcttgaacccgggaggcggaggttgcagtgagctgagaccatgccactgcactccagcctgggcgacaagagtgaaactccgtctcaaaaataaaaataaataaaaataaaattgcagtcttgcactaccagccccctttctgttctgcacagcacttagcattttctcaggacctattgtttattgatggtcccttccttccctcccccagccagcatgtaagttccagaagaacagaaattttgtatcctctcttcttctttctttcctgcaacaaacatttagtgagcacttacatgtatcggatatgtgtgatatcccgtgtgaaggagggagggagggagggaattgatggaaacagggcaacagaggacaattgcaggccctattagggcttgggacctcaatttgcaactgctggcccagatgtgcagtcagtagatgctgcagatagtaggctaaaatgtttggtcgctgcccctacttaatcccacctccattatctgttgctgcagtttggtctgacttagctcagtcttcatggatcagagggcaggtgcaggcagatacaacttgactctctgtcacaattctaagaggtcacaggacccatgcagtcgacctttgtcatagtccccagacctgacatcagcaagagggcaggttccaggctgtagatggggcgcaggtgatgggatcgtagtccgactcccaggctcctagagggtccctgatctgagctgttcttccctccacaggggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtgggtgagggcgcccagcctgtcccttggagagtgatagggacctcaggcgcccatctaacaggggcacctgccatcctctttgtgatcctaattctcctacttgaccaagcattgaaaactactttgagatactgaaagtaatcatcaccatcaaatcttacaactatttagtgctttaactttacaaatattttaattatgaaatgtttcaagcatacagaaaactacagacgtcacacacccatgtagcccaattcagatttaacaaatgttaacagtaggcatatctgcttggtatttttttaaagaaatttctcatgttataattttcctccttattttaaagggtttactttatggttcatttttttttaactttcggaggtgaatcttagcttagtaattatcagttttagtcaatgcaattaaagctacccatttccctttcagtatcacattagctacagggcaaatcttttgtttgacatgcataatttttattatcattccggtctaaatatttagtacttttaattatgacattattgtttgttctttcatccatgaatatttggaagtagctttccaaatgaatgttttggagatttgtttgctttcggcatttacttattatgttttttaaaactgtagtcagaaaacatactttatgtgatatcaattctttggaatttgttggcatttcttagattgcccatggttgaaaagaatgcatgctttccatccaggcatggtggatcacacctgtaatcccagcattttggaaggccaatgcgggcaggtcacttgaggtcaggagttcgagaccagcctggccacagtgaaaccccatctccactaaaaatacaaaaaaaaaaaaaaaaaaaaaaaatagcctggcatggtggcacgcacctgtaatcccagctactcgggagactgaggcaggaaaatctcttgaacctgggaggcagaggttgcagtgagctgagattgcaccactgcactccagcctgagagacagagtgagactctatctcatgctttccattgctgaatgcagacttctatataggtcctttagatctagcttatgacttcttgctcggataattttttgtttggtctctcaatttctgagtaaaatatcttaaaatctcctgctctgaatggatatttatcccttgttccttgcaattctgtaaattttgtttttaatattttgaggctatgttattgggaacatataagtgcataatattatattttattggattgttcttttattattaagtaataatcttcatttctgaaagttatctttgtcttaaagccttttgtctgacattaatagggctttagtcctcctttgtcagcattttcctgacgtaccctctctactctttatttactagcatgtgttattaaattgtacccattacaggcagcacaaagctaaaagacaaaaatgaaagaagcaacaatgatataatgatcttcaagggtttgataatatcttggaactaaacttccagacactaataataggggagattatttcttttgtttttgtttttacaatctatgcaaattaaaccctattaatataattaacaaattttttcaccattgctacttgatttcctgttcttccttctggattcattttccttcttacagaaatacatcttttagtactttttcagaaagggtttatgagtgacaaatttttctcagactttgtctaaaaccatttctgtttcacctccatgcttgaatgggatgcttgacacttgttttccttagttctgctgtcttcttacctgttgttgctgatgagaagtctctgtcagttattgattttttgttgttaatgatctgttttctgtttgcatgcatgaattttgtttgttcattttcttttgatttccctttcagtggcttttaagactttctcttggttcatagtatccttcagtttctctttgatgtatctgtgtatagaattctttttttctatgatttttttattatactttaagttctagggtacatgtgcacaacatgcaggtttgttacatatgtatacatgtgccatgttggtgtgctgcacccattaactcgtcatttacattaggtatatcttctaatgctatccctccccactccccccaccccacgacaggccctggtgtgtgatgttccccaccccgtgtccaagtgttctcattgttcagttcccacctatgagtgagaacatgtggtgtttggttttctgctcctgtgttcgtttgctcagaatgatggtttccaacttcatccatgtccctacaaaggacatgaactcatccttttttatggctgcatagtattccatagtgtatatgtgccacattttcttaatccagtctgtcattgatggacatttgggttggttccaagtctttgctcttgtgaatagtgccacaataaacatatgtgtgcatgtgtctttctagcagcatgatttataatcctttgggtatatacccggtaatgggatggctgggtcaaatggtatttctagttctagatccttgaggaatcaccacactgtcttccacaatggctgaactagtttacagtcccaccaacagtgtaaaagtgttcctatttctccacatcctctccagcacctgttgtttcctgactttttaatgatcgccattctaactggtgtgagatggtatctcattgtggttttgatttgcatttctctgatggccagtgatgatgagcattttttcatgtgtctgttggctgcataaatgtcttcttttgagaagtgtctgttcatatcctttacccactttttgatggggttgtttgatttttttcttataaatttgtttaagttctttgtagattctggatattagccctttgtcagatgagtagattgtaaaaattttctcccattctgtaggttgcttgttcactctgatggtagtttcttttgctgtgcagaaactctttagtttaattagatcccatttgtcagttttggcttctgttgccattgcttttggtgttttagtcatgaagtccttgcccatgcctatgtcctgaatggtattgcctaggttttcttctagggtttttatggttttaggtctaagatgtaagtctttaatccatcttgaattaattttgtataaggtgtaaggaaaggatacagtttcagctttctagatatggctagccagttttcccagcactatttattaaatagggaatcctttccccatttcttgtttgtctgtttgtttgtttgttgttgttgttgttgttgttgtttgagatggagtctcgctctgttgcctaggctggagtgcagtgacgcgatctcggctcactgcaagctccacctcccaggttcacaccattctcctgcctcagcttccctagtagctgggactacaggtgcccgccaccacatctggctaatttttttgtattttttagtagagacagacagggtttcaccatgttagccaggatggtcttgatctcccgacctcgtgatccacccacctcggcctcccaaagtgctgggattacaggcgtgagccactgcgcctggccccatttcttgtttttgtcaggtttgtcaaagatcagatggttgtagacatgtggtgttatttctgagggctctgttctattccattggtctatatctctgttttggtacagtaccatgctgttttggttactgtagtatagtttgaagtcaggtagcgtgatgcctccagctttgttcttttggcttaggattgtcttggcaatgcaggctcttttttggtttcatatgaactttaaagtagttttttccaattctgtgaagaaagtcattggtagcttgttggggatggcattgaatctataaattaccttgggcaatatggccattttcacaatattgattcttcctatccatgagcatggaatgttcttccatttgtttgtgtcctgttttatttcactgagcagtggtttgtagttctccttgaagaagtccttcacatccattgtaagttggattcctgggtatttcattctctttgaagcaattgtgaatgggagttcactcatgatttggctctctgtttgtttgttattggtgtgtaggaatgcttgtgatttttgcacagtgattttgtatcctgagactttgctgaagttgcttatcagcttaagaacattttgggctgagacgatggggttttctaaatatacaatcatgtcatttgcaaacagggacaatttgacttcctcgtttcctaattgaataccctttatttctttctcctgccttattgccctggccagaacttccaacactatgttgaataggagtggtgagagagggcatccctgtcttgtgccagtttccaaagggaatgcttccagtttttgcccattcagtacgatattggctgtgggtttgtcataaataactcattattttgagatgcctcccatcaatacctagtttattgagagtttttagcatgaagggctgttgaattttctcaaaggcctttttggcatctattgaaataatcatgtggtttttgtctttggttctgcttttatgatggattacgtttattgatttgtgtatgttgaaccagccttgcatcccaaggatgaagccaacttgatcagggtggataagctttttgatgtgctgctggattcagtttgccagtattttattgaggatttttgcatagatgttttattgaggattttcgcatagatgttcatcagggatattggtctttttttgttgtgtctctgccaggctttggtatcaggatgatgctggcctcataaaatgagttatggaggattccctctttttctattgattggaaaagtttcagaaggaatggtaacagctcctctttgtacctctggtagaattcggctgtgaatccatctggtcctggactttttttggttggtaggctattaattattgcctcaatttcagagtctgttattggtctattcagggattcaacttcttcctggtttagtcttgggagcgtgtctgtgtccgggaatttatccattttatctagattttctagtttatttgtgtagaggtgtttatagtattctctgatggtagtttgtatttctgtgtaatcggtggtgatatcccctttatcattttttattgtgtctattctatccttctctcttttcttctttactagtcttgctagcagtctatcaattttgttcatcttttcaaaaaaccagctctcggattcattgattttttgaagggttttttgtgtctctgtctccttcaattctgctctgatcttagttatttcctgccttctgctagcttttgaatgtgtttgctcttgcttctctagttcttttcattgtgatgttagggtgtcaattttagatctttcttgctttctcctgtggtcatttaatgctataaatttccctctacacactgctttaaatgtgtcccagagattctggtatgttgtgtctttgttctcattggtttcaaagaacatctttatttctgccttcatttagttatgtaccaagtagtcattcaggagcaggttgttcagtttccatgtagttgagtggttttgagtgagtttcttaatcctgagttctagtttgattgcactgtggtctgagagacagcttgttataatttctgttcttttacatttgctgaggagtgcttttcttccaaccatgtggtcaattttggaataagtgtaatgtggtgctgagaagaatgtatattctgttgatttggggtggacagttctgtagatgtctattaggtccgcttggtgcagagctgagttcaattcttggatatccttgttaactttctgtctcattgatctgtttaatgttgacagtggggtgttacagtctcccattattattgtgtgggagtctaagtatctttgtaggtctctaaggacttgctttatgaatctgggtgttcctgtattgggcgcatatatatttaggacagttagctcttcttgttgaattgatccctttaccattatgtaatggccttctttgtctcttttgatctttgttggtttaaagtcccttttatcagagactaggattgcaacccttgcttttttttgttttccatttgcttggtagatcttcctccatccctttattttcagtctatgtgtgtctctgcatgtgagctgggtctcctgaatacagcacactgatgggtcttgactctatccaatttgccagtctgtgtcttttaattggagcatttagtccatttacatttaacgttaatattgttatgtgtaaatttgatcatgtcattatgatgttggctggttattttgctcgttagttgatgcagtttcttcctagcatcgatggtctttacaattaggcatgtttttgcagtggctggtaccgattgttcctttccatgtttagtgcttccttcaggagctcttgtaaggcaggcctggtggtgacaaaatctctcagcatttgcttgtctgtaaaggattgtatttctccttcacttatgaagcttaggttgactggatatgaaattctgggttgaaaattcttttctttaagaacgtcgaatattggcccccactctcttctggcttgtagagtttctgccgagagatctgctgttagtctgatgggcttccctttgtgggtaactcgagctttctctctggctgcccttaacattttttccttcatttcaactttggtgaatctgacaattatgtgtcttggagttggtcttctcgaggagtatctttgtggtgttctctgtatttcctgaattttaatgttggcctgccttgctaggttggggaagttctcctggataatatcctgcagagtgttttccaacttggttccattctccctgtcactttccggtacaccaatcagacatagatttgatcttttcatatagtcccatctttcttggaggctttgttcatttctttttactcttttttctctaaacttctcttctcacttcatttcattcatttgatcttcaatcactgataccctttcttccacttgatagaatcggccactgaagcttgtgcatgcatcacgtagttcttgtgccatgattttcagctccatcaggtcatttaaggtcttttctatgctgttcattctagttagccattcgtttaatcttttctcaaggtttttagcttctttgtgataggtttgaacatcctcctttagctcggagaagtttgttattaccaattgtctgaagccttcttctctcaactcatcaaagtcattctccatccagctttgttccattgcttgcgaggagctgcgttcctttggaggagaagaggcgctctgatttttagaattttcagcttttctgctctggttgccccccatctttgtggctttatctacctttggtctttgatgatggtgacgtacagatgggattttggtgtggatgtcctttctgtttgttagttttccttctaacagtcaggaccctcagctgcaggtctgttggagtttgctggaggtccactccagaccctgtttgcctggatatcaccagtggaggctgcagaacagcaaatattgcagaactgcaaatattgctgcctgatccttcctttggaagcttcgtctcagaggggcacccagccatatgaggtgtcagtcggcccctacggggaagtgcctcccagttaggctactcgggggtcagggacccacttgaggaggcagtctgtccgttctcagatctcaaactccatgctgggagaaccactactctcttcaaagctgtcagacagggacgtttaagtctgcagaagtttctgctgccttttgttcagctatgccctgcctccagagatggagtctacagaggcaggcaggcctccttgagctgcagtgggctccacccagttcgagcttcccagccactttgtttacctactcaagcctcagcaatggtggatgcccctcccctagcctcgcttctgccttgcagttcaatctcagactgctgtgctaacagtgaatgaggctccgtgggcgtgtgaccctccgtgccaggtgcaggatataatctcctggtgtgccgtttgctaagaccattggaaaagcacagtattagggtgggagtgtctcgattttccaggtaccatctgtcatggcttcctttggataggaaagggaattccccgaccccttgcacttcccaggtgaggcgacgccctgccctgcttcggctcatggtccgtgggttgtacccactgtccaacaagccacagtgagaggagtgagaggaacccagtacctcagttggaaatgcagaaatcacccatcttctgtgtcactcacgctgggagctgtagactggagctgttcctattcagccatcttggaacctcctctctggatttctttttaatttgtcctctttgaaatgtgctgtccctcctgaatctgagagctcaattcttcctttagttttagggagaggtcttccaatagctcttcaaatactgcctctcccctattttgtctattctctacttctggaactcctgttagatggtttcttatctcatcctccatggcttttcatctctctcattgttcccatctctttgtctatcagggctgtctgtttttcagtttcttggacctgtcttctggtttataaactctattcaaataatgtctactctgctgcttatccttaattttttaaaatttcagtgactttttcacctctaaaagttatgtttctctctttcttaaatcaatcagttatttgttcataggcttcgattctttcactgtggtttcagtttctttgatgtcttcaataatttccaacatgcttattttagtgtctcagattgtcctattaactcaaattcttgaggtgctaattttcctatttgttgtgtcacctgactctccctcatggtcaatcatttcctcatatagtttgtaattttttattgtgaactcatctttggggtttgtggagattttgttgtctttgcttttcctgtggaagttccttgtgccctgggttggagaaatattacccttgggaccaagttttcacttcatttctacctggactccaagagtttcactaattcccaggctagattatgcattaatttaacagtctgggatttcctcaccatgcagatactgtaaatttggacttcacatccacatctagcacaggatcggggtcccagtttctcacaggggattttcttttttcccccacccagagccctagcagcaagcttccttgaagcccctctctgccagtggacaaccttttgcagtcacttttcatgaagaaggcatttattttagaggccagccttatgaaagtgaaggacaagactcaattgtagcttcctctctgtgtcttgtttttctttttctggaagataacgttaataatagcaataattactgcttattgagcatatgatgtattctacatactgttctaagcacttcacatgtagatttcaattgattcttacaatagccttagaaggataggtgctatttatgatctcccttttacagatgaggaaactagacccgaagaggtaaaaatcacacagcttggatttgaacccaggcttatctgtctccaaagctcactcctttaaccactatgtgacacagcttttctggctcaagattatccatctttcagctttcagggaaaatttaaaaaacaaaaaataaatagaagattatccactgtgcagttactggatggattagcaaaaaatattgaaaagcatccagtacagaattgatactgaaaaaatgatagctctgattgttattcaatgttatagtgtattattgattgcaatgttaggtgattaatgttagaatattcataaaggctggtgaatggatgggttggtaaatgctgctgtcttcaattggagttgcacactcagggtgtttcaaacttctacagggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggtgagttgcccctggctcctcccaggaagccaccactgtcatggcaaccaccccagccaatcagttcctcctctacacccacatctcccctcctttgcttcttattggtcatccagagcagaaggaccggcctcctccatcctccatttcctgcccagatctggaagccactgttagaaaaaaatctcttctccatcaagtctaaagtcttcatttcttgtacctggggttcactttagcccatccacttctctcttttgacactgcaaatgttttctctgtttttcccccacctccaagccgttgcttatgatattacccccaccacatgtctatttttagaaaagaaaactcccctttctggaagcctagagctggcaatgaccaccatatggtaagggcctgtccaagacagaaaaaccagagcacttgaacagaaaagatctgaacagaaaagctgatgactccttatgggcttctggatcaagctgtgcctgaaagcagatctactccaaggcttttgggttacatcagccaatacattctttcatagccttttttttttttgagaaagtgtctccctttgtcatccaggctggagtgctgtggcacaattacagctcgctgcagcctcgaccttctgggctcaagtagtcctcctgcctcagcccccgtagtagctaagactacaggcatgcactactacacctggctgatttttgtactttttgtagagacaggagtcttaccatgttgcccaggttagtctagaacttctgggctcaagtaatccacccaccttggcctcccaaaatgctagaattacaggcacaagccaccatgccccagccttgttgcctcttttgagttgcactaaattctgaacatccttcggagctcccttactaaatacttctctgaattctgacttgtattcatgcatccctatattcaacaaacatctatcgagcacctgctatttctaacttgtgatggacactgggataccaagatggatacattgcagcccctattcctgtacaaccaccccatgttatgtgattaatgttaaaatattcctaaaggctgctgatggatggattcgtcaatgcttccgtcttcaattagagttacacactcaagatgtttcaaatttccaatttccttgaggggagacactaggcaaaagccacgtctgagctctcagtctcactgtatgaccttagacaagtcactaccctcctctgaacctcagtttacccacctgtaaaatgagaagcatcagcaagtttctattctttctgcgcttctattttctatattcccttcctgccccaagtgcttatggccacactcagctcacatccactaatcactcatctttggttggccttgtgtgacacaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccaggtgaagacccgcttccctttgcctggcttcattatcctccccctccccactgtcaccctggagtcagtcatccaggaggagtccaaggtagggtttggggtggcaatcccactcctcactctgcttccctctggactctttgctgaggaagtgtggacataaggagtcccaaaagaaaccagggccagttttattagcatgataaaatagtatttctcagttgaaggggccacccaatagctttccaaccaaggcagccaattgagatcgcttctgcacttgggcaagactgagccaaccctgaggtcctgacactctttccagccctcacgccccttttcagcccttccacccgcctcctctttcactgactcccaccttccccacccaccttcctgctgtgcccccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatggtaagatggagggtcctggggggcagggggccctgcaccctgccttctagtcaggttcccttaacctgccggtgcacccatccccagctgctaggagtgttggtggctgacaactcatagccaccccttctctggagacttgcctttcatgaaatgcacagattgctacgtcccagccagtgcctgagtgacacagggttacaaaaagcctaactctgtctccaggcggaaccgattctgtgatgcaactcacgttccagcgctccctgtggaatcaggcaaacacttgtctccggctgagcacccagctttgctgagcctcttctctgccctctgctgctgtccttgttccacttctcctccaagcactgccccaattaatcatatgcacaagaattcctgccatggaccctgcttctaggaaaactgagacataagccacttgcagctcccaaaaggatatgattttatcacatttactattttgcagcagggtctcataaccagcatttaatactcaggacaatccattgagacccggacttcattattgcactcatttaaacatggggaaactgagactgtgtattgatgctggaaccaaaattcaatctcaggtccttctgatgctacctcagaacctacccaccagctgagaaggaaagagggacatgggagacagtgggagtcttgtcctcagaggacatcaaggggcagggcttgggtgagcactgggagtcccgtctcaagctggccccacctggattctctctgcagccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaaggtgagtcctcagagagctgtaggcaagcagtgtcctgcaagctggtgatctctcccagcccagggccaggcttgacccacttccgccctcgtagcaaacagcaaaaagccaggcatagagaaagagctggaaagtggtcatgggaggatggcagagagagggcccagatatgtccaacaagtctcttttggttgctagtgacacctaactcaaaagacacaacaaaggaatgtattgactgatgtaactggaaagtcctgggtagggctgcttaccggcatagctagatccaggagcccaggcagcatcatcaggactcagtttttctctccagttctcggttctgctttttaatgtcttggataggcccttaacatatggtggtctttggcagctctaagcttccagaaaggggaacttgttttttcttaaaattcaaataaaagtcttggaattgagtctcattggcctggcttgggtaacctaccaacccccaaactagttactggggctagggagacattatgctctggatggccaagactgggtcacatggtgaagtggcccccgcctagtcctcatgggctgagtgtggggtaggagtggtccctccaaagaacaccaggatgctgatcccaggtggaaaggacgctgggggtgtacaaaaggtcacactgtccacggccctggatacagcctcagtccacagttggagagacaagtgagacccagatcagctcagaaagagtgttagcataaagccagccagtgaacagtgccagggatgctggtttgctgtgtgaccataggcatgtaccttcccctctctgatcctcacgttcctgatttctaacacttgaggagagtggcttggatcactggctctcaactctggctcccctggcttgaccttggaattacagttttttaaaaatatgctgatgcccaagccccaacccaggaaaacttaaatcacagtcttgtggggtgggagccagccattagtcgttggtaaaagccttccaggtgcagacaggcttgggagctgcaggcccaaaaaaccaaccaacaaacaaaacacttctcaaatcctctccagtcacaaaacatgatggaatgagaaattttgcatttgttgaagggtttaaacttacctcttgcacgcttctttttcttacaatcttaaatacagtctttcttaccatattaaagatctaacacagagaggttagatgacttgtccaaggtcacacagtaggttttctaactcacagtccagaaccgacaggctaagccatgcttcaagggttgagccacctgccatgtcctctccagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggtgaggtgccctggcgtagacctgagagggggaaatacagaggcagggccgccatgggcagttgtagaggttgcacagtacacaaccaggccacatctgttcgcatcattgcaggcattgtagttgtgtatgttcatcacaactttcctgcaaagtatctaaagaagagggcccctttttctaatttgcgcaggcactctgtgggctaacagtggcctcaagctgggttccagtctgagcaattccaccaacatgctggatgaccttgaacaagggacttccccgtcctgagccccagtgtctttatctcacatctgacagtaaggacactgatgtttcttgcacattcccagctttgaatggtttgtgtgccacccacctcctccacctgctgcagatccattcattcagttcattcaatacatgcatctactctgtgcccagtgctcttccaggcaccaaaaataaagccttgaacaaaatagacacaactctcttcatatcttttcaactctcagtttgattagcagttttcagagtgagaaattcccttgtatccgaatttatttggtttctgagtttgagggagcaggtggccaagggagggattcatgcagaatttttttttaagagacagggtcttactttatcacctggcctggagtgcagtggcaccatcatgactcaatgcagcctcaaactcctgggctcaagtgatcctccagccccagcgtcctgagaaactgggactacaggtgcacaccaccacacctggctaatttatagaattttttgtagagatggggatatgactatgttgcccaggctgatctgaaactcctggccacatatgatcctcctgcagtggccttcgagagtgctgggattacagacgtaagccactgcacccagcccagaaatttatctgaatctactcagttcttcagttcagagagcaagaatttggatattaaggaatgcctttaagtgcaatgtaaccagaatggtgatgtcaactccatacacagctctgttacctgcaggaggatgtaagactgaggcctgccctccctcggttagacagaaagataagtaagtattagagaggtgttaaagacaggctagctcccagctgagactttttccaagataggtaagcagatggtttgaaagggagcagaaaagggaggatgactgtcaccagggatttaatgtggatcaggccacatctgtgttccacctaaaaacaccctgtggcctcccagtggatcccagaccacccttaggaaaacacccaagaggtaggagatctcagaagtcctttctaagttggccccactgggacaacatgggagccggagtgatggtaaccatctccccatctccaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcaggttcggcacatggataggccaccttcctaagttgccctgggatctgcctctggagcactttcctgggaggaagcagggcccagccctggccaagatcctgggttggtggagcagagcagaaagagtgctatatctcagctgtgggaccttagttttcttatctgtaagatgggggtgataaaactatgtcacaggatgtgatgggataatgcatggcaaggcatctggcacatgtaggtgctcaataaaagttttggggttgctttgccaagtccagaataatcccttctgtacctcatcagtgccaatatgaaccaacatatctttcttctcgttctccagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggttcaggtctacccccaatgttccagaatttcaaacctgggatcactcactctccccactttctagattgcagagcagagatgggaaaacactcttcctagaacggattccttcctagaagttatatttgtagtacctgagggacaaacggtcaattttctggtcacccaggaatagggttgccagataaaacgcaagaccccaagttaaatttgaatttcagataaacaatgaataacttcttagtataagtatgctcgatgccatatttgagacataattacgcttaaaaatttattcactgtttatctgaaagtcaaatttaactgggcatcctggttttttgttgttgttgttgtttgtttgtttgtttttgtcacccaggctggagtgcaatggcgcgatcttggctcactgcaaccctccgcctcccgggttcaagcaattcttctgcctcagcctcctgagtagctgggaccagaggcgcatgccaccaggtccagctaatttttgtatttttagttgagacaggggtttcaccatgttggccaggctgatctcaaactcctggcctcaagtgatcgcctgcctaggcctcccaaagtgctgggattacaggcatgagccactgtgcccagccttgtatttgtatttgttaaatctgaccaccctacctgtgaacccacccaggtgccataagcatgtttcattctttgggattttgcctacctctgaaatgggtacagagataatagagatgcttttgcaaactcaagatgcatctccagtcagtggggagtggctacttagaatgatgtgttgaaaaagcttctaaggttgtgatttgactcagctggaaagagtaggctaaccagctagtagtgtccaaaggttgagcatcccaaatgcaaaaattcaaaatctgaaaatggcccagaatttgaagctttttgaccaccaaaaaatatgctcaaggaaaatgcttattggagcattttggattttcaaattagggattcttaactggtaagtaaggcaaaggtccaaaatctgaaaaaatttgaagtccaaaacacttctggtcccaagcattttagataagaaatactcaacctgtatcatggatgcaggaggggagagttgtggaaccagtgttgtgattgattaacaatgcctcccctgaataaggaaggggtaagcggtagcaccgtggttgattagtaatgtttgcctgaatgcagaatggcaaagtggccatgtgtgtcttattttctatacctgccctgtagtgtataggagcactgaggaatctctgagccctggctctagcccctgcaaagtgttagataaaaggggaaaatagtccaaccagtgccacaggtggacacctagatgttgccaggaataagactgtccctgggtgggaattgcaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggtgagagatctgtggtgccaaaggaagtaccctttaggggtaaggggggagcatggtcaggggagggacatgattcccactaaaggggcagggcccagtgatggccccaggtatgcccctgtgcttccattttcccatccggctgtgtggtctcagcttctgcagaaagaatggggttaccaacatctcttataatacttccccaggctgctgtgtgaagttgagaaaatcagcggtcctactggatgaagagaagatggacaccagccctcagcatgaggaaattcagggtcccctaccagatgagagagattgtgtacatgtgtgtgtgagcacatgtgtgcatgtgtgtgcacacgtgtgcatgtgtgtgttttagtgaatctgctctcccagctcacacactcccctgcctcccatggcttacacactaggatccagactccatggtttgacaccagcctgcgtttgcagcttctctgtcacttccatgactctatcctcataccaccactgctgcttcccacccagctgagaatgccccctcctccctgactcctctctgcccatgcaaattagctcacatctttcctcctgctgcaatccatcccttcctcccattggcctctccttgccaaatctaaatagtttatatagggatggcagagagttcccatctcatctgtcagccacagtcatttggtactggctacctggagccttatcttctgaagggttttaaagaatggccaattagctgagaagaattatctaatcaattagtgatgtctgccatggatgcagtagaggaaagtggtggtacaagtgccatgattgattagcaatgtctgcactggatacggaaaaaagaaggtgcttgcaggtttacagtgtatatgtgggctattgaagagccctctgagctcggttgctagcaggagagcatgcccatattggcttactttgtctgccacagacacagacagagggagttgggacatgcatgctatggggaccctcttgttggacacctaattggatgcctcttcatgagaggcctccttttcttcaccttttatgctgcactcctcccctagtttacacatcttgatgctgtggctcagtttgccttcctgaatttttattgggtccctgttttctctcctaacatgctgagattctgcatccccacagcctaaactgagccagtggccaaacaaccgtgctcagcctgtttctctctgccctctagagcaaggcccaccaggtccatccaggaggctctcctgacctcaagtccaacaacagtgtccacactagtcaaggttcagcccagaaaacagaaagcactctaggaatcttaggcagaaagggattttatctaaatcactggaaaggctggaggagcagaaggcagaggccaccactggactattggtttcaatattagaccactgtagccgaatcagaggccagagagcagccactgctactgctaatgccaccactacccctgccatcactgccccacatggacaaaactggagtcgagacctaggttagattcctgcaaccacaaacatccatcagggatggccagctgccagagctgcgggaagacggatcccacctccctttcttagcagaatctaaattacagccagacctctggctgcagaggagtctgagacatgtatgattgaatgggtgccaagtgccagggggcggagtccccagcagatgcatcctggccatctgttgcgtggatgagggagtgggtctatctcagaggaaggaacaggaaacaaagaaaggaagccactgaacatcccttctctgctccacaggagtgccttagacagcctgactctccacaaaccactgttaaaacttacctgctaggaatgctagattgaatgggatgggaagagccttccctcattattgtcattcttggagagaggtgagcaaccaagggaagctcctctgattcacctagaacctgttctctgccgtctttggctcagcctacagagactagagtaggtgaagggacagaggacagggcttctaatacctgtgccatattgacagcctccatccctgtcccccatcttggtgctgaaccaacgctaagggcaccttcttagactcacctcatcgatactgcctggtaatccaaagctagaactctcaggaccccaaactccacctcttggattggccctggctgctgccacacacatatccaagagctcagggccagttctggtgggcagcagagacctgctctgccaagttgtccagcagcagagtggccctggcctgggcatcacaagccagtgatgctcctgggaagaccaggtggcaggtcgcagttgggtaccttccattcccaccacacagactctgggcctccccgcaaaatggctccagaattagagtaattatgagatggtgggaaccagagcaactcaggtgcatgatacaaggagaggttgtcatctgggtagggcagagaggagggcttgctcatctgaacaggggtgtatttcattccaggccctcagtctttggcaatggccaccctggtgttggcatattggccccactgtaacttttgggggcttcccggtctagccacaccctcggatggaaagacttgactgcataaagatgtcagttctccctgagttgattgataggcttaatggtcaccctaaaaacacccacatatgcttttcgatggaaccaggtaagttgacgctaaagttcttatggaaaaatacacacgcaatagctaggaaaacacagggaaagaagagttctgagcagggcctagtcttagccaatattaaaacatactatgaagcctctgatacttaaacagcatggcgctggtacgtaaatagaccaatgcagttaggtggctctttccaagactctggggaaaaaagtagtaaaaagctaaatgcaatcaatcagcaattgaaagctaagtgagagagccagagggcctccttggtggtaaaagagggttgcatttcttgcagccagaaggcagagaaagtgaagaccaagtccagaactgaatcctaagaaatgcaggactgcaaagaaattggtgtgtgtgtgtgtgtgtgtgtgtgtgtgtgtgtttaatttttaaaaagtttttattgagatacaagtcaataccataaagctctcacccttctaaagtgtacaattcagtggtgtgagtatattcataagatttatacttggtgtctattcataagacttatatccagcatattcataactagagccatatcacagatgcattcatcataataattccagacattttcatcaccctaaaaggaaaccctgaaacccattagcagtcattccccattcctccaacccattctctccctaatccctagaaaccaccaatctgctgtgtatttcatctattgccaacatttcatataaatggcatcatacaatatgtggccttttgtgtctggcttctttaacttaacatgttttcaagattcattcatgttatagtatatgttgaggtttcattcctttttatttccgaaagacatttccactgcataaattgactacattttgtttatccattcttccattgataggtatttggatattctccacttttttgctattttgctgcattttttgctattatgactaatagctgctatggacactcttgtatgagattttgtgcagacatatgttttcatttttcttacgtataaaaggcgatccacaattgatgaccccatcataagtcaaggagcatctgtgtgctgtattaatcagagttctccagaaaaaccaaaccattagaatagaggaagagatatataaagagatttattatgagggtttgacttgtgtgattatggagtct

SEQ ID NO: 2 - DNA, 34980 bp, Homo sapiens
tggctcacacacttggggtgtgttttcttgcccttgcgaggctggacaagtgcctcatacggtccctcggctgtccacaagtgtgctgagaaactggacacacagaggttcccacccgctgaccacacgggggcctgtgtttacccagaagcagaccaaaacctgagtccagctaggtccctgctttgctgtgtgtccctgagcaagtcagttccctctctgggtctctttttcctctctgccgctttccctgaatgtgagttccctggccactgagaacagggggaaagggacagagcctcaaaagatgacctgtgcgcccctgcttctaagcactttgcaggtattatttaatcatcaagctaatcctacgagagagctgccattttcccccagcttacagatgggggaattgaggcgcgaagagggcaggcgatgtgctcaaggacagacatctagcaggtatgaagccctcacaatggggttctagaggctgtttagttaacctcaagttttggggagcccctgaagggctggtcaccacgctgccggggacagggaaagcctctgagcttgagtcagttttggtttccctgctggggtgcaggagtcagtaaaccttgctgcaaggggcggggaagagcatttgagcttaagttagttttggtttccctgccccggatgcaggagttggtaaactcactgcaaggggcagggcagagcctctgagcttaagttagttttgtttccctgtcccggatacaagagttggtaagctcgctgcagtgggtggagagaggcctctagacttcagtttcagtttcctggctctgggcagcagcaagaattcctctgcctcccatcctaccattcactgtcttgccggcagccagctgagagcaatgggaaatggggagtcccagctgtcctcggtgcctgctcagaagctgggttggtttatccaggaatacctgaagccctacgaagaatgtcagacactgatcgacgagatggtgaacaccatctgtgacgtcctgcaggaacccgaacagttccccctggtgcagggagtggccatagtgagtccagggctgaggttgggtctctgggaggcaggagattccacggcggcagcaaggccgagctactgggtgctgggtgcctattatgtgcgaggcccacacttgggtgggatgtggtgtaggagtctcaggctctggagcaggcgcttgctccagagctgtgtgacactgggcaggctacttaacctctctgtgcctcagtctctgactctgtaaaatggggagaggcataatacccacttcagagtatcgtaaggcttgagcacatcatgttcttagcaaaagactggcagtgctcagtgaattcactgtgattactcactgcgattttcttctatcctctccacagtacagaggagtaaacttagggaggctagagaactttgttcaaactacccaggttgccttgtggtttctccgcagcaagaaggagtggcttttatcagttagaaatatttggttttgtggacacaaatctcaggactgaggctgaaaattctggacctctggggaggaagggggactggggagatgccaggaccctagaattgggggcgtgggggtcctgatggcccaatactatcctcttagcctcctgagctgctgacgtcccttctctgctgctgccacactttttggtctctaccccttgcacacatccgctagtgcctcaataaagaaatctagacacgtggtgtccccagccctggcccaggccagcaagctaaggcaagtttggttaatagctattgtcagaactgggattttaagcctgggtaattggcttgagggcatgcatgtttaaccactacactatcctgcctctcaatttttttttcctcatggaagtaaaacagaaaaagtgcccagatcaaagtgtccagcttgataggtagccacaaacaatgtaactgtgtaacccccacccagatccagaaacaaaacattctcagccccccagagccctgggcccccgatccagtcagcacctccaaacccaggtaggcaatctcctgacttctaacagcatagacacgttttgcctgtttttaaacgttttacaattatagggtgaccaactatcccagttcatccaagattggggtgtttcctgggatgtgggactcttggtgtgaaagctgggaatgtaaaccaggatgcatgggtgaaccctacataaatggcatcatgggctctgtgtgctttagtgcgtggtttctctgacctatgatgtttatgaaatttatgttgtggggtacagttgtttatcttttcattgttgtgtcaattccattatgtgaatattccacatttgtccattctactgtgtttttttatttttacataattgtgcatatttatatggggtacatgtgatatttggacacatggatacaatgtgaaatgatcagggtagttaggatatccatcacctcaaacattgatcatttatttgtgttgagaacagttcagatcttctcttctaactattttgaaatatataataaattattgttaactatagtcaccccattgtgctatcaaacactagaatttatcccttctatctaactgtatgtttgtatccattaaccaacttctcttcatttcctcctctacttcccagcctctggtaactatgatcctactctctacctccatgagatcaactttttttaacttccacataaacacatgcaatattgtctttctatgcctgacttatttcatttaacataatgacctctagttccatgcacattgctgcaaatgacagaattccattctttttgtgtctgaatagtattctattgtgtatatatactacattgtctttatccattcagctgctgatggatacgttggttgattccatatcttggcttttgtgaatagtgctgcaataaacatgagggtgcagttatcccttttttggataaatacctagtagtgggattgctggattgaatggcagtgccatttttattttttttagaaacctccatactgttttccgtaatgactgtgctaatttacattcccaccaacagtgtgtaagattccctttactccaaatccttgctagcgtttattttttgtctttttgataatagctattctaactggggtgagatggtatctcattgtggttttgatttgcatttccctagtgattagtgatgtcaagcattttttcatatacccattggccattttgtatgtcttcttttgagaaatgtctatttaggccctttggctgctttttaatgggatgggttgttttctttgctgttgagttgttcacgttccttgtatattctggatattaaacccttgttggaaaaatagtttccaaatattttctcccattctacagcttgtcttttcactcttgattgttccctttgatgtgcagaagctttttcatttaatatagtcccaattgtctattttttgtttttgttgcctgtgcttttgaagtcttagccacaaaatacttgcatagaccaatatcatgaagcattactcttctgctttattctattaattttatcatttcaggccttatgtttaactctttaacccatttttagttaattttttataagatgacagacgggggcctagtttcattcttctacatatggatatccagttttcccaacaccatttattgaagagagtatcctttccctaatgtgtgttcatggtgctttctcaatcagttggctgtaaatttgtggatttgtttctgggttctcgattctgttcctttggtctatatgtctgtttttataccaatcccatgctgctttggttactatagctttgtagtatattttgaagtcacgtagcatgatgcctctagctttgttctttttcctcagtactatttggctatttgagatcttttgtgtttccatacaaattttaggattgttttttctatttctgtgaaaaaaaatgtcactggtatttataaggattgcattgattctgtagattgctttgggtagtatggttattttaacaatattaattcttccaatccatgagcatgggatgtctttccatttgcttgtgtcaccttcagtttcttttatcagtgttttgtagttttcactgtagagatctttcacttccttagttaaatgcattcctaggtatttttgttaaatactataagattgctttcttgatttctttttcagctagttcattactgatgtgtagaatatgttggtattttggtatgttggtatggtatgttgattttggtattttgattttgtatcctgcaactttaccaaatttatcagttctaagaggttttcttggtggagtctttaggtttttctgtatagaagatcatatggtctgcaaagaggggcaatttgactttttcttttcctatttggatgccttttatttctttatcttttgggattgctttggctaggacttccaggatacaggctgaaagcctttcccaattcagtatgatgttaactgtgggtttgtcatatatggactttattatgttgaggtatgttccttacatgcctaatttgttgagggtttttagcataaagggatgttgaattttaccaaatgttcttctacatctaccaggataatcatatgatttgtccttcattatgttgatgtgatgtgcaaatgttcaacatttgttgacttgctcatgttgaaccatccttgcatccctgggataaatcccacttgatcatgtgttatcttttcgaagtattattggatttggtttgctagcattttgttgaaggtatttgtatctatgttcatcagggatattggcctgtagttttcttttttgttgtgtctttggtctagttttggtatcagggtaatgctgacttcatagaagtggttaggagtaattccctcctcttcaatttttttggaatagtttgagaagaattggtgttaattcttctttataagtttggtagagttcagcagtaaagccatctagtcctgggctattctttgttgaggagttttttattactgattcaatctactcactcactattggtctgttcaggttttctgtttcttcctggatcaatcttagtaggttgtatgtgttcaggaatttatccatctcctctaggctttccaatttgttcacatgtagttgttcatcatagtctctaatgaccccttttatttctgtggtatcaatggtaatgtctccttttttatccctgattttattttacttgggtcttctctttttttagtctatttagtctaagtagcggttcatccattttgtttatctttttaagaaaccaactttttattttgttaatttttagcctctattatgtttagttctgttctgatttttattatttctttccttctactaattttaggtttggtttattcttgcttttttgtttgtttttgagatggatttttgcttttgttgcccaagctggagtgcaatgacgtgatctcagctcactgcaacctctgccccctgggttcaagtgattctcctgcctcagcctcctgagtagttgggatcacaggcatgcaccaccacgcccggctaattttgtatttttagtagagacggggtttcaccatgttggtcaggctggtctcaaactcctgacctcaggtgatccacccgcctcagcctcccaaagtactggaatttcaggtgtgagccactatgcctggcttttcttgcttttctaattccttaagatgaaccgttaggttgtttatttgaaattgttctactttttttgatgtaagcatgtattgcttcaaactttgctgtatcccttaggttttggtatgttgtgtttccattttcatttgtttccagaaattttttgatttccttttaaatttcttcatcagcccagtggttgtccaggagcgtgttgtttaattttcacgtatttgtacaatttccaaagttcctcttgttccattgtggtctgtatccattgtggtaagtatccattaagatacctgacagtatttcaatttttaaaaatttgttgagccttgttttgtgacctaccatagggtctatcctggagaacattccctgtgctgataagaatgtgtattctgtagctgaataaataatctgcaaatattcattaggtccatttggactataatgcagactaagtccagtattctttgctgaataaataatctgcaaatattcattaggtccatttggactataatgcagactaagtccagtatttctttgctgattttctgtctacgtgatctgcccaataccaaaagtgagatgaagttcccagctattactgtattggcagtctctcggtctctgtttagctctaatatttgctttacatatctcagtgctctaccgttgggtggtcatacatatgtgtataaatatacatatacatatatatgtctatatacatatatacacatacttatatcctctggctgaattgatctcttgatcattaattataaaatgaccttctttgtctctttttatgtttttgacttaaagtctactctgtctgatataagtatagctactcctgcacacttttggtttccatttgtgtggaatattttttccattccttcacattcagtctatatgtatcttcacagatgcagtgagtttattggaggcagcatatagttggaccttattttttatccattcagcttgtctgtatcttttaactagagaatttaaaccgttgttaaattcagggttgttattgataggtaacaacttactcctgtcattttgttatttgttttgattgttttgtacattctttgttcttttctttctctttattgcctacctttccaattgttgggggttttgtaatgataacatttgactcctttctctttgtcatttgtgtatctgctctaccagtgagttttatacttttgtgtgttttcatgatggtagacatcatccctttgcttccagatgtaacactctcttaagcattttctgtagaactagtctagtggtgatgaattccctctgtttttgcttgtctgggaaagactttgtttctccttcatttttgaaggatagctttgctgggtatcgtattcttgtctgaaagggtttttttttagcactttgagcatatcatcctattctcttctgacctgtaagttttctgctcagaaatctgctattagtctgaggagaattcccttatatgtgactttatacttttgtcttgctattttcagaattatatgtttgtctttgacttttgacagtttgactataatatgcctgcagaagacattttggggttgaatctatttgagaatctttgagcttcctgtatctgggtgtctaaatctcttgtaagacatgggacgttttcagctattacttcattaaatagattttctatgcctttgcccatctcttctcctagatctttccaaatttaaatatttggttactttatgattaccaatatggcacataggctttcttcactcatttttatttatttttctctccttttgtctggctggattatttcaaaagaactgtcttcaagtttagaaattctttcttctccttgacctagtctattgttaaagcagtgagttgtatttttattttattcattgatttcttcagtttcaggaattctctttggttctttttagcaatatctatctctttgttgagtttctcattcagagcataaattattttcctgatttatttgtattgtttatctgtgttttcttgtatcttactgaatttctttaatatcatggttttgaatttttcttaaccattgcacagatatcattttctttgggatctgtactaaagaattattgtgctcctttggaagtgccatgtttcccttgctttttcatgtttctgtgtccttacattgatatcagcacagctaatgtaacagtcacttctccaattttatttattgactttcatagggaaagacattttcctctgctgggcatgatggctcatgcctataatcccagtgactcaggaggctgaggtgggaagattgcttctggcaggagtttgaggttacagtgagatatgattgtgccactatgctccagcctaggtgacagagcaagaccctgtctctaaaaaattaaataaaaataaaataaaaaagatgttttcctgtagatgtgtctatagtgttggttgagtggagcgctttggctttgattctgtgtgggcacagttagtggagcctccacatgatttcattagcatcagtgttgtctgtgagctcctcagtggctgacagtgtagttgttaatggagtctgtggaaaggctgtgctgggaacagggacatcaggcaggttggtcttcaagcaccagtggtgacagcagtatgccaggcatgctggtcctcaggcccccaggcagtgtatgtgggtgtcaatgctggtgtgtccaggtgggcagggtatcagacttcaagatgacatgttcgggtgccagtggtagcagcagtggactgaacaggcaggtccttgggcccccaggtggcttgtttggaagcggcagcagcaggccaaccctcaggccctcaagtggtgtacacaggcatcagtgatggcaagctgggtaggctggtccccaggcccttgggagacacatgtggtcactagtggcaggcagagtgggcccatccttaggcctctggatggtgtgtgcctgtgctgctggtggtaaagaggacgggttaatctcctggcccctgggcagcacatgtggatgctggtggcaggtggggcgagcctgtcttcaggttccctaatggtgtgtgcaggtgctggccgcaggtgggacagttttacccccattcccccagatgcgcacaggcaccagcaacaatggcaggtgaggcgatcctatcctcaggccctgggacagtaagcacaggtgctggtggcaggtggggcagttcaatccgtttctattgccagcggccatttgagtcacctccaggttggggctagtatgaaaagtgttactatgagcatcccaatcaggtctttgtggggcaaatggatgcctgccactcaatgttaagaccatttttggcttttacttgagttgagaaataccactgcctgctagagatccctgtaaccccagaatctagtgtctcatatctgcttctttgtcactggcagggtggctcctatggacggaaaacagtcttaagaggcaactccgatggtacccttgtcctcttcttcagtgacttaaaacaattccaggatcagaagagaagccaacgtgacatcctcgataaaactggggataagctgaagttctgtctgttcacgaagtggttgaaaaacaatttcgagatccagaagtcccttgatgggttcaccatccaggtgttcacaaaaaatcagagaatctctttcgaggtgctggccgccttcaacgctctgagtaagcattgctgggtgtcaggagagaaaagccaaagaagcgggtgccagacagctctgtgcaacctctaggccatgagtgggatagataccactgctgctttaaaaaatgggagaccatagaccctcaggagagaagaatcccttctaccctggactcgctctcttctctggaactaacttctcccccataccctgattgtctttggagaaaatgttctggattctagaatctaaggcagagccttttaagccatactgtacacataaatcacctggaaccttgttaaaatgcagatcctgactcaggaggtctgagttagagcccaggatttcatatttctagccagctccatgatgagctgctggtccgcagatcatgcttgcaggttttgaccagagtcagtgttggttagagtaagaggatgaggcagacatctgggaaaagtccagctggggcaagcatttgaagtctgccttcctaccaggtcaaaatcaaggcaacgaccttccatagataactatcaaagcttgagggggtgccttgaacccaactcctaaatccctaagacctgcccacctcttgtgtctcctgtctcagcaaacattcccacactcttgcatattgttaaagtaacctctgcttaccaggcttctggtttaataaaagatggctagagtgactccatcttaaagcaagtagctaggcactcaaaaggaacctacaggcttaatacttgggtctgaaaatagccacagtctaagctgaccaccaattataattgcagaatatttaaggccatacaaaacatctcccactaagcctacaaaatgtccaggtgtcctaaaagttcagcccacttaaaggcagcattaatgagcaggtttaggttgaaggattaatggtcatcaataccactgttaagaagaaaattcttggccaaattgaatttaatggagtttaactgagcagacaattcacaaatctagaagcctcctgagccagagtaggttcagagagtcttgaacacagccacgtggtggaagaagatttatggacaggaaaaggaaaatgatgtactgaaaatgaaagtgaggtacagaaacagccagactggttatagctcagcattggccttatttgaacgagatttgaacagttggccacctttgattggccgaaactcagtgattggcacaagagtaggttgcagtctgtttacacatccttttaggttatagttcaccatgtacagagaaattttaggccaaacttaaaatatgtaaggaggcagctttaggctaaacttgatttaacagcaccaataccccctacctttagtgagcacatctgcacattccaattttaatgacagctccttagaatttcttatcaacgaagacactaacaaagaatggcgcattcctccttctcctttctgaggatgccctaccctgtaacaaagtcgtttctaataaatttgcttctttcaccatactctgtacctgccttgaattctttcctgcatgagatccaagaaccctctcttgggatctgaatcaggaccctcttttccagcaacgctatcacctgtaaaccaaaagtatctgagacaggtctcaatctatttagaaagtatatttgccaaggtgaaggatgtacccatgacacagcctcaggaagttctgatgacatgtacccaaagtgctcagggtacagcttggttgtatacgtggtatggtttggctgtgtccccaccaaatctcatcttgaattgtagctcccacaatttccacatgtcatgggagggacccagtgggaggtaattgaatcatggaggcaggtatttcccatgctgttctcatgataatgaataaatctcacgagatctgatggtgtcataaaggggagttcccctgcacaagctctctctggcctcccaccatgtaacatgtcccttgctcttccaccatgattgtgaggcctccccaaccatgtggaactgtgagtcaatcaaacctctttcctttataaactactcagtctcaggtatgtctttattaacagcatgagaacagactaatacaatacattttagggagacatgagacatcaatcaatgcaggtaagatgtacattggtttggtttggaaaggcaggacaactcaaagtgggggcttccaggtcattggtagatttaaagattttctgattggcaattgatggaaagagttatcatcaatagaagggaatgtctgggttatgatgataaagggttgtggagaccaaagttttatcatgcagctgaaaactccaggtagcagacttcagagagaatagattgtaaatgtttcttatcagacaagagcctgttctatcactaattccaaaagagagggaggtatcatgaggcatgtccagctcctccttcccatcatggcctgaactggtttttcaggtcaactttggaatgcctttgctgagaggagaggtccattcagatgactgggggtgcttagaattttgtttttggtttatatacctaagccaggattgtatctccagctcccccacctcctcactcacagcccatcactaagtcctgccagttctgcctaacttgctcctaaatctgcatcatttctctcccttcccactgctacttccctagtcaacgtcaccaccagggatggggcaatggcttcctaattgtctccctgcctcctgtctcactccaccctgcagctggtgctctctttccatgcacaaatattgtgtcacacactctgcctctaatggcttttcactgcatttaggaagttgaccaaaaaggccaaactctgtaaaatattttaagaggttttttctgagccaaatatgaggaccatgacctgtgacacaatctcagtagttcctgagaacgtgtgccccaggtgattaggttacagcttgatttgtatacattttagggggacagaatttacaggcagaaacgtaaatcagtacacataagatgtgcattgattcagcctggaaaggcaaaatgtctcaaagcagggtcttccaggtcatagatggttataaagatttcctgattagcagttagttgaaggagttcagcttaagaaaaggggagttgtggaagacaaggttcttattatgtagatgaagcctccaggtagcaggcttcaaggagaataggtggtaaatgtcccttatcagaccttaaagatgtcagacgattagttaaaatctctcctgaatcaggaaaagatctggaaggggaaggggattctccacagaatgtgaatttttccacaggacacagctttgcagggccattccaatatatgtcaaagaaatatattttggggtaaaatacttttgatttcctttagggcctgttatctgtcaggtgatgccagagttaggctggaatttggtgtcatactgctacaaagagtctgttctgtcagtcttggggtctctattttaatgttagtgtttgtcgattgtgcctgaattccaaagggaggagggtataatgaggtgtctgagctcccccttcccatcatgacctgagctagtttttcaggtttctttgggggttccccttggccaagagaggggttcattcagtaggttgggaaggcttagaattttatttttggtttacaaggataaggatcaaactcctcagcaggacaggactttgatgatctggcagcccctacttacttacctctccagctgtaccactcaccaccaactaactctctatgcttcagccactctggccttccctgagctcctcaaacccacatgatgctagctcaacttgagactttggcctatactgttcctttgcctggaatatacttcccccagccttttatttttggataactccaactcatcctttaagtcctctggctctctatgtctgggttagatccactttccacatgctccagtagcaatttgtgctgtctgaatcatgggatttatcactttgcaaaccccaactatctgagacaggtctcagttagtttagaaagtttattttgccaaggttgaggatgcgcacccgtaacacagcctcaggaagtcctgatgacatgtgcccaaggcgctcagagcacagtttgcttttgtacgttttagggagacctgagacatcaatcaacatatgtaaggtgaacaatggttcactccggaaaggcgggacaactcgaagcaaagtgggacaattcaaagtggggagagggcttccagatcataggtagataagagaccaatggttgcattcctctgagtttctgattaacctcttccaaaggaggcaatcaaatacacatttatctcagcagagggatgactttgaatagaatgggaggcaggtttgtcctgagaagttcccagcttgacttttccctttaaccgagtgattttggggccccaagatttattttcctttcacaacttatatggaaataatgtatttagctttctcctctcctagactataagctccatgaggacaggtatcatgagtatcttgttcttctatggtctctagtacaatgttcattattaataatagttgttggttggatggatggatggagcatgatggataaatggatggaaggtaaatggatggaaaaatgaatgtagatggatggatggatagatagatattaaatatatgtatatatttatggatgaggtagatagatggatagtctcaagcacaaagtctcaagtacaggtgcacatataatcacaaacttaaataattccaggctgggcacagtggttcttgcctataatcccagcgctttgggaggctgaggcaagaggattacttgaggccaggagtttaagaccagcctgggcaacatagtgaaaccccatctctacaaaagaattaaaaattagatgtggtggcatgcagctgtattcctaactactggggagactgaggcaggaggatcacttgagcacaggagttcaaggttgcagtgagctattattacaccactatactccagcctggacaacggagcaagatcttgtcaaaagaaaaaaaaaaaagaaagaaagaaaagaaaaggggaggggaggggaagagaggggaggggaagggaaaattcccaagctgaaacttaagccatcattaaacaaatggggaaacagaaaagcccaggtcttctaagcccatggccagagtaccttccatcacctatagttttaaggaatttctgggttaccaggaaccatgaagaacaattagcccaggcatcactgataaggaggagacatacagacatcctgataactcacattcacagaaaggtggcctggcttggggaaacatgtaccagtttgggaggttggcagttctgagttcttatcctggtcctactactcacttatgcctgccagctgtacagtcagactagaaactagattccagggccattaggatgctcagggttgggggtgggggcctgcttcaaacaagggctggagtcaaggagggagacaggtgcatcataattctcataggaaagtcctctcttcctgttccaagtgccatgctgggagacctggccagagcgccctctactgggttcaccccatcatgcaaggatggtgtgagaatgagaaataaactcttccccacatgtgctacccagaagctgccactagatggtggtaaagctgcacattccaaattattataagctcctttttctgcaaggaaacacagggtcactttctgagaaaagcaacctacaggggaattggtaacttgcttttcgttttgttttgttttttaaactaaatattagcctaactaatctggcctatttttacaccaggtccaggctggctgtctccaattctgcatctctttccaccactgtgtgccactacaactctccccctagggcctttgacccaaagcaagaagtacctatttacttgactggctggtatttccattatcctcatctgtaaattagtaattgcaattgcacttgccagctaagagttttgtgaggcttgtatgtttcactgtacataaaaggcttagagtaaacacaccataaacgttagctatttattatcattatgggggaagatagaatgctctttccgtcaacctccctgcatgccaagtgcccagaaccatgcctagggagtcagtaaatatccactggggtgtatgaaaggaacaagctagctgctttctcctggtttcttatctaaatgtgtttatgggccagggagctggctcacgtctgtaattccaacactttggaaggccaaggtgggaggatcacttgaggccaggagttcaagaccagcctgggcaacaggcttttttttttctccaaaaaaaaagccaaacatggtggcacacgcctgtggtcccaggtactcagaaggctgaggcaggaggatcacttgagccttgaaatatatttgagggtacagtgagctatgattgtgccattgcagtccagcctgagcgacagagaagactctgtctctaaaagtaagaataatgatgaaacaaactcagtagtcctatagaccattttttgtttgtttttgtttctatttgtttggttgtttttttgtgggggggggagggttgttttgttttttaatgaacatagaaattgacccttctgatcttaaagcttgaaacttacatttgttttatctaagttccttcctcagaaaaggaacctcagacctctcaaaaagtatcaaagaactgaaactcaccagtcactgcatccagacaatgggatgtcaaatcccttattcctcatgattacctccttacccctccctaattcccgttttcccacacatagttacatttcttccctgctatatagaaccctaactttagtctgtcagggagaaggatttgagactgatctcccatctcctcagctgcagcacctgattaaagccttcttcttggcaacacttgtttccgccattggctttctatgtggtgagcagcaggacctagactgaacccctggtgtttcagtaacagtaatcaatcaataaatgtgtttattttatttttatttttattttgagacaaagtctcactctgtcacccaggctggagtgcagtggtgcaattatggcccagtgcaacatccacctctcggggtcaaggagttcgcatgccttagcctcctgagtagctgggattacaggcacctgccaccacgcccagctaatttttgtatttttagtacagacggggtttcatcatgttggccaggctggtctcaaactcctggcctcaggtgatccgcctgcctcagactcccaaagtgctgggattacaggcatgagccaccgtgcctggctaaataaatgtgtttataaacagtggaactcagctagcaagaatggaaacccattgattaaaccagcccctgtccccagacctccagggccctgctgccaccttgcctgcctcctcaaagtccctgtaccctgacattctgaagtaaaatattcacaggatggactcaatgacagaatatattgcatgtaaaatataaactttcctttcttccagatgtatgctaactatgccaacacaaacaattttttaaagaacccacttgctccctactaatttggataattccctttctcgtttataattccatctcttgtccctccattaggttctgctgatcttgtcttgggttaacaccatactgttttcattacagtcagtttagactacactttattatctaatgggccaagtcacccttccctactatgttttatgtaaaggtacctgtcctctgaccgattaagcaagagtcgtgtgctatcaaactataactgaccttcattgtagagcaaccctggctctctaaaaccaggttacataaataacaacgttccaatttctgtttcacatcaggcttaaatgataatcccagcccctggatctatcgagagctcaaaagatccttggataagacaaatgccagtcctggtgagtttgcagtctgcttcactgaactccagcagaagttttttgacaaccgtcctggaaaactaaaggatttgatcctcttgataaagcactggcatcaacaggtaattttccaactgtctatatatggttatcattcatttcctttttatcgagataattgacatccagtaaagtgcaacaatcttaggtatacagctcaacgcattttttacaatcttaccagcaacacccaaatcaacatagggaacgtttcgtgtaccccagaaggtgcctcaagcccattgtccccctagtaatcctcccaaacaaacccctattctaacatctattaccaccgcagattagttttgcttgattttaaacaaacaatgtaagtgggattatatggtacatacttcatgtctgccttctttggctcaacgggatgtctgagagactcattcatatggttgcagggtcttgctctgctgcccaggctggagtgcagtggtgggatcttggctcactgcaacctgtctcctgggctcaagcaatcctcccatctcagcctcccaagtagctggaactacaggagtgtgccaccatacctggctaatttttgtattttttgtagagatgaggtttcaccatgttgcccaggctggtcttgaactcctgggctcaagctatgtgcccacctcagcctcccaaagtgttgggattacagacatgagccatggcacccagccatagtagcactttttttcattgttttcccatattcaattgtattaatgtaacacacatttatccattctgctgctttgggtcattttcaaattggggcaattttcaaatagatgattgggtcattttcaaattggggcaattatgaattaggttactatgggtatttccctacatgtctttttgtgaatgtaagcattcatttctcttggataaatatgtagcagtagaattgctgggctatccagtgcacatatatgaatgttgtgtacatatatgcgtgtaaatatataaaatatatatgtagtttgtttttttgtttaaccatagcaaatcttgccgaatcatttccaaaatcttaccaaattatactcccatcagcaagatgtgagtttcaggcatattctcaaacccaactggggatctgcccacccagcgcagcaaagccaaacagactttgggattgcagtgagagagtgaagcatttattgcagggagccaagcagggagaattgggcagctcatgtttaagagccgaactccccgatggcttacaggtaagggttttaaaggtgaggggacagaggttacaggcaaagccataaatcaacacatggaggctgtacattggtttgacctaaaaaggtgggacgtctccaagcaggagggaggaggaggatgggtggcgctggtcacaggattcaaagactttccaatttgtgatgggtgaaatgggcgaagctttatctaaaaatttcagatcagcagaagggaacgttagctctggctcgtaggtgtgacctcctccaaaaccctcaggaagaaatttagaacaaagaacgaggtcagggttcaatcctcagtttcccctcatctgaggtctacctgccagtggatttgcttggtgggggcctgagtttctgaaaatcaactcagggacatatgctaagatgttatctttagtttctatagggaacaaaacatcttttgactctaacttccttggctactgttttaagtgactattaccttcttttttgttgtttttgttttattgggttttttttcactgctcctgtagaccctgaactactgtatttttgcttatcgagttgctcattgacttttcaaggctagtaaggtgcctggaatttccctcgaaagaactccagattttcctttattttcatgcttgtgggatgggagcagtgggtggcctgcagacacctaagagaggtccctgctccatcccagttgttccacatctttgtcaagacttggtattgtcagtccactgaactgtaggcattctagcaaaggacaatgacttcctgtagacctctggtttcttgatttcagatccctgacatggtaagaactagatcccccaatgagctgctaccctttccttatcccacagtgccagaaaaaaatcaaggatttaccctcgctgtctccgtatgccctggagctgcttacggtgtatgcctgggaacaggggtgcagaaaagacaactttgacattgctgaaggcgtcagaaccgtactggagctgatcaaatgccaggagaagctgtgtatctattggatggtcaactacaactttgaagatgagaccatcaggaacatcctgctgcaccagctccaatcagcgaggtgccaagcttctcacaccctatccctgtacctcatcatccgcatgccaggcatacctctttggaatgagatctcagccttgccctatcaagccagtgctggaccagaagaatggcagccaccatgggtggggaggcagatgggtctgtctgaaagaggggaggtccagccagtgcttttcacattccaaaccatagtcacattttgtcccctaaacaaacactggagagacacttccccttatagaaggaaggagctaagagagaaaggggatggggggcaggaagaggagaccactttgttattttattaatagaatgtctctgtaaagcagcaaacccagggccagaggggcttgatgtcgtagacaaaatccctcagcagcttggaggagctgcaggaatccaaattcaagttgccggattcccactccaggcctctttccccaaaacagattttgcctcccacaagcagctcaactgactttttttaatctaatggaatacacaggccagtaatcttggatccagttgacccaaccaataatgtgagtggagataaaatatgctggcaatggctgaaaaaagaagctcaaacctggttgacttctcccaacctggataatgagttacctgcaccatcttggaatgttctggtaagagggttttccaaatacagggtgtttcatgctgcaggaaggtcttcctgacacccaggctaggtcttatctgagagtactctatattcgtttcctgtattgctgttacaaagttccacaacacagatggcttaaagccacagaaatttattctcttaccattctggctattacaagttcaaaatcagggtgttggcagagccaagctctctgcaggccctagggaagaattattccaggcttcctgcagcttctggtggttgccagcagtccttggcttgtggctgcatcactccagcctctgtccctgaggtcacatgcccttctccctacatgtcttcacatcaccttccctctgtgcatgtctgtcttatcatctcctcttcttctaagaacaacagtcatactgaattagggcccaacataatgacctaatcttcactagatcccatttgaaaagacactatttccaagtaacacacattcatcgatactgtaggttggaaccccagcatcttttagggagacacagttcaacccatcacacaccatcagggcaatgactgtgaagtgcaaaatggaacaaagagaaaggggggtacccaaagatgcgagatctcaattaatttttgcaagagagaaagctggtgagcaggtggagacatgaaacggagcattgctcagagaagggggtggcagtcaggcctgtggatgccaggacaggctctgagctccgttcaggcaggtgccgggggcaacagtcagaagtgcggccgaaacaagagaaccaattaaggcaccagctgtgaaacaggtgcactggccttccccctcccatccccaaaaggagaagtaaatctgtcaacacaaactcccctgcatgctcagtgcttattagagaaaagccatccaagaagaaccttcccaggttagaccactgagccctctcagctttcctgggaaccgcactggagacaactgtattaattgaggccaaagaggccaatcaaattccacagttcacagacagattaatttatattcaagttatgggaggcggaatctataccccgtttgcacagaagtttgaaggctaactgagtaccaatctggcatgtaaatgaagatgtaaatgagtttagcaaacttcacagaggttggggagtctatgaatggggaacttccctaactggacccagaaggatcaggacgctctgagcagtcacccctgtgcccaccctaacccctactttcactggacccctggtctttgctctctgaccccaattttcccggcttcttcagacagattttttcaaaaaattatccccccgacctgacatcttattctgaagctttgaaacatttaaaaagttagaaaaatgattcagtaaactcctatgatcccctcacatagatgcagtacccatgtgcaacatttgctatgttacagtttgtgtgtgcacacgttattattgttgttgttacagttgtttttaatctttttttaagagatggggctataactcgctacattggccaggctggtctcaaactcctgggctcaagtgatcctcccacctggcctcccaaagctctgggattacaagcgtgagccacctcacctggcccattgttatagttgttgtttatactgagccctttgagagtagactgccagacatctctttacccttaaatgctttcatagggatctctgagaacaagaatattctcttcaataacctccatgccatgatcaaattcaggagactaggagtcatatattattgtcccaatacagcccattgggatgatttcgacagtcattccaagaagggcctttataggacattcttttccaacccagaagcacatgtggcatttagttccaaacatctcctaaaccatcttttgatactcactgtggctgtttttgttctgctatcccatcaacttctattttgagaactgtgagaccatccctgtctctgcagttgttgaagaaaatttaggcccaggaaccactgcataaggcggtccaaacattgggggctcatgcacacatacacacacacacacacacgtacatgcatacacaaacatgcacacacacactcacacatgcacacacatgtactcacaccatgcacacacgcacacacgcacacacatacatgcatacacacatgcatacatgcactcacacacatgcacacacgtactcacaccatgcacacacatgcactcacacacatgcacacatgcactcatgcacacacatgcatgcacaccatgcacacacatgcactcacacacatgcacacacatgcactcataccacgcacacatatgcactcacacaccacaaatattctgccctgtggaaggggcagtgttggcaatgaatattccttcaataggcaccgtcctcagcaactataaggccaacttacaacatctgagaacaaccaaggttggccttcaactttcaagctgagtgcccttccccacctccaggtgagctagcccatcttgtctgtgttgccagcaaaagtacaatatttgtatagaacagtaattacttgcctcttactgctgttttaaaaaaatgccctaaacttagtggcttaaaacacatgaatttgagccaggcacagtggctcacacctgtaatcccaacactttgggaggctgaggcgggaggatcacttgagaacaggagtttgagaccagcctgagcaacataacaataccccgtctctacaaaaataaataaataaattagctgtagtcccagctagttgggaggctgaggcaggacgatcacttgagcaagggagatcaaggttgcagtgagccatgattgtgccactgcattcagcctgggtgacagaacaagaccctgtctcaaaattacttttgcaccaaccaaaaaatatatatatacatatatatacacacacacatatacacatatatatacacatatatacatatatacacatatatgcacatatatatacacatatatacatatatacacatatatacatatatacacatatatacacatatatatacacatctatacacatatatatatacacatatatatatatacatatatatgcttcctcaccttttcaagctccttgaggttgtccaaatttcttggctcatggccccttcctccatcttcaaagccaatagcatagcatcttccagcctctctctctacttccaacatcacaccacctaactctaactccaacaccaccacactccaactctaaccctcctgcctccatcttataagtacccttgtccttataagcacagtggctcacgcctgtaattacagcactttgagaggctgaggtgggaggattgcttaagaccaagagtttgagaccagcctggggaacctagtgagactcccatctctacaaaaaaaaaaaaaaaaaaaaaaaaagctaggcgtggtggcacaaacctgtagtcccagctactcaggagactgaggctggaggatcccttgagcccaggaattggagactgcagtgagctataatggcaccactgccccccagcttgggtgacagagcgagacactatttctgaaaaaacatgggttagaatcatgggttggaaggctacctgttgggttctatgctcactacctaggtgatgggatcattcatacaccaagcctcagcgacatgcaatttacccatgtaaaaccccacacatgtacctccttgaacctaaagtaaaagtggagaaaaatgtattaattaattcaattaaaaattaaaattaagaggaaaaagacccctgtgataacatcaggccaaaccaggtaatgcagaataatcctcccatctctagagacttaaccacgtgtgcttcagagggagcacagctgtgccaaaacctggactttgaactgttggcctccagaactgggagagaataaacttctgttgttttaagccacctagttgtggtcacttgttatggcagcctttggaaaccaacacacccgcacatggcgtgtttaacgcaggctgatacaaccttaagaaaggaatggatgtggtcatcagcaatctccaatacctacagcaaatgggaagacagggaaggaccagaggtgtaggtaaagcaaaaagccacaggtcattaggaagtgatgctccaactgggcatggaaaaggagtttggagttaggaacacgacagatctgtctggacaaggatccagatctctcctagggggaaggaggggcaactaggacagtttttgtgtctgtggggggtcttgtgtgccactccaaactctcaggtgtgttcttgggatgtatttgtgcaatgacaaaagacggaaaagatgccagcccacgagcaggagggcagttgggagaggcagggtagggggcccagtacaggcaggaaaagaagccttgggtgggcttacaggtgccactcacacttggggtcttccttccttccccagcctgcaccactcttcacgaccccaggccaccttctggataagttcatcaaggagtttctccagcccaacaaatgcttcctagagcagattgacagtgctgttaacatcatccgtacattccttaaagaaaactgcttccgacaatcaacagccaagatccagattgtccgggtgagcactggcctttctcatgtcttgttggaatgatgtaatattgggcattcctggaagggaggtaagtgggcattgtagctctccaggatccatctaccttcagaaacttgaggaagggaagccaggagttaagggaactaggacactagttttcaggggggttgtggatagactgcctaggtcattaacaacacaactttgattctgtattgtttttcttggagctgttggccgtccttggggctcttgggcctggagacacatcactcccgtctctgcctctgtcatcaccttttcttctgtgtcctcccccttctcttcttttctaagaacacttgtcataggatttagggcccagcctgatccagaatgatctcatctccaggtccttaactcagttacatctgcgaagaccctttttccaaataagggcacatccacaggcccccagagcatgtatttttaggggtccaccattgaacccactgcagattctaaggagcttagaatctccttctcctttgagtgactggagcccggcttgccccatcagctgagctagagatttagaggcttttgtcattcttgtccgtggctcaaaggccacggggctgacatgtttctctctgtttactggtcacacgaccagataaacgtgtccccgtggtcacaagaagggaaggaggaaaatgttattatgtacgcgtaaatttctgcgttgatgccagaacaaatctgtgcaactagagaagaggggcctgggaaatggcaccaatagccccagggcaatgagtgaactgtctcattgaaggtcactggagtgaaaacaggactgagtttctgatcagtgttagaattgtcaaagcatgtgctttctcctctccttcctgaaaccccattcacatgatagtcagtgaaaaataaaaggtatatgacacatcaaagaagaaaagagagccatcggcagataagacattgctttccatcttcctgaagttggttgaaggtttctctgcttaggccagaggaaggtgacacctagagattgtaaggaaggggctacagtctgagaggagccgccgaagcccacctcttaaagacaaaggtggcctgcatgaatgaagggggctggaacagggatgaattgaggatctgcatacaggaaatagaacataccctttcctcacctccttaacccatgtaatcaggcatttaatacccatggataaaataaaacatttttctctacagaaactaaatggaggagaaagaggaggaagaggaggagggagggaagggagaagccttaatggcagagacataaatagcccacaaagcctaaaatacctactatctggcccttttgcagaaagtttaccaaccctgtacctcaattatggatattcacccaatgatcgccagacctgcaggagaaaacacacatcaaagaacagatgagcaccttcaccataattattgcattccgctcgatttatttggttacaggaatccaaataaagctaaatatgtctttaggaaaatctttaggaaattgacctgagaagcaggtaacttgtaactagcccatcagttgttcatgtataaaaggcaggagaataattgccttgggaactgtattgtttaataactgcattatctattagtttgtaaaagaaaaccctttgctttggactcagccctcactgaaccctaacgcagaacttggccttggaaacagtgtcagttagcccaactggtgccagcccacgggggcacgggactcctcggttaaggaaattctggatctcaaccttcctttcttccttagggaggatcaaccgccaaaggcacagctctgaagactggctctgatgccgatctcgtcgtgttccataactcacttaaaagctacacctcccaaaaaaacgagcggcacaaaatcgtcaaggaaatccatgaacagctgaaagccttttggagggagaaggaggaggagcttgaagtcagctttgagcctcccaagtggaaggctcccagggtgctgagcttctctctgaaatccaaagtcctcaacgaaagtgtcagctttgatgtgcttcctgcctttaatgcactgggtaaggctccccagaccttagcttggaagtgatggtggacagaaggtggagggagagccacgtgacttgattacgggctctgaaaacatgtgccactcatgggtccctggccccagcaagtggcattagtcatggcagtaatacttcacacttgtagacaattttggtcccatttctcttttccaagcatggggctgtgtgcctataatcccagtaatcagggagactgaggcaggagaattgcttgaacccgggaggtggaggttgcagtgcgagatcgtgccactgcactccagcctgggtaacagagtgagactccgtctgaaaacaaacaaacaaacacacaaacaaaacaagcagttgggctgggcatagaggctcacacctgtaatcccagtatcttgggaggccgaggcgggaagattgcttgatccaggagttcgagaccagcctgggcaacatagtgagacttcatcccaaaaataaataaataaataaataaataaataataaacaaagttagccaggcatggtggcacacgcctgtagtcccagatacccaggaggctgaggtgggaggattgcttgagcccaggagatggaggctgcagtgagctgtgatcatgccactgcactccagtctggggcaacagagcaaaactctgtctcaaaaaagcaaaaaacaacaacaacaacaaaaaaaaaaaaaaaaaaaaaaaaaaaaaaaaacaagaagcagcgcaccagatttagcccagggggcggtagttcacaaagtccagatcagtgtcgtcccagagatctttctgtgatcatgaaaatgttctatgtctgggttctacaactactgagagcttgacatgtggctacagcaactgaggaactaaatttttaattttcattccctttaatgaatgtgagtgcaaatggccacattgctggtggctgctctgttagtttaagtctagataatactttaataataccagctgcacctgtgtagaccacataatttttacatatctcattattctggagatgctccctgtgtcttagacatcagtcttttaaatatgctaatactattcacagtaatttccaagaattaaatcatatttgtcaaaaatgttttggaatctgttacttactatgtgtattaaagcaggatgtcaatctctccctcatgccaggtcagctgagttctggctccacacccagccccgaggtttatgcagggctcattgatctgtataaatcctcggacctcccgggaggagagttttctacctgtttcacagtcctgcagcgaaacttcattcgctcccggcccaccaaactaaaggatttaattcgcctggtgaagcactggtacaaagaggtaaggacagtctttgttctgaccatggggttattatttttaccagtaagccatgaacattaagccctgttgcccacaatctcccatgctgagggctgagccactttggagaatctgcccactggatatctttacgacgttcccatcactaaccctattcatttgggtcaggcgttgatggcttctgcccatcaaagctgctacatgtggtttactttgccctcacactggccaaagaattctgcaggaatccaagtgaccttgcctacagagggtgggtggaagctgggattcacatgcccacacattgccaataggacttttctgcttcccggcgcacatgctctctctcagaaggcaggttgaagctgtaggtggaaccggggctgcattttctatacacccagctcacaggagcttgaacaactggtcagcccctgagcccctactacaagtgatcctcaggcaggtaaccccagattcatgcactgtagggtgctgagcagcatccctagtctctacccagtttcccctcacttgcacacaaagtagcttctgaacatcctcatgacagtaaacatcaagcacgatatcagctcttcctgagatgcctcctttctgtgtccagctccctgaaatgtgaaaccaagaccaaactctgtagttttcatcccagaaaatcagagagctggcatggcgtgtcagaagacttggggctgtggcgtgaccttgagtgagtcactccacttcccgcagcttcaaagtcttcatctaaaatgcaggtcatgctgcctgcattgtggggaagattaggaatgatgtgggtaaatgcctgggcccatagctggcatgcagtgggtttctagtaaactgtagctatgttattgtcctaggaaggcatatatggtcaaaagcacaacaaagaaactcaaatgtgctgctgccaggtggactcctgtaggctgtacaatttagacatccaagctgcagagtgtgagcacaccagcacgacactgtgactcagttgccttgctgtggtccctgtgcggctctcactgagctcctaggctaaccagttcgtctgatgttcccactcttacctagtgtgaaaggaaactgaagccaaaggggtctttgcccccaaagtatgccttggagctgctcaccatctatgcctgggagcaggggagtggagtgccggattttgacactgcagaaggtttccggacagtcctggagctggtcacacaatatcagcagctctgcatcttctggaaggtcaattacaactttgaagatgagaccgtgaggaagtttctactgagccagttgcagaaaaccaggtgccttcaccctagccccgtacttttcttaacctgattcccttgaacactgtctcagcaacctggattttcctctgctggggtcacgattcattccttgcatgacgggggaaagtgctagccaacagaacctgtcacagtctccagggataaaacctagcaggggcaggagaaaatgctgttcagaaaaaatagagaaggaaggttcaagataccacgtccctgagctgctttcatctaagaggtgttagagttggaagtgtgtccctaagaattaacgctatcattcacgggccgatttatcctgcagggtttttattgggttagggtttatctactctttcagaccttcaagaatagtaagaaatcatatccatctgcttagggccaccagcaagggacaaaaacaatggaaaccagaggaaaggaaagagagaaaataaaggagcgttgaaaaagaatagaggagaagtaagagagaaggagacccccggtgggttgcctaccctgggttcttggcaacattgtatttgattaaattatgtatcatattttagctgacatagcaaggaggagatatttagactatgccttcattatgccttcatgagtatagcaattaacagctttcaaaacgcttttggcaaatacaatttaattctaggaagttgctttggttttggcttttgtttttgtttttgagacagagcctcactctgtcacccaggctggagtgcagtggcacaatctcagctcactgcaacctctgcctcccgggttcaagctggttctcctgcctcagcctccgaagtagctgggactacaggtgcgcaccaccacgcccagctaatttttgtatttttagtagagatggggtttctgcatgttggccaggatgttctcgaactcctggccctcaagtgatcctcccacttcagcctcccaaagtgctgggattacaggcatgagccactgcacgcggcctctagaaagttactttgaaccctactagtaaatttgagttgctgacatctaagctgtaagaacacattgtcttattgctgaaagtattataggatggaccccaaattctgaagtcactgggatttggaatctcctaatgccttctaacctctcattcaggcctgtgatcttggacccagccgaacccacaggtgacgtgggtggaggggaccgttggtgttggcatcttctggcaaaagaagcaaaggaatggttatcctctccctgcttcaaggatgggactggaaacccaataccaccttggaaagtgccggtaaaagtcatctaaaggaggcgttgtctggaaatagccctgtaacaggcttgaatcaaagaacttctcctactgtagcaacctgaaattaactcagacacaaataaaggaaacccagctcacaggagcttaaacagctggtcagccccctaagcccccactacaagtgatcctcaggcaggtaaccccagattcatgcactgtagggtgctgcgcagcatccctagtctctacccagtagatgccactagccctcctctcccagtgacaaccaaaagtcttcagacattgtcaaacgttcccctgggttcacagatctttctgcctttggcttttggctccaccctctttagctgttaatttgagtacttatggccctgaaagcggccacggtgcctccagatggcaggtttgcaatccaagcaggaagaaggaaaagatacccaaaggtcaagaacacagtgattttattagaagtttcatccgcaaattttcttccatttcattgctcagaaatgtcatgtggctacctgtaacttgaaggtggctacaaagatgactgtggacgtgggttgcactggccacccaaggatgtctgccacacctctccaaagccctccctacctaccaagatatacctgatatattccaccaggatatcctccctccagatatacttggttctctccaccaggttctttctttaaagcaggatttctcaactttgatacttactcacatttggggctagacagttctttgtttggaggctctcttgtgcattgtaggatgttgagcagcatctctggcctgtacccagtagatgccacccagttgtgacaattaaaagtgtcttgagactttatcatgtgtcttctgccctaggtgagaacccttgcactagaggaaccctacaccccaaccctggggggaatgtagggaagaggtggccaagccaaccgtggggttagctctaattattaagatatgcattataaataaataccaaaaaattgtctctggcaatagttaccttcccagatacaggtccccccttttttcccctaactcttttaagcaatgattgtaactattaggagacattgctctcccacgtatgtttttctttttagacaatgcagacaccaggaagttgtggagctaggatccatcctattgtcaatgagatgttctcatccagaagccatagaatcctgaataataattctaaaagaaacttctagagatcatctggcaatcgcttttaaagactcggctcaccgtgagaaagagtcactcacatccattcttcccttgatggtccctattcctccttcccttgcttcttggacttcttgaaatcaatcaagactgcaaaccctttcataaagtcttgccttgctgaactccctctctgcaggcagcctgcctttaaaaatagttgctgtcatccactttatgtgcatcttatttctgtcaacttgtattttttttcttgtatttttccaattagctcctcctttttccttccagtctaaaaaaggaatcctctgtgtcttcaaagcaaagctctttactttccccttggttctcataactctgtgatcttgctctcggtgcttccaactcatccacgtcctgtctgtttcctctgtatacaaaaccctttctgcccctgctgacacagacatcctctatgccagcagccagccaaccctttcattagaacttcaagctctccaaaggctcagattataactgttgtcatatttatatgaggctgttgtcttttccttctgagcctgcctttctcccccccacccaggagtatcctcttgccaaatcaaaagactttttccttgggctttagccttaaagatacttgaaggtctaggtgctttaacctcacataccctcacttaaacttttatcactgttgcatataccagttgtgatacaataaagaatgtatctggattttgtgcctagttcctagcacacagcttcaaaaattctagagtttcctgataggagtgtcttttgtattcataacaagcccttttcacccatgcctgggtttatgctaacaaggttacccatggtgggcccttagtttcaaggaaggagttggccaagccagaaagaccaagcatgtggttaaagcattggaattttcagccccatcccacccccaatctccaaggaggtgatggggctggaaattgagttcaattttaacatggccagtgatttaagcaatgctgcctatgtaaagaaaccccaataaaaactctggacagtgaggcttggggagcttcctgattggcagacattccaatgtactaggaaggtagcgcatcttgattccacagggacaaaggctcctgagctctgggcccttccagtgcttgccaccctacatactctttgtctggctcttcatttgtattctttataataaaatggtgattgtaagtagagcattttcctgagttctatgagtcattctgcaaattatcaaaactgaaggaaagtcatggaaactcccaagtttgtaaccaagtcggacagaggtgtgggtagcctggggaccccatgtatggctggagtctgaaagaagggtggacttggtgaggaccttgctctttaaattgtgggatctacacacattccaggtagtcagtgccagacttgacttaaattgcaggacagtcagctggtgtcacagattaggtgttggaatgcaccactactcttgtccaggatgcaggcaaaagaaatatatatgaagatgttgaaaaatggaagaatacaggaaacaagcatgagaagaaaaggcaaaaataatttttaaaaagttttttaaatgataaaaaacatttaatgaaagaatgaccactttaagtacacatctactcagtctacaactagttaccagggccttctggatgcccaaacttgcactagatacataggcaggaaacacggaagcagaaactgtgagacgctgtccccatcctcacagggcttggctggaggaactggaacacatagccctaaatcccttaaagaaaaatgaagggccaggcttgatggctcacaccagtaatcccagcactttgggaggccgaggcagacagatcactagagattaagagtttgagaccagcctggacaacatgatgaaactccacctctactaaaactacaaaaaattagctgggcatggttgcagatgcctgcaatcccagctacccaggaggctgagacaggagaattacttaagcctgggaggcggaggttgcagtgagccgagatcgcgccactgtactccagcctgggcaacaaagtgaggctccgtctcaaaaaaaaaaaaaaagaaaagaaaagaaaagaaaaatgaagggcagtttaccacacgtgccaagtgagatgtatatgatgag

SEQ ID NO: 3 - RNA, 3084 nt, Homo sapiens
cggcagccagcugagagcaaugggaaauggggagucycagcuguccucggugccugcucagaagcuggguugguuuauccaggaauaccugaagcccuacgaagaaugucagacacugaucgacgagauggugaacaccaucugugacguccugcaggaacccgaacaguucccccuggugcagggaguggccauagguggcuccuauggacggaaaacagucuuaagaggcaacuccgaugguacscuuguccucuucuucagugacuuaaaacaauuccaggaucagaagagaagccaacgugacauccucgauaaaacuggggauaagcugaaguucugucuguucacgaagugguugaaaaacaauuucgagauccagaagucccuugauggguucaccauccagguguucacaaaaaaucagagaaucucuuucgaggugcuggccgccuucaacgcucugagcuuaaaugauaaucccagccccuggaucuaucgagagcucaaaagaucyuuggauaagacaaaugccaguccuggugaguuugcagucugcuucacugaacuccagcagaaguuuuuugacaaccguccuggaaaacuaaaggauuugauccucuugauaaagcacuggcaucaacagugccagaaaaaaaucaaggauuuacccucgcugucuccguaugcccuggagcugcuuacgguguaugccugggaacaggggugcagaaaagacaacuuugacauugcugaaggcgucagaaccguwcuggagcugaucaaaugccaggagaagcuguguaucuauuggauggucaacuacaacuuugaagaugagaccaucaggaacauccugcugcaccagcuccaaucagcgaggccaguaaucuuggauccaguugacccaaccaauaaugugaguggagauaaaauaugcuggcaauggcugaaaaaagaagcucaaaccugguugacuucucccaaccuggauaaugaguuaccugcaccaucuuggaauguucugccugcaccacucuucacgaccccaggccaccuucuggauaaguucaucaaggaguuucuccagcccaacaaaugcuuccuagagcagauugacagugcuguuaacaucauccguacauuccuuaaagaaaacugcuuccgacaaucaacagccaagauccagauuguccggggaggaucaaccgccaaaggcacagcucugaagacuggcucugaugccgaucucgucguguuccauaacucacuuaaaagcuacaccucccaaaaaaacgagcggcacaaaaucgucaaggaaauccaugaacagcugaaagccuuuuggagggagaaggaggaggagcuugaagucagcuuugagccucccaaguggaaggcucccagggugcugagcuucucucugaaauccaaaguccucaacgaaagugucagcuuugaugugcuuccugccuuuaaugcacugggucagcugaguucuggcuccacacccagccccgagguuuaugcagggcucauugaucuguauaaaucyucggaccucccgggaggagaguuuucuaccuguuucacaguccugcagcgaaacuucauucgcucccggcccaccaaacuaaaggauuuaauucgccuggugaagcacugguacaaagagugugaaaggaaacugaagccaaaggggucuuugcccccaaaguaugccuuggagcugcucaccaucuaugccugggagcaggggaguggagugccrgauuuugacacugcagaagguuuccggacaguccuggagcuggucacacaauaucagcagcucugcaucuucuggaaggucaauuacaacuuugaagaugagacygugaggaaguuucuacugagccaguugcagaaaaccaggccugugaucuuggacccagccgaacccacaggugacguggguggaggggaccguugguguuggcaucuucuggcaaaagaagcaaaggaaugguuauccucucccugcuucaaggauggkacuggaaacccaauaccaccuuggaaagugccgguaaaagucaucuaaaggaggcguugucuggaaauagcccuguaacaggcuugaaucraagaacuucuccuacuguagcaaccugaaauuaacucagacacaaauaaaggaaacccagcucacaggagcuuaaacagcuggucagcccccuaagcccccacuacaagugauccucaggcagguaaccccagauucaugcacuguagggugcugcgcagcaucccuagucucuacccaguagaugccacuagcccuccucucccagugacaaccaaaagucuucagacauugucaaacguuccccuggguucacagaucuuucugccuuuggcuuuuggcuccacccucuuuagcuguuaauuugaguacuuauggcccugaaagcggccacggugccuccagauggcagguuugcaauccaagcaggaagaaggaaaagauacccaaaggucaagaacacagugauuuuauuagaaguuucauccgcaaauuuucuuccauuucauugcucagaaaugucauguggyuaccuguaacuugaagguggcuacaaagaugacuguggacguggguugcacuggccacccaaggaugucugccacaccucuccaaagcccucccuaccuaccaagauauaccugauauauuccaccaggauauccucccuccagauauacuugguucucuccaccagguucuuucuuuaaagcaggauuucucaacuuugauacuuacucacauuuggggcuagacaguucuuuguuuggaggcucucuugugcauuguaggauguugagcagcaucucuggccuguacccaguagaugccacccaguugugacaauuaaaagugucuugagacuuuaucaugugucuucugcccuaggugagaacccuugcacuagaggaacccuacaccccaacccuggggggaauguagggaagagguggccaagccaaccgugggguuagcucuaauuauuaagauaugcauuauaaauaaauaccaaaaaauug

SEQ ID NO: 4 - RNA, 2911 nt, Homo sapiens
cggcagccagcugagagcaaugggaaauggggagucycagcuguccucggugccugcucagaagcuggguugguuuauccaggaauaccugaagcccuacgaagaaugucagacacugaucgacgagauggugaacaccaucugugacguccugcaggaacccgaacaguucccccuggugcagggaguggccauagguggcuccuauggacggaaaacagucuuaagaggcaacuccgaugguacscuuguccucuucuucagugacuuaaaacaauuccaggaucagaagagaagccaacgugacauccucgauaaaacuggggauaagcugaaguucugucuguucacgaagugguugaaaaacaauuucgagauccagaagucccuugauggguucaccauccagguguucacaaaaaaucagagaaucucuuucgaggugcuggccgccuucaacgcucugagcuuaaaugauaaucccagccccuggaucuaucgagagcucaaaagaucyuuggauaagacaaaugccaguccuggugaguuugcagucugcuucacugaacuccagcagaaguuuuuugacaaccguccuggaaaacuaaaggauuugauccucuugauaaagcacuggcaucaacagugccagaaaaaaaucaaggauuuacccucgcugucuccguaugcccuggagcugcuuacgguguaugccugggaacaggggugcagaaaagacaacuuugacauugcugaaggcgucagaaccguwcuggagcugaucaaaugccaggagaagcuguguaucuauuggauggucaacuacaacuuugaagaugagaccaucaggaacauccugcugcaccagcuccaaucagcgaggccaguaaucuuggauccaguugacccaaccaauaaugugaguggagauaaaauaugcuggcaauggcugaaaaaagaagcucaaaccugguugacuucucccaaccuggauaaugaguuaccugcaccaucuuggaauguucugccugcaccacucuucacgaccccaggccaccuucuggauaaguucaucaaggaguuucuccagcccaacaaaugcuuccuagagcagauugacagugcuguuaacaucauccguacauuccuuaaagaaaacugcuuccgacaaucaacagccaagauccagauuguccggggaggaucaaccgccaaaggcacagcucugaagacuggcucugaugccgaucucgucguguuccauaacucacuuaaaagcuacaccucccaaaaaaacgagcggcacaaaaucgucaaggaaauccaugaacagcugaaagccuuuuggagggagaaggaggaggagcuugaagucagcuuugagccucccaaguggaaggcucccagggugcugagcuucucucugaaauccaaaguccucaacgaaagugucagcuuugaugugcuuccugccuuuaaugcacugggucagcugaguucuggcuccacacccagccccgagguuuaugcagggcucauugaucuguauaaaucyucggaccucccgggaggagaguuuucuaccuguuucacaguccugcagcgaaacuucauucgcucccggcccaccaaacuaaaggauuuaauucgccuggugaagcacugguacaaagagugugaaaggaaacugaagccaaaggggucuuugcccccaaaguaugccuuggagcugcucaccaucuaugccugggagcaggggaguggagugccrgauuuugacacugcagaagguuuccggacaguccuggagcuggucacacaauaucagcagcucugcaucuucuggaaggucaauuacaacuuugaagaugagacygugaggaaguuucuacugagccaguugcagaaaaccaggccugugaucuuggacccagccgaacccacaggugacguggguggaggggaccguugguguuggcaucuucuggcaaaagaagcaaaggaaugguuauccucucccugcuucaaggauggkacuggaaacccaauaccaccuuggaaagugccgacaaugcagacaccaggaaguuguggagcuaggauccauccuauugucaaugagauguucucauccagaagccauagaauccugaauaauaauucuaaaagaaacuucurgagaucaucuggcaaucgcuuuuaaagacucggcucaccgugagaaagagucacucacauccauucuucccuugauggucccuauuccuccuucccuugcuucuuggacuucuugaaaucaaucaagacugcaaacccuuucauaaagucuugccuugcugaacucccucucugcaggcagccugccuuuaaaaauaguugcugucauccacuuuaugugcaucuuauuucugucaacuuguauuuuuuuucuuguauuuuuccaauuagcuccuccuuuuuccuuccagucuaaaaaaggaauccucugugucuucaaagcaaagcucuuuacuuuccccuugguucucauaacucugugaumuugcucucggugcuuccaacucauccacguccugucuguuuccucuguauacaaaacccuuucugccccugcugacacagacauccucuaugccagcagccagccaacccuuucauuagaacuucaagcucuccaaaggcucagauuauaacuguugucauauuuauaugaggcuguugucuuuuccuucugagccugccuuucucccccccacccaggaguauccucuugccaaaucaaaagacuuuuuccuugggcuuuagccuuaaagauacuugaaggucuaggugcuuuaaccucacauacccucacuuaaacuuuuaucacuguugcauauaccaguugugauacaauaaagaauguaucugg

SEQ ID NO: 5 - RNA, 6646 nt, Homo sapiens
guucggagagccgggcgggaaaacgaaaccagaaauccgaaggccgcgccagagcccugcuuccccuugcaycugcgccgggmggccauggacuuguacagcaccccggccgcugcgcuggacagguucguggccagaargcugcagccgcggaaggaguucguagagaaggcgcggcgcgcucugggcgcccuggccgcugcycugagggagcgcgggggccgccucggugcugcugccccgcgggugcugaaaacugucaagggaggcuccucgggcyggggcacagcucucaaggguggcugugauucugaacuugucaucuuccucgacugcuucaagagcuauguggaccagagggcccgccgugcagagauccucagugagaugcgggcaucgcuggaauccugguggcagaacccagucccuggucugagacucacguuuccugagcagagcgugccuggggcccugcaguuccgccugacauccguagaucuugaggacuggauggauguuagccuggugccugccuucaauguccugggucaggccggcuccggcgucaaacccaagccacaagucuacucuacccuccucaacaguggcugccaagggggcgagcaugcggccugcuucacagagcugcggaggaacuuugugaacauucgcccagccaaguugaagaaccuaaucuugcuggugaagcacugguaccaccaggugugccuacagggguuguggaaggagacgcugcccccggucuaugcccuggaauugcugaccaucuucgccugggagcagggcuguaagaaggaugcuuucagccuagccgaaggccuccgaacuguccugggccugauccaacagcaucagcaccuguguguuuucuggacugucaacuauggcuucgaggacccugcaguugggcaguucuugcagcggcagcuuaagagacccaggccugugauccuggacccagcugaccccacaugggaccuggggaauggggcagccuggcacugggauuugcuagcccaggaggcagcauccugcuaugaccacccaugcuuucugagggggaugggggacccagugcagucuuggaaggggccgggccuuccacgugcuggaugcucagguuugggccaccccauccagcuagacccuaaccagaagaccccugaaaacagcaagagccucaaugcuguguacccaagagcagggagsaaaccucccucaugcccagcuccuggccccacuggggcagccagcaucguccccucugugccgggaauggccuuggaccugucucagauccccaccaaggagcuggaccgcuucauccaggaccaccugaagccgagcccccaguuccaggagcaggugaaaaaggccauygacaucaucuugcgcugccuccaugagaacuguguucacaaggccucaagagucaguaaagggggcucauuuggccggggcacagaccuaagrgauggcugugauguugaacucaucaucuuccucaacugcuucacggacuacaaggaccaggggccccgccgcgcagagauccuugaugagaugcgagcgcagcuagaauccugguggcaggaccaggugcccagccugagccuucaguuuccugagcagaaugugccugaggcucugcaguuccagcugguguccacagcccugaagagcuggacggauguuagccugcugccugccuucgaugcuguggggcagcucaguucuggcaccaaaccaaauccccaggucuacucraggcuccucaccaguggcugccaggagggcgagcauaaggccugcuucgcagagcugcggaggaacuucaugaacauucgcccugucaagcugaagaaccugauucugcuggugaagcacugguaccgccagguugcggcucagaacaaaggaaaaggaccagccccugccucucugcccccagccuaugcccuggagcuccucaccaucuuugccugggagcagggcugcaggcaggauuguuucaacauggcccaaggcuuccggacggugcuggggcucgugcaacagcaucagcagcucugugucuacuggacggucaacuauagcacugaggacccagccaugagaaugcaccuucuuggccagcuucgaaaacccagaccccugguccuggaccccgcugaucccaccuggaacgugggccacgguagcugggagcuguuggcccaggaagcagcagcrcuggggaugcagkccugcuuucugaguagagacgggacaucugugcagcccugggaugugaugccagcccuccuuuaccaaaccccagcuggggaccuugacaaguucaucagugaauuucuccagcccaaccgccaguuccuggcccaggugaacaaggccguugauaccaucuguucauuuuugaaggaaaacugcuuccggaauucucccaucaaagugaucaagguggucaaggguggcucuucagccaaaggcacagcucugcgaggccgcucagaugccgaccucgugguguuccucagcugcuucagccaguucacugagcagggcaacaagcgggccgagaucaucuccgagauccgagcccagyuggaggcaugucaacaggagcggcaguucgaggucaaguuugaagucuccaaaugggagaauccccgcgugcugagcuucucacugacaucccagacgaugcuggaccagaguguggacuuugaugugcugccagccuuugacgcccuaggccagcuggucucuggcuccaggcccagcucucaagucuacgucgaccucauccacagcuacagcaaugcgggcgaguacuccaccugcuucacagagcuacaacgggacuucaucaucucucgcccuaccaagcugaagagccugauccggcuggugaagcacugguaccagcaguguaccaagaucuccaaggggagaggcucccuacccccacagcaygggcuggaacuccugacuguguaugccugggagcagggcgggaaggacucccaguucaacauggcugagggcuuccgcacgguccuggagcuggucacccaguaccgccagcucuguaucuacuggaccaucaacuacaacgccaaggacaagacuguuggagacuuccugaaacagcagcuucagaagcccaggccuaucauccuggauccggcugacccgacaggcaaccugggccacaaugcccgcugggaccugcuggccaaggaagcugcagccugcacaucugcccugugcugcaugggacggaauggcauccccauccagccauggccagugaaggcugcugugugaaguugagaaaaucagcgguccuacuggaugaagagaagauggacaccagcccucagcaugaggaaauucaggguccccuaccagaugagagagauuguguacaugugugugugagcacaugugugcaugugugugcacacgugugcauguguguguuuuagugaaucugcucucccagcucacacacuccccugccucccauggcuuacacacurggauccagacuccaugguuugacaccagccugcguuugcagcuucucugucacuuccaugacucuauccucauaccaccacugcugcuucccacccagcugagaaugcccccuccucccugacuccucucugcccaugcaaauuagcucacaucuuuccuccugcugcaauccaucccuuccucccauuggccucuccuugccaaaucuaaauasuuuauauagggauggcagagaguucccaucucaucugucagccacagucauuugguacuggcuaccuggagccuuaucuucugaaggguuuuaaagaauggccaauuagcugagaagaauuaucuaaucaauuagugaugucugccauggaugcaguagaggaaaguggugguacaagugccaugauugauuagcaaugucugcacuggauacggaaaaaagaaggugcuugcagguuuacaguguauaugugggcuauugaagagcccucugagcucgguugcuagcaggagagcaugcccauauuggcuuacuuugucugccacagacacagacagagggaguugggacaugcaugcuauggggacccucuuguuggacaccuaauuggaugccucuucaugagaggccuccuuuucuucaccuuuuaugcugcacuccuccccuaguuuacacaucuugaugcuguggcucaguuugccuuccugaauuuuuauugggucccuguuuucucuccuaacaugcugagauucugcauccccacagccuaaacugagccaguggccaaacaaccgugcucagccuguuucucucugcccucuagagcaaggcccaccagguccauccaggaggcucuccugaccucaaguccaacaacaguguccrcacuagucaagguucagcccagaaaacagaaagcacucuaggaaucuuaggcagaaagggauuuuaucuaaaucacuggaaaggcuggaggagcagaaggcagaggccaccacuggacuauusguuucaauauuagaccacuguagccgaaucagaggccagagagcagccacugcuacugcuaaugccaccacuaccccugccaucacugccccacauggacaaaacuggagucgagaccuagguuagauuccugcaaccacaaacauccaucagggauggccagcugccagagcugcggraagacggaucccaccucccuuucuuagcagaaucuaaauuacagccagaccucuggcugcagaggagucugagacauguaugayugaaugggugccaagugycagggggyggaguccccagcagaugcauccuggccaucuguugcguggaugagggagugggucuaucucagaggaaggaacaggaaacaaagaaaggaagccacugaacaucccuucucugcuccacaggagugycuuagacagccugacucuccacaaaccacuguuaaaacuuaccugcuaggaaugcuagauugaaugggaugggaagagccuucccucauuauugucauucuuggagagaggugagcaaccaagggaagcuccucugauucaccuagaaccuguucucugccgucuuuggcucagccuacagagacuagaguaggugaagggacagaggacagggcuucuaauaccugugccauauugacagccuccaucccugucccccaucuuggugcugaaccaacgcuaagggcaccuucuuagacucaccucaucgauacugccugguaauccaaagcuagaacucucaggaccccaaacuccaccucuuggauuggcccuggcugyugccacacacauauccaagagcucagggccaguucuggugggcagcagagaccugcucugccaaguuguccagcagcagaguggcccuggccugggcaucacaagccagugaugcuccugggaagaccagguggcaggucgcaguuggguaccuuccauucccaccacacagacuyugggccucccygcaaaauggcuccagaauuagaguaauuaugagauggugggaaccagagcaacucaggugcaugauacaaggagagguugucaucuggguagggcagagaggagggcuugcucaucugaacagggguguauuucauuccaggcccucagucuuuggcaauggccacccugguguuggcauauuggccccacuguaacuuuugggggcuucccggucuagccacacccucggauggaaagacuugacugcauaaagaugucaguucucccugaguugauugauaggcuuaauggucacccuaaaaacacccacauaugcuuuucgauggaaccagruaaguugacgcuaaaguucuuauggaaaaauacacacgcaauagcuaggaaaacacagggaaagaagaguucugagcagggccuagucuuagccaauauuaaaacauacuaugaagccucugauacuuaaacagcauggcgcugguayguaaauagaccaaugcaguuagguggcucuuuccaagacucuggggaaaaaaguaguaaaaagcuaaaugcaaucaaucagcaauugaaagcuaagugagagagccagagggccuccuuggugguaaaagaggguugcauuucuugcagccagaaggcagagaaagugaagaccaaguccagaacugaauccuaagaaaugcaggacugcaaagaaauugguguguguguguguguguguguguguguguguguuuaauuuuuaaaaaguuuuuauugagauacaagucaauaccauaaagcucucacccuucuaaaguguacaauucaguggugugaguauauucauaagauuuauacuuggugucuauucauaagacuuauauccagcauauucauaacuagagccauaucacagaugcauucaucauaauaauuccagacauuuucaucacccuaaaaggaaacccugaaacccauuagcagucauuccccauuccuccaacccauucucucccuaaucccuagaaaccaccaaucugcuguguauuucaucuauugccaacauuucauauaaauggcaucauacaaaaaaaaaaaaaaaaaaaaa

SEQ ID NO: 6 - protein, 687 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPVKVI

SEQ ID NO: 7 - protein, 727 aa, Homo sapiens
  VARIANT 720: Xaa is Trp or termination
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNFXRSSGNRF

SEQ ID NO: 8 - protein, 1087 aa, Homo sapiens
  VARIANT 18: Xaa is Arg or Lys
  VARIANT 65: Xaa is Arg or Trp
  VARIANT 381: Xaa is Ser or Arg
  VARIANT 727: Xaa is Ala or Ser
MDLYSTPAAALDRFVARXLQPRKEFVEKARRALGALAAALRERGGRLGAAAPRVLKTVKGGSSGXGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGXKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQXCFLSRDGTSVQPWDVMPALLYQTPAGDLDKFISEFLQPNRQFLAQVNKAVDTICSFLKENCFRNSPIKVIKVVKGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 9 - protein, 172 aa, Homo sapiens
  VARIANT 163: Xaa is Arg or Ser
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSKHCWVSGEKSQRXGCQTALCNL

SEQ ID NO: 10 - protein, 60 aa, Homo sapiens
MGNGESQLSSVPAQKLGWFIQEYLKPYEECQTLIDEMVNTICDVLQEPEQFPLVQGVAIA

SEQ ID NO: 11 - protein, 612 aa, Homo sapiens
MGNGESQLSSVPAQKLGWFIQEYLKPYEECQTLIDEMVNTICDVLQEPEQFPLVQGVAIGGSYGRKTVLRGNSDSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPVKVI

SEQ ID NO: 12 - protein, 652 aa, Homo sapiens
  VARIANT 705: Xaa is Trp or termination
MGNGESQLSSVPAQKLGWFIQEYLKPYEECQTLIDEMVNTICDVLQEPEQFPLVQGVAIGGSYGRKTVLRGNSDSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNFXRSSGNRF

SEQ ID NO: 13 - RNA, 2020 nt, Homo sapiens
agcagcaagaauuccucugccucccauccuaccauucacugucuugccggcagccagcugagagcaaugggaaauggggagucccagcuguccucggugccugcucagaagcuggguugguuuauccaggaauaccugaagcccuacgaagaaugucagacacugaucgacgagauggugaacaccaucugugacguccugcaggaacccgaacaguucccccuggugcagggaguggccauagguggcuccuauggacggaaaacagucuuaagaggcaacuccgaugguacccuuguccucuucuucagugacuuaaaacaauuccaggaucagaagagaagccaacgugacauccucgauaaaacuggggauaagcugaaguucugucuguucacgaagugguugaaaaacaauuucgagauccagaagucccuugauggguucaccauccagguguucacaaaaaaucagagaaucucuuucgaggugcuggccgccuucaacgcucugaguaagcauugcugggugucaggagagaaaagccaaagaagsgggugccagacagcucugugcaaccucuaggccaugagugggauagauaccacugcugcuuuaaaaaaugggagaccauagacccucaggagagaagaaucccuucuacccuggacucgcucucuuckcuggaacuaacuucucccccauacccugauugucuuuggagaaaauguucuggauucuagaaucuaaggcagagccuuuuaagccauacuguacacauaaaucaccuggaaccuuguuaaaaugcagauccugacucaggaggucugaguuagagcccaggauuucauauuucuagccagcuccaugaugagcugcugguccgcagaucaygcuugcagguuuugaccagagucaguguugguuagaguaagaggaugaggcagacaucugggaaaaguccagcuggggcaagcauuugaagucugccuuccuaccaggucaaaaucaaggcaacgaccuuccauagauaacuaucaaagcuugagggggugccuugaacccaacuccuaaaucccyaagaccugcccaccucuugugucuccugucucagcaaacauucccacacucuugcauauuguuaaaguaaccucugcuuaccaggcuucugguuuaauaaaagauggcuagagugacuccaucuuaaagcaaguagcuaggcacucaaaaggaaccuacaggcuuaauacuugggucugaaaauagccacagucuaagcugaccaccaauuauaauugcagaauauuuaaggccauacaaaacaucucccacuaagccuacaaaauguccagguguccuaaaaguucagcccacuuaaaggcagcauuaaugagcagguuuagguugaaggauuaauggucaucaauaccacuguuaagaagaaaauucuuggccaaauugaauuuaauggaguuuaacugagcagacaauucacaaaucuagaagccuccugakccagaguagguucagagagucuugaacacagccacgugguggaagaagauuuauggacaggaaaaggaaaaugauguacugaaaaugaaagugagguacagaaacagccagacugguuauagcucagcauuggccuuauuugaacgagauuugaacaguuggccaccuuugauuggccgaaacucagugauuggcacaagaguagguugcagucuguuuacacauccuuuuagguuauaguucaccauguacagagaaauuuuaggccaaacuuaaaauauguaaggaggcagcuuuaggcuaaacuugauuuaacagcaccaauacccccuaccuuuagugagcacaucugcacauuccaauuuuaaugacagcuccuuagaauuucuuaucaacgaagacacuaacaaagaauggcgcauuccuccuucuccuuucugaggaugcccuacccuguaacaaagucguuucuaauaaauuugcuucuuucaccaaaaaaaaaaaa

SEQ ID NO: 14 - protein, 177 aa, Homo sapiens
  VARIANT 18: Xaa is Arg or Lys
  VARIANT 65: Xaa is Arg or Trp
MDLYSTPAAALDRFVARXLQPRKEFVEKARRALGALAAALRERGGRLGAAAPRVLKTVKGGSSGXGTALKGGCDSELVIFLDCFKSYVDQRARRAEILSEMRASLESWWQNPVPGLRLTFPEQSVPGALQFRLTSVDLEDWMDVSLVPAFNVLGEGFLDHSRVGGKRSLGTTQDFQF

SEQ ID NO: 15 - protein, 223 aa, Homo sapiens
  VARIANT 18: Xaa is Arg or Lys
  VARIANT 65: Xaa is Arg or Trp
MDLYSTPAAALDRFVARXLQPRKEFVEKARRALGALAAALRERGGRLGAAAPRVLKTVKGGSSGXGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVKPLGRVSPDM

SEQ ID NO: 16 - DNA, 20 bp, Artificial Sequence
  Primer A
gcaggagttggtaaactcac

SEQ ID NO: 17 - DNA, 20 bp, Artificial Sequence
  Primer B
gaggttaagtagcctgccca

SEQ ID NO: 18 - DNA, 20 bp, Artificial Sequence
  Primer A
gcctgccactcaatgttaag

SEQ ID NO: 19 - DNA, 20 bp, Artificial Sequence
  Primer B
actcatggcctagaggttgc

SEQ ID NO: 20 - DNA, 20 bp, Artificial Sequence
  Primer A
atctaatgggccaagtcacc

SEQ ID NO: 21 - DNA, 20 bp, Artificial Sequence
  Primer B
ggtacacgaaacgttcccta

SEQ ID NO: 22 - DNA, 20 bp, Artificial Sequence
  Primer A
tcttgtgtgccactccaaac

SEQ ID NO: 23 - DNA, 20 bp, Artificial Sequence
  Primer B
gagctacaatgcccacttac

SEQ ID NO: 24 - DNA, 20 bp, Artificial Sequence
  Primer A
tattctggagatgctccctg

SEQ ID NO: 25 - DNA, 20 bp, Artificial Sequence
  Primer B
tgggcagattctccaaagtg

SEQ ID NO: 26 - DNA, 20 bp, Artificial Sequence
  Primer A
gacatccaagctgcagagtg

SEQ ID NO: 27 - DNA, 20 bp, Artificial Sequence
  Primer B
ctgttggctagcactttccc

SEQ ID NO: 28 - DNA, 20 bp, Artificial Sequence
  Primer A
actacaagtgatcctcaggc

SEQ ID NO: 29 - DNA, 20 bp, Artificial Sequence
  Primer B
gtgcaagggttctcacctag

SEQ ID NO: 30 - DNA, 20 bp, Artificial Sequence
  Primer A
actcacatttggggctagac

SEQ ID NO: 31 - DNA, 20 bp, Artificial Sequence
  Primer B
ggagttcagcaaggcaagac

SEQ ID NO: 32 - DNA, 20 bp, Artificial Sequence
  Primer A
gttgtggagctaggatccat

SEQ ID NO: 33 - DNA, 20 bp, Artificial Sequence
  Primer B
gaggttaaagcacctagacc

SEQ ID NO: 34 - DNA, 20 bp, Artificial Sequence
  Primer A
gacatcctctatgccagcag

SEQ ID NO: 35 - DNA, 20 bp, Artificial Sequence
  Primer B
ccatgggtaaccttgttagc

SEQ ID NO: 36 - DNA, 22 bp, Artificial Sequence
  Primer A
gttactttgaaccctactagta

SEQ ID NO: 37 - DNA, 20 bp, Artificial Sequence
  PrimerB
gctttcagggccataagtac

SEQ ID NO: 38 - DNA, 23 bp, Artificial Sequence
  Primer A
tttcttgatttcagatccctgac

SEQ ID NO: 39 - DNA, 20 bp, Artificial Sequence
  Primer B
tggaatgtgaaaagcactgg

SEQ ID NO: 40 - DNA, 20 bp, Artificial Sequence
  Primer A
tgtcaggtccaagagctgct

SEQ ID NO: 41 - DNA, 20 bp, Artificial Sequence
  Primer B
tgaggtgcacaagcggataa

SEQ ID NO: 42 - DNA, 20 bp, Artificial Sequence
  Primer A
cgtggcttcaatgcctacag

SEQ ID NO: 43 - DNA, 20 bp, Artificial Sequence
  Primer B
ctgggctagaattggaagtc

SEQ ID NO: 44 - DNA, 20 bp, Artificial Sequence
  Primer A
gtgcagccagggttgacaat

SEQ ID NO: 45 - DNA, 20 bp, Artificial Sequence
  Primer B
acctcaggtaatctgcccac

SEQ ID NO: 46 - DNA, 20 bp, Artificial Sequence
  Primer A
aagatggccatgtgcgttag

SEQ ID NO: 47 - DNA, 20 bp, Artificial Sequence
  Primer B
cagctccattgctgtaactc

SEQ ID NO: 48 - DNA, 20 bp, Artificial Sequence
  Primer A
ttctaagaggtcacaggacc

SEQ ID NO: 49 - DNA, 20 bp, Artificial Sequence
  Primer B
acaaagaggatggcaggtgc

SEQ ID NO: 50 - DNA, 21 bp, Artificial Sequence
  Primer A
tccagtacagaattgatactg

SEQ ID NO: 51 - DNA, 18 bp, Artificial Sequence
  Primer B
gcttccagatctgggcag

SEQ ID NO: 52 - DNA, 20 bp, Artificial Sequence
  Primer A
ctctgaacctcagtttaccc

SEQ ID NO: 53 - DNA, 20 bp, Artificial Sequence
  Primer B
ttgggactccttatgtccac

SEQ ID NO: 54 - DNA, 20 bp, Artificial Sequence
  Primer A
cagccaattgagatcgcttc

SEQ ID NO: 55 - DNA, 20 bp, Artificial Sequence
  Primer B
gctatgagttgtcagccacc

SEQ ID NO: 56 - DNA, 20 bp, Artificial Sequence
  Primer A
caggtccttctgatgctacc

SEQ ID NO: 57 - DNA, 20 bp, Artificial Sequence
  Primer B
catgaccactttccagctct

SEQ ID NO: 58 - DNA, 20 bp, Artificial Sequence
  Primer A
gatgacttgtccaaggtcac

SEQ ID NO: 59 - DNA, 20 bp, Artificial Sequence
  Primer B
cgaacagatgtggcctggtt

SEQ ID NO: 60 - DNA, 20 bp, Artificial Sequence
  Primer A
gatgactgtcaccagggatt

SEQ ID NO: 61 - DNA, 20 bp, Artificial Sequence
  Primer B
ctcagccatgttgaactggg

SEQ ID NO: 62 - DNA, 20 bp, Artificial Sequence
  Primer A
tcagctgtgggaccttagtt

SEQ ID NO: 63 - DNA, 20 bp, Artificial Sequence
  Primer B
ctattcctgggtgaccagaa

SEQ ID NO: 64 - DNA, 20 bp, Artificial Sequence
  Primer A
atcagcggtcctactggatg

SEQ ID NO: 65 - DNA, 20 bp, Artificial Sequence
  Primer B
agggctcttcaatagcccac

SEQ ID NO: 66 - DNA, 20 bp, Artificial Sequence
  Primer A
gccacagtcatttggtactg

SEQ ID NO: 67 - DNA, 20 bp, Artificial Sequence
  Primer B
ctgattcggctacagtggtc

SEQ ID NO: 68 - DNA, 20 bp, Artificial Sequence
  Primer A
acaaccgtgctcagcctgtt

SEQ ID NO: 69 - DNA, 20 bp, Artificial Sequence
  Primer B
atcagaggagcttcccttgg

SEQ ID NO: 70 - DNA, 20 bp, Artificial Sequence
  Primer A
attacagccagacctctggc

SEQ ID NO: 71 - DNA, 20 bp, Artificial Sequence
  Primer B
atggaaggtacccaactgcg

SEQ ID NO: 72 - DNA, 20 bp, Artificial Sequence
  Primer A
tcgatactgcctggtaatcc

SEQ ID NO: 73 - DNA, 20 bp, Artificial Sequence
  Primer B
gccacctaactgcattggtc

SEQ ID NO: 74 - DNA, 20 bp, Artificial Sequence
  Primer A
cgatggaaccaggtaagttg

SEQ ID NO: 75 - DNA, 20 bp, Artificial Sequence
  Primer B
cagggtttccttttagggtg

SEQ ID NO: 76 - DNA, 22 bp, Artificial Sequence
  Primer A
aatagcacctacaccatggtcg

SEQ ID NO: 77 - DNA, 22 bp, Artificial Sequence
  Primer B
tacgaactccttccgcggctgc

SEQ ID NO: 78 - DNA, 22 bp, Artificial Sequence
  Primer A
tgaatattccaagtgatgcagc

SEQ ID NO: 79 - DNA, 22 bp, Artificial Sequence
  Primer B
tcagtcagtttaggatggtacc

SEQ ID NO: 80 - DNA, 25 bp, Homo sapiens
tacagacccagcmtctctccctcta

SEQ ID NO: 81 - DNA, 25 bp, Homo sapiens
tatgtacccataygttctgtgggta

SEQ ID NO: 82 - DNA, 25 bp, Homo sapiens
cttccccttgcayctgcgccgggcg

SEQ ID NO: 83 - DNA, 25 bp, Homo sapiens
cttccccttgcayctgcgccgggcg

SEQ ID NO: 84 - DNA, 25 bp, Homo sapiens
acctgcgccgggmggccatggactt

SEQ ID NO: 85 - DNA, 25 bp, Homo sapiens
acctgcgccgggmggccatggactt

SEQ ID NO: 86 - DNA, 25 bp, Homo sapiens
tcgtggccagaargctgcagccgcg

SEQ ID NO: 87 - DNA, 25 bp, Homo sapiens
tcgtggccagaargctgcagccgcg

SEQ ID NO: 88 - DNA, 25 bp, Homo sapiens
cctggccgctgcyctgagggagcgc

SEQ ID NO: 89 - DNA, 31 bp, Homo sapiens
gtgtccaaagggcaaaggggagtcctgggag

SEQ ID NO: 90 - DNA, 25 bp, Homo sapiens
ggctcctcgggcyggggcacagctc

SEQ ID NO: 91 - DNA, 25 bp, Homo sapiens
taagtgagggggycccaggaccctt

SEQ ID NO: 92 - DNA, 25 bp, Homo sapiens
gcattgggttgaygcagaaaccact

SEQ ID NO: 93 - DNA, 25 bp, Homo sapiens
ttgatgcagaaaycactgcgcctgg

SEQ ID NO: 94 - DNA, 25 bp, Homo sapiens
aagagcagggagsaaacctccctca

SEQ ID NO: 95 - DNA, 25 bp, Homo sapiens
gaaaaaggccatygacatcatcttg

SEQ ID NO: 96 - DNA, 25 bp, Homo sapiens
agtggagacacagggggggacccta

SEQ ID NO: 97 - DNA, 25 bp, Homo sapiens
cacagacctaagrgatggctgtgat

SEQ ID NO: 98 - DNA, 25 bp, Homo sapiens
ccaggtctactcraggctcctcacc

SEQ ID NO: 99 - DNA, 25 bp, Homo sapiens
gagcagaaggacyggcctcctccat

SEQ ID NO: 100 - DNA, 26 bp, Homo sapiens
atcccactcctcactctgcttccctc

SEQ ID NO: 101 - DNA, 25 bp, Homo sapiens
ggaagcagcagcrctggggatgcag

SEQ ID NO: 102 - DNA, 25 bp, Homo sapiens
ctggggatgcagkcctgctttctga

SEQ ID NO: 103 - DNA, 25 bp, Homo sapiens
ttgacccacttcygccctcgtagca

SEQ ID NO: 104 - DNA, 25 bp, Homo sapiens
cagtccagaaccracaggctaagcc

SEQ ID NO: 105 - DNA, 25 bp, Homo sapiens
atccgagcccagytggaggcatgtc

SEQ ID NO: 106 - DNA, 25 bp, Homo sapiens
ctaaaaacacccygtggcctcccag

SEQ ID NO: 107 - DNA, 25 bp, Homo sapiens
cccactgggacamcatgggagccgg

SEQ ID NO: 108 - DNA, 25 bp, Homo sapiens
acccccacagcaygggctggaactc

SEQ ID NO: 109 - DNA, 25 bp, Homo sapiens
acccccacagcaygggctggaactc

SEQ ID NO: 110 - DNA, 25 bp, Homo sapiens
ggcttacacactrggatccagactc

SEQ ID NO: 111 - DNA, 25 bp, Homo sapiens
caaatctaaatastttatataggga

SEQ ID NO: 112 - DNA, 25 bp, Homo sapiens
acaacagtgtccrcactagtcaagg

SEQ ID NO: 113 - DNA, 25 bp, Homo sapiens
acaacagtgtccrcactagtcaagg

SEQ ID NO: 114 - DNA, 25 bp, Homo sapiens
cactggactattsgtttcaatatta

SEQ ID NO: 115 - DNA, 25 bp, Homo sapiens
cactggactattsgtttcaatatta

SEQ ID NO: 116 - DNA, 25 bp, Homo sapiens
ccagagctgcggraagacggatccc

SEQ ID NO: 117 - DNA, 25 bp, Homo sapiens
agacatgtatgaytgaatgggtgcc

SEQ ID NO: 118 - DNA, 25 bp, Homo sapiens
gggtgccaagtgycagggggcggag

SEQ ID NO: 119 - DNA, 25 bp, Homo sapiens
agtgccagggggyggagtccccagc

SEQ ID NO: 120 - DNA, 25 bp, Homo sapiens
tccacaggagtgycttagacagcct

SEQ ID NO: 121 - DNA, 25 bp, Homo sapiens
tccacaggagtgycttagacagcct

SEQ ID NO: 122 - DNA, 25 bp, Homo sapiens
tggccctggctgytgccacacacat

SEQ ID NO: 123 - DNA, 25 bp, Homo sapiens
tggccctggctgytgccacacacat

SEQ ID NO: 124 - DNA, 25 bp, Homo sapiens
accacacagactytgggcctccccg

SEQ ID NO: 125 - DNA, 25 bp, Homo sapiens
tctgggcctcccygcaaaatggctc

SEQ ID NO: 126 - DNA, 25 bp, Homo sapiens
cgatggaaccagrtaagttgacgct

SEQ ID NO: 127 - DNA, 25 bp, Homo sapiens
atggcgctggtaygtaaatagacca

SEQ ID NO: 128 - DNA, 25 bp, Homo sapiens
aaatggggagtcycagctgtcctcg

SEQ ID NO: 129 - DNA, 25 bp, Homo sapiens
ggcagcaaggccragctactgggtg

SEQ ID NO: 130 - DNA, 25 bp, Homo sapiens
ctccgatggtacscttgtcctcttc

SEQ ID NO: 131 - DNA, 25 bp, Homo sapiens
aagccaaagaagsgggtgccagaca

SEQ ID NO: 132 - DNA, 25 bp, Homo sapiens
gctcaaaagatcyttggataagaca

SEQ ID NO: 133 - DNA, 25 bp, Homo sapiens
aactagatccccmaatgagctgcta

SEQ ID NO: 134 - DNA, 25 bp, Homo sapiens
cgtcagaaccgtwctggagctgatc

SEQ ID NO: 135 - DNA, 25 bp, Homo sapiens
tgagcactggccyttctcatgtctt

SEQ ID NO: 136 - DNA, 25 bp, Homo sapiens
taatactattcasagtaatttccaa

SEQ ID NO: 137 - DNA, 25 bp, Homo sapiens
tctgtataaatcytcggacctcccg

SEQ ID NO: 138 - DNA, 25 bp, Homo sapiens
gtaaggacagtcyttgttctgacca

SEQ ID NO: 139 - DNA, 25 bp, Homo sapiens
gagtggagtgccrgattttgacact

SEQ ID NO: 140 - DNA, 25 bp, Homo sapiens
tgaagatgagacygtgaggaagttt

SEQ ID NO: 141 - DNA, 25 bp, Homo sapiens
caccctagccccrtacttttcttaa

SEQ ID NO: 142 - DNA, 25 bp, Homo sapiens
gtctcagcaaccyggattttcctct

SEQ ID NO: 143 - DNA, 25 bp, Homo sapiens
cttcaaggatggkactggaaaccca

SEQ ID NO: 144 - DNA, 25 bp, Homo sapiens
caggcttgaatcraagaacttctcc

SEQ ID NO: 145 - DNA, 25 bp, Homo sapiens
cccctaagcccccactacaagtgat

SEQ ID NO: 146 - DNA, 25 bp, Homo sapiens
aatgtcatgtggytacctgtaactt

SEQ ID NO: 147 - DNA, 25 bp, Homo sapiens
aaagaaacttctrgagatcatctgg

SEQ ID NO: 148 - DNA, 25 bp, Homo sapiens
aaagaaacttctrgagatcatctgg

SEQ ID NO: 149 - DNA, 25 bp, Homo sapiens
taactctgtgatmttgctctcggtg

SEQ ID NO: 150 - DNA, 25 bp, Homo sapiens
ctttctcccccccacccaggagtat

SEQ ID NO: 151 - DNA, 25 bp, Homo sapiens
caaaagactttttccttgggcttta

SEQ ID NO: 152 - DNA, 25 bp, Homo sapiens
cttttcacccatscctgggtttatg

SEQ ID NO: 153 - DNA, 101 bp, Homo sapiens
cagcattcactactcaaaatattagctggtgtacatattacagacccagcmtctctccctctagttgaccatgacctctgaaattcacactctgatcctat

SEQ ID NO: 154 - DNA, 101 bp, Homo sapiens
cctgcacgtttctgaaatgctcagagtacgttactcagtatgtacccataygttctgtgggtatactttgttaggttgtgattagttcgttggagctggtg

SEQ ID NO: 155 - DNA, 101 bp, Homo sapiens
aacgaaaccagaaatccgaaggccgcgccagagccctgcttccccttgcayctgcgccgggcggccatggacttgtacagcaccccggccgctgcgctgga

SEQ ID NO: 156 - DNA, 101 bp, Homo sapiens
aaatccgaaggccgcgccagagccctgcttccccttgcacctgcgccgggmggccatggacttgtacagcaccccggccgctgcgctggacaggttcgtgg

SEQ ID NO: 157 - DNA, 101 bp, Homo sapiens
ggacttgtacagcaccccggccgctgcgctggacaggttcgtggccagaargctgcagccgcggaaggagttcgtagagaaggcgcggcgcgctctgggcg

SEQ ID NO: 158 - DNA, 101 bp, Homo sapiens
aaggagttcgtagagaaggcgcggcgcgctctgggcgccctggccgctgcyctgagggagcgcgggggccgcctcggtgctgctgccccgcgggtgctgaa

SEQ ID NO: 159 - DNA, 107 bp, Homo sapiens
aaaactgtcaaggtgaggtcccacctcggggtctttatgtgtccaaagggcaaaggggagtcctgggaggacgcttaagcctcacataggcttacggtgggggtggc

SEQ ID NO: 160 - DNA, 101 bp, Homo sapiens
cagtatgaaattctgagttatttttctttgcccagggaggctcctcgggcyggggcacagctctcaagggtggctgtgattctgaacttgtcatcttcctc

SEQ ID NO: 161 - DNA, 101 bp, Homo sapiens
ggggatgggggacccagtgcagtcttggaaggggccggtaagtgagggggycccaggacccttgggttttgcactttgtttatgtgtccagtgtttcctga

SEQ ID NO: 162 - DNA, 101 bp, Homo sapiens
accctgactgtacaaagggcgggagctggggagagaaggcattgggttgaygcagaaaccactgcgcctggctgaggcagctccttcaatgaccttccagg

SEQ ID NO: 163 - DNA, 101 bp, Homo sapiens
tgtacaaagggcgggagctggggagagaaggcattgggttgatgcagaaaycactgcgcctggctgaggcagctccttcaatgaccttccagggccttcca

SEQ ID NO: 164 - DNA, 101 bp, Homo sapiens
acccctgaaaacagcaagagcctcaatgctgtgtacccaagagcagggagsaaacctccctcatgcccagctcctggccccactggggcagccagcatcgt

SEQ ID NO: 165 - DNA, 101 bp, Homo sapiens
gaccacctgaagccgagcccccagttccaggagcaggtgaaaaaggccatygacatcatcttgcgctgcctccatgagaactgtgttcacaaggcctcaag

SEQ ID NO: 166 - DNA, 101 bp, Homo sapiens
ttcacaaggcctcaagagtcagtaaagtgagttgggccagtggagacacagggggggaccctatcgagggatcagcgtggggaagggaaggagttacagca

SEQ ID NO: 167 - DNA, 101 bp, Homo sapiens
ctgttcttccctccacaggggggctcatttggccggggcacagacctaagrgatggctgtgatgttgaactcatcatcttcctcaactgcttcacggacta

SEQ ID NO: 168 - DNA, 101 bp, Homo sapiens
tctacagggcagctcagttctggcaccaaaccaaatccccaggtctactcraggctcctcaccagtggctgccaggagggcgagcataaggcctgcttcgc

SEQ ID NO: 169 - DNA, 101 bp, Homo sapiens
cccacatctcccctcctttgcttcttattggtcatccagagcagaaggacyggcctcctccatcctccatttcctgcccagatctggaagccactgttaga

SEQ ID NO: 170 - DNA, 102 bp, Homo sapiens
tcatccaggaggagtccaaggtagggtttggggtggcaatcccactcctcactctgcttccctctggactctttgctgaggaagtgtggacataaggagtcc

SEQ ID NO: 171 - DNA, 101 bp, Homo sapiens
tggaacgtgggccacggtagctgggagctgttggcccaggaagcagcagcrctggggatgcaggcctgctttctgagtagagacgggacatctgtgcagcc

SEQ ID NO: 172 - DNA, 101 bp, Homo sapiens
acggtagctgggagctgttggcccaggaagcagcagcgctggggatgcagkcctgctttctgagtagagacgggacatctgtgcagccctgggatgtgatg

SEQ ID NO: 173 - DNA, 101 bp, Homo sapiens
cctgcaagctggtgatctctcccagcccagggccaggcttgacccacttcygccctcgtagcaaacagcaaaaagccaggcatagagaaagagctggaaag

SEQ ID NO: 174 - DNA, 101 bp, Homo sapiens
tgacttgtccaaggtcacacagtaggttttctaactcacagtccagaaccracaggctaagccatgcttcaagggttgagccacctgccatgtcctctcca

SEQ ID NO: 175 - DNA, 101 bp, Homo sapiens
ctgagcagggcaacaagcgggccgagatcatctccgagatccgagcccagytggaggcatgtcaacaggagcggcagttcgaggtcaagtttgaagtctcc

SEQ ID NO: 176 - DNA, 101 bp, Homo sapiens
agggatttaatgtggatcaggccacatctgtgttccacctaaaaacacccygtggcctcccagtggatcccagaccacccttaggaaaacacccaagaggt

SEQ ID NO: 177 - DNA, 101 bp, Homo sapiens
aagaggtaggagatctcagaagtcctttctaagttggccccactgggacamcatgggagccggagtgatggtaaccatctccccatctccaggccagctgg

SEQ ID NO: 178 - DNA, 101 bp, Homo sapiens
ctccagtgtaccaagatctccaaggggagaggctccctacccccacagcaygggctggaactcctgactgtgtatgcctgggagcagggcgggaaggactc

SEQ ID NO: 179 - DNA, 101 bp, Homo sapiens
atctgctctcccagctcacacactcccctgcctcccatggcttacacactrggatccagactccatggtttgacaccagcctgcgtttgcagcttctctgt

SEQ ID NO: 180 - DNA, 101 bp, Homo sapiens
ctgcaatccatcccttcctcccattggcctctccttgccaaatctaaatastttatatagggatggcagagagttcccatctcatctgtcagccacagtca

SEQ ID NO: 181 - DNA, 101 bp, Homo sapiens
ccaggtccatccaggaggctctcctgacctcaagtccaacaacagtgtccrcactagtcaaggttcagcccagaaaacagaaagcactctaggaatcttag

SEQ ID NO: 182 - DNA, 101 bp, Homo sapiens
tcactggaaaggctggaggagcagaaggcagaggccaccactggactattsgtttcaatattagaccactgtagccgaatcagaggccagagagcagccac

SEQ ID NO: 183 - DNA, 101 bp, Homo sapiens
tcctgcaaccacaaacatccatcagggatggccagctgccagagctgcggraagacggatcccacctccctttcttagcagaatctaaattacagccagac

SEQ ID NO: 184 - DNA, 101 bp, Homo sapiens
ctaaattacagccagacctctggctgcagaggagtctgagacatgtatgaytgaatgggtgccaagtgccagggggcggagtccccagcagatgcatcctg

SEQ ID NO: 185 - DNA, 101 bp, Homo sapiens
tctggctgcagaggagtctgagacatgtatgattgaatgggtgccaagtgycagggggcggagtccccagcagatgcatcctggccatctgttgcgtggat

SEQ ID NO: 186 - DNA, 101 bp, Homo sapiens
cagaggagtctgagacatgtatgattgaatgggtgccaagtgccagggggyggagtccccagcagatgcatcctggccatctgttgcgtggatgagggagt

SEQ ID NO: 187 - DNA, 101 bp, Homo sapiens
aaacaaagaaaggaagccactgaacatcccttctctgctccacaggagtgycttagacagcctgactctccacaaaccactgttaaaacttacctgctagg

SEQ ID NO: 188 - DNA, 101 bp, Homo sapiens
gctagaactctcaggaccccaaactccacctcttggattggccctggctgytgccacacacatatccaagagctcagggccagttctggtgggcagcagag

SEQ ID NO: 189 - DNA, 101 bp, Homo sapiens
accaggtggcaggtcgcagttgggtaccttccattcccaccacacagactytgggcctccccgcaaaatggctccagaattagagtaattatgagatggtg

SEQ ID NO: 190 - DNA, 101 bp, Homo sapiens
ggtcgcagttgggtaccttccattcccaccacacagactctgggcctcccygcaaaatggctccagaattagagtaattatgagatggtgggaaccagagc

SEQ ID NO: 191 - DNA, 101 bp, Homo sapiens
gcttaatggtcaccctaaaaacacccacatatgcttttcgatggaaccagrtaagttgacgctaaagttcttatggaaaaatacacacgcaatagctagga

SEQ ID NO: 192 - DNA, 101 bp, Homo sapiens
attaaaacatactatgaagcctctgatacttaaacagcatggcgctggtaygtaaatagaccaatgcagttaggtggctctttccaagactctggggaaaa

SEQ ID NO: 193 - DNA, 101 bp, Homo sapiens
attcactgtcttgccggcagccagctgagagcaatgggaaatggggagtcycagctgtcctcggtgcctgctcagaagctgggttggtttatccaggaata

SEQ ID NO: 194 - DNA, 101 bp, Homo sapiens
ctgaggttgggtctctgggaggcaggagattccacggcggcagcaaggccragctactgggtgctgggtgcctattatgtgcgaggcccacacttgggtgg

SEQ ID NO: 195 - DNA, 101 bp, Homo sapiens
ggtggctcctatggacggaaaacagtcttaagaggcaactccgatggtacscttgtcctcttcttcagtgacttaaaacaattccaggatcagaagagaag

SEQ ID NO: 196 - DNA, 101 bp, Homo sapiens
aacgctctgagtaagcattgctgggtgtcaggagagaaaagccaaagaagsgggtgccagacagctctgtgcaacctctaggccatgagtgggatagatac

SEQ ID NO: 197 - DNA, 101 bp, Homo sapiens
ggcttaaatgataatcccagcccctggatctatcgagagctcaaaagatcyttggataagacaaatgccagtcctggtgagtttgcagtctgcttcactga

SEQ ID NO: 198 - DNA, 101 bp, Homo sapiens
cctctggtttcttgatttcagatccctgacatggtaagaactagatccccmaatgagctgctaccctttccttatcccacagtgccagaaaaaaatcaagg

SEQ ID NO: 199 - DNA, 101 bp, Homo sapiens
caggggtgcagaaaagacaactttgacattgctgaaggcgtcagaaccgtwctggagctgatcaaatgccaggagaagctgtgtatctattggatggtcaa

SEQ ID NO: 200 - DNA, 101 bp, Homo sapiens
cttccgacaatcaacagccaagatccagattgtccgggtgagcactggccyttctcatgtcttgttggaatgatgtaatattgggcattcctggaagggag

SEQ ID NO: 201 - DNA, 101 bp, Homo sapiens
tgctccctgtgtcttagacatcagtcttttaaatatgctaatactattcasagtaatttccaagaattaaatcatatttgtcaaaaatgttttggaatctg

SEQ ID NO: 202 - DNA, 101 bp, Homo sapiens
tccacacccagccccgaggtttatgcagggctcattgatctgtataaatcytcggacctcccgggaggagagttttctacctgtttcacagtcctgcagcg

SEQ ID NO: 203 - DNA, 101 bp, Homo sapiens
aggatttaattcgcctggtgaagcactggtacaaagaggtaaggacagtcyttgttctgaccatggggttattatttttaccagtaagccatgaacattaa

SEQ ID NO: 204 - DNA, 101 bp, Homo sapiens
gccttggagctgctcaccatctatgcctgggagcaggggagtggagtgccrgattttgacactgcagaaggtttccggacagtcctggagctggtcacaca

SEQ ID NO: 205 - DNA, 101 bp, Homo sapiens
cagcagctctgcatcttctggaaggtcaattacaactttgaagatgagacygtgaggaagtttctactgagccagttgcagaaaaccaggtgccttcaccc

SEQ ID NO: 206 - DNA, 101 bp, Homo sapiens
agtttctactgagccagttgcagaaaaccaggtgccttcaccctagccccrtacttttcttaacctgattcccttgaacactgtctcagcaacctggattt

SEQ ID NO: 207 - DNA, 101 bp, Homo sapiens
agccccgtacttttcttaacctgattcccttgaacactgtctcagcaaccyggattttcctctgctggggtcacgattcattccttgcatgacgggggaaa

SEQ ID NO: 208 - DNA, 101 bp, Homo sapiens
ctggcaaaagaagcaaaggaatggttatcctctccctgcttcaaggatggkactggaaacccaataccaccttggaaagtgccggtaaaagtcatctaaag

SEQ ID NO: 209 - DNA, 101 bp, Homo sapiens
tcatctaaaggaggcgttgtctggaaatagccctgtaacaggcttgaatcraagaacttctcctactgtagcaacctgaaattaactcagacacaaataaa

SEQ ID NO: 210 - DNA, 101 bp, Homo sapiens
ggaaacccagctcacaggagcttaaacagctggtcagccccctaagcccccactacaagtgatcctcaggcaggtaaccccagattcatgcactgtagggt

SEQ ID NO: 211 - DNA, 101 bp, Homo sapiens
gtttcatccgcaaattttcttccatttcattgctcagaaatgtcatgtggytacctgtaacttgaaggtggctacaaagatgactgtggacgtgggttgca

SEQ ID NO: 212 - DNA, 101 bp, Homo sapiens
ctcatccagaagccatagaatcctgaataataattctaaaagaaacttctrgagatcatctggcaatcgcttttaaagactcggctcaccgtgagaaagag

SEQ ID NO: 213 - DNA, 101 bp, Homo sapiens
cttcaaagcaaagctctttactttccccttggttctcataactctgtgatmttgctctcggtgcttccaactcatccacgtcctgtctgtttcctctgtat

SEQ ID NO: 214 - DNA, 101 bp, Homo sapiens
atttatatgaggctgttgtcttttccttctgagcctgcctttctcccccccacccaggagtatcctcttgccaaatcaaaagactttttccttgggcttta

SEQ ID NO: 215 - DNA, 101 bp, Homo sapiens
ctttctcccccccacccaggagtatcctcttgccaaatcaaaagactttttccttgggctttagccttaaagatacttgaaggtctaggtgctttaacctc

SEQ ID NO: 216 - DNA, 101 bp, Homo sapiens
tttcctgataggagtgtcttttgtattcataacaagcccttttcacccatscctgggtttatgctaacaaggttacccatggtgggcccttagtttcaagg

SEQ ID NO: 217 - DNA, 100 bp, Homo sapiens
ccaagccacaagtctactctaccctcctcaacagtggctgccaagggggcragcatgcggcctgcttcacagagctgcggaggaactttgtgaacattcg

SEQ ID NO: 218 - DNA, 100 bp, Homo sapiens
gaaggggccggtaagtgagggggccccaggacccttgggttttgcactttgtttatgtgtccagtgtttcctgagcatctactatgtgccatatggtgtg

SEQ ID NO: 219 - DNA, 100 bp, Homo sapiens
atggcatccccatccagccatggccagtgaaggtgagagatctgtggtgcyaaaggaagtaccctttaggggtaaggggggagcatggtcaggggaggga

SEQ ID NO: 220 - DNA, 100 bp, Homo sapiens
gggcccagtgatggccccaggtatgcccctgtgcttccattttcccatccrgctgtgtggtctcagcttctgcagaaagaatggggttaccaacatctct

SEQ ID NO: 221 - DNA, 100 bp, Homo sapiens
cagaaagaatggggttaccaacatctcttataatacttccccaggctgctrtgtgaagttgagaaaatcagcggtcctactggatgaagagaagatggac

SEQ ID NO: 222 - DNA, 100 bp, Homo sapiens
gttgagaaaatcagcggtcctactggatgaagagaagatggacaccagccctcagcatgaggaaattcagggtcccctaccagatgagagagattgtgta

SEQ ID NO: 223 - DNA, 100 bp, Homo sapiens
ctactggatgaagagaagatggacaccagccctcagcatgaggaaattcakggtcccctaccagatgagagagattgtgtacatgtgtgtgtgagcacat

SEQ ID NO: 224 - DNA, 100 bp, Homo sapiens
atgaagagaagatggacaccagccctcagcatgaggaaattcagggtcccytaccagatgagagagattgtgtacatgtgtgtgtgagcacatgtgtgca

SEQ ID NO: 225 - DNA, 100 bp, Homo sapiens
acaccagccctcagcatgaggaaattcagggtcccctaccagatgagagasattgtgtacatgtgtgtgtgagcacatgtgtgcatgtgtgtgcacacgt

SEQ ID NO: 226 - DNA, 100 bp, Homo sapiens
taccctttccttatcccacagtgccagaaaaaaatcaaggatttaccctcrctgtctccgtatgccctggagctgcttacggtgtatgcctgggaacagg

SEQ ID NO: 227 - protein, 683 aa, Homo sapiens
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVP

SEQ ID NO: 228 - DNA, 20 bp, Artificial Sequence
  Primer A
tctagcccctgcaaagtgtt

SEQ ID NO: 229 - DNA, 20 bp, Artificial Sequence
  Primer B
gcacacatgtgctcacacac

SEQ ID NO: 230 - DNA, 25 bp, Homo sapiens
tgccaagggggcragcatgcggcct

SEQ ID NO: 231 - DNA, 25 bp, Homo sapiens
gttttgcactttgtttatgtgtcca

SEQ ID NO: 232 - DNA, 25 bp, Homo sapiens
gatctgtggtgcyaaaggaagtacc

SEQ ID NO: 233 - DNA, 25 bp, Homo sapiens
attttcccatccrgctgtgtggtct

SEQ ID NO: 234 - DNA, 25 bp, Homo sapiens
ccccaggctgctrtgtgaagttgag

SEQ ID NO: 235 - DNA, 25 bp, Homo sapiens
tggacaccagccctcagcatgagga

SEQ ID NO: 236 - DNA, 25 bp, Homo sapiens
tgaggaaattcakggtcccctacca

SEQ ID NO: 237 - DNA, 25 bp, Homo sapiens
attcagggtcccytaccagatgaga

SEQ ID NO: 238 - DNA, 25 bp, Homo sapiens
ccagatgagagasattgtgtacatg

SEQ ID NO: 239 - DNA, 25 bp, Homo sapiens
ggatttaccctcrctgtctccgtat

SEQ ID NO: 240 - DNA, 24 bp, Homo sapiens
gtgtccaaaggggagtcctgggag

SEQ ID NO: 241 - DNA, 24 bp, Homo sapiens
agtggagacacaggggggacccta

SEQ ID NO: 242 - DNA, 25 bp, Homo sapiens
atcccactcctcttctgcttccctc

SEQ ID NO: 243 - DNA, 24 bp, Homo sapiens
cccctaagccccactacaagtgat

SEQ ID NO: 244 - DNA, 24 bp, Homo sapiens
ctttctccccccacccaggagtat

SEQ ID NO: 245 - DNA, 24 bp, Homo sapiens
caaaagacttttccttgggcttta

SEQ ID NO: 246 - DNA, 100 bp, Homo sapiens
aaaactgtcaaggtgaggtcccacctcggggtctttatgtgtccaaaggggagtcctgggaggacgcttaagcctcacataggcttacggtgggggtggc

SEQ ID NO: 247 - DNA, 100 bp, Homo sapiens
ttcacaaggcctcaagagtcagtaaagtgagttgggccagtggagacacaggggggaccctatcgagggatcagcgtggggaagggaaggagttacagca

SEQ ID NO: 248 - DNA, 101 bp, Homo sapiens
tcatccaggaggagtccaaggtagggtttggggtggcaatcccactcctcttctgcttccctctggactctttgctgaggaagtgtggacataaggagtcc

SEQ ID NO: 249 - DNA, 100 bp, Homo sapiens
ggaaacccagctcacaggagcttaaacagctggtcagccccctaagccccactacaagtgatcctcaggcaggtaaccccagattcatgcactgtagggt

SEQ ID NO: 250 - DNA, 100 bp, Homo sapiens
atttatatgaggctgttgtcttttccttctgagcctgcctttctccccccacccaggagtatcctcttgccaaatcaaaagactttttccttgggcttta

SEQ ID NO: 251 - DNA, 100 bp, Homo sapiens
ctttctcccccccacccaggagtatcctcttgccaaatcaaaagacttttccttgggctttagccttaaagatacttgaaggtctaggtgctttaacctc

SEQ ID NO: 252 - DNA, 96 bp, Homo sapiens
gaaggggccggtaagtgagggggccccaggacccttgggttttgcactttatgtgtccagtgtttcctgagcatctactatgtgccatatggtgtg

SEQ ID NO: 253 - DNA, 97 bp, Homo sapiens
gttgagaaaatcagcggtcctactggatgaagagaagatggacaccagccagcatgaggaaattcagggtcccctaccagatgagagagattgtgta

SEQ ID NO: 254 - DNA, 21 bp, Homo sapiens
gttttgcactttatgtgtcca

SEQ ID NO: 255 - DNA, 22 bp, Homo sapiens
tggacaccagccagcatgagga

SEQ ID NO: 256 - protein, 1087 aa, Homo sapiens
  VARIANT 18: Xaa = Arg OR Ser
  VARIANT 801: Xaa = any amino acid
MDLYSTPAAALDRFVARXLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLHENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKGPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 257 - protein, 1087 aa, Pan paniscus
  VARIANT 801: Xaa = any amno acid
  VARIANT 18: Xaa = Ser OR Arg
MDLYSTPAAALDRFVARXLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPCAGCSGLGHPIQLDPNQKTPENSKSLSAVYPRAGSKPPSCPAPGPTGAASIVPSVPGMALDLSQIPTKELDRFIQDHLKPSPQFQEQVKKAIDIILRCLRENCVHKASRVSKGGSFGRGTDLRDGCDVELIIFLNCFTDYKDQGPRRAEILDEMRAQLESWWQDQVPGLSLQFPEQNVPEALQFQLVSTALKSMTDVSLLPAFDAVGQLSSGTKPNPQVYSRLLTSGCQEGEHKACFAELRRNFMNIRPVKLKNLILLVKHWYHQVAAQNKGKRPAPASLPPAYALELLTIFAWEQGCGQDCFNMAQGFRTVLGLVQQHQQLCVYWTVNYSTEDPAMRMHLLGQLGKPRPLVLDPADPTWNVGHGSWELLAREAAALGMQACFLSRDGTSVQPWDVMPALLYQTPAGDLDKFISEFLQPNRQFLAQVNKIVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 258 - protein, 1087 aa, Pan troglodytes troglodytes
  VARIANT 801: Xaa = any amino acid
  VARIANT 18: Xaa = Ser OR Arg
MDLYSTPAAALDRFVARXLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPCAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLRENCVHKASRVSKGGSFGRGTDLRDGCDVELIIFLNCFTDYKDQGPRRAEILDEMRAQLESWWQDQVPGLSLQFPEQNVPEALQFQLVSTALKSMTDVSLLPAFDAVGQLSSGTKPNPQVYSRLLTSGCQEGEHKACFAELRRNFMNIRPVKLKNLILLVKHWYHQVAAQNKGKRPAPASLPPAYALELLTIFAWEQGCRQDCFNMAQGFRTVLGLVQQHQQLCVYWTVNYSTEDPAMRMHLLGQLGKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 259 - protein, 1087 aa, Pan troglodytes verus
  VARIANT 801: Xaa = any amino acid
  VARIANT 18: Xaa = Ser OR Arg
MDLYSTPAAALDRFVARXLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPCAGCSGLGHPIQLDPNQKTPENSKSLSAVYPRAGSKPPSCPAPGPTGAASIVPSVPGMALDLSQIPTKELDRFIQDHLKPSPQFQEQVKKAIDIILRCLRENCVHKASRVSKGGSFGRGTDLRDGCDVELIIFLNCFTDYKDQGPRRAEILDEMRAQLESWWQDQVPGLSLQFPEQNVPEALQFQLVSTALKSMTDVSLLPAFDAVGQLSSGTKPNPQVYSRLLTSGCQEGEHKACFAELRRNFMNIRPVKLKNLILLVKHWYHQVAAQNKGKRPAPASLPPAYALELLTIFAWEQGCRQDCFNMAQGFRTVLGLVQQHQQLCVYWTVNYSTEDPAMRMHLLGQLGKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 260 - protein, 1087 aa, Pan troglodytes troglodytes
  VARIANT 801: Xaa = any amino acid
  VARIANT 683: Xaa = Ile OR Met
  VARIANT 18: Xaa = Ser OR Arg
MDLYSTPAAALDRFVARXLQPRKEFVEKARRALGALAAALRERGGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLSAVYPRAGSKPPSCPAPGPTGAASIVPSVPGMALDLSQIPTKELDRFIQDHLKPSPQFQEQVKKAIDIILRCLRCNCVHKASRVSKGGSFGRGTDLRDGCDVELIIFLNCFTDYKDQGPRRAEILDEMRAQLESWWQDQVPGLSLQFPEQNVPEALQFQLVSTALKSMTDVSLLPAFDAVGQLSSGTKPNPQVYSRLLTSGCQEGEHKACFAELRRNFMNIRPVKLKNLILLVKHWYHQVAAQNKGKRPAPASLPPAYALELLTIFAWEQGCRQDCFNMAQGFRTVLGLVQQHQQLCVYWTVNYSTEDPAXRMHLLGQLGKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 261 - protein, 1087 aa, Gorilla gorilla
  VARIANT 801: Xaa = any amino acid
MDLYSTPAAALDRFVARSLQPRTEFVEKARRALGALAAALRERAGRLGAAAPRVLKTVKGGSSGRGTALKGGCDSELVIFLDCFKSYVDQRARRAEILSEMRASLESWWQNPVPGLRLTFPEQSVPGALQFRLTSVDLEDWMDVSLVPAFNVLGQAGSGVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKRPRPVILDPADPTWDLGNGAAWHWDLLAQEAASCYDHPCFLRGMGDPVQSWKGPGLPRAGCSGLGHPIQLDPNQKTPENSKSLNAVYPRAGSKPPSCPAPGPTGAASIVPSVPGMALDLSQIPTKELDRFIQDHLKPSPQFQEQVKKAIDIILRCLRENCVHKASRVSKGGSFGRGTDLRDGCDVELIIFLNCFTDYKDQGPRRAEILDEMRAQLESWWQDQVPSLSLQFPEQNVPEALQFQLVSTALKSWTDVSLLPAFDAVGQLSSGTKPNPQVYSRLLTSGCQEGEHKACFAELRRNFMNIRPVKLKNLILLVKHWYRQVAAQNKGKRPAPASLPPAYALELLTIFAWEQGCRQDCFNMAQGFRTVLGLVQQHQQLCVYWTVNYSTEDPAMRMHLLGQLRKPRPLVLDPADPTWNVGHGSWELLAQEAAALGMQACFLSRDGTSVQPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGSLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 262 - protein, 971 aa, Pongo abelii
  VARIANT 603: Xaa = Met OR Val
  VARIANT 849: Xaa = Ala OR Ser
GGSSGRGTALKGGCDSELVIFLDCFKSYVDQRARRAEILSEMRASLESWWQNPVPGLRLTFPEQSVPGALQFRLTSVDLEDWMDVSLVPAFDVLGQASSSVKPKPQVYSTLLNSGCQGGEHAACFTELRRNFVNIRPAKLKNLILLVKHWYHQVCLQGLWKETLPPVYALELLTIFAWEQGCKKDAFSLAEGLRTVLGLIQQHQHLCVFWTVNYGFEDPAVGQFLQRQLKQPRPVILDPADPTWDLGNGAAWHWDLLAQEAASCYDHPCFLKGMGDPVQSWKGPGLPRAGCSGLGHPIQLDPNQKTPENSKTLNAVYPKAGSKPPSRPAPGPTRAASIVPSVPGMALDLSQIPTKELDRFIQDHLKPSPQFQEQVKKAIDIILRCLRENCVHKASRVSKGGSFGRGTDLRDGCDVELIIFLNCFTDYKDQGPCHAEILDEMRAQLESWWQDQVPGLSLQFPEQNVPEALQFRLVSTALKSWTDVSLLPAFDAVGQLSSGTKPNPQVYSRLLTSGCQEGEHKACFAELRRNFVNIRPAKLKNLILLVKHWYRQVAAQNKGKRPAPASLPPAYALELLTIFAWEQGCRQDCFNMAQGFRTVLGLXQQHQQLCVFWTVNYSTEDTAMRMHLLGQLRKPRPLVLDPADPTWNVGHGSWELLAQEAAALGMQACFLSRDGTSVQPWDVMXGGSSAKGTALRGRSDADLVVFLSCFSQFTEQGNKRAEIISEIRAQLEACQQERQFEVKFEVSKWENPRVLSFSLTSQTMLDQSVDFDVLPAFDALGQLVSGSRPSSQVYVDLIHSYSNAGEYSTCFTELQRDFIISRPTKLKSLIRLVKHWYQQCTKISKGRGXLPPQHGLELLTVYAWEQGGKDSQFNMAEGFRTVLELVTQYRQLCIYWTINYNAKDKTVGDFLKQQLQKPRPIILDPADPTGNLGHNARWDLLAKEAAACTSALCCMGRNGIPIQPWPVKAAV

SEQ ID NO: 263 - protein, 1028 aa, Macaca mulatta
  VARIANT 18: Xaa = Ser OR Arg
  VARIANT 801: Xaa = any amino acid
MDLYRTPASALDRFVATXLQPRKEFTETARRALGALAAALRERGGRPGALAPRVLKIVKGGSSGRGTALKGGCDSELVIFLDCFKSYMDQRARRAEILSKMRALLESWWQNPVPGLSLKFPQQSVPGALQFRLTSIDLEDWTDVSLVPAFDVLGQAGSGVKPKPQVYSTLLNSGCQGGEHAACFTELRRDFVNIRPAKLKNLILLVKHWYHQVCLQGLWEETLPPVYALELLTIFAWEQGCKKDAFSLAEGLRTVLDLIQQHQHLCVFWTVNYGFEDPAVGQFLQRQLERPRPVILDPADPTWDLGNGAAWHWDLLAQEAASCCDHPCFLNGMGDPVQPWQVPGLPRARCSGLGHPIQLNPNQKTPENSKSDNASYPRAGSKAPSCPAPGPAGAASVAPSVPGMALDLSQIPTKELDRFIQDHLKPSPRFQEQVKKAIDIILRRLHENCVHKVSRVSKGGSFGRGTDLRDGCDVELIIFLNCFTDYKDQGPRRAEILDEMRAQLESWWQGQVPGLSLQFPQQNVPEALQFQPVSTALKSWTDVSLLPAFDAVGQLSSGTKPNPQVYSRLLSSGCQEGEHKACFAELRRNFVNIRPAKLKNLILLVKHWYRQVAAQNKRKRPAPASLPPAYALELLTIFAWEQGCRQDCFDMAQGFRTVLGLVQQHQQLCVYWTVNYSTEDPAMRMHLLGQLRKPRPLVLDPADPTWNVGQGSWELLAQEAAVLGMQACFLSRDGTSMPPWDVMPALLYQTPAGDLDKFISEFLQPNRQFLAQVNKAVDTICSFLKENCFRNSPIKVIKVVXGGSSAKGTALRGRSDADLVVFLSCFSQFTEQGNKRAEIISEIRAQLEACQRERQFEVKFEVSKWENPRVLSFSLTSQTMLDQSVDFDVLPAFDALCHKISKGRGSLPPKHGLELLTVYAWEQGGKDPQFNMAEGFRTVLELVTQYRQLCIYWTINYNTKDKTVGDFLKQQLQKPRPIILDPADPTGNLGHSARWDLLAKEAAACMSALCCVGRNGIPIQPWPVKAAV

SEQ ID NO: 264 - protein, 727 aa, Homo sapiens; identical to SEQ ID NO: 7
  VARIANT 720: Xaa = any amino acid

SEQ ID NO: 265 - protein, 687 aa, Homo sapiens; identical to SEQ ID NO: 6

SEQ ID NO: 266 - protein, 719 aa, Pan paniscus
  VARIANT 441: Xaa = Thr OR Ala
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWMENNFEIQKSLDGFTIQVFTKNQSISFKVLAAFNALSLNDNPSPWIYRELKRSLDKTNASPGEFAVCFTELQQKFFDNRPGKLKDLILLIKHWHQQCQKKIKNLPSLSPYALELLTVYAWEQGCRKDDFDIAEGVRTVLKLIKCQEQLCVYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIICTFLKENCFRQSTAKIQIVRGGSTAKGTALKTGSDADLVVFHNSLKSYTSQKNERHKIVKEIHEQLKXFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 267 - protein, 719 aa, Pan troglodytes verus
  VARIANT 31: Xaa = Lys OR Gln
MGNGESQLSSVPAQKLGWFIQEYLKPYEECXTLIDEMVNTICDVLQEPEQFPLVQGVAIGGSYGRKTVLRGNSDGTLVLFFSDLKQFQDQKRSQRDILDKTGDKLKFCLFTKWMENNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKNLPSLSPYALELLTVYAWEQGCRKDNFDIAEGVRTVLKLIKCQEQLCVYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIICTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 268 - protein, 719 aa, Pan troglodytes troglodytes
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWMENNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKNLPSLSPYALELLTVYAWEQGCRKDNFDIAEGVRTVLKLIKCQEQLCVYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIICTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 269 - protein, 719 aa, Gorilla gorilla
MGNGESQLSSVPAQKLGWFIQEYLKPYEECQTLIDEMVNTICDVLQEPEQFPPVQGVAIGGSYGRKTVLRGNSDGTLVLFFSDLKQFQDQKRSQRDILDKTGDKLKFCLFTKWMKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEQLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKFFLEQIDSTVNIIRTFLKENCFRQSTAKIQIVRGGSTAKGTALKTGSDADLIVFHNSLKSYTSQKNERHKIVKEIHEQLKAFSREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 270 - protein, 719 aa, Pongo abelii
  VARIANT 535: Xaa = Gln OR Arg
  VARIANT 650: Xaa = His OR Arg
MGNGESQLSSVPAQKLGWFIQEYLKPYKECQTLIDETVNTICDVLQEPEQFPLVQGVAIGGSYGRKTVLRGNSDGTLVLFFSDLKQFQDQKRSQHDILDKTGDKLKFYLFTKLMNNNFEIQKSHDGFTIQVFTRNQRISFEVLAAFNALSLNDNPSPWIYRELKRSLDKTNASPGEFAVCFTELQQKFFDNRPRKLKDLILLIKHWHLQCQKKNKDLPSLSPYALELLTVYAWEQGCRKDNFDIAEGVRTVLELIKRQEQLCIYWMVNYNFEDETIRNILLHQLQSARPVILDPADPTNNVSGDKICWQRLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHKQLEAFWRENKEELEVSFEPPKWKAPRVLSFSLKSKVLNESVSFDVLPAFNALGQRSSGSTPSPEVYAGLIDLYKSSDLPGGEFSTCFTVLQRNFIRSXPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDLGGGDXWCWHLLAKEAKEWLSSLCFKDGTGNPIPPWKVPAMQTPGSCGARIHPIVNEMFSSRSHRILNNNSRRNF

SEQ ID NO: 271 - protein, 719 aa, Macaca mulatta
MGNGGSQLSSVPAQKLGGFIQEYLKPYEECQTLIDDMVNTICDVLQAPNQFPLVQGVAIGGSYGRKTVLRGNSDGTLVLFFSNLKQFQDQKKSQHDILDKTGHKLEFCLYTKWMKDSFEIQKSHDGFTIQLFTKNQRVSFEVLAAFNALSLNDNPSPWIYRELKRSLDKTSASPGEFAVCFTELQQKFFDNRPRKLKDLILLIKHWHQQCEKKMKDLPLLSPYALELLTVYAWEQGCRRDNFDIAEGVRTILELIKCHEQLCVYWMVNYNFEDETIRNILLPQLQSARPVILDPTDPTNNVSGDKRCWQWLKKEAQTWLTSPNLDNELPAPSWNVLPAPLFMTPGHLLDKFIKEFLQPNKFFLEQIDSAVDIICTFLKENCFRQSTAKIQIVQGGSTAKGTALKTGSDANLVVFHNSLKSYTSQKNERYRIIKEIHEQLETFWREKKEELEVSFEPPMWKAPRVLSFSLKSKVLNESVSFDVLPAFNALGQLSSGSTPSPEVYAGLLDLYKSSDFPGGEFSTCFTVLQQNFIRSRPTKLKDLIRLVKHWYKECKRKLKPKGSLPPKYALELLTVYAWEQGSGAPDFDTAEGFRTVLELVTQYRQLCIFWKVNYNFEDETVRKFLLSQLQKTRPVILDPAEPTGDVGGGDRWCWHLLAKEAKEWSYSLCFKDETGNPISPWKVPTMQTLGSCRARIHPIVNEMFSSRSHRILNNNSRRNF

SEQ ID NO: 272 - protein, 292 aa, Macaca mulatta
MGNGGSQLSSVPAQKLGGFIQEYLKPYEECQTLIDDMVNTICEVLQAPEQFPLVQGVAIGGSYGRKTVLRGNSDGTLVLFFSDLKQFQDQKRSQHDILDKTGDKLKFCLYTKWMKNNFEIQKSHDGFTIQVFTKNQRISFEVLAAFNALGQLSSGSTPSPEVYAGLLDLYKSSDFPGGEFSTCFTVLQQNFIRSRPTKLKDLIRLVKHWYKECKRKLKPKGSLPPKYALELLTVYAWEQGSGAPDFDTAEGFRTVLELVTQYRQLCIFWKVNYNFEDETVRKFLLSQLQKTR